SEQUENCE LISTING (318 sequences)

SEQ ID NO: 1 - protein, 153 aa, Equus caballus
GLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 2 - protein, 153 aa, Equus caballus; identical to SEQ ID NO: 1

SEQ ID NO: 3 - protein, 153 aa, Equus caballus; identical to SEQ ID NO: 1

SEQ ID NO: 4 - protein, 153 aa, Equus caballus; identical to SEQ ID NO: 1

SEQ ID NO: 5 - protein, 153 aa, Equus caballus; identical to SEQ ID NO: 1

SEQ ID NO: 6 - protein, 153 aa, Equus caballus; identical to SEQ ID NO: 1

SEQ ID NO: 7 - protein, 153 aa, Equus caballus; identical to SEQ ID NO: 1

SEQ ID NO: 8 - protein, 153 aa, Equus caballus; identical to SEQ ID NO: 1

SEQ ID NO: 9 - protein, 153 aa, Equus caballus; identical to SEQ ID NO: 1

SEQ ID NO: 10 - protein, 153 aa, Equus caballus; identical to SEQ ID NO: 1

SEQ ID NO: 11 - protein, 153 aa, Equus caballus; identical to SEQ ID NO: 1

SEQ ID NO: 12 - protein, 153 aa, Equus caballus; identical to SEQ ID NO: 1

SEQ ID NO: 13 - protein, 153 aa, Equus caballus; identical to SEQ ID NO: 1

SEQ ID NO: 14 - protein, 153 aa, Equus caballus; identical to SEQ ID NO: 1

SEQ ID NO: 15 - protein, 199 aa, Bos taurus
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 16 - protein, 199 aa, Bos taurus; identical to SEQ ID NO: 15

SEQ ID NO: 17 - protein, 199 aa, Bos taurus; identical to SEQ ID NO: 15

SEQ ID NO: 18 - protein, 199 aa, Bos taurus; identical to SEQ ID NO: 15

SEQ ID NO: 19 - protein, 199 aa, Bos taurus; identical to SEQ ID NO: 15

SEQ ID NO: 20 - protein, 199 aa, Bos taurus; identical to SEQ ID NO: 15

SEQ ID NO: 21 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVYDEALEKFDKALKALPMHIRLSFNPTQLEGQCHI

SEQ ID NO: 22 - protein, 162 aa, Bos taurus
LIVTQTMKGLDIQKVAGTWYSLAMAASDISLLDAQSAPLRVYVEELKPTSEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEGQCHI

SEQ ID NO: 23 - protein, 153 aa, Equus caballus; identical to SEQ ID NO: 1

SEQ ID NO: 24 - protein, 153 aa, Equus caballus; identical to SEQ ID NO: 1

SEQ ID NO: 25 - protein, 153 aa, Equus caballus; identical to SEQ ID NO: 1

SEQ ID NO: 26 - protein, 153 aa, Equus caballus; identical to SEQ ID NO: 1

SEQ ID NO: 27 - protein, 153 aa, Equus caballus; identical to SEQ ID NO: 1

SEQ ID NO: 28 - protein, 153 aa, Equus caballus; identical to SEQ ID NO: 1

SEQ ID NO: 29 - protein, 153 aa, Equus caballus; identical to SEQ ID NO: 1

SEQ ID NO: 30 - protein, 153 aa, Equus caballus; identical to SEQ ID NO: 1

SEQ ID NO: 31 - protein, 153 aa, Equus caballus; identical to SEQ ID NO: 1

SEQ ID NO: 32 - protein, 153 aa, Equus caballus; identical to SEQ ID NO: 1

SEQ ID NO: 33 - protein, 153 aa, Equus caballus; identical to SEQ ID NO: 1

SEQ ID NO: 34 - protein, 153 aa, Equus caballus; identical to SEQ ID NO: 1

SEQ ID NO: 35 - protein, 153 aa, Equus caballus; identical to SEQ ID NO: 1

SEQ ID NO: 36 - protein, 153 aa, Equus caballus; identical to SEQ ID NO: 1

SEQ ID NO: 37 - protein, 153 aa, Equus caballus; identical to SEQ ID NO: 1

SEQ ID NO: 38 - protein, 153 aa, Equus caballus; identical to SEQ ID NO: 1

SEQ ID NO: 39 - protein, 153 aa, Equus caballus; identical to SEQ ID NO: 1

SEQ ID NO: 40 - protein, 153 aa, Equus caballus; identical to SEQ ID NO: 1

SEQ ID NO: 41 - protein, 153 aa, Equus caballus; identical to SEQ ID NO: 1

SEQ ID NO: 42 - protein, 153 aa, Equus caballus; identical to SEQ ID NO: 1

SEQ ID NO: 43 - protein, 153 aa, Equus caballus; identical to SEQ ID NO: 1

SEQ ID NO: 44 - protein, 153 aa, Equus caballus; identical to SEQ ID NO: 1

SEQ ID NO: 45 - protein, 153 aa, Equus caballus; identical to SEQ ID NO: 1

SEQ ID NO: 46 - protein, 153 aa, Equus caballus; identical to SEQ ID NO: 1

SEQ ID NO: 47 - protein, 153 aa, Equus caballus; identical to SEQ ID NO: 1

SEQ ID NO: 48 - protein, 153 aa, Equus caballus; identical to SEQ ID NO: 1

SEQ ID NO: 49 - protein, 153 aa, Equus caballus; identical to SEQ ID NO: 1

SEQ ID NO: 50 - protein, 153 aa, Equus caballus; identical to SEQ ID NO: 1

SEQ ID NO: 51 - protein, 153 aa, Equus caballus; identical to SEQ ID NO: 1

SEQ ID NO: 52 - protein, 153 aa, Equus caballus; identical to SEQ ID NO: 1

SEQ ID NO: 53 - protein, 153 aa, Equus caballus; identical to SEQ ID NO: 1

SEQ ID NO: 54 - protein, 199 aa, Bos taurus; identical to SEQ ID NO: 15

SEQ ID NO: 55 - protein, 199 aa, Bos taurus; identical to SEQ ID NO: 15

SEQ ID NO: 56 - protein, 199 aa, Bos taurus; identical to SEQ ID NO: 15

SEQ ID NO: 57 - protein, 199 aa, Bos taurus; identical to SEQ ID NO: 15

SEQ ID NO: 58 - protein, 199 aa, Bos taurus; identical to SEQ ID NO: 15

SEQ ID NO: 59 - protein, 199 aa, Bos taurus; identical to SEQ ID NO: 15

SEQ ID NO: 60 - protein, 199 aa, Bos taurus; identical to SEQ ID NO: 15

SEQ ID NO: 61 - protein, 199 aa, Bos taurus; identical to SEQ ID NO: 15

SEQ ID NO: 62 - protein, 199 aa, Bos taurus; identical to SEQ ID NO: 15

SEQ ID NO: 63 - protein, 199 aa, Bos taurus; identical to SEQ ID NO: 15

SEQ ID NO: 64 - protein, 199 aa, Bos taurus; identical to SEQ ID NO: 15

SEQ ID NO: 65 - protein, 199 aa, Bos taurus; identical to SEQ ID NO: 15

SEQ ID NO: 66 - protein, 199 aa, Bos taurus; identical to SEQ ID NO: 15

SEQ ID NO: 67 - protein, 199 aa, Bos taurus; identical to SEQ ID NO: 15

SEQ ID NO: 68 - protein, 199 aa, Bos taurus; identical to SEQ ID NO: 15

SEQ ID NO: 69 - protein, 199 aa, Bos taurus; identical to SEQ ID NO: 15

SEQ ID NO: 70 - protein, 199 aa, Bos taurus; identical to SEQ ID NO: 15

SEQ ID NO: 71 - protein, 199 aa, Bos taurus; identical to SEQ ID NO: 15

SEQ ID NO: 72 - protein, 199 aa, Bos taurus; identical to SEQ ID NO: 15

SEQ ID NO: 73 - protein, 199 aa, Bos taurus
RPKHPIKHQGLPQEVLNENLLRFFVAPFPEVFGKEKVNELSKDIGSESTEDQAMEDIKKMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 74 - protein, 199 aa, Bos taurus; identical to SEQ ID NO: 73

SEQ ID NO: 75 - protein, 199 aa, Bos taurus; identical to SEQ ID NO: 73

SEQ ID NO: 76 - protein, 199 aa, Bos taurus; identical to SEQ ID NO: 73

SEQ ID NO: 77 - protein, 199 aa, Bos taurus; identical to SEQ ID NO: 73

SEQ ID NO: 78 - protein, 199 aa, Bos taurus; identical to SEQ ID NO: 73

SEQ ID NO: 79 - protein, 199 aa, Bos taurus; identical to SEQ ID NO: 73

SEQ ID NO: 80 - protein, 199 aa, Bos taurus; identical to SEQ ID NO: 73

SEQ ID NO: 81 - protein, 199 aa, Bos taurus; identical to SEQ ID NO: 73

SEQ ID NO: 82 - protein, 199 aa, Bos taurus; identical to SEQ ID NO: 73

SEQ ID NO: 83 - protein, 199 aa, Bos taurus; identical to SEQ ID NO: 73

SEQ ID NO: 84 - protein, 199 aa, Bos taurus; identical to SEQ ID NO: 73

SEQ ID NO: 85 - protein, 199 aa, Bos taurus; identical to SEQ ID NO: 73

SEQ ID NO: 86 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMGNSAEPEQSLACQCLVRTPEVDDEALEKFDKALKALPMHIRLSFNPTQLEEQCHI

SEQ ID NO: 87 - protein, 162 aa, Bos taurus; identical to SEQ ID NO: 86

SEQ ID NO: 88 - protein, 162 aa, Bos taurus; identical to SEQ ID NO: 86

SEQ ID NO: 89 - protein, 162 aa, Bos taurus; identical to SEQ ID NO: 86

SEQ ID NO: 90 - protein, 162 aa, Bos taurus; identical to SEQ ID NO: 86

SEQ ID NO: 91 - protein, 162 aa, Bos taurus; identical to SEQ ID NO: 86

SEQ ID NO: 92 - protein, 162 aa, Bos taurus; identical to SEQ ID NO: 86

SEQ ID NO: 93 - protein, 162 aa, Bos taurus; identical to SEQ ID NO: 86

SEQ ID NO: 94 - protein, 162 aa, Bos taurus; identical to SEQ ID NO: 86

SEQ ID NO: 95 - protein, 162 aa, Bos taurus; identical to SEQ ID NO: 86

SEQ ID NO: 96 - protein, 162 aa, Bos taurus; identical to SEQ ID NO: 86

SEQ ID NO: 97 - protein, 162 aa, Bos taurus; identical to SEQ ID NO: 86

SEQ ID NO: 98 - protein, 162 aa, Bos taurus; identical to SEQ ID NO: 86

SEQ ID NO: 99 - protein, 162 aa, Bos taurus; identical to SEQ ID NO: 86

SEQ ID NO: 100 - protein, 162 aa, Bos taurus; identical to SEQ ID NO: 86

SEQ ID NO: 101 - protein, 162 aa, Bos taurus; identical to SEQ ID NO: 86

SEQ ID NO: 102 - protein, 162 aa, Bos taurus; identical to SEQ ID NO: 86

SEQ ID NO: 103 - protein, 162 aa, Bos taurus; identical to SEQ ID NO: 86

SEQ ID NO: 104 - protein, 162 aa, Bos taurus; identical to SEQ ID NO: 86

SEQ ID NO: 105 - protein, 162 aa, Bos taurus; identical to SEQ ID NO: 86

SEQ ID NO: 106 - protein, 162 aa, Bos taurus; identical to SEQ ID NO: 86

SEQ ID NO: 107 - protein, 162 aa, Bos taurus; identical to SEQ ID NO: 86

SEQ ID NO: 108 - protein, 162 aa, Bos taurus; identical to SEQ ID NO: 86

SEQ ID NO: 109 - protein, 162 aa, Bos taurus; identical to SEQ ID NO: 86

SEQ ID NO: 110 - protein, 162 aa, Bos taurus; identical to SEQ ID NO: 86

SEQ ID NO: 111 - protein, 162 aa, Bos taurus; identical to SEQ ID NO: 86

SEQ ID NO: 112 - protein, 199 aa, Bos taurus
RPKHPIKHQGLPQEVLNENLLRFFVAPFPEVFGKEKVNELSKDIGSESTEDQAMEDIKQMEAESILSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 113 - protein, 199 aa, Bos taurus; identical to SEQ ID NO: 112

SEQ ID NO: 114 - protein, 199 aa, Bos taurus; identical to SEQ ID NO: 112

SEQ ID NO: 115 - protein, 199 aa, Bos taurus; identical to SEQ ID NO: 112

SEQ ID NO: 116 - protein, 199 aa, Bos taurus; identical to SEQ ID NO: 112

SEQ ID NO: 117 - protein, 199 aa, Bos taurus; identical to SEQ ID NO: 112

SEQ ID NO: 118 - protein, 199 aa, Bos taurus; identical to SEQ ID NO: 112

SEQ ID NO: 119 - protein, 199 aa, Bos taurus; identical to SEQ ID NO: 112

SEQ ID NO: 120 - protein, 199 aa, Bos taurus; identical to SEQ ID NO: 112

SEQ ID NO: 121 - protein, 199 aa, Bos taurus
RPKHPIKHQGLPQEVLNENLLRFFVAPFPEVFGKEKVNELSKDIGSESTEDQTMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 122 - protein, 199 aa, Bos taurus; identical to SEQ ID NO: 121

SEQ ID NO: 123 - protein, 199 aa, Bos taurus; identical to SEQ ID NO: 121

SEQ ID NO: 124 - protein, 199 aa, Bos taurus; identical to SEQ ID NO: 121

SEQ ID NO: 125 - protein, 199 aa, Bos taurus; identical to SEQ ID NO: 121

SEQ ID NO: 126 - protein, 199 aa, Bos taurus; identical to SEQ ID NO: 121

SEQ ID NO: 127 - protein, 199 aa, Bos taurus; identical to SEQ ID NO: 121

SEQ ID NO: 128 - protein, 199 aa, Bos taurus; identical to SEQ ID NO: 121

SEQ ID NO: 129 - protein, 199 aa, Bos taurus; identical to SEQ ID NO: 121

SEQ ID NO: 130 - protein, 199 aa, Bos taurus; identical to SEQ ID NO: 121

SEQ ID NO: 131 - protein, 199 aa, Bos taurus; identical to SEQ ID NO: 121

SEQ ID NO: 132 - protein, 199 aa, Bos taurus; identical to SEQ ID NO: 121

SEQ ID NO: 133 - protein, 199 aa, Bos taurus; identical to SEQ ID NO: 121

SEQ ID NO: 134 - protein, 199 aa, Bos taurus; identical to SEQ ID NO: 121

SEQ ID NO: 135 - protein, 199 aa, Bos taurus; identical to SEQ ID NO: 121

SEQ ID NO: 136 - protein, 199 aa, Bos taurus; identical to SEQ ID NO: 121

SEQ ID NO: 137 - protein, 199 aa, Bos taurus; identical to SEQ ID NO: 121

SEQ ID NO: 138 - protein, 162 aa, Bos taurus; identical to SEQ ID NO: 22

SEQ ID NO: 139 - protein, 162 aa, Bos taurus; identical to SEQ ID NO: 22

SEQ ID NO: 140 - protein, 162 aa, Bos taurus; identical to SEQ ID NO: 22

SEQ ID NO: 141 - protein, 162 aa, Bos taurus; identical to SEQ ID NO: 22

SEQ ID NO: 142 - protein, 162 aa, Bos taurus; identical to SEQ ID NO: 22

SEQ ID NO: 143 - protein, 162 aa, Bos taurus; identical to SEQ ID NO: 22

SEQ ID NO: 144 - protein, 162 aa, Bos taurus; identical to SEQ ID NO: 22

SEQ ID NO: 145 - protein, 162 aa, Bos taurus; identical to SEQ ID NO: 22

SEQ ID NO: 146 - protein, 162 aa, Bos taurus; identical to SEQ ID NO: 22

SEQ ID NO: 147 - protein, 162 aa, Bos taurus
LIVTQTMKGLDIQKVAGTWYSLAMAASDISLLDAQSAPLRVYVEELKPTPEGDLEILLHKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 148 - protein, 162 aa, Bos taurus; identical to SEQ ID NO: 22

SEQ ID NO: 149 - protein, 162 aa, Bos taurus; identical to SEQ ID NO: 147

SEQ ID NO: 150 - protein, 162 aa, Bos taurus; identical to SEQ ID NO: 22

SEQ ID NO: 151 - protein, 162 aa, Bos taurus; identical to SEQ ID NO: 22

SEQ ID NO: 152 - protein, 162 aa, Bos taurus; identical to SEQ ID NO: 22

SEQ ID NO: 153 - protein, 162 aa, Bos taurus; identical to SEQ ID NO: 147

SEQ ID NO: 154 - protein, 162 aa, Bos taurus; identical to SEQ ID NO: 22

SEQ ID NO: 155 - protein, 162 aa, Bos taurus; identical to SEQ ID NO: 22

SEQ ID NO: 156 - protein, 162 aa, Bos taurus; identical to SEQ ID NO: 22

SEQ ID NO: 157 - protein, 162 aa, Bos taurus; identical to SEQ ID NO: 22

SEQ ID NO: 158 - protein, 162 aa, Bos taurus; identical to SEQ ID NO: 22

SEQ ID NO: 159 - protein, 162 aa, Bos taurus; identical to SEQ ID NO: 22

SEQ ID NO: 160 - protein, 162 aa, Bos taurus; identical to SEQ ID NO: 22

SEQ ID NO: 161 - protein, 162 aa, Bos taurus; identical to SEQ ID NO: 22

SEQ ID NO: 162 - protein, 162 aa, Bos taurus
LIVTQTMKGLDIQKVAGTWYSLAMAASDISLLDAQSAPLRVYVEELKPTPEGDLEILLQKWENGECAQKKIIAEKTKMPAVFKIDALNENKVLVLDTDYKKYLLFCMENSAEPEQSLACQCLVRTPEVDDEALEKFDKALKALPMHIRLSFNPTQLEGQCHI

SEQ ID NO: 163 - protein, 162 aa, Bos taurus; identical to SEQ ID NO: 162

SEQ ID NO: 164 - protein, 162 aa, Bos taurus; identical to SEQ ID NO: 162

SEQ ID NO: 165 - protein, 162 aa, Bos taurus; identical to SEQ ID NO: 162

SEQ ID NO: 166 - protein, 162 aa, Bos taurus; identical to SEQ ID NO: 162

SEQ ID NO: 167 - protein, 162 aa, Bos taurus; identical to SEQ ID NO: 162

SEQ ID NO: 168 - protein, 162 aa, Bos taurus; identical to SEQ ID NO: 162

SEQ ID NO: 169 - protein, 162 aa, Bos taurus; identical to SEQ ID NO: 162

SEQ ID NO: 170 - protein, 162 aa, Bos taurus; identical to SEQ ID NO: 162

SEQ ID NO: 171 - protein, 162 aa, Bos taurus; identical to SEQ ID NO: 162

SEQ ID NO: 172 - protein, 162 aa, Bos taurus; identical to SEQ ID NO: 162

SEQ ID NO: 173 - protein, 162 aa, Bos taurus; identical to SEQ ID NO: 162

SEQ ID NO: 174 - protein, 162 aa, Bos taurus; identical to SEQ ID NO: 162

SEQ ID NO: 175 - protein, 162 aa, Bos taurus; identical to SEQ ID NO: 162

SEQ ID NO: 176 - protein, 162 aa, Bos taurus; identical to SEQ ID NO: 162

SEQ ID NO: 177 - protein, 162 aa, Bos taurus; identical to SEQ ID NO: 162

SEQ ID NO: 178 - protein, 162 aa, Bos taurus; identical to SEQ ID NO: 162

SEQ ID NO: 179 - protein, 162 aa, Bos taurus; identical to SEQ ID NO: 162

SEQ ID NO: 180 - protein, 162 aa, Bos taurus; identical to SEQ ID NO: 162

SEQ ID NO: 181 - protein, 162 aa, Bos taurus; identical to SEQ ID NO: 162

SEQ ID NO: 182 - protein, 162 aa, Bos taurus; identical to SEQ ID NO: 162

SEQ ID NO: 183 - protein, 162 aa, Bos taurus; identical to SEQ ID NO: 162

SEQ ID NO: 184 - protein, 162 aa, Bos taurus; identical to SEQ ID NO: 162

SEQ ID NO: 185 - protein, 162 aa, Bos taurus
LIVTQTMKGLDIQKVAGTWYSLAMAASDISLLDAQSAPLRVYVEQLKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 186 - protein, 162 aa, Bos taurus; identical to SEQ ID NO: 185

SEQ ID NO: 187 - protein, 162 aa, Bos taurus; identical to SEQ ID NO: 185

SEQ ID NO: 188 - protein, 162 aa, Bos taurus; identical to SEQ ID NO: 185

SEQ ID NO: 189 - protein, 162 aa, Bos taurus; identical to SEQ ID NO: 185

SEQ ID NO: 190 - protein, 162 aa, Bos taurus; identical to SEQ ID NO: 185

SEQ ID NO: 191 - protein, 162 aa, Bos taurus; identical to SEQ ID NO: 185

SEQ ID NO: 192 - protein, 162 aa, Bos taurus; identical to SEQ ID NO: 185

SEQ ID NO: 193 - protein, 162 aa, Bos taurus; identical to SEQ ID NO: 185

SEQ ID NO: 194 - protein, 162 aa, Bos taurus; identical to SEQ ID NO: 185

SEQ ID NO: 195 - protein, 162 aa, Bos taurus; identical to SEQ ID NO: 21

SEQ ID NO: 196 - protein, 162 aa, Bos taurus; identical to SEQ ID NO: 21

SEQ ID NO: 197 - protein, 162 aa, Bos taurus; identical to SEQ ID NO: 21

SEQ ID NO: 198 - protein, 162 aa, Bos taurus; identical to SEQ ID NO: 21

SEQ ID NO: 199 - protein, 162 aa, Bos taurus; identical to SEQ ID NO: 21

SEQ ID NO: 200 - protein, 162 aa, Bos taurus; identical to SEQ ID NO: 21

SEQ ID NO: 201 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTPEVDDEALEKFDKALKALPMHIRLSFNPTQLEEQCHI

SEQ ID NO: 202 - protein, 162 aa, Bos taurus; identical to SEQ ID NO: 201

SEQ ID NO: 203 - protein, 162 aa, Bos taurus; identical to SEQ ID NO: 201

SEQ ID NO: 204 - protein, 162 aa, Bos taurus; identical to SEQ ID NO: 201

SEQ ID NO: 205 - protein, 162 aa, Bos taurus; identical to SEQ ID NO: 201

SEQ ID NO: 206 - protein, 162 aa, Bos taurus; identical to SEQ ID NO: 201

SEQ ID NO: 207 - protein, 162 aa, Bos taurus; identical to SEQ ID NO: 201

SEQ ID NO: 208 - protein, 162 aa, Bos taurus
LIVTQTMKGLDIQKVAGTWYSLAMAASDISLLDAQSAPLRVYVEELKPTPEGDLEILLQKWENDECAQKKIIAEKTKIPAVFKIDALNENKVLVLDTDYKKYLLFCMENSAEPEQSLVCQCLVRTPEVDDEALEKFDKALKALPMHIRLSFNPTQLEEQCHI

SEQ ID NO: 209 - protein, 162 aa, Bos taurus; identical to SEQ ID NO: 208

SEQ ID NO: 210 - protein, 162 aa, Bos taurus
LIVTQTMKGLDIQKVAGTWYSLAMAASDISLLDAQSAPLRVYVEELKPTPEGDLEILLQKWENGECAQKKIIAEKTKIPAVFKIDALNENKVLVLDTDYKKYLLFCMENSAEPEQSLACQCLVRTLEVDDEALEKFDKALKALPMHIRLSFNPTQLEEQCHI

SEQ ID NO: 211 - protein, 162 aa, Bos taurus; identical to SEQ ID NO: 210

SEQ ID NO: 212 - protein, 162 aa, Bos taurus; identical to SEQ ID NO: 210

SEQ ID NO: 213 - protein, 162 aa, Bos taurus; identical to SEQ ID NO: 210

SEQ ID NO: 214 - protein, 153 aa, Equus burchelli; identical to SEQ ID NO: 1

SEQ ID NO: 215 - protein, 153 aa, Equus burchelli; identical to SEQ ID NO: 1

SEQ ID NO: 216 - protein, 154 aa, Equus burchelli
MGLSDGEWQQVLNVWGKVEADIAGHGQEVLIRLFTGHPETLEKFDKFKHLKTEAEMKASEDLKKHGTVVLTALGGILKKKGHHEAELKPLAQSHATKHKIPIKYLEFISDAIIHVLHSKHPGDFGADAQGAMTKALELFRNDIAAKYKELGFQG

SEQ ID NO: 217 - protein, 153 aa, Equus burchelli; identical to SEQ ID NO: 1

SEQ ID NO: 218 - protein, 153 aa, Equus burchelli; identical to SEQ ID NO: 1

SEQ ID NO: 219 - protein, 154 aa, Equus burchelli; identical to SEQ ID NO: 216

SEQ ID NO: 220 - protein, 153 aa, Equus burchelli; identical to SEQ ID NO: 1

SEQ ID NO: 221 - protein, 154 aa, Equus burchelli; identical to SEQ ID NO: 216

SEQ ID NO: 222 - protein, 153 aa, Equus burchelli; identical to SEQ ID NO: 1

SEQ ID NO: 223 - protein, 153 aa, Equus burchelli; identical to SEQ ID NO: 1

SEQ ID NO: 224 - protein, 153 aa, Equus burchelli; identical to SEQ ID NO: 1

SEQ ID NO: 225 - protein, 154 aa, Equus burchelli; identical to SEQ ID NO: 216

SEQ ID NO: 226 - protein, 153 aa, Equus burchelli; identical to SEQ ID NO: 1

SEQ ID NO: 227 - protein, 153 aa, Equus burchelli; identical to SEQ ID NO: 1

SEQ ID NO: 228 - protein, 153 aa, Equus burchelli; identical to SEQ ID NO: 1

SEQ ID NO: 229 - protein, 153 aa, Equus burchelli; identical to SEQ ID NO: 1

SEQ ID NO: 230 - protein, 154 aa, Equus burchelli; identical to SEQ ID NO: 216

SEQ ID NO: 231 - protein, 153 aa, Equus burchelli; identical to SEQ ID NO: 1

SEQ ID NO: 232 - protein, 153 aa, Equus burchelli; identical to SEQ ID NO: 1

SEQ ID NO: 233 - protein, 153 aa, Equus burchelli; identical to SEQ ID NO: 1

SEQ ID NO: 234 - protein, 153 aa, Equus burchelli; identical to SEQ ID NO: 1

SEQ ID NO: 235 - protein, 154 aa, Equus burchelli; identical to SEQ ID NO: 216

SEQ ID NO: 236 - protein, 153 aa, Equus burchelli; identical to SEQ ID NO: 1

SEQ ID NO: 237 - protein, 153 aa, Equus burchelli; identical to SEQ ID NO: 1

SEQ ID NO: 238 - protein, 153 aa, Equus burchelli; identical to SEQ ID NO: 1

SEQ ID NO: 239 - protein, 153 aa, Equus burchelli; identical to SEQ ID NO: 1

SEQ ID NO: 240 - protein, 153 aa, Equus burchelli; identical to SEQ ID NO: 1

SEQ ID NO: 241 - protein, 153 aa, Equus burchelli; identical to SEQ ID NO: 1

SEQ ID NO: 242 - protein, 154 aa, Equus burchelli; identical to SEQ ID NO: 216

SEQ ID NO: 243 - protein, 154 aa, Equus burchelli; identical to SEQ ID NO: 216

SEQ ID NO: 244 - protein, 153 aa, Equus burchelli; identical to SEQ ID NO: 1

SEQ ID NO: 245 - protein, 153 aa, Equus burchelli; identical to SEQ ID NO: 1

SEQ ID NO: 246 - protein, 153 aa, Equus burchelli; identical to SEQ ID NO: 1

SEQ ID NO: 247 - protein, 153 aa, Equus burchelli; identical to SEQ ID NO: 1

SEQ ID NO: 248 - protein, 153 aa, Equus burchelli; identical to SEQ ID NO: 1

SEQ ID NO: 249 - protein, 154 aa, Equus burchelli; identical to SEQ ID NO: 216

SEQ ID NO: 250 - protein, 153 aa, Equus burchelli; identical to SEQ ID NO: 1

SEQ ID NO: 251 - protein, 154 aa, Equus burchelli; identical to SEQ ID NO: 216

SEQ ID NO: 252 - protein, 153 aa, Equus burchelli; identical to SEQ ID NO: 1

SEQ ID NO: 253 - protein, 153 aa, Equus burchelli; identical to SEQ ID NO: 1

SEQ ID NO: 254 - protein, 154 aa, Equus burchelli; identical to SEQ ID NO: 216

SEQ ID NO: 255 - protein, 153 aa, Equus burchelli; identical to SEQ ID NO: 1

SEQ ID NO: 256 - protein, 154 aa, Equus burchelli; identical to SEQ ID NO: 216

SEQ ID NO: 257 - protein, 153 aa, Equus burchelli; identical to SEQ ID NO: 1

SEQ ID NO: 258 - protein, 153 aa, Equus burchelli; identical to SEQ ID NO: 1

SEQ ID NO: 259 - protein, 153 aa, Equus burchelli; identical to SEQ ID NO: 1

SEQ ID NO: 260 - protein, 154 aa, Equus burchelli; identical to SEQ ID NO: 216

SEQ ID NO: 261 - protein, 153 aa, Equus burchelli; identical to SEQ ID NO: 1

SEQ ID NO: 262 - protein, 153 aa, Equus burchelli; identical to SEQ ID NO: 1

SEQ ID NO: 263 - protein, 153 aa, Equus burchelli; identical to SEQ ID NO: 1

SEQ ID NO: 264 - protein, 153 aa, Equus burchelli; identical to SEQ ID NO: 1

SEQ ID NO: 265 - protein, 153 aa, Equus burchelli; identical to SEQ ID NO: 1

SEQ ID NO: 266 - protein, 153 aa, Equus burchelli; identical to SEQ ID NO: 1

SEQ ID NO: 267 - protein, 153 aa, Equus burchelli; identical to SEQ ID NO: 1

SEQ ID NO: 268 - protein, 153 aa, Equus burchelli; identical to SEQ ID NO: 1

SEQ ID NO: 269 - protein, 154 aa, Equus burchelli; identical to SEQ ID NO: 216

SEQ ID NO: 270 - protein, 153 aa, Equus burchelli; identical to SEQ ID NO: 1

SEQ ID NO: 271 - protein, 153 aa, Equus burchelli; identical to SEQ ID NO: 1

SEQ ID NO: 272 - protein, 153 aa, Equus burchelli; identical to SEQ ID NO: 1

SEQ ID NO: 273 - protein, 153 aa, Equus burchelli; identical to SEQ ID NO: 1

SEQ ID NO: 274 - protein, 154 aa, Equus burchelli; identical to SEQ ID NO: 216

SEQ ID NO: 275 - protein, 153 aa, Equus burchelli; identical to SEQ ID NO: 1

SEQ ID NO: 276 - protein, 153 aa, Equus burchelli; identical to SEQ ID NO: 1

SEQ ID NO: 277 - protein, 154 aa, Equus burchelli; identical to SEQ ID NO: 216

SEQ ID NO: 278 - protein, 154 aa, Equus burchelli; identical to SEQ ID NO: 216

SEQ ID NO: 279 - protein, 154 aa, Equus burchelli; identical to SEQ ID NO: 216

SEQ ID NO: 280 - protein, 153 aa, Equus burchelli; identical to SEQ ID NO: 1

SEQ ID NO: 281 - protein, 153 aa, Equus burchelli; identical to SEQ ID NO: 1

SEQ ID NO: 282 - protein, 153 aa, Equus burchelli; identical to SEQ ID NO: 1

SEQ ID NO: 283 - protein, 165 aa, Tachyglossus aculeatus aculeatus
MYFIYLLSVMLVLSFMAFASNPSPIYGGLSLVLSGGVGCGIIVSLGGSFLGLIVFLVYLGGMLVVFGYTAAMATEEYPETWVDYTVVLNLAIMVGMLGVVWYEFFEEVDLGVGYDLLDLGGMEVLGGDFNGVSLLYACGGWELVFSGWILFLTIFVVLEVTRGEH

SEQ ID NO: 284 - protein, 174 aa, Hippopotamus amphibious
MTYVVFILSIVFVIGLIGSPSKPSPIYGGLGLIVSGGAGCGMVLNFGGSFLGLMVFLVYLGGMLVVFGYTTAMATEQYPEVWVSNKVVLGAFLLGLMMEFLAVLYVLKEGEVELVFKFSGLGDWVVYGMSDFGVFSGEAMGVAALYSYGVWLVVVTGWSLFVGVVVIMEVTRGG

SEQ ID NO: 285 - protein, 82 aa, Cannabis sativa
SGSTGERSFADIITSIRYWVIHSITIPSLFIAGWLFVSTGLAYDVFGSPRPNEYFTESRQGVPLITGRFDSLEQLDEFSRSF

SEQ ID NO: 286 - protein, 82 aa, Cannabis sativa; identical to SEQ ID NO: 285

SEQ ID NO: 287 - protein, 80 aa, Cannabis sativa
SHSVKIYDTCIGCTQCVRACPTDVLEMIPWDGCKAKQIASAPRTEDCVGCKRCESACPTDFLSVRVYLWHETTRSMGLAY

SEQ ID NO: 288 - protein, 80 aa, Cannabis sativa; identical to SEQ ID NO: 287

SEQ ID NO: 289 - protein, 80 aa, Cannabis sativa; identical to SEQ ID NO: 287

SEQ ID NO: 290 - protein, 33 aa, Cannabis sativa subsp. sativa
MEALVYTFLLVSTLGIIFFAIFFREPPKVPTKK

SEQ ID NO: 291 - protein, 72 aa, Cannabis sativa
ATQTVEGSSRSGSRRTGAGSLLKPLNSEYGKVAPGWGTTPLMGIAMALFAVFLSIILEIYNSSILLDGISMN

SEQ ID NO: 292 - protein, 72 aa, Cannabis sativa; identical to SEQ ID NO: 291

SEQ ID NO: 293 - protein, 82 aa, Humulus lupulus
SGSTGERSFADIITSIRYWVIHSITIPSLFIAGWLFVSTGLAYDVFGSPRPNEYFTESRQGIPLITGRFDSLEQLDEFSRSF

SEQ ID NO: 294 - protein, 36 aa, Cannabis sativa
MLTLKLFVYTVVIFFVSLFIFGFLSNDPGRNPGREE

SEQ ID NO: 295 - protein, 36 aa, Cannabis sativa; identical to SEQ ID NO: 294

SEQ ID NO: 296 - protein, 81 aa, Cannabis sativa
MNPIISAASVIAAGLAVGLASIGPGVGQGTAAGQAVEGIARQPEAEGKIRGTLLLSLAFMEALTIYGLVVALALLFANPFV

SEQ ID NO: 297 - protein, 81 aa, Cannabis sativa; identical to SEQ ID NO: 296

SEQ ID NO: 298 - protein, 81 aa, Cannabis sativa; identical to SEQ ID NO: 296

SEQ ID NO: 299 - protein, 99 aa, Humulus lupulus
ARKSLIQREKKRQKLEQKYHLIRGSSKKEIRKVPSLSEKWQIHGKLQSPPRNSAPTRLHRRCFLTGRPRANYRDFGLSGHILREMVHACLLPGATRSSW

SEQ ID NO: 300 - protein, 99 aa, Humulus lupulus; identical to SEQ ID NO: 299

SEQ ID NO: 301 - protein, 39 aa, Cannabis sativa
MTIDRTYPIFTVRWLAVHGLAVPTVSFLGSISAMQFIQR

SEQ ID NO: 302 - protein, 100 aa, Cannabis sativa
AVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 303 - protein, 100 aa, Cannabis sativa; identical to SEQ ID NO: 302

SEQ ID NO: 304 - protein, 89 aa, Cannabis sativa
MVKLRLKRCGRKQRPIYRIVAIDVRSRREGRALRKVGFYDPIKNQTYLNVPVILSFLEKGAQPTGTVHDISKKAGVLMELRLNHQPKFN

SEQ ID NO: 305 - protein, 89 aa, Cannabis sativa
MVKLRLKRCGRKQRPIYRIVAIDVRSRREGRALRKVGFYDPIKNQTYLNVPVILSFLEKGAQPTGTVHDISKKAGVFMELRLNHQPKFN

SEQ ID NO: 306 - protein, 89 aa, Cannabis sativa; identical to SEQ ID NO: 305

SEQ ID NO: 307 - protein, 160 aa, Cannabis sativa
GVFTYESEFTSSIAPARLFKAFVLDGDNLVPKIAPQAVEKVEILEGNGGVGTIKKITFGQGVPFKYVKHKIEAIDKESLTYSYSIIEGDALEGNQLEKITHESKLVASGDGGNVIKTVSKYYSAGDAQVNEEKVKEGEKQATQMLKTVEAYLKDHPEAYN

SEQ ID NO: 308 - protein, 161 aa, Cannabis sativa
MGVFTYESEFTSSIAPARLFKAFVLDGDNLVPKIAPQAVEKVEILEGNGGVGTIKKITFGQGVPFKYVKHKIEAIDKESLTYSYSIIEGDALEGNQLEKITHESKLVASGDGGNVIKTVSKYYSAGDAQVNEEKVKEGEKQATQMLKTVEAYLKDHPEAYN

SEQ ID NO: 309 - protein, 161 aa, Cannabis sativa; identical to SEQ ID NO: 308

SEQ ID NO: 310 - protein, 160 aa, Cannabis sativa; identical to SEQ ID NO: 307

SEQ ID NO: 311 - protein, 40 aa, Cannabis sativa
MADTTGRIPLWIIGTVAGILVIGLIGIFFYGSYSGLGSSL

SEQ ID NO: 312 - protein, 65 aa, Cannabis sativa
ARGKDARVTIILECTSCVRNRVNKESIGISRYITQKNRHNTPSRLELRKFCPSCYKHTIHGEIKK

SEQ ID NO: 313 - protein, 132 aa, Cannabis sativa
TLNLCVLTPNRIVWDSEVKEIILSTNSGQIGILPNHAPIATAVDIGILRIRLQDQWLTMALMGGFARIGNNEITVLVNDAEKGSDIDPQEAQQTLEIAEVNLSKAEGKRQTIEANLALRRARTRVEAINMMS

SEQ ID NO: 314 - protein, 37 aa, Cannabis sativa
MIEVFLFGIVLGLIPITLAGLFVTAYLQYRRGDQLDL

SEQ ID NO: 315 - protein, 91 aa, Cannabis sativa
ITCGQVASSLAPCLSYLKVGGAVPDGCCNDIKSLSGAAKTPADRQAACKCLKSAASSIKGVNFNLASGLPGKCGVSIPYKISPSTDCSSVK

SEQ ID NO: 316 - protein, 37 aa, Cannabis sativa
TQSNPNEQNVELNRTSLYWGLLLIFVLAVLFSNYFFN

SEQ ID NO: 317 - protein, 159 aa, Cannabis sativa
GVTKKPDLNDPVLRAKLAKGMGHNYYGEPAWPNDLLYIFPVVILGTIACNVGLAVLEPSMIGEPADPFATPLEILPEWYFFPVFQILRTVPNKLLGVLLMVSVPAGLLTVPFLENVNKFQNPFRRPVATTVFLIGTAVALWLGIGATLPIDKSLTLGLF

SEQ ID NO: 318 - protein, 42 aa, Cannabis sativa
MRDLKTYLSVAPVVSTIWFGSLAGLLIEINRLFPDALIFPFF

(19) United States
(12) Patent Application Publication
(10) Pub. No.: US 2023/0027592 A1
(43) Pub. Date: Jan. 26, 2023

(54) Method of Protein Extraction from Cannabis Plant Material

(71) Applicant: Agriculture Victoria Services Pty Ltd, Bundoora (AU)

(72) Inventors: Delphine Elsie Michelle Vincent, Epping (AU); Simone Jane Rochfort, Reservoir (AU); German Carlos Spangenberg, Bundoora (AU)

(21) Appl. No.: 17/297,730

(22) PCT Filed: Nov. 8, 2019
(86) PCT No.: PCT/AU2019/051228

§ 371 Date: May 27, 2021

(30) Foreign Application Priority Data
Dec. 20, 2018 (AU) 2018904869
Jul. 25, 2019 (AU) 2019902643

(51) Int. Cl. C07K 1/14; C07K 1/30; C12N 9/50; G01N 33/68; G01N 30/72

(52) U.S. Cl. CPC C07K 1/145; C07K 1/30; G01N 2030/027; G01N 33/6842; G01N 30/7233; C12N 9/50

(57) ABSTRACT

The present invention relates generally to a method for extracting cannabis-derived proteins from cannabis plant material, including the preparation of samples of extracted cannabis-derived proteins for proteomic analysis and methods for analysing a cannabis plant proteome.

The present application claims priority from both Australian Provisional Patent Application 2018904869 filed 20 Dec. 2018 and Australian Provisional Patent Application 2019902643 filed 25 Jul. 2019, the disclosure of which is hereby expressly incorporated herein by reference in its entirety.

### FIELD

The present invention relates generally to a method for extracting cannabis-derived proteins from cannabis plant material, including the preparation of samples of extracted cannabis-derived proteins for proteomic analysis and methods for analysing a cannabis plant proteome.

### BACKGROUND

Cannabis is an herbaceous flowering plant of the Cannabis genus (Rosale) that has been used for its fibre and medicinal properties for thousands of years. The medicinal qualities of cannabis have been recognised since at least 2800 BC, with use of cannabis featuring in ancient Chinese and Indian medical texts. Although use of cannabis for medicinal purposes has been known for centuries, research into the pharmacological properties of the plant has been limited due to its illegal status in most jurisdictions.

The chemistry of cannabis is varied. It is estimated that cannabis plants produce more than 400 different molecules, including phytocannabinoids, terpenes and phenolics. Cannabinoids, such as Δ-9-tetrahydrocannabinol (THC) and cannabidiol (CBD) are the most well-known and researched cannabinoids. CBD and THC are naturally present in their acidic forms, Δ-9-tetrahydrocannabinolic acid (THCA) and cannabidiolic acid (CBDA), in planta which are alternative products of a shared precursor, cannabigerolic acid (CBGA). Since different cannabinoids are likely to have different therapeutic potential, it is important to be able to identify and extract different cannabinoids that are suitable for medicinal use.

Quantitative proteomic techniques allow for the quantitation of abundance, form, location, or activity of proteins that are involved in developmental changes or responses to alterations in environmental conditions. Initially, proteomic techniques included traditional two-dimensional (2D) gel electrophoresis and protein staining. While these techniques have been, and continue to be, informative about biological systems, there are a number of problems with sensitivity, throughput and reproducibility which limits their application for comparative proteomic analysis. Advancements in platform technology have allowed mass spectroscopy (MS) to develop into the primary detection method used in proteomics, which has greatly expanded depth and improved reliability of proteomic analysis when compared to 2D techniques.

The ability for MS-based techniques to accurately resolve the diversity and complexity of cellular proteomes is associated with the development of different protocols to support analysis by MS. For the most part, these protocols have been developed to improve the depth of proteome coverage through the optimisation of conditions that are favourable for proteolytic digestion and sample recovery. The careful selection of solutions and enrichment methods during sample preparation is essential to ensure compatibility with downstream workflows and detection platforms. In the context of cannabis, this also includes the sampling of appropriate plant material at different stages of plant development.

Previous studies of the cannabis proteome have largely focused on the analysis of non-reproductive organs from immature cannabis plants such as roots and hypocotyls (Bona et al. 2007, Proteomics 7:1121-30; Behr et al. 2018, BMC Plant Biol. 18:1) or processed seeds from hemp (Aiello et al. 2016, J. Proteomics 147:187-96). Furthermore, these previous studies did not employ any standardised sample preparation method to maximise the recovery of cannabis-derived proteins for proteomic analysis. This is reflected in the types of analysis methods employed. For example, in the study conducted by Bona et al., protein extracts were then analysed by two-dimensional electrophoresis (2-DE), while Aiello et al. used one-dimensional polyacrylamide gel electrophoresis (1-D PAGE).

There remains, therefore, an urgent need for improved methods for extracting cannabis-derived proteins from cannabis plant material in a manner that optimises the recovery of cannabis-derived proteins for proteomic analysis.

### SUMMARY

In an aspect disclosed herein, there is provided a method of extracting cannabis-derived proteins from cannabis plant material, the method comprising:
(a) suspending cannabis plant material in a solution comprising a charged chaotropic agent for a period of time to allow for extraction of cannabis-derived proteins into the solution; and
(b) separating the solution comprising the cannabis-derived proteins from residual plant material.

In another aspect disclosed herein, there is provided a method of extracting cannabis-derived proteins from cannabis plant material, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent for a period of time to allow for extraction of cannabis-derived proteins into the solution; and
(c) separating the solution comprising the cannabis-derived proteins from residual plant material.

In another aspect disclosed herein, there is provided a method of preparing a sample of cannabis-derived proteins from cannabis plant material for proteomic analysis, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent for a period of time to allow for extraction of cannabis-derived proteins into the solution;
(c) separating the solution comprising the cannabis-derived proteins from residual plant material; and
(d) digesting the solution of (c) with a protease.

In another aspect disclosed herein, there is provided a method of preparing a sample of cannabis-derived proteins from cannabis plant material for proteomic analysis, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent for a period of time to allow for extraction of cannabis-derived proteins into the solution; and
(c) separating the solution comprising the cannabis-derived proteins from residual plant material.

In an embodiment, the charged chaotropic acid is guanidine hydrochloride.

The present disclosure also extends to methods of analysing a cannabis plant proteome, the methods comprising preparing a sample of cannabis-derived proteins in accordance with the methods disclosed herein; and subjecting the sample to proteomic analysis.

### BRIEF DESCRIPTION OF THE DRAWINGS

FIG. 1 is a graphical representation of intact proteins extracted using urea- or guanidine-HCl-based extraction methods, data was compared by Principal Component Analysis (PCA) of PC1 (60.7% variance; x-axis) against PC2 (32.9% variance; y-axis) using top-down proteomics data from 571 proteins.

FIG. 2 is a graphical representation of peptides extracted using urea- or guanidine-HCl-based extraction methods, data was compared by PCA of PC1 (65.2% variance; x-axis) against PC2 (11.6% variance; y-axis) using bottom-up proteomics data from 43,972 proteomic clusters.

FIG. 3 is a graphical representation of the comparison of the number of tryptic peptides identified from (A) trichomes and apical buds, extraction methods 1 and 2 (AB1, AB2, T1 and T2); (B), apical buds, extraction methods 1-6 (AB1-AB6); and (C) AB1-AB6 and T1-T2.

FIG. 4 is a graphical representation of a pathway analysis of cannabis proteins identified from (A) apical buds; and (B) trichomes.

FIG. 5 is a graphical representation of the distribution of UniprotKB entries from C. sativa entries (y-axis) from 1986 to 2018 (x-axis).

FIG. 6 shows the impact of extraction methods on enzymes involved in cannabinoid biosynthesis: (A) The cannabinoid biosynthesis pathway; (B) Two-dimensional hierarchical clustering of enzymes involved in cannabinoid synthesis. Columns represent extraction method per tissue types (AB, apical bud; T, trichomes), rows represent the peptides identified from enzymes of interest. Peptides from the same enzymes bear the same shade of grey.

FIG. 7 is a graphical representation of FTMS and FTMS/MS spectra from infused myoglobin. (A) Fragmentation of all ions by SID; (B) Fragmentation of ion 942.68 m/z (z=+18) by ETD, CID and HCD; (C) Fragmentation of ion 1211.79 m/z (z=+14) by ETD, CID and HCD.

FIG. 8 shows the matching ions achieved for myoglobin using Prosight Lite. (A-C) A graphical representation of the number of ions (y-axis) against myoglobin amino acid position (x-axis) for every MS/MS parameter tested (A) summed across all five charge states listed in Table 5; (B) summed by MS/MS mode along myoglobin amino acid sequence; (C) summed globally across all the data obtained for myoglobin along its amino acid sequence; (D) A schematic representation of global amino acid sequence coverage when all MS/MS data is considered; and (E) a graphical representation of sequence coverage achieved for each of the five myoglobin charge states.

FIG. 9 shows excerpts of results for β-lactoglobulin (β-LG), α-S1-casein (α-S1-CN), and bovine serum albumin (BSA). (A) Graphical representations of examples of FTMS and FTMS/MS spectra using SID, ETD, CID and HCD; and (B) global AA sequence coverage when all MS/MS data is considered.

FIG. 10 is a graphical representation of the relationship between the observed mass (kD; left y-axis) and coverage (%; right y-axis) of the protein standards (x-axis) analysed and their sequencing results by top-down proteomics.

FIG. 11 shows the Mascot search results of protein standards MS/MS peak lists using (A) the homemade database and (B) Swissprot database.

FIG. 12 shows the profiles of medicinal cannabis protein samples. (A) Graphical representations of total ion chromatograms (TIC) representing elution time (min; x-axis) and signal intensity (x-axis) for each biological replicate (buds 1 to 3), n=2; (B) Graphical representations of LC-MS pattern representing elution time (min; y-axis) and mass range (500-2000 m/z; x-axis) of each biological replicate (buds 1 to 3), n=1; (C) Graphical representations of deconvoluted LC-MS map representing elution time (min; y-axis) and mass range (3-30 kDa; x-axis) of each biological replicate (buds 1 to 3), n=1; (D) Graphical representations of zoom-in the area boxed in (C) representing elution time (15-45 min; y-axis) and mass range (9-11.5 kDa; x-axis) corresponding to abundant proteins; and (E) Graphical representations of triplicated LC-MS/MS patterns from biological replicate bud 1; dots represents MS/MS events.

FIG. 13 is a graphical representation of the distribution of cannabis proteins according to their accurate masses (Da; y-axis) and occurrence (x-axis).

FIG. 14 shows multivariate statistical analyses using LC-MS data from cannabis protein samples using (A) PCA; and (B) Hierarchical Clustering Analysis (HCA).

FIG. 15 shows the statistics on parent ions from cannabis proteins analysed by LC-MS/MS. (A) A graphical representation on the distribution of deconvoluted mass (Da; y-axis) according to their charge state (z; x-axis); (B) A graphical representation of the distribution of deconvoluted masses (Da; y-axis) according to their base peak intensity (x-axis); and (C) A graphical representation of the distribution of deconvoluted masses (Da; y-axis) according to their elution times (min; x-axis).

FIG. 16 shows the top-down sequencing results from Mascot for C. sativa Cytochrome b559 subunit alpha (A0A0C5ARS8). (A) Protein view; and (B) Peptide view.

FIG. 17 shows the top-down sequencing summary for C. sativa Photosystem I iron-sulphur centre (PS I Fe—S centre, accession A0A0C5AS17). (A) A graphical representation of FTMS spectra showing relative abundance (y-axis) and mass (m/z; x-axis) at 30.8 min, lightning bolts depicts the two most abundant charge states chosen for MS/MS fragmentation; (B) Graphical representations of FTMS/MS spectra showing relative abundance (y-axis) and mass (m/z; x-axis) for “low”, “mid” and “high” charge states using each of the three MS/MS methods; spectra in grey represent the energy level for a particular MS/MS mode that yields the best sequencing information; and (C) AA sequence coverage for each of the charge state and then combined.

FIG. 18 shows the experimental design for a multiple protease strategy to optimise shotgun proteomics.

FIG. 19 shows the LC-MS patterns of BSA. Graphical representations of elution time (min; y-axis) and mass (m/z; x-axis) for BSA digested with various proteases on their own or in combination. A graphical representation of the number of MS peaks (y-axis) observed using the various proteases on their own or in combination (x-axis; in triplicate) is provided in the bottom right-hand panel.

FIG. 20 is a graphical representation of MS peak statistics from BSA samples. Percentage of MS peaks that underwent MS/MS fragmentation (light grey bars), MS/MS spectra that were annotated in Mascot (black bars) and MS peaks that led to an identification in SEQUEST (dark grey bars) (%; left-hand y-axis) are shown relative to the protease digestion strategy (x-axis). The number of MS peaks obtained for each protease digestion strategy (right-hand y-axis) is also shown.

FIG. 21 shows the amino acid composition of BSA. (A) A graphical representation of the theoretical amino acid composition (x-axis) and abundance (%; y-axis) of BSA mature protein sequence using Expasy ProtParam. (B) A graphical representation of predicted (black bars) and observed (grey bars) cleavage sites (%; y-axis) for amino acids targeted by proteases (x-axis).

FIG. 22 shows that each protease on their own or combined yield high sequence coverage of BSA. (A) A graphical representation of PCA of the identified peptides. (B) A graphical representation of HCA of the identified peptides. (C) A schematic representation of the sequence alignment of identified peptides to the amino acid sequence of the mature BSA protein. (D) A graphical representation of the percentage sequence coverage (%; x-axis) achieved using the various proteases on their own or in combination (y-axis). (E) A graphical representation of the average mass (peptide mass, Da; y-axis) of identified proteins using the various proteases on their own or in combination (x-axis). (F) A graphical representation of the distribution of the number of identified peptides (y-axis) and the number of miscleavages that they contain (x-axis). Vertical bars denote standard deviation (SD). Downward arrowhead denotes the minimum peptide mass and upward arrowhead denotes the maximum peptide mass.

FIG. 23 is a graphical representation of the distribution of BSA peptides (y-axis) according to the number of miscleavages per digestion combination (x-axis).

FIG. 24 shows that the LC-MS patterns of cannabis are protein-rich and complex. Graphical representations of elution time (min; y-axis) and mass (m/z; x-axis) in cannabis-derived protein samples digested with various proteases on their own or in combination. A graphical representation of the number of MS peaks (y-axis) observed using the various proteases on their own or in combination (x-axis; in triplicate) is also provided in the bottom right-hand panel.

FIG. 25 shows that peptides isolated from cannabis can be grouped by digestion type. (A) A graphical representation of PCA projection of PC1 (x-axis) and PC2 (y-axis) for the 42 digest samples resulting from the action of one protease (T, G or C), or two (T->G, T->C, or G-C), or three proteases (T->G->C) applied sequentially. (B) A graphical representation of PCA loading of PC1 (x-axis) and PC2 (y-axis) for the 27,635 cannabis peptides identified and coloured according to their deconvoluted masses. (C) A graphical representation of PLS score of LV1 (x-axis) and LV2 (y-axis) featuring the 42 digest samples using the digestion type as a response. (D) A graphical representation of PLS loading of LV1 (x-axis) and LV2 (y-axis) featuring the 3,349 most significant peptides from the linear model testing the response to proteases, and coloured according to their retention time (min) and m/z values. T, trypsin; G, GluC; C, chymotrypsin; RT, retention time.

FIG. 26 is a graphical representation of MS peak statistics from medicinal cannabis samples. Percentage of MS peaks that underwent MS/MS fragmentation (light grey bars), MS/MS spectra that were annotated in Mascot (black bars) and MS peaks that led to an identification in SEQUEST (dark grey bars) (%; left-hand y-axis) are shown relative to the protease digestion strategy (x-axis). The number of MS peaks obtained for each protease digestion strategy (right-hand y-axis) is also shown.

FIG. 27 shows that each protease behaves differently when applied to cannabis-derived samples. (A) A graphical representation of the ion score (average score; y-axis) per amino acid residue targeted by the three proteases (x-axis). Maximum is represented by the triangles. Vertical bars denote SD. (B) A graphical representation of the distribution (occurrence; y-axis) of the number of missed cleavages (x-axis) per protease. (C) A graphical representation of the distribution of the average peptide mass (y-axis) of the cannabis peptides according to the number of missed cleavages (x-axis). Vertical bars denote SD. (D) A graphical representation of extreme peptide mass (y-axis) according to the number of missed cleavages (x-axis). Minimum peptide mass is represented as circles and maximum peptide mass is represented as triangles.

FIG. 28 shows the annotated MS/MS spectra of the illustrative example peptides from ribulose bisphosphate carboxylase large chain (RBCL, UniProtID A0A0C5B2I6). (A) Features of the peptides selected to illustrate MS/MS annotation. (B) Comparison of the same sequence area (peptide alignment provided) resulting from the action of GluC, chymotrypsin, trypsin/LysC proteases. (C) Example post-translational modification (PTM) annotation such as oxidation or phosphorylation.

FIG. 29 is a graphical representation of the pathways in which identified cannabis proteins are involved.

### DETAILED DESCRIPTION OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Unless specifically defined otherwise, all technical and scientific terms used herein shall be taken to have the same meaning as commonly understood by one of ordinary skill in the art.

Unless otherwise indicated the molecular biology, cell culture, laboratory, plant breeding and selection techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present); Janick, J. (2001) Plant Breeding Reviews, John Wiley & Sons, 252 p.; Jensen, N. F. ed. (1988) Plant Breeding Methodology, John Wiley & Sons, 676 p., Richard, A. J. ed. (1990) Plant Breeding Systems, Unwin Hyman, 529 p.; Walter, F. R. ed. (1987) Plant Breeding, Vol. I, Theory and Techniques, MacMillan Pub. Co.; Slavko, B. ed. (1990) Principles and Methods of Plant Breeding, Elsevier, 386 p.; and Allard, R. W. ed. (1999) Principles of Plant Breeding, John-Wiley & Sons, 240 p. The ICAC Recorder, Vol. XV no. 2: 3-14; all of which are incorporated by reference. The procedures described are believed to be well known in the art and are provided for the convenience of the reader. All other publications mentioned in this specification are also incorporated by reference in their entirety.

As used in the subject specification, the singular forms “a”, “an” and “the” include plural aspects unless the context clearly dictates otherwise. Thus, for example, reference to “a protein” includes a single protein, as well as two or more proteins; reference to “an apical bud” includes a single apical bud, as well as two or more apical buds; and so forth.

The present disclosure is predicated, at least in part, on the unexpected finding that an optimised protein extraction methods for cannabis bud and trichome material improves proteomic analysis of cannabis plant by enhancing the coverage of proteins of relevance to the biosynthesis of cannabinoids and terpenes that underpin the therapeutic value of medicinal cannabis.

Therefore, in an aspect disclosed herein, there is provided a method of extracting cannabis-derived proteins from cannabis plant material, the method comprising:
(a) suspending cannabis plant material in a solution comprising a charged chaotropic agent for a period of time to allow for extraction of cannabis-derived proteins into the solution; and
(b) separating the solution comprising the cannabis-derived proteins from residual plant material.

### Cannabis

As used herein, the term “cannabis plant” means a plant of the genus Cannabis, illustrative examples of which include Cannabis sativa, Cannabis indica and Cannabis ruderalis. Cannabis is an erect annual herb with a dioecious breeding system, although monoecious plants exist. Wild and cultivated forms of cannabis are morphologically variable, which has resulted in difficulty defining the taxonomic organisation of the genus. In an embodiment, the cannabis plant is C. sativa.

The terms “plant”, “cultivar”, “variety”, “strain” or “race” are used interchangeably herein to refer to a plant or a group of similar plants according to their structural features and performance (i.e., morphological and physiological characteristics).

The reference genome for C. sativa is the assembled draft genome and transcriptome of “Purple Kush” or “PK” (van Bakal et al. 2011, Genome Biology, 12:R102). C. sativa, has a diploid genome (2n=20) with a karyotype comprising nine autosomes and a pair of sex chromosomes (X and Y). Female plants are homogametic (XX) and males heterogametic (XY) with sex determination controlled by an X-to-autosome balance system. The estimated size of the haploid genome is 818 Mb for female plants and 843 Mb for male plants.

As used herein, the terms “plant material” or “cannabis plant material” are to be understood to mean any part of the cannabis plant, including the leaves, stems, roots, and buds, or parts thereof, as described elsewhere herein, as well as extracts, illustrative examples of which include kief or hash, which includes trichomes and glands. In a preferred embodiment, the plant material is an apical bud. In another preferred embodiment, the plant material comprises trichomes.

In an embodiment, the plant material is derived from a female cannabis plant. In another embodiment, the plant material is derived from a mature female cannabis plant.

### Cannabis-Derived Proteins

As used herein, the term “cannabis-derived protein” refers to any protein produced by a cannabis plant. Cannabis-derived proteins will be known to persons skilled in the art, illustrative examples of which include cannabinoids, terpenes, terpinoids, flavonoids, and phenolic compounds.

The term “cannabinoid”, as used herein, refers to a family of terpeno-phenolic compounds, of which more than 100 compounds are known to exist in nature. Cannabinoids will be known to persons skilled in the art, illustrative examples of which are provided in Table 1, below, including acidic and decarboxylated forms thereof.

TABLE 1
Cannabinoids and their properties.

| | | Chemical |
|---|---|---|
| | | properties/ |
| | | [M + H]⁺ ESI |
| Name | Structure | MS |
| Δ9-tetrahydrocannabinol (THC) | 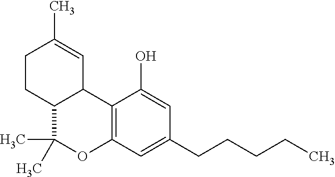 | Psychoactive, decarboxylation product of THCA m/z 315.2319 |
| Δ9- tetrahydrocannabinolic acid (THCA/THCA-A) | 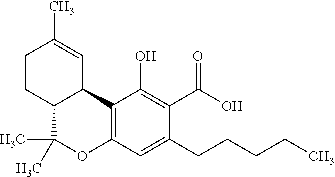 [SMILES: CCCCCc1cc2c(c(O)c1C(=O)O)[C@@H]1C=C(C)CC[C@H]1C(C)(C)O2] | m/z 359.2217 |
| cannabidiol (CBD) | 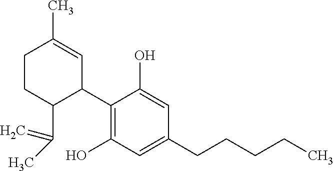 [SMILES: C=C(C)C1CCC(C)=CC1c1c(O)cc(CCCCC)cc1O] | decarboxylation product of CBDA m/z 315.2319 |
| cannabidiolic acid (CBDA) | 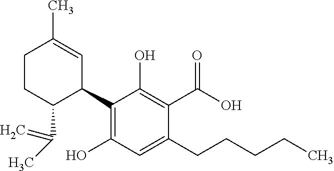 [SMILES: C=C(C)[C@@H]1CCC(C)=C[C@H]1c1c(O)cc(CCCCC)c(C(=O)O)c1O] | m/z 359.2217 |
| cannabigerol (CBG) | 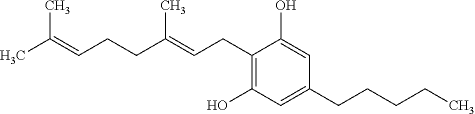 [SMILES: CCCCCc1cc(O)c(C/C=C(\C)CCC=C(C)C)c(O)c1] | Non- intoxicating, decarboxylation product of CBGA m/z 317.2475 |
| cannabigerolic acid (CBGA) | 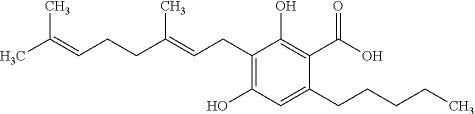 [SMILES: CCCCCc1cc(O)c(C/C=C(\C)CCC=C(C)C)c(O)c1C(=O)O] | m/z 361.2373 |
| cannabichromene (CBC) | 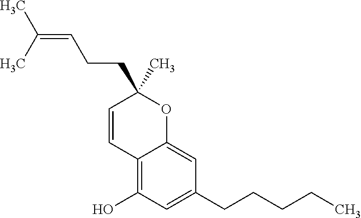 [SMILES: CCCCCc1cc(O)c2c(c1)O[C@](C)(CCC=C(C)C)C=C2] | Non- psychotropic, converts to cannabicyclol upon light exposure m/z 315.2319 |
| cannabichromene acid (CBCA) | 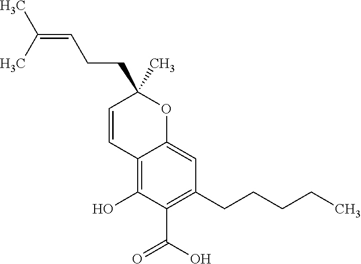 [SMILES: CCCCCc1cc2c(c(O)c1C(=O)O)C=C[C@@](C)(CCC=C(C)C)O2] | m/z 359.2217 |
| cannabicyclol (CBL) | 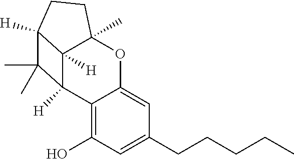 | Non- psychoactive, 16 isomers known. Derived from non-enzymatic conversion of CBC m/z 315.2319 |
| cannabinol (CBN) | 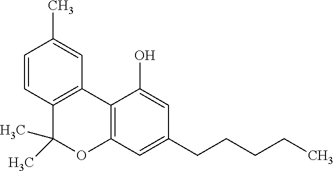 [SMILES: CCCCCc1cc(O)c2c(c1)OC(C)(C)c1ccc(C)cc1-2] | Likely degradation product of THC m/z 311.2006 |
| cannabinolic acid (CBNA) | 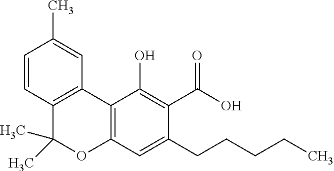 [SMILES: CCCCCc1cc2c(c(O)c1C(=O)O)-c1cc(C)ccc1C(C)(C)O2] | m/z 355.1904 |
| tetrahydrocannabivarin (THCV) | 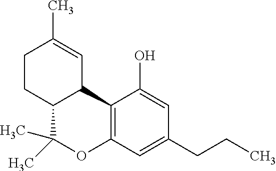 | decarboxylation product of THCVA m/z 287.2006 |
| tetrahydrocannabivarinic acid (THCVA) | 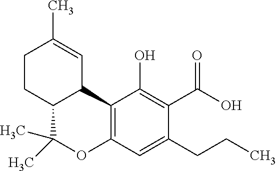 | m/z 331.1904 |
| cannabidivarin (CBDV) | 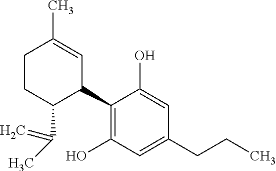 | m/z 287.2006 |
| cannabidivarinic acid (CBDVA) | 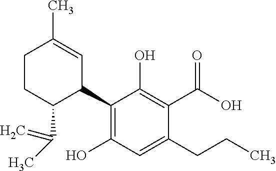 | m/z 331.1904 |
| Δ8-tetrahydrocannabinol (d8-THC) | 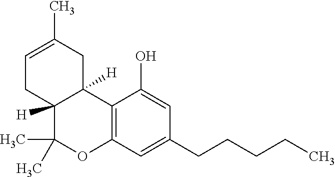 [SMILES: CCCCCc1cc(O)c2c(c1)OC(C)(C)[C@@H]1CC=C(C)C[C@@H]21] | m/z 315.2319 |

Cannabinoids are synthesised in cannabis plants as carboxylic acids. Acid forms of cannabinoids will be known to persons skilled in the art, illustrative examples of which are described in Papaset et al. (Int. J. Med. Sci., 2018; 15(12): 1286-1295) and Cannabis and Cannabinoids (PDQ®): Health Professional Version; PDQ Integrative, Alternative, and Complementary Therapies Editorial Board; Bethesda (Md.): National Cancer Institute (US); 2002-2018).

The precursors of cannabinoids originate from two distinct biosynthetic pathways: the polyketide pathway, giving rise to olivetolic acid (OLA) and the plastidal 2-C-methyl-D-erythritol 4-phosphate (MEP) pathway, leading to the synthesis of geranyl diphosphate (GPP). OLA is formed from hexanoyl-CoA, derived from the short-chain fatty acid hexanoate, by aldol condensation with three molecules of malonyl-CoA. This reaction is catalysed by a polyketide synthase (PKS) enzyme and an olivetolic acid cyclase (OAC). The geranylpyrophosphate:olivetolate geranyltransferase catalyses the alkylation of OLA with GPP leading to the formation of CBGA, the central precursor of various cannabinoids. Three oxidocyclases are responsible for the diversity of cannabinoids: THCA synthase (THCAS) converts CBGA to THCA, while CBDA synthase (CBDAS) forms CBDA, and CBCA synthase (CBCAS) produces CBCA. Propyl cannabinoids (cannabinoids with a C3 side-chain, instead of a C5 side-chain), such as tetrahydrocannabivarinic acid (THCVA), are synthetised from a divarinolic acid precursor.

“Δ-9-tetrahydrocannabinolic acid” or “THCA-A” is synthesised from the CBGA precursor by THCA synthase. The neutral form “Δ-9-tetrahydrocannabinol” or “THC” is associated with psychoactive effects of cannabis, which are primarily mediated by its activation of CB1G-protein coupled receptors, which result in a decrease in the concentration of cyclic AMP (cAMP) through the inhibition of adenylate cyclase. THC also exhibits partial agonist activity at the cannabinoid receptors CB1 and CB2. CB1 is mainly associated with the central nervous system, while CB2 is expressed predominantly in the cells of the immune system. As a result, THC is also associated with pain relief, relaxation, fatigue, appetite stimulation, and alteration of the visual, auditory and olfactory senses. Furthermore, more recent studies have indicated that THC mediates an anti-cholinesterase action, which may suggest its use for the treatment of Alzheimer's disease and myasthenia (Eubanks et al., 2006, Molecular Pharmaceuticals, 3(6): 773-7).

“Cannabidiolic acid” or “CBDA” is also a derivative of cannabigerolic acid (CBGA), which is converted to CBDA by CBDA synthase. Its neutral form, “cannabidiol” or “CBD” has antagonist activity on agonists of the CB1 and CB2 receptors. CBD has also been shown to act as an antagonist of the putative cannabinoid receptor, GPR55. CBD is commonly associated with therapeutic or medicinal effects of cannabis and has been suggested for use as a sedative, anti-inflammatory, anti-anxiety, anti-nausea, atypical anti-psychotic, and as a cancer treatment. CBD can also increase alertness, and attenuate the memory impairing effect of THC.

The terms “terpene” and “terpenoids” as used herein, refer to a family of non-aromatic compounds that are typically found as components of essential oil present in many plants. Terpenes contain a carbon and hydrogen scaffold, while terpenoids contain a carbon, hydrogen and oxygen scaffold. Terpenes and terpenoids will be known to persons skilled in the art, illustrative examples of which include α-pinene, α-bisabolol, β-pinene, guaiene, guaiol, limonene, myrcene, ocimene, α-mumulene, terpinolene, 3-carene, myercene, α-terpineol and linalool.

Terpenes are classified according to the number of repeating units of 5-carbon building blocks (isoprene units), such as monoterpenes with 10 carbons, sesquiterpenes with 15 carbons, and triterpenes derived from a 30-carbon skeleton. Terpene yield and distribution in the plant vary according to numerous parameters, such as processes for obtaining essential oil, environmental conditions, or maturity of the plant. Mono- and sesqui-terpenes have been detected in flowers, roots, and leaves of cannabis, while triterpenes have been detected in hemp roots, fibers and in hempseed oil.

Two different biosynthetic pathways contribute, in their early steps, to the synthesis of plant-derived terpenes. The cytosolic mevalonic acid (MVA) pathway is involved in the biosynthesis of sesqui-, and tri-terpenes, and the plastid-localized MEP pathway contributes to the synthesis of mono-, di-, and tetraterpenes. MVA and MEP are produced through various and distinct steps, from two molecules of acetyl-coenzyme A and from pyruvate and D-glyceraldehyde-3-phosphate, respectively. They are further converted to isopentenyl diphosphate (IPP) and isomerised to dimethylallyl diphosphate (DMAPP), the end point of the MVA and MEP pathways. In the cytosol, two molecules of IPP (C5) and one molecule of DMAPP (C5) are condensed to produce farnesyl diphosphate (FPP, C15) by farnesyl diphosphate synthase (FPS). FPP serves as a precursor for sesquiterpenes (C15), which are formed by terpene synthases and can be decorated by other various enzymes. Two FPP molecules are condensed by squalene synthase (SQS) at the endoplasmic reticulum to produce squalene (C30), the precursor for triterpenes and sterols, which are generated by oxidosqualene cyclases (OSC) and are modified by various tailoring enzymes. In the plastid, one molecule of IPP and one molecule of DMAPP are condensed to form GPP (C10) by GPP synthase (GPS). GPP is the immediate precursor for monoterpenes.

The term “chemotype”, as used herein, refers to a representation of the type, amount, level, ratio and/or proportion of cannabis-derived proteins that are present in the cannabis plant or part thereof, as typically measured within plant material derived from the plant or plant part, including an extract therefrom.

The chemotype of a cannabis plant typically predominantly comprises the acidic form of the cannabinoids, but may also comprise some decarboxylated (neutral) forms thereof, at various concentrations or levels at any given time (e.g., at propagation, growth, harvest, drying, curing, etc.) together with other cannabis-derived proteins such as terpenes, flavonoids and phenolic compounds.

The terms “level”, “content”, “concentration” and the like, are used interchangeably herein to describe an amount of the cannabis-derived protein, and may be represented in absolute terms (e.g., mg/g, mg/ml, etc.) or in relative terms, such as a ratio to any or all of the other proteins in the cannabis plant material or as a percentage of the amount (e.g., by weight) of any or all of the other proteins in the cannabis plant material.

As noted elsewhere herein, cannabinoids are synthesised in cannabis plants predominantly in acid form (i.e., as carboxylic acids). While some decarboxylation may occur in the plant, decarboxylation typically occurs post-harvest and is increased by exposing the plant material to heat.

### Protein Extraction

Protein extraction methods are typically optimised based on the intended use of the extract, such as whether the extract is to be further processed to isolate specific constituents, produce an enriched extract or for use in proteomic analysis. For example, methods for the extraction of specific constituents of plant material may include steps such as maceration, decotion, and extraction with aqueous and non-aqueous solvents, distillation and sublimation. By contrast, methods for the extraction of plant-derived proteins for proteomic analysis desirably require the preservation of proteins and peptides, including post-translational modifications, hydrophobic membrane proteins and low-abundance proteins. Such methods typically include steps such as the homogenisation, cell lysis, solubilisation, precipitation, separation, enrichment, etc., depending on the starting material and downstream analysis method.

In an embodiment, the methods described herein comprise suspending cannabis plant material in a solution comprising a charged chaotropic agent for a period of time to allow for extraction of cannabis-derived proteins into the solution.

The term “chaotropic agent” as used herein refers to a substance that disrupts the structure of proteins to enable proteins to unfold with all ionisable groups exposed to solution. Chaotropic agents are used during the sample solubilisation process to break down interactions involved in protein aggregation (e.g., disulphide/hydrogen bonds, van der Waals forces, ionic and hydrophobic interactions) to enable the disruption of proteins into a solution of individual polypeptides, thereby promoting their solubilisation. Suitable chaotropic agents would be known to persons skilled in the art, illustrative examples of which include n-butanol, ethanol, guanidine hydrochloride, guanidine isothiocyanate, lithium perchlorate, lithium acetate, magnesium chloride, phenol, 2-propanol, sodium dodecyl sulphate, thiourea and urea.

In an embodiment, the chaotropic agent is a charged chaotropic agent selected from the group consisting of guanidine hydrochloride, guanidine isothiocyanate. In another embodiment, the charged chaotropic agent is guanidine hydrochloride.

In an embodiment, the solution comprises from about 5.5M to about 6.5M, preferably about 5.6 M to about 6.5 M, preferably about 5.7 M to about 6.5M, preferably about 5.8M to about 6.5M, preferably about 5.9M to about 6.5M, preferably about 6.0M to about 6.5M, preferably about 5.5M to about 6.4M, preferably about 5.5M to about 6.3M, preferably about 5.5M to about 6.2M, preferably about 5.5M to about 6.1M, preferably about 5.5M to about 6.0M, or more preferably about 6.0M guanidine hydrochloride.

In an embodiment, the solution further comprises a reducing agent.

The terms “reducing agent” and “reductant” may be used interchangeably herein to refer to substances that disrupt disulphide bonds between cysteine residues, thereby promoting unfolding of proteins to enable analysis of single subunits of proteins. Suitable reducing agents would be known to persons skilled in the art, illustrative examples of which include dithiothreitol (DTT) and dithioerythritol (DTE).

In an embodiment, the reducing agent is DTT.

In an embodiment, the solution comprises from about 5 mM to about 20 mM, preferably about 5 mM to about 19 mM, about 5 mM to about 18 mM, about 5 mM to about 17 mM, about 5 mM to about 16 mM, about 5 mM to about 15 mM, about 5 mM to about 14 mM, about 5 mM to about 13 mM, about 5 mM to about 12 mM, about 5 mM to about 11 mM, about 5 mM to about 10 mM, about 6 mM to about 20 mM, about 7 mM to about 20 mM, about 8 mM to about 20 mM, about 9 mM to about 20 mM, about 10 mM to about 20 mM, or more preferably about 10 mM DTT.

In an embodiment, the cannabis plant material is pre-treated with an organic solvent before step (a) for a period of time to precipitate the cannabis-derived proteins.

Protein precipitation followed by resuspension in sample solution is commonly used to remove contaminants such as salts, lipids, polysaccharides, detergents, nucleic acids, etc. thereby promoting unfolding of proteins to enable analysis of single subunits of proteins. Suitable protein precipitation agents and methods would be known to persons skilled in the art, illustrative examples of which include precipitation with organic solvents such as trichloroacetic acid, acetone, chloroform, methanol, ammonium sulphate, ethanol, isopropanol, diethylether, polyethylene glycol or combinations thereof.

In an embodiment, the organic solvent is selected from the group consisting of trichloroacetic acid (TCA)/acetone and TCA/ethanol.

In an embodiment, the organic solvent comprises from about 5% to about 20%, preferably about 5% to about 19%, about 5% to about 18%, about 5% to about 17%, about 5% to about 16%, about 5% to about 15%, about 5% to about 14%, about 5% to about 13%, about 5% to about 12%, about 5% to about 11%, about 5% to about 10%, about 6% to about 20%, about 7% to about 20%, about 8% to about 20%, about 9% to about 20%, about 10% to about 20%, or more preferably about 10% TCA/acetone or TCA/ethanol.

In an embodiment, the cannabis-derived proteins separated by step (b), as described elsewhere herein, are subsequently digested by a protease in preparation for proteomic analysis.

The process of protein digestion is an important step in the preparation of samples for bottom-up proteomic analysis (also referred to as “shotgun” proteomics), as described elsewhere herein. The process of protein digestion is also an important step in the preparation of samples for middle-down proteomic analysis, as described elsewhere herein. The digestion of proteins into peptides by a protease facilitates protein identification using proteomic techniques and allows coverage of proteins that would be problematic due to, for example, poor solubility and heterogeneity.

The term “protease” as used herein refers to an enzyme that catabolise protein by hydrolysis of peptide bonds. Suitable proteases would be known to persons skilled in the art, illustrative examples of which include trypsin, trypsin/LysC, chymotrypsin, GluC, pepsin, Proteinase K, enterokinase, ficin, papain and bromelain.

As described elsewhere herein, the use of multiple proteases of various specificity can result in higher coverage of amino acid sequences. In particular, the generation of peptides using multiple proteases can increase the resolution of bottom-up and middle-down proteomic analysis to enable discrimination between closely related protein isoforms and detection of various post-translational modification (PTM) sites.

Thus, in an embodiment, the cannabis-derived proteins separated by step (b) are digested by two or more proteases, preferably two or more proteases, preferably three or more proteases, preferably four or more proteases, or more preferably five or more proteases.

In an embodiment, the two or more proteases comprise orthogonal proteases.

In accordance with the methods disclosed herein, the cannabis-derived proteins separated by step (b) may be digested by the two or more proteases sequentially or simultaneously, as part of the same digestion or as separate digestions (e.g., single-, double-, and triple-digests).

In an embodiment, the cannabis-derived proteins separated by step (b) are digested by the two or more proteases sequentially.

By “sequentially” it is meant that there is an interval between digestion with a first protease and digestion with a second protease. The interval between the sequential digestions may be seconds, minutes, hours, or days. In a preferred embodiment, the interval between sequential protease digestions is at least 18 hours (i.e., overnight). The sequential digestions may be in any order.

In an embodiment, the cannabis-derived proteins separated by step (b) are digested by trypsin/LysC followed by GluC (“T→G”).

In an embodiment, the cannabis-derived proteins separated by step (b) are digested by trypsin/LysC followed by chymotrypsin (“T→C”).

In an embodiment, the cannabis-derived proteins separated by step (b) are digested by GluC followed by chymotrypsin (“G→C”).

In an embodiment, the cannabis-derived proteins separated by step (b) are digested by trypsin/LysC followed by GluC followed by chymotrypsin (“T→G→C”).

In an embodiment, the cannabis-derived proteins separated by step (b) are digested by the two or more proteases simultaneously (i.e., multiple proteases in a single digest).

In an embodiment, the cannabis-derived proteins separated by step (b) are digested by trypsin/LysC and GluC simultaneously (“T:G”).

In an embodiment, the cannabis-derived proteins separated by step (b) are digested by trypsin/LysC and chymotrypsin simultaneously (“T:C”).

In an embodiment, the cannabis-derived proteins separated by step (b) are digested by GluC digest and chymotrypsin simultaneously (“G:C”).

In an embodiment, the cannabis-derived proteins separated by step (b) are digested by trypsin/LysC, GluC and chymotrypsin simultaneously (“T:G:C”).

The skilled person would appreciate that the amounts of each protease used simultaneously may vary according to the intended use of the digested protein sample (i.e., incomplete digestion for middle-down proteomics). In a preferred embodiment, however, the same volume of each protease is applied to the the cannabis-derived proteins separated by step (c).

In an embodiment, the protease is selected from the group consisting of trypsin, trypsin/LysC, chymotrypsin, GluC and pepsin. In another embodiment, the protease is selected from the group consisting of trypsin/LysC, chymotrypsin and GluC.

In yet another embodiment, the protease is trypsin/LysC.

In an embodiment, the cannabis-derived proteins separated by step (b), as described elsewhere herein, are subsequently alkylated in preparation for proteomic analysis.

The process of alkylation is typically desirable in the preparation of samples for top-down proteomic analysis, as described elsewhere herein. The alkylation of protein thiols reduces disulphide bonds and generally improves the resolution of proteomic techniques by reducing, for example, the generation of artefacts from disulphide-bonded dipeptides that are not selected and fragmented.

Reagents for the alkylation of proteins would be known to persons skilled in the art, illustrative examples of which include iodoacetamide (IAA), iodoacetic acid, acrylamide monomers and 4-vinylpyridine.

In an embodiment, the cannabis-derived proteins separated by step (b) are alkylated by IAA.

In another aspect, there is provided a method of extracting cannabis-derived proteins from cannabis plant material, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent for a period of time to allow for extraction of cannabis-derived proteins into the solution; and
(c) separating the solution comprising the cannabis-derived proteins from residual plant material.

### Proteomic Analysis and Sample Preparation

The methods disclosed herein may also suitably be used to prepare a sample for proteomic analysis that will enhance coverage of proteins of relevance to the biosynthesis of cannabis-derived proteins of therapeutic value (e.g., cannabinoids and terpenes). The advantageously allows for the improvement of genome annotation and genomic selective breeding strategies to enable the production of cannabis plants with desirable chemotype(s).

Thus, in an aspect disclosed herein, there is provided a method of preparing a sample of cannabis-derived proteins from cannabis plant material for proteomic analysis, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent from a period of time to allow for extraction of cannabis-derived proteins into the solution;
(c) separating the solution comprising the cannabis-derived proteins from residual plant material; and
(d) digesting the solution of (c) with a protease.

In an embodiment, step (d) comprises digesting the solution of (c) with two or more proteases.

In another aspect disclosed herein, there is provided a method of preparing a sample of cannabis-derived proteins from cannabis plant material for proteomic analysis, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent from a period of time to allow for extraction of cannabis-derived proteins into the solution; and
(c) separating the solution comprising the cannabis-derived proteins from residual plant material.

In an embodiment, the charged chaotropic acid is guanidine hydrochloride.

Proteomic analysis methods would be known to persons skilled in the art, illustrative examples of which include two-dimensional gel electrophoresis (2DE), capillary electrophoresis, capillary isoelectric focusing, Fourier-transform mass spectrometry (FT-MS), liquid chromatography-mass spectrometry (LC-MS), isotope coded affinity tag (ICAT) analysis, ultra-performance LC-MS (UPLC-MS), nano liquid chromatography-tandem mass spectrometry (nLC-MS/MS), MALDI-MS, SELDI, and electrospray ionisation.

In an embodiment, the proteomic analysis method is selected from the group consisting of LC-MS, UPLC-MS and nLC-MS/MS.

LC-based proteomic methods may be used for top-down, middle-down and bottom-up proteomics methods, as described elsewhere herein.

The term “top-down proteomics” as used herein refers to a proteomic method where a protein sample is separated and then individual, intact proteins are identified directly by means of tandem mass spectrometry. Using this approach, liquid chromatography may be used for separation of proteins prior to mass spectrometry analysis. Persons skilled in the art would be aware of suitable top-down proteomic approaches, illustrative embodiments of which include the methods of Wang et al. (2005, Journal of Chromatography A, 1073(1-2): 35-41) and Moritz et al. (2005, Proteomics 5, 3402: 1746-1757).

The term “bottom-up proteomics” or “shotgun proteomics” as used herein refers to a proteomic method where a protein, or protein mixture is digested. Single- or multidimensional liquid chromatography coupled to mass spectrometry is then used for separation of peptide mixtures and identification of their compounds. Persons skilled in the art would be aware of suitable bottom-up proteomic approaches, illustrative embodiments of which include the method of Rappsilber et al. (2003, Analytical Chemistry, 75(3): 663-670).

The term “middle-down proteomics”, as used herein, refers to a hybrid technique that incorporates aspects of both top-down and bottom-up proteomics approaches. While top-down proteomics typically explores intact proteins of about 10-30 kDa and trypsin-based bottom-up proteomics generally yields short peptides of about 0.7-3 kDa, middle-down proteomics is used to analyse peptide fragments of about 3-10 kDa. Middle-down proteomics can be achieved by, for example, performing limited proteolysis through reduced incubation times and/or increased protease:proteins ratio to achieve partial digestion, or by using proteases with greater specificity and/or lesser efficiency, which cleave less frequently. Persons skilled in the art would be aware of suitable middle-down proteomics approaches, an illustrative example of which is described by Pandeswaria and Sabareesh (2019, RSC Advances, 9: 313-344).

In another aspect disclosed herein, there is provided a method of analysing a cannabis plant proteome, the method comprising:
(a) preparing a sample of cannabis-derived proteins in accordance with the methods described herein; and
(b) subjecting the sample to proteomic analysis.

The skilled person will appreciate that when a sample of cannabis-derived proteins is digested using one, two, three or more proteases, proteolysis is often incomplete, and non-standard protease cleavages (i.e., miscleavages) can occur.

Number of miscleavages is commonly used in proteomics analysis to discriminate between correct and incorrect matches based upon the protease used. For example, up to four miscleavages are recommended for chymotrypsin and GluC, and other two for trypsin (see, e.g., Giansanti et al., 2016, Nature Protocols, 11: 993-1006).

In an embodiment, the proteomic analysis comprises a parameter setting the maximum number of missed cleavages to between about 2 and about 10. In another embodiment, the proteomic analysis comprises a parameter setting the maximum number of missed cleavages to between about 6 and about 10.

In an embodiment, the method of analysing a cannabis plant proteome comprises subjecting the sample to a first proteomic analysis, followed by one or more additional proteomic analyses (i.e., re-analysis of the sample). The re-analysis of the sample may deepen the proteome analysis and increase the proportion of annotated MS/MS spectra (i.e., successful hits), as described elsewhere herein. Such re-analysis may be achieved using iterative exclusion lists from the precursor ions already fragmented.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

### EXAMPLES

### Materials and Methods

### Plant Materials

### Apical Bud Sampling and Grinding

Fresh plant material was obtained from the Victorian Government Medicinal Cannabis Cultivation Facility. The top three centimetres of the apical bud was excised using secateurs, placed into a labelled paper bag, snap frozen in liquid nitrogen and stored at −80° C. until grinding. Samples were collected in triplicates. Frozen buds were ground in liquid nitrogen using a mortar and pestle. The ground frozen powder was transferred into a 15 mL tube and stored at stored at −80° C. until protein extraction.

### Trichome Recovery

The top three centimetres of the apical bud was cut using secateurs and placed into a labelled paper bag. Samples were collected in triplicates. Trichome recovery was performed using the procedure of Yerger et al. (1992, Plant Physiology, 99: 1-7), with modifications. The bud was further trimmed with the secateurs into smaller pieces and placed into a 50 mL tube. Approximately 10 mL liquid nitrogen was added to the tube and the cap was loosely attached. The tube was then vortexed for 1 min. The cap was removed, and the content of the tube was discarded by inverting the tube and tapping it on the bench, while the trichomes stuck to the walls of the tube. The process was repeated in the same tube until all the apical bud was trimmed. Tubes were stored at −80° C. until protein extraction.

### Protein Extraction Methods

For the apical bud extraction, one 50 mg scoop of ground frozen powder was transferred into a 2 mL microtube kept on ice pre-filled with 1.8 mL precipitant or 0.5 mL resuspension buffer depending on the extraction method employed, as described elsewhere herein. All six extraction methods described hereafter were applied to the apical bud samples. For the trichome extraction, all trichomes stuck to the walls of the tubes were resuspended into the solutions and volumes specified below. Due the limited amount of trichomes recovered, only extraction methods 1 and 2 were attempted.

### Extraction 1: Resuspension in Urea Buffer

Plant material was resuspended in 0.5 mL of urea buffer (6M urea, 10 mM DTT, 10 mM Tris-HCl pH 8.0, 75 mM NaCl, and 0.05% SDS). The tubes were vortexed for 1 min, sonicated for 5 min, vortexed again for 1 min. The tubes were centrifuged for 10 min at 13,500 rpm. The supernatant was transferred into fresh 1.5 mL tubes and stored at −80° C. until protein assay.

### Extraction 2: Resuspension in Guanidine-Hydrochloride Buffer

Plant material was resuspended in 0.5 mL of guanidine-HCl buffer (6M guanidine-HCl, 10 mM DTT, 5.37 mM sodium citrate tribasic dihydrate, and 0.1 M Bis-Tris). The tubes were vortexed for 1 min, sonicated for 5 min, vortexed again for 1 min. The tubes were centrifuged for 10 min at 13,500 rpm and at 4° C. The supernatant was transferred into fresh 1.5 mL tubes and stored at −80C until protein assay.

### Extraction 3: TCA/Acetone Precipitation Followed by Resuspension in Urea Buffer

Plant material was resuspended in 1.8 mL ice-cold 10% TCA/10 mM DTT/acetone (w/w/v) by vortexing for 1 min. Tubes were left at −20° C. overnight. The next day, tubes were centrifuged for 10 min at 13,500 rpm and at 4° C. The supernatant was removed, and the pellet was resuspended in ice-cold 10 mM DTT/acetone (w/v) by vortexing for 1 min. Tubes were left at −20° C. for 2 h. The tubes were centrifuged as specified before and the supernatant removed. This washing step of the pellet was repeated once more. The pellets were dried for 30 min under a fume hood. The dry pellet resuspended in 0.5 mL of urea buffer as described in Extraction 1.

### Extraction 4: TCA/Acetone Precipitation Followed by Resuspension in Guanidine-Hydrochloride Buffer

Plant material was processed as detailed in Extraction 3, except that the dry pellet was resuspended in 0.5 mL of guanidine-HCl buffer.

### Extraction 5: TCA/Ethanol Precipitation Followed by Resuspension in Urea Buffer

Plant material was processed as detailed in Extraction 3, except that acetone was replaced with ethanol.

### Extraction 6: TCA/Ethanol Precipitation Followed by Resuspension in Guanidine-Hydrochloride Buffer

Plant material was processed as detailed in Extraction 4, except that acetone was replaced with ethanol.

### Protein Assay

Protein extracts from apical buds were diluted ten times into their respective resuspension buffer and protein extracts from trichomes were diluted four times. The protein concentrations were measured in triplicates using the Microplate BCA protein assay kit (Pierce) following the manufacturer's instructions. Bovine Serum Albumin (BSA) was used a standard.

### Trypsin/LysC Protein Digestion and Desalting

### Protease Digestion

An aliquot corresponding to 100 μg of plant proteins was used for protein digestion as follows. The DTT-reduced and IAA-alkylated proteins were diluted six times using 50 mM Tris-HCl pH 8 to drop the resuspension buffer molarity below 1 M. Trypsin/LysC protease (Mass Spectrometry Grade, 100 μg, Promega) was carefully solubilised in 1 mL of 50 mM Tris-HCl pH 8. A 40 μL aliquot of trypsin/LysC solution was added and gently mixed with the plant extracts thus achieving a 1:25 ratio of protease:plant proteins. The mixture was left to incubate overnight (19 h) at 37° C. in the dark. The digestion reaction was stopped by lowering the pH of the mixture using a 10% formic acid (FA) in H₂O (v/v) to a final concentration of 1% FA.

Bovine serum albumin (BSA) was also digested under the same conditions to be used as a control for digestion and nLC-MS/MS analysis.

### Desalting

The 25 tryptic digests were desalted using solid phase extraction (SPE) cartridges (Sep-Pak C18 1 cc Vac Cartridge, 50 mg sorbent, 55-105 μm particle size, 1 mL, Waters) by gravity as described in (Vincent et al. 2015, 2015, Frontiers in Genetics, 6: 360).

A 90 μL aliquot of peptide digest was mixed with 10 μL 1 ng/μL Glu-Fibrinopeptide B (Sigma), as an internal standard. The peptide/internal standard mixture was transferred into a 100 μL glass insert placed into a glass vial. The vials were positioned into the autosampler at 4° C. for immediate analyses by nLC-MS/MS.

### Intact Protein Analysis by Ultra Performance Liquid Chromatography Mass Spectrometry (UPLC-MS)

### UPLC Separation

The UPLC-MS analyses of the 24 plant protein extracts were performed in duplicates for a total of 48 MS files. Protein extracts were chromatographically separated using the UHPLC 1290 Infinity Binary LC system (Agilent) and a Aeris™ WIDEPORE XB-C8 column (Phenomenex) kept at 75° C. as described in Vincent et al. (2016, PLoS One, 11: e0163471). Mobile phase A contained 0.1% formic acid in water and mobile phase B contained 0.1% formic acid in acetonitrile. UPLC gradient was as follows: starting conditions 3% B, held for 2.5 min, ramping to 60% B in 27.5 min, ramping to 99% B in 1 min and held at 99% B for 4 min, lowering to 3% B in 0.1 min, equilibration at 3% B for 4.9 min. A 10 uL injection volume was applied to each protein extract, irrespective of their protein concentration. Each extract was injected twice.

### MS Acquisition

During the 40 min chromatographic separation, plant intact proteins were analysed using an Orbitrap Velos hybrid ion trap-Orbitrap mass spectrometer (ThermoFisher Scientific) online with the UPLC and fitted with a heated electrospray ionisation (HESI) source. HESI parameters were: capillary heated to 300° C., source heated to 250° C., sheath gas flow 30, auxiliary gas flow 10, sweep gas flow 2, 3.6 kV, 100 μL, and S-Lens RF level 60%. SID was set at 15V.

For the first 2.5 min, nLC flow was sent to waste, then switched to source from 2.5 to 38 min, and finally switched back to waste for the last minute of the 40 min run. Spectra were acquired in positive ion mode using the full MS scan mode of the Fourier Transform (FT) Orbitrap mass analyser at a resolution of 60,000 using a 500-2000 m/z mass window and 6 microscans. FT Penning gauge difference was set at 0.05 E-10 Torr.

All LC-MS files will be available from the stable public repository MassIVE at the following URL: http://massive.ucsd.edu/ProteoSAFe/datasets.jsp with the accession number MSV000083191.

Peptide Analysis by Nano Liquid Chromatography-Tandem Mass Spectrometry (nLC-MS/MS)

The nLC-ESI-MS/MS analyses were performed on 25 peptide digests in duplicates thus yielding 50 MS/MS files. Chromatographic separation of the peptides was performed by reverse phase (RP) using an Ultimate 3000 RSLCnano System (Dionex) online with an Orbitrap Velos hybrid ion trap-Orbitrap mass spectrometer (ThermoFisher Scientific). The parameters for nLC and MS/MS have been described in Vincent et al., supra. Each digest was injected twice. Blanks (1 μL of mobile phase A) were injected in between each set of six extraction replicates and analysed over a 20 min nLC run to minimise carry-over.

### Database Search for Protein Identification

Database searching of the 50 MS .RAW files was performed in Proteome Discoverer (PD) 1.4 using MASCOT 2.6.1. All 589 C. sativa protein sequences publicly available on 13 Dec. 2018 from UniprotKB (www.uniprot.org; key word used “Cannabis sativa”) were downloaded as a FASTA file. These also included 77 sequences from the European hop, Humulus lupulus, the closest relative to C. sativa, as well as 72 sequences from the Chinese grass, Boehmeria nivea, which also closely related to C. sativa. The GOT sequence was retrieved from WO 2011/017798 A1 and included in the FASTA file (590 entries). The FASTA file was imported and indexed in PD 1.4. The SEQUEST algorithm was used to search the indexed FASTA file. The database searching parameters specified trypsin as the digestion enzyme and allowed for up to two missed cleavages. The precursor mass tolerance was set at 10 ppm, and fragment mass tolerance set at 0.5 Da. Peptide absolute Xcorr threshold was set at 0.4 and protein relevance threshold was set at 1.5. Carbamidomethylation (C) was set as a static modification. Oxidation (M), phosphorylation (STY), conversion from Gln to pyro-Glu (N-term Q) and Glu to pyro-Glu (N-term E), and deamination (NQ) were set as dynamic modifications. The target decoy peptide-spectrum match (PSM) validator was used to estimate false discovery rates (FDR). At the peptide level, peptide confidence value set at high was used to filter the peptide identification, and the corresponding FDR on peptide level was less than 1%. At the protein level, protein grouping was enabled.

All nLC-MS/MS files will be available from the stable public repository MassIVE at the following URL: http://massive.ucsd.edu/ProteoSAFe/datasets.jsp with the accession number MSV000083191.

### Data Processing and Statistical Analyses

The data files obtained following UPLC-MS analysis were processed in the Refiner MS module of Genedata Expressionist® 11.0 with the following parameters: 1/RT Structure Removal using a 5 scan minimum RT length, 2/m/z Structure Removal using 8 points minimum m/z length, 3/Chromatogram Chemical Noise Reduction using 7 scan smoothing, and a moving average estimator, 4/Spectrum Smoothing using a Savitzky-Golay algorithm with 5 points m/z window and a polynomial order of 3, 5/Chromatogram RT Alignment using a pairwise alignment-based tree and 50 RT scan search interval, 6/Chromatogram Peak Detection using a 0.3 min minimum peak size, 0.02 Da maximum merge distance, a boundaries merge strategy, a 30% gap/peak ratio, a curvature-based algorithm, using both local maximum and inflection points to determine boundaries, 7/Chromatogram Isotope Clustering using a 4 scan RT tolerance, a 20 ppm m/z tolerance, a peptide isotope shaping method with protonation, charges from 2-25, mono-isotopic masses and variable charge dependency, 8/Singleton Filter, 9/Charge and Adduct Grouping (i.e., deconvolution) using a 50 ppm mass tolerance, a 0.1 min RT tolerance, a dynamic adduct list containing ions (H), and neutrals (—H₂O, K—H, and Na—H), 10/Export Analyst using group volumes.

The data files obtained following nLC-MS/MS analysis were processed in the Refiner MS module of Genedata Expressionist® 11.0 with the following parameters: 1/RT Structure Removal applying a minimum of 4 scans, 2/m/z Structure Removal applying a minimum of 8 points, 3/Chromatogram Chemical Noise Reduction using 5 scan smoothing, a moving average estimator, a 25 scan RT window, a 30% quantile, and clipping an intensity of 20, 4/Grid using an adaptive grid with 10 scans and 10% deltaRT smoothing, 5/Chromatogram RT Alignment using a pairwise alignment-based tree and 50 RT scan search interval, 6/Chromatogram Peak Detection using a 0.1 min minimum peak size, 0.03 Da maximum merge distance, a boundaries merge strategy, a 20% gap/peak ratio, a curvature-based algorithm, intensity-weighed and using inflection points to determine boundaries, 7/Chromatogram Isotope Clustering using a 0.3 min RT tolerance, a 0.1 Da m/z tolerance, a peptide isotope shaping method with protonation, charges from 2-6 and mono-isotopic masses; 8/Singleton Filter, 9/MS/MS Consolidation, 10/Proteome Discoverer Import using a Xcorr above 1.5, 11/Peak Annotation, 12/Export Analyst using cluster volumes.

Statistical analyses were performed using the Analyst module of Genedata Expressionist® 11.0 where columns denote plant samples and rows denote intact proteins or tryptic digest peptides. Principal Component Analyses (PCA) were performed on rows using a covariance matrix with 50% valid values and row mean as imputation. Two-dimension hierarchical clustering (2-D HCA) was performed on both columns and rows using positive correlation and Ward linkage method. Venn diagrams were produced by exporting quantitative data of the identified peptides to Microsoft Excel 2016 (Office 365) spreadsheet and using the Excel function COUNT to establish the frequency of the peptides in the samples and across extraction methods. Venn diagrams were drawn in Microsoft Powerpoint 2016 (Office 365).

### Protein Standards for Top-Down Proteomics

Protein standards were purchased from Sigma and include: α-casein (α-CN 23.6 kDa) from bovine milk (C6780-250MG, 70% pure), β-lactoglobulin (β-LG, 18.7 kDa) from bovine milk (L3908-250MG, 90% pure), albumin from bovine serum (BSA, 66.5 kDa, A7906-10G, 98% pure), and myoglobin from horse skeletal muscle (Myo, 16.9 kDa, M0630-250MG, 95-100% pure and salt-free.

Lyophilised protein standards were solubilised at a 10 mg/mL concentration in 50% acetonitrile (ACN)/0.1% formic acid (FA)/10 mM dithiothreitol (DTT). Standards were dissolved by vortexing for 1 min and sonication for 10 min followed by another 1 min vortexing. An iodoacetamide (IAA) solution was added to reach a final concentration of 20 mM, vortexed for 1 min, and left to incubate for 30 min at room temperature in the dark. Apart from BSA and β-lactoglobulin, none of the standards needed reduction and alkylation steps as they bear no disulfide bridges; yet, these steps were still performed to emulate plant sample processing.

Standard solutions were then desalted using a solid phase extraction (SPE) cartridges (Sep-Pak C18 1 cc Vac Cartridge, 50 mg sorbent, 55-105 μm particle size, 1 mL, Waters) by gravity as described in Vincent et al., supra. Bound intact proteins were desalted using 1 mL of 0.1% FA solution and eluted into a 2 mL microtube using 1 mL of 80% ACN/0.1% FA solution.

### Up-Scaled Cannabis Protein Extraction for Top-Down Proteomics

Protein extraction for Cannabis mature apical buds was performed according to the method of Extraction 4, as described at [00132] above. This method was up-scaled for top-down proteomics, as detailed below.

One 500 mg scoop of ground frozen powder of plant material from apical buds was transferred into a 15 mL tube kept on ice prefilled with 12 mL ice-cold 10% trichloroacetic acid (TCA)/10 mM dithiothreitol (DTT)/acetone (w/w/v). The tubes were vortexed for 1 min and left at −20° C. overnight. The next day, tubes were centrifuged for 30 min at 4° C. and at maximum speed (5000 rpm) using a swing rotor centrifuge (Sigma 4-16k). The supernatant was removed, and the pellet was resuspended in 12 mL ice-cold 10 mM DTT/acetone (w/v) by vortexing for 1 min. Tubes were left at −20° C. for 2 h. The tubes were centrifuged as specified before and the supernatant removed. This washing step of the pellet was repeated once more. The pellets were dried for 30 min under a fume hood. The dry pellet resuspended in 2 mL of guanidine-HCl buffer (6 M guanidine-HCl, 10 mM DTT, 5.37 mM sodium citrate tribasic dihydrate and 0.1 M Bis-Tris).

### Protein Assay and Cannabis Protein Alkylation

Protein extracts from apical buds were diluted ten times in guanidine-HCl buffer. The protein concentrations were measured in triplicates using the Microplate BCA protein assay kit (Pierce) following the manufacturer's instructions. Bovine Serum Albumin (BSA) from the kit was used as a standard as per instructions. Protein extract concentrations ranked from 2.84 to 3.72 mg of proteins per mL of extract.

Following protein assay, the concentrations of the DTT-reduced protein samples were adjusted to the least concentrated one (2.84 mg/mL) by adding an appropriate volume of guanidine-HCl buffer. The protein extracts were then alkylated by adding a volume of 1M iodoacetamide (IAA)/water (w/v) solution to reach a 20 mM final IAA concentration. The tubes were vortexed for 1 min and left to incubate at room temperature in the dark for 60 min.

### Cannabis Protein Desalting and Evaporation

A volume of 0.5 mL of alkylated protein extract (1.42 mg proteins) was then desalted, as described above at [0138] above.

The 1 mL eluates were then evaporated using a SpeedVac concentrator (Savant SPD2010) for 90 min until the volume reached 0.2 mL. The evaporated samples were transferred into a 100 μL glass insert placed into a glass vial. The vials were positioned into the autosampler at 4° C. for immediate analyses by UPLC-MS.

### Mass Spectrometry Analyses for Top-Down Proteomics

MS analyses were performed on an Orbitrap Elite hybrid ion trap-Orbitrap mass spectrometer (Thermo Fisher Scientific) composed of a Linear Ion Trap Quadrupole (ITMS) mass spectrometer hosting the source and a Fourier-Transform mass spectrometer (FTMS) with a resolution of 240,000 at 400 m/z. Both ITMS and FTMS were calibrated in positive mode and the ETD was tuned prior to all MS and MS/MS experiments. All MS and MS/MS files (RAW, mzXML, MGF) and fasta files from known protein standards and cannabis samples are available from the stable public repository MassIVE at the following URL: http://massive.ucsd.edu/ProteoSAFe/datasets.jsp with the accession number MSV000083970.

Protein standard solutions were individually infused using a 0.5 mL Gastight #1750 syringe (Hamilton Co.) at a 20-30 μL/min flow rate using the built-in syringe pump of the LTQ mass spectrometer, to achieve at least 1e6 ion signal intensity. Protein standard solutions were pushed through first a 30 cm red PEEK tube (0.005 in. ID), then through a metal union and a PEEK VIPER tube (6041-5616, 130 μm×150 mm, Thermo Fischer Scientific), eventually to the heated electrospray ionisation (HESI) source where proteins were electrosprayed through a HESI needle insert 0.32 gauge (Thermo Fisher Scientific 70005-60155).

The source parameters were: capillary temperature 300° C., source heater temperature 250° C., sheath gas flow 30, auxiliary gas flow 10, sweep gas flow 2, FTMS injection waveforms on, FTMS full AGC target 1e6, FTMS MSn AGC target 1e6, positive polarity, source voltage 4 kV, source current 100 μA, S-lens RF level 70%, reagent ion source CI pressure 10, reagent vial ion time 200 ms, reagent vial AGC target 5e5, supplemental activation energy 15V, FTMS full micro scans 16, FTMS full max ion time 100 ms, FTMS MSn micro scans 8, and FTMS MSn max ion time 1000 ms. SID was set at 15V and FT Penning gauge pressure difference was set at 0.01 E-10 Torr to improve signal intensity. Mass window was 600-2000 m/z for FTMS1 and 300-2000 m/z for FTMS2.

Various fragmentation parameters were tested on individual protein standards. In-source fragmentation (SID) potentials varied from 0 to 100 V (maximum potential). Collision-Induced Dissociation (CID) normalized collision energy (NCE) varied from 30 to 50 eV with constant activation Q of 0.400 and an activation time of 100 ms. High energy CID (HCD) NCE varied from 10 to 30 eV with constant activation time of 0.1 ms. Electron Transfer Dissociation (ETD) activation times varied from 5 to 25 ms with constant activation Q of 0.250. Data files were acquired on the fly using the Acquire Data function of Tune Plus software 2.7 (Thermo Fisher Scientific) for up to 3 min at a time.

### Separation of Cannabis Intact Proteins by UPLC

Intact proteins from cannabis mature buds were chromatographically separated using a UHPLC 1290 Infinity Binary LC system (Agilent) and a bioZen XB-C4 column (3.6 μm, 200 Å, 150×2.1 mm, Phenomenex) kept at 90° C. Flow rate was 0.2 mL/min and total duration was 120 min. Mobile phase A contained 0.1% FA in water and mobile phase B contained 0.1% FA in acetonitrile.

Chromatographic separation was optimised and optimum UPLC gradient for cannabis proteins was as follows: starting conditions 3% B, ramping to 15% B in 2 min, ramping to 40% B in 89 min, ramping to 50% B in 5 min, ramping to 99% B in 5 min and held at 99% B for 10 min, lowering to 3% B in 1.1 min, equilibration at 3% B for 7.9 min. A 20 μL injection volume was applied to each protein extract. Each extract was injected five times with blank in between the extracts.

Analyses of Cannabis Intact Protein Extracts Using MS Online with UPLC

The UPLC outlet line was connected to the switching valve of the LTQ mass spectrometer. During the 119 min acquisition time by mass spectrometry, the first two minutes and the last minute of the run were directed to the waste whereas the rest of the run was directed to the source.

### Full Scan FTMS1

Tune parameters have been described above. Data was acquired in positive polarity with profile and normal scan modes at a resolution of 240,000 at 400 m/z along a mass window of 500-2000 m/z. SID was set at 15V. Full scan files were acquired in duplicate at the first and last injections of the 5 sample injections. The three intermediate injections were dedicated to tandem MS (see below).

### FTMS2

Three MS/MS methods were applied in which the energy applied to each fragmentation modes varied between what we call “Low”, “High”, and intermediate “Mid”. SID was set to 15V throughout. One segment was defined with four scan events. The first scan event applied full scan FTMS in profile and normal modes at a resolution of 120,000 for 400 m/z, scanning a mass window of 500-2000 m/z. The most abundant ion whose intensity was above 500 and m/z above 700 from the first scan was selected for subsequent fragmentation in a data-dependent manner with an isolation width of 15 and a default charge state of 10. FTMS2 spectra were acquired along a mass window of 300-2000 m/z at a resolution of 60,000 at 400 m/z. Scan events 2 to 4 are described below as their energy levels varied. The parameters that changed are in bold.

In the “Low” energy FTMS2 method, the precursor underwent an ETD fragmentation during the second scan event with an activation time of 5 ms and an activation Q of 0.250; a CID fragmentation in the third scan event with a NCE of 35 eV, an activation Q of 0.400 and an activation time of 100 ms; and a HCD fragmentation with a NCE of 19 eV and an activation time of 0.1 ms.

In the “Mid” energy FTMS2 method, the precursor underwent an ETD fragmentation during the second scan event with an activation time of 10 ms and an activation Q of 0.250; a CID fragmentation in the third scan event with a NCE of 42 eV, an activation Q of 0.400 and an activation time of 100 ms; and a HCD fragmentation with a NCE of 23 eV and an activation time of 0.1 ms.

In the “High” energy FTMS2 method, the precursor underwent an ETD fragmentation during the second scan event with an activation time of 15 ms and an activation Q of 0.250; a CID fragmentation in the third scan event with a NCE of 50 eV, an activation Q of 0.400 and an activation time of 100 ms; and a HCD fragmentation with a NCE of 27 eV and an activation time of 0.1 ms.

### Data Processing and Statistical Analyses for Top-Down Proteomics

### Analysis of Infusion MS/MS Spectra

Given the MW of myoglobin, β-lactoglobulin, α-S1-casein and the 240,000 resolution of the instrument, the spectra of these proteins were isotopically resolved. BSA is too large for isotopic resolution, therefore only average mass was obtained. Isotopically resolved RAW files were opened using the Qual Browser module of Xcalibur software version 3.1 (Thermo scientific) and deconvoluted using Xtract algorithm (Thermo scientific) with the following parameters: M masses mode, 60000 resolution at 400 m/z 3 S/N threshold, 44 fit factor, 25% remainder, averagine method and 40 max charges. In the deconvoluted spectra, the second scan corresponding to the monoisotopic zero-charge (deisotoped) mass spectrum was selected for export as explained in DeHart et al. Methods Mol. Biol. 2017, 1558: 381-394.

Deconvoluted exact masses were then exported to Excel 2016 (Microsoft) to generate pivot tables and charts. VBA macros were used to compile lists of masses corresponding to different MS/MS modes and parameters, and parent ions from the same protein. The deconvoluted deisotoped masses were copied and pasted into ProSight Lite version 1.4 (Northwestern University, USA) with the following parameters: S-carboxamidomethyl-L-cysteine as a fixed modification, monoisotopic precursor mass type, and fragmentation tolerance of 50 ppm. The AA sequence varied according to the standards analysed; where needed the initial methionine residue (myoglobin), the signal peptide (β-LG, α-S1-CN, BSA) and the pro-peptide (BSA) were removed. The fragmentation method chosen was either SID, HCD, CID, or ETD, depending on how the MS/MS data was acquired. When multiple MS/MS spectra were used including ETD data, the BY and CZ fragmentation method was selected.

Raw MS/MS files were imported into Proteome Discoverer version 2.2 (Thermo Fisher Scientific) through the Spectrum Files node and the following parameters were used in the Spectrum Selector node: use MS1 precursor with isotope pattern, lowest charge state of 2, precursor mass ranging from 500-50,000 Da, minimum peak count of 1, MS orders 1 and 2, collision energy ranging from 0-1000, full scan type. The selected spectra were then deconvoluted through the Xtract node with the following parameters: S/N threshold of 3, 300-2000 m/z window, charge from 1-30 (maximum value), resolution of 60,000, and monoisotopic mass. When not specified, default parameters were used. Deconvoluted spectra (MH+) were then exported as a single Mascot Generic Format (MGF) file.

The MGF file was searched in Mascot version 2.6.1 (MatrixScience) with Top-Down searches license. A MS/MS Ion Search was performed with the NoCleave enzyme, Carbamidomethyl (C) as fixed modification and Oxidation (M), Acetyl (Protein N-term), and Phospho (ST) as variable modifications, with monoisotopic masses, 1% precursor mass tolerance, ±50 ppm or ±2 Da fragment mass tolerance, precursor charge of +1, 9 maximum missed cleavages, and instrument type that accounted for CID, HCD and ETD fragments (i.e. b-, c-, y-, and z-type ions) of up to 110 kDa. The first database searched was a fasta file containing the AA sequences of all the known variants of cow's milk most abundant proteins (all caseins, alpha-lactalbumin, beta-lactoglobulin, and BSA) along with horse's myoglobin (59 sequences in total). The decoy option was selected. The second database searched was SwissProt (all 559,228 entries, version 5) using all the entries or just the “other mammalia” taxonomy.

Analysis of LC-MS and LC-MS/MS Data from Cannabis Samples

The RAW files were loaded and processed in the Refiner modules of Genedata Expressionist® version 12.0.6 using the following steps and parameters: profile data cutoff of 10,000, R window of 3-99 min, m/z window of 500-1800 Da, removal of RT structures <4 scans, removal of m/z structures <5 points, smoothing of chromatogram using a 5 scans window and moving average estimator, spectrum smoothing using a 3 points m/z window, a chromatogram peak detection using a summation window of 15 scans, a minimum peak size of 1 min, a maximum merge distance of 10 ppm, and a curvature-based algorithm with local maximum and FWHM boundary determination, isotope clustering using a peptide isotope shaping method with charges ranging from 2-25 (maximum value) and monoisotopic masses, singleton filtering, and charges and adduct grouping using a 50 ppm mass tolerance, positive charges, and dynamic adduct list containing protons, H₂O, K—H, and Na—H. The protein groups were used for statistical analyses.

Spectral deconvolution from 3-70 kDa was performed using manual deprecated mode and harmonic suppression deconvolution method with a 0.04 Da step, as well as curvature-based peak detection, intensity-weighed computation and inflection points to determine boundaries. This step generated LC-MS maps of protein deisotoped masses.

Group volumes were exported to the Analyst module of Genedata Expressionist to perform statistical analyses Parameters for Principal Component Analysis (PCA) were analysis of rows, covariance matrix, 70% valid values, and row mean imputation. Parameters for Hierarchical Clustering Analysis (HCA) were clustering of columns, shown as tree, positive correlation distances, Ward linkage, 70% valid values.

### Identification of Cannabis Proteins by Mascot

The RAW files were processed in Proteome Discoverer version 2.2 (Thermo Fisher Scientific) as detailed above for the known protein standards to create a single MGF file containing 11,250 MS/MS peak lists.

The MGF file was searched in Mascot version 2.6.1 (MatrixScience) with Top-Down searches license. A MS/MS Ion Search was performed with the NoCleave enzyme, Carbamidomethyl (C) as fixed modification and Oxidation (M), Acetyl (Protein N-term) and Phosphorylation (ST) as variable modifications, with monoisotopic masses, ±1% precursor mass tolerance, ±50 ppm or ±2 Da fragment mass tolerance, precursor charge of 1+, 9 maximum missed cleavages, and instrument type that accounted for CID, HCD and ETD fragments (i.e. b-, c-, y-, and z-type ions) of up to 110 kDa. The database searched was a fasta file previously compiled to contain all UniprotKB AA sequences from C. sativa and close relatives, amounting to 663 entries in total (i.e. 73 sequences added in 6 months). The decoy option was selected. The error tolerant option was tested as well but not pursued as search times proved much longer and number of hits diminished. The other database searched was SwissProt viridiplantae (39,800 sequences; version 5).

### Chemicals for Multiple Protease Strategy

All proteases were purchased from Promega: Trypsin/LysC mix (V5072, 100 μg), GluC (V1651, 50 μg), and Chymotrypsin (V106A, 25 μg). Albumin from bovine serum (BSA, A7906-10G, 98% pure) was purchased from Sigma and analysed by MS.

### Protein Extraction Methods

The protein extraction described above at [00132] was up-scaled to prepare sufficient amount of sample to undergo various protease digestions. Briefly, 0.5 g of ground frozen powder was transferred into a 15 mL tube kept on ice pre-filled with 12 mL ice-cold 10% TCA/10 mM DTT/acetone (w/w/v). Tubes were vortexed for 1 min and left at −20° C. overnight. The next day, tubes were centrifuged for 10 min at 5,000 rpm and 4° C. The supernatant was discarded, and the pellet was resuspended in 10 mL of ice-cold 10 mM DTT/acetone (w/v) by vortexing for 1 min. Tubes were left at −20° C. for 2 h. The tubes were centrifuged as specified before and the supernatant discarded. This washing step of the pellets was repeated once more. The pellets were dried for 60 min under a fume hood. The dry pellets were resuspended in 2 mL of guanidine-HCl buffer (6M guanidine-HCl, 10 mM DTT, 5.37 mM sodium citrate tribasic dihydrate, and 0.1 M Bis-Tris) by vortexing for 1 min, sonicating for 10 min and vortexing for another minute. Tubes were incubated at 60° C. for 60 min. The tubes were centrifuged as described above and 1.8 mL of the supernatant was transferred into 2 mL microtubes. 40 μL of 1M IAA/water (w/v) solution was added to the tubes to alkylate the DTT-reduced proteins. The tubes were vortexed for 1 min and left to incubate at room temperature in the dark for 60 min.

1.1 mL of BSA solution (2 mg/mL, Pierce) was transferred into a 2 mL microtube and 10 uL of 1 M DTT/water (w/v) solution was added. The tube was vortexed for 1 minute and incubated at 60° C. for 60 min. 20 μL of 1M IAA/water (w/v) solution was added to the tube. The BSA tube was vortexed for 1 min and left to incubate at room temperature in the dark for 60 min.

### Protein Assay

Protein extracts were diluted ten times using the guanidine-HCl buffer prior to the assay. The protein concentrations were measured in triplicates using the Pierce Microplate BCA protein assay kit (ThermoFisher Scientific) following the manufacturer's instructions. The BSA solution supplied in the kit (2 mg/mL) was used a standard.

### Protein Digestion

An aliquot corresponding to 100 μg of BSA or plant proteins was used for protein digestion as follows.

### Digestion 1: Trypsin/LysC Protease Mix (T)

DTT-reduced and IAA-alkylated proteins were diluted six times using 50 mM Tris-HCl pH 8.0 to drop the resuspension buffer molarity below 1 M. Trypsin/LysC protease (Mass Spectrometry Grade, 100 μg, Promega) was carefully solubilised in 1 mL of 50 mM acetic acid and incubated at 37° C. for 15 min. A 40 μL aliquot of trypsin/LysC solution was added and gently mixed with the protein extracts thus achieving a 1:25 ratio of protease:proteins. The mixture was left to incubate overnight (18 h) at 37° C. in the dark.

### Digestion 2: GluC (G)

DTT-reduced and IAA-alkylated proteins were diluted six times using 50 mM Ammonium bicarbonate (pH 7.8) to drop the resuspension buffer molarity below 1 M. GluC protease (Mass Spectrometry Grade, 50 μg, Promega) was carefully solubilised in 0.5 mL of ddH₂O. A 10 μL aliquot of GluC solution was added and gently mixed with the protein extracts thus achieving a 1:100 ratio of protease:proteins. The mixture was left to incubate overnight (18 h) at 37° C. in the dark.

### Digestion 3: Chymotrypsin (C)

DTT-reduced and IAA-alkylated proteins were diluted six times using 100 mM Tris/10 mM CaCl₂ pH 8.0 to drop the resuspension buffer molarity below 1 M. Chymotrypsin protease (Sequencing Grade, 25 μg, Promega) was carefully solubilised in 0.25 mL of 1M HCl. A 10 μL aliquot of chymotrypsin solution was added and gently mixed with the protein extracts thus achieving a 1:100 ratio of protease:proteins. The mixture was left to incubate overnight (18 h) at 25° C. in the dark.

### Sequential Digestion 1: Trypsin/LysC Followed by GluC (T→G)

Digestion using trypsin/LysC was performed as described above at [00185]. The next day, a 10 μL aliquot of GluC solution (50 μg in 0.5 mL ddH₂O) was added and gently mixed with the trypsin/LysC digest. The tubes were incubated again at 37° C. in the dark for 18 h.

### Sequential Digestion 2: Trypsin/LysC Followed by Chymotrypsin (T→C)

Digestion using trypsin/LysC was performed as described above at [00185]. The next day, a 10 μL aliquot of chymotrypsin solution (25 μg in 0.25 mL 1M HCl) was added and gently mixed with the trypsin/LysC digest. The tubes were then incubated at 25° C. in the dark for 18 h.

### Sequential Digestion 3: GluC Followed by Chymotrypsin (G→C)

Digestion using GluC was performed as described above at [00186]. The next day, a 10 μL aliquot of chymotrypsin solution (25 μg in 0.25 mL 1M HCl) was added and gently mixed with the GluC digest. The tubes were then incubated at 25° C. in the dark for 18 h.

### Sequential Digestion 4: Trypsin/LysC Followed by GluC Followed by Chymotrypsin (T→G→C)

Digestion using trypsin/LysC was performed as described above at [00185]. The next day, a 10 μL aliquot of GluC solution (50 μg in 0.5 mL ddH₂O) was added and gently mixed with the trypsin/LysC digest. The tubes were incubated again at 37° C. in the dark for 18 h. The next day, a 10 μL aliquot of chymotrypsin solution (25 μg in 0.25 mL 1M HCl) was added and gently mixed with the trypsin/LysC digest. The tubes were then incubated at 25° C. in the dark for 18 h.

### Equimolar Mixtures of Digests (T:G, T:G, G:C, T:G:C)

In an effort to assess the efficiency of the sequential digestions (T→G, T→G, G→C, T→G→C), individual BSA digests resulting from the independent activity of trypsin/LysC, GluC and chymotrypsin were pooled together using the same volumes. Thus, the trypsin/LysC digest was pooled with the GluC digest (T:G), the trypsin/LysC digest was pooled with the chymotrypsin digest (T:C), the GluC digest was pooled with the chymotrypsin digest (G:C), and the three trypsin/Lys-, GluC and chymotrypsin were also pooled together (T:G:C).

### Desalting

All of the digestion reactions were stopped by lowering the pH of the mixture using a 10% formic acid (FA) in H₂O (v/v) to a final concentration of 1% FA.

All digests were desalted using solid phase extraction (SPE) cartridges (Sep-Pak C18 1 cc Vac Cartridge, 50 mg sorbent, 55-105 μm particle size, 1 mL, Waters) by gravity, followed by Speedvac evaporation.

The digest was transferred into a 100 μL glass insert placed into a glass vial. The vials were positioned into the autosampler at 4° C. for immediate analyses by nLC-MS/MS.

Peptide Digest Analysis by Nano Liquid Chromatography-Tandem Mass Spectrometry (nLC-MS/MS)

The nLC-ESI-MS/MS analyses were performed on all the peptide digests in duplicate. Chromatographic separation of the peptides was performed by reverse phase (RP) using an Ultimate 3000 RSLCnano System (Dionex) online with an Elite Orbitrap hybrid ion trap-Orbitrap mass spectrometer (ThermoFisher Scientific). The parameters for nLC and MS/MS have been described in Vincent et al., supra. A 1 μL aliquot (0.1 μg peptide) was loaded using a full loop injection mode onto a trap column (Acclaim PepMap100, 75 μm×2 cm, C18 3 μm 100 Å, Dionex) at a 3 μL/min flow rate and switched onto a separation column (Acclaim PepMap100, 75 μm×15 cm, C18 2 μm 100 Å, Dionex) at a 0.4 μL/min flow rate after 3 min. The column oven was set at 30° C. Mobile phases for chromatographic elution were 0.1% FA in H₂O (v/v) (phase A) and 0.1% FA in ACN (v/v) (phase B). Ultraviolet (UV) trace was recorded at 215 nm for the whole duration of the nLC run. A linear gradient from 3% to 40% of ACN in 35 min was applied. Then ACN content was brought to 90% in 2 min and held constant for 5 min to wash the separation column. Finally, the ACN concentration was lowered to 3% over 0.1 min and the column reequilibrated for 5 min. On-line with the nLC system, peptides were analysed using an Orbitrap Velos hybrid ion trap-Orbitrap mass spectrometer (Thermo Scientific). Ionisation was carried out in the positive ion mode using a nanospray source. The electrospray voltage was set at 2.2 kV and the heated capillary was set at 280° C. Full MS scans were acquired in the Orbitrap Fourier Transform (FT) mass analyser over a mass range of 300 to 2000 m/z with a 60,000 resolution in profile mode. MS/MS spectra were acquired in data-dependent mode. The 20 most intense peaks with charge state ≥2 and a minimum signal threshold of 10,000 were fragmented in the linear ion trap using collision-induced dissociation (CID) with a normalised collision energy of 35%, 0.25 activation Q and activation time of 10 msec. The precursor isolation width was 2 m/z. Dynamic exclusion was enabled, and peaks selected for fragmentation more than once within 10 sec were excluded from selection for 30 sec. Each digest was injected twice, with first injecting all the digests (technical replicate 1) and then fully repeating the injections in the same order (technical replicate 2).

### Database Search for Protein Identification

Database searching of the .RAW files was performed in Proteome Discoverer (PD) 1.4 using SEQUEST algorithm as described above at [00145]. The database searching parameters specified trypsin, or GluC, or chymotrypsin or their respective combinations as the digestion enzymes and allowed for up to ten missed cleavages. The precursor mass tolerance was set at 10 ppm, and fragment mass tolerance set at 0.8 Da. Peptide absolute Xcorr threshold was set at 0.4, the fragment ion cutoff was set at 0.1%, and protein relevance threshold was set at 1.5. Carbamidomethylation (C) was set as a static modification and oxidation (M), phosphorylation (STY), and N-Terminus acetylation were set as dynamic modifications The target decoy peptide-spectrum match (PSM) validator was used to estimate false discovery rates (FDR). At the peptide level, peptide confidence value set at high was used to filter the peptide identification, and the corresponding FDR on peptide level was less than 1%. At the protein level, protein grouping was enabled.

All nLC-MS/MS files are available from the stable public repository MassIVE at the following URL: http://massive.ucsd.edu/ProteoSAFe/datasets.jsp with the accession number MSV000084216.

### Data Processing and Statistical Analyses

nLC-MS/MS Data Processing

The data files obtained following nLC-MS/MS analysis were processed in the Refiner MS module of Genedata Expressionist® 12.0 with the following parameters: 1) Load from file by restricted the range from 8-45 min, 2) Metadata import, 3) Spectrum smoothing using Moving Average algorithm and a minimum of 5 points, 4) RT structure removal using a minimum of 3 scans, 5) m/z grid using an adaptative grid method with a scan count of 10 and a 10% smoothing, 6) chromatogram RT alignment with a pairwise alignment based tree, a maximum shift of 50 scans and no gap penalty, 7) chromatogram peak detection using a 10 scan summation window, a 0.1 min minimum peak size, 0.04 Da maximum merge distance, a boundaries merge strategy, a 20% gap/peak ratio, a curvature-based algorithm, intensity-weighed and using inflection points to determine boundaries, 8) MS/MS consolidation, 9) Proteome Discoverer Import accepting only top-ranked database matches and no decoy results, 10) Peak Annotation, 11) Export Analyst using peak volumes.

A Peptide Mapping activity for BSA digest samples was also performed using the mature AA sequence of the protein (P02769|25-607) following step 8 (MS/MS consolidation) as follows: 12) Selection of the relevant protease digests, 13) Peptide Mapping using the following parameters: 10 ppm mass tolerance, ESI-CID/HCD instrument, 0.8 Da fragment tolerance, min fragment score of 30, top-ranked only, discard mass-only matches, enzymes varied according to the protease(s) used, 6 max missed cleavages, min peptide length of 3, fixed Carbamidomethyl (C) modification, and variable Oxidation (M) modification.

### Statistical Analyses

Statistical analyses were performed using the Analyst module of Genedata Expressionist® 12.0 where columns denote plant samples and rows denote digest peptides. Principal Component Analyses (PCA) were performed on rows using a covariance matrix with 40% valid values and row mean as imputation. A linear model performed on rows and testing the digestion type. Partial Least Square (PLS) analyses were run on the most significant rows resulting from the linear model. PLS response was the digestion type with three latent factors, 50% valid values and row mean as imputation. Hierarchical clustering analysis (HCA) was performed on columns using positive correlation and Ward linkage method. Histograms were generated by exporting number of peaks, number of MS/MS spectra, masses of the identified peptides to Microsoft Excel 2016 (Office 365) spreadsheet.

### Example 1—Intact Protein Analysis

This experiment aimed to optimise protein extraction from mature reproductive tissues of medicinal cannabis. A total of six protein extractions were tested with methods varying in their precipitation steps with the use of either acetone or ethanol as solvents, as well as changing in their final pellet resuspension step with the use of urea- or guanidine-HCL-based buffers. The six methods were applied to liquid N2 ground apical buds. Trichomes were also isolated from apical buds. Because of the small amount of trichome recovered, only the single step extraction methods 1 and 2 were attempted. Extractions were performed in triplicates. Extraction efficiency was assessed both by intact protein proteomics and bottom-up proteomics each performed in duplicates. Rigorous method comparisons were then drawn by applying statistical analyses on protein and peptide abundances, linked with protein identification results.

The intact proteins of the 18 apical bud extracts and the 6 trichome extracts were separated by UPLC and analysed by ESI-MS in duplicates. LC-MS profiles are complex with many peaks both retention time (RT) in min and m/z axes, particularly between 5-35 min and 500-1300 m/z. Prominent proteins eluted late (25-35 min), probably due to high hydrophobicity, and within low m/z ranges (600-900 m/z), therefore bearing more positive charges. Outside this area, many proteins eluting between 5 and 25 min were resolved in samples processed using extraction methods 2, 4 and 6, irrespective of tissue types (apical buds or trichomes). Protein extracts from apical buds and trichomes overall generated 26,892 intact protein LC-MS peaks (ions), which were then clustered into 5,408 isotopic clusters, which were in turn grouped into 571 proteins of up to 11 charge states. The volumes of all the peaks comprised into a group were summed and the sum was used as a proxy for the amounts of the intact proteins. Statistical analyses were performed on the summed volumes of the 571 protein groups.

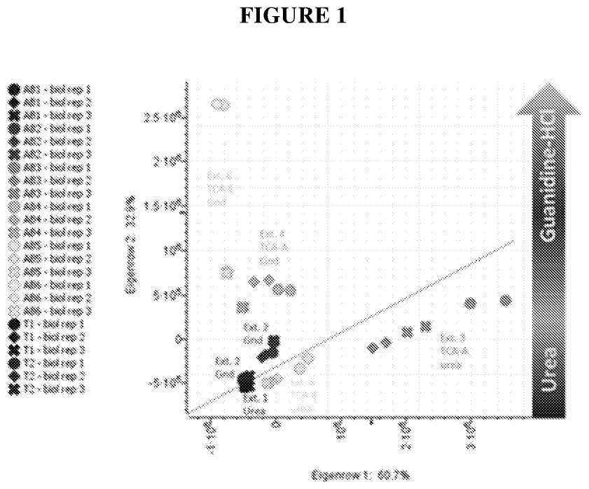

A Principal Component (PC) Analysis (PCA) was performed to verify whether the different extraction methods impacted protein LC-MS quantitative data. A plot of PC1 (60.7% variance) against PC2 (32.9% variance) clearly separates urea-based methods from guanidine-HCl-based methods (FIG. 1). Each of the six methods are well defined and do not cluster together. Extraction methods 3-6, which include an initial precipitation step, are further isolated.

Table 2 indicates the concentration of the protein extracts as well as the number of protein groups quantified in Genedata expressionist. Extraction method 1 yields the greatest protein concentrations: 6.6 mg/mL in apical buds and 3.5 mg/mL in trichomes, followed by extraction methods 2, 4, 6, 3 and 5. Overall, 571 proteins were quantified and the extraction methods recovering most intact proteins in apical buds are methods 2 (335±15), 4 (314±16) and 6 (264±18). In our experiment, method 1 yielding the highest protein concentrations did not equate larger numbers of proteins resolved by LC-MS. Perhaps C. sativa proteins recovered by method 1 are not compatible with our downstream analytical techniques (LC-MS). In trichomes, the method yielding the highest number of intact proteins is extraction method 2 (249±45). Extraction methods 2, 4, and 6 all conclude by a resuspension step in a guanidine-HCl buffer, which consequently is the buffer we recommend for intact protein analysis.

These data demonstrate that suspension of cannabis-derived proteins in a solution comprising a charged chaotropic agent is effective for preparing cannabis plant material for top-down proteomic analysis.

TABLE 2
Proteins quantified by top-down proteomics.

| | | | | Protein | Protein | | | | |
|---|---|---|---|---|---|---|---|---|---|
| | | | | concentration | concentration | Number | Number | Number | Number |
| | Extraction | Extraction | Extraction | (mg/mL) | (mg/mL) | of proteins | of proteins | of proteins | of proteins |
| Tissue | number | method | code | Average | SD | Average | Percent | SD | CV |
| apical | extraction 1 | Urea | AB1 | 6.58 | 0.89 | 254 | 44.51 | 12 | 4.80 |
| bud | | | | | | | | | |
| apical | extraction 2 | Gnd-HCl | AB2 | 3.50 | 0.99 | 335 | 58.58 | 15 | 4.47 |
| bud | | | | | | | | | |
| apical | extraction 3 | TCA-A/urea | AB3 | 0.63 | 0.15 | 247 | 43.23 | 21 | 8.69 |
| bud | | | | | | | | | |
| apical | extraction 4 | TCA-A/Gnd- | AB4 | 1.50 | 0.28 | 314 | 54.90 | 16 | 5.13 |
| bud | | HCl | | | | | | | |
| apical | extraction 5 | TCA-E/urea | AB5 | 0.60 | 0.11 | 201 | 35.11 | 5 | 2.64 |
| bud | | | | | | | | | |
| apical | extraction 6 | TCA-E/Gnd- | AB6 | 0.76 | 0.48 | 264 | 46.18 | 18 | 6.84 |
| bud | | HCl | | | | | | | |
| trichome | extraction 1 | Urea | T1 | 3.67 | 0.39 | 170 | 29.83 | 5 | 2.97 |
| trichome | extraction 2 | Gnd-HCl | T2 | 2.28 | 1.17 | 249 | 43.61 | 45 | 18.12 |
| TOTAL | | | | | | 571 | | | |

As far as we know, this is the first time a gel-free intact protein analysis is presented. The old-fashioned technique 2-DE separates intact proteins based first on their isoelectric point and second on their molecular weight (MW). Because it is time-consuming, labour-intensive, and of low throughput, 2-DE has now been superseded by liquid-based techniques, such as LC-MS. In the present study we have chosen to separate intact proteins of medicinal cannabis based on their hydrophobicity using RP-LC and a C8 stationary phase online with a high-resolution mass analyser which separates ionised intact proteins based on their mass-to-charge ratio (m/z).

### Example 2—Tryptic Peptides Analysis

The 25 tryptic digests of medicinal cannabis extracts and BSA sample were separated by nLC and analysed by ESI-MS/MS in duplicates. BSA was used as a control for the digestion with the mixture of endoproteases, trypsin and Lys-C, cleaving arginine (R) and lysine (K) residues. BSA was successfully identified with overall 88 peptides covering 75.1% of the total sequence, indicating that both protein digestions and nLC-MS/MS analyses were efficient.

nLC-MS/MS profiles are very complex with altogether 105,249 LC-MS peaks (peptide ions) clustered into 43,972 isotopic clusters, with up to 11,540 MS/MS events. If we consider apical bud patterns only, guanidine-HCl-based extraction methods (2, 4, and 6) generate a lot more peaks than urea-based methods (1, 3, and 5). As far as trichomes are concerned, extraction methods 1 and 2 yield comparable patterns, albeit with less LC-MS peaks than those of apical buds.

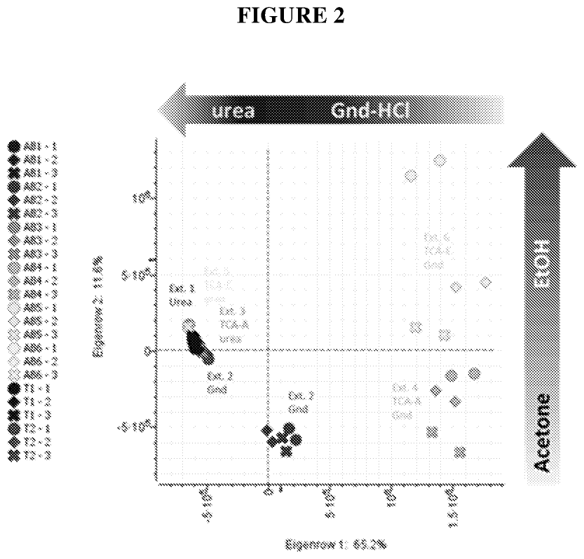

The volumes of all the peaks comprised into a cluster were summed and the sum was used as a proxy for the amounts of the tryptic peptides. PCA were performed on the summed volumes of the 43,972 peptide clusters. A biplot of PC 1 against PC 2 illustrates the separation of guanidine-HCl based-methods from urea-based methods along PC 1 (65.2% variance), and the distinction between acetone (method 4) and ethanol (method 6) precipitations along PC 2 (11.6% variance) (FIG. 2).

Table 3 indicates the number of peptides identified with high score (Xcorr>1.5) by SEQUEST algorithm and matching one of the 590 AA sequences we retrieved from C. sativa and closely related species for the database search. Overall, 488 peptides were identified and the extraction methods yielding the greatest number of database hits in apical buds were methods 4 (435±9), 6 (429±6) and 2 (356±20). In trichomes, the method yielding the highest number of identified peptides was extraction method 2 (102±23). Similar to our conclusions from intact protein analyses, we also recommend guanidine-HCl-based extraction methods (2, 4, and 6) for trypsin digestion followed by shotgun proteomics.

Accordingly, these data demonstrate that suspension of cannabis-derived proteins in a solution comprising a charged chaotropic agent is effective for preparing cannabis plant material for bottom-up proteomic analysis.

TABLE 3
Peptides identified with by bottom-up proteomics.

| | | | | Number | Number | Number | Number |
|---|---|---|---|---|---|---|---|
| | Extraction | Extraction | Extraction | of hits | of hits | of hits | of hits |
| Tissue | number | method | code | Average | Percent | SD | CV |
| apical | extraction 1 | Urea | AB1 | 211 | 43.24 | 34 | 16.09 |
| bud | | | | | | | |
| apical | extraction 2 | Gnd-HCl | AB2 | 356 | 72.88 | 20 | 5.51 |
| bud | | | | | | | |
| apical | extraction 3 | TCA-A/urea | AB3 | 265 | 54.23 | 55 | 20.70 |
| bud | | | | | | | |
| apical | extraction 4 | TCA-A/Gnd- | AB4 | 435 | 89.07 | 9 | 2.09 |
| bud | | HCl | | | | | |
| apical | extraction 5 | TCA-E/urea | AB5 | 41 | 8.33 | 15 | 35.71 |
| bud | | | | | | | |
| apical | extraction 6 | TCA-E/Gnd- | AB6 | 429 | 87.91 | 6 | 1.33 |
| bud | | HCl | | | | | |
| trichome | extraction 1 | Urea | T1 | 97 | 19.88 | 22 | 22.27 |
| trichome | extraction 2 | Gnd-HCl | T2 | 102 | 20.83 | 23 | 22.78 |
| TOTAL | | | | 488 | | | |

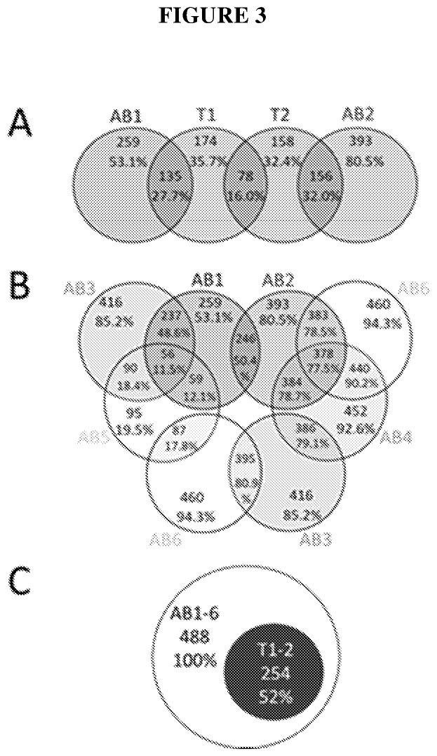

In an attempt to further compare the extraction methods with each other, Venn diagrams were produced on the 488 identified peptides (FIG. 3).

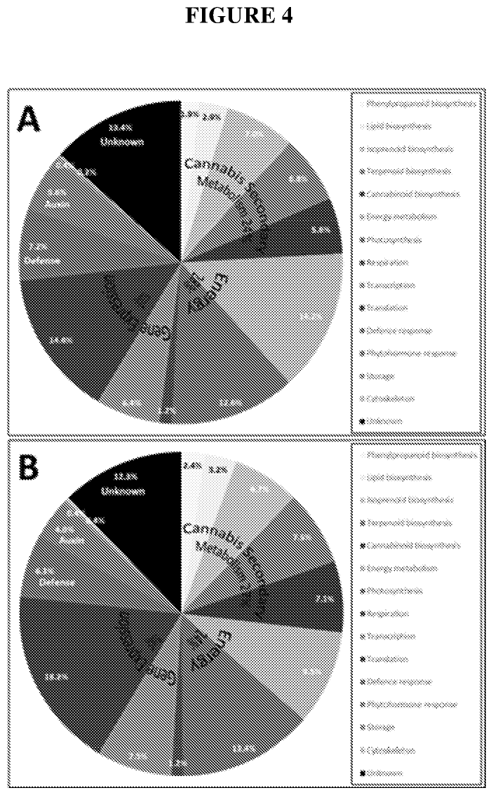

If we start with the trichomes and compare the simplest methods, extraction methods 1 and 2 which only involve a single resuspension step of the frozen ground plant powder into a protein-friendly buffer, we observe similar identification success 35.7% (174 out of 488 peptides) for T1 and 32.4% (158 peptides) for T2 and little overlap (16.0%; 78 peptides) between the two. Therefore, both methods are complementary (FIG. 4A). If we compare trichomes and apical buds, an overlap of 27.7% (135 peptides) is observed with extraction method 1 (urea-based buffer) while 32.0% (156 peptides) of database hits are shared between both tissues when extraction method 2 (guanidine-HCl) is employed (FIG. 4A). Whilst both outcomes are comparable, we would thus advice employing method 2 when handling cannabis trichomes. If we now turn our attention to just apical buds, we can see that about half of the identified peptides are common between methods 1 and 2 (AB1-AB2, 246 peptides; 50.4%). Guanidine-HCL-based methods (AB2, AB4, and AB6) share a majority of hits (77.5%; 378 peptides) whereas urea-based methods (AB1, AB3, and ABS) only share 11.5% (56) of identified peptides (FIG. 4B). This indicates that guanidine-HCl-based methods not only yield more identified peptides but also more consistently. Interestingly, the two most different methods (AB3 and AB6 employing different precipitant solvents and different resuspension buffers) share 80.9% (395) of the identified peptides (FIG. 4B), suggesting that the initial precipitation step would make the subsequent resuspension step more homogenous, irrespective of the buffer used. All the 254 peptides identified from trichomes were also identified in apical buds (FIG. 4C). Therefore, in our hands protein extraction from trichome did not yield unique protein identification. This might be explained by the fact that due to limited sample recovery only two extraction methods were tested on trichomes.

### Example 3—Proteins Identified by Bottom-Up Proteomics

Table 4 lists the 160 protein accessions from the 488 peptides identified from cannabis mature apical buds and trichomes in this study. These 160 accessions correspond to 99 protein annotations (including 56 enzymes) and 15 pathways (Table 4). Most proteins (83.1%) matched a C. sativa accession, 5% of the accessions came from European hop, and 11.8% of the accessions came from Boehmeria nivea, all of them annotated as small auxin up-regulated (SAUR) proteins.

TABLE 4
Proteins identified in medicinal cannabis apical buds and trichomes.

| | | Uniprot | | | | | | |
|---|---|---|---|---|---|---|---|---|
| Protein | | Accession or | | Length | No. of | | Function | |
| annotation | Abbreviation | Patent | Species | (AA) | peptides | EC No. | [CC] | Pathway |
| Small auxin | SAUR03 | A0A172J1X8 | Boehmeria nivea | 93 | 1 | | response to | Phytohormone |
| up regulated | | | | | | | auxin | response |
| protein | | | | | | | | |
| Small auxin | SAUR20 | A0A172J1Z7 | Boehmeria nivea | 147 | 1 | | response to | Phytohormone |
| up regulated | | | | | | | auxin | response |
| protein | | | | | | | | |
| Small auxin | SAUR23 | A0A172J212 | Boehmeria nivea | 99 | 1 | | response to | Phytohormone |
| up regulated | | | | | | | auxin | response |
| protein | | | | | | | | |
| Small auxin | SAUR24 | A0A172J211 | Boehmeria nivea | 102 | 1 | | response to | Phytohormone |
| up regulated | | | | | | | auxin | response |
| protein | | | | | | | | |
| Small auxin | SAUR28 | A0A172J206 | Boehmeria nivea | 108 | 1 | | response to | Phytohormone |
| up regulated | | | | | | | auxin | response |
| protein | | | | | | | | |
| Small auxin | SAUR30 | A0A172J210 | Boehmeria nivea | 100 | 1 | | response to | Phytohormone |
| up regulated | | | | | | | auxin | response |
| protein | | | | | | | | |
| Small auxin | SAUR31 | A0A172J276 | Boehmeria nivea | 152 | 1 | | response to | Phytohormone |
| up regulated | | | | | | | auxin | response |
| protein | | | | | | | | |
| Small auxin | SAUR40 | A0A172J219 | Boehmeria nivea | 105 | 1 | | response to | Phytohormone |
| up regulated | | | | | | | auxin | response |
| protein | | | | | | | | |
| Small auxin | SAUR44 | A0A172J227 | Boehmeria nivea | 152 | 4 | | response to | Phytohormone |
| up regulated | | | | | | | auxin | response |
| protein | | | | | | | | |
| Small auxin | SAUR48 | A0A172J226 | Boehmeria nivea | 133 | 1 | | response to | Phytohormone |
| up regulated | | | | | | | auxin | response |
| protein | | | | | | | | |
| Small auxin | SAUR54 | A0A172J237 | Boehmeria nivea | 118 | 5 | | response to | Phytohormone |
| up regulated | | | | | | | auxin | response |
| protein | | | | | | | | |
| Small auxin | SAUR55 | A0A172J229 | Boehmeria nivea | 97 | 3 | | response to | Phytohormone |
| up regulated | | | | | | | auxin | response |
| protein | | | | | | | | |
| Small auxin | SAUR58 | A0A172J236 | Boehmeria nivea | 97 | 1 | | response to | Phytohormone |
| up regulated | | | | | | | auxin | response |
| protein | | | | | | | | |
| Small auxin | SAUR59 | A0A172J243 | Boehmeria nivea | 106 | 5 | | response to | Phytohormone |
| up regulated | | | | | | | auxin | response |
| protein | | | | | | | | |
| Small auxin | SAUR60 | A0A172J238 | Boehmeria nivea | 105 | 1 | | response to | Phytohormone |
| up regulated | | | | | | | auxin | response |
| protein | | | | | | | | |
| Small auxin | SAUR70 | A0A172J249 | Boehmeria nivea | 183 | 1 | | response to | Phytohormone |
| up regulated | | | | | | | auxin | response |
| protein | | | | | | | | |
| Small auxin | SAUR71 | A0A172J2A4 | Boehmeria nivea | 183 | 1 | | response to | Phytohormone |
| up regulated | | | | | | | auxin | response |
| protein | | | | | | | | |
| Small auxin | SAUR51 | A0A172J290 | Boehmeria nivea | 97 | 1 | | response to | Phytohormone |
| up regulated | | | | | | | auxin | response |
| protein | | | | | | | | |
| Small auxin | SAUR52 | A0A172J241 | Boehmeria nivea | 149 | 1 | | response to | Phytohormone |
| up regulated | | | | | | | auxin | response |
| protein | | | | | | | | |
| Cannabidiolic acid | CBDAS | A6P6V9 | Cannabis sativa | 544 | 8 | 1.21.3.8 | oxidative | Cannabinoid |
| synthase | | | | | | | cyclization of | biosynthesis |
| | | | | | | | CBGA, producing | |
| | | | | | | | CBDA | |
| Geranylpyro- | GOT | WO | Cannabis sativa | 395 | 4 | | alkylation of | Cannabinoid |
| phosphate:olivetolate | | 2011/017798 | | | | | OLA with | biosynthesis |
| geranyltransferase | | A1 | | | | | geranyldiphosphate | |
| | | | | | | | to form CBGA | |
| Olivetolic | OAC | I1V0C9 | Cannabis sativa | 545 | 1 | 4.4.1.26 | functions in | Cannabinoid |
| acid cyclase | | | | | | | concert with | biosynthesis |
| | | | | | | | OLS/TKS to | |
| | | | | | | | form OLA | |
| Olivetolic | OAC | I6WU39 | Cannabis sativa | 101 | 5 | 4.4.1.26 | functions in | Cannabinoid |
| acid cyclase | | | | | | | concert with | biosynthesis |
| | | | | | | | OLS/TKS to | |
| | | | | | | | form OLA | |
| 3,5,7- | OLS | B1Q2B6 | Cannabis sativa | 385 | 7 | 2.3.1.206 | olivetol | Cannabinoid |
| trioxododecanoyl- | | | | | | | biosynthesis | biosynthesis |
| CoA synthase | | | | | | | | |
| Tetrahydro- | THCAS | A0A0H3UZT7 | Cannabis sativa | 325 | 1 | 1.21.3.7 | oxidative | Cannabinoid |
| cannabinolic | | | | | | | cyclization of | biosynthesis |
| acid synthase | | | | | | | CBGA, producing | |
| | | | | | | | THCA | |
| Tetrahydro- | THCAS | Q33DP7 | Cannabis sativa | 545 | 1 | 1.21.3.7 | oxidative | Cannabinoid |
| cannabinolic | | | | | | | cyclization of | biosynthesis |
| acid synthase | | | | | | | CBGA, producing | |
| | | | | | | | THCA | |
| Tetrahydro- | THCAS | Q8GTB6 | Cannabis sativa | 545 | 4 | 1.21.3.7 | oxidative | Cannabinoid |
| cannabinolic | | | | | | | cyclization of | biosynthesis |
| acid synthase | | | | | | | CBGA, producing | |
| | | | | | | | THCA | |
| Putative kinesin | kin | Q5TIP9 | Cannabis sativa | 145 | 1 | | microtubule-based | Cytoskeleton |
| heavy | | | | | | | movement | |
| chain | | | | | | | | |
| Betv1-like | Betv1 | I6XT51 | Cannabis sativa | 161 | 38 | | | Defence |
| protein | | | | | | | | response |
| ATP synthase | atp1 | A0A0M5M1Z3 | Cannabis sativa | 509 | 12 | | Produces ATP | Energy |
| subunit alpha | | | | | | | from ADP | metabolism |
| ATP synthase | atp1 | E5DK51 | Cannabis sativa | 349 | 1 | | Produces ATP | Energy |
| subunit alpha | | | | | | | from ADP | metabolism |
| ATP synthase | atp4 | A0A0M4S8F3 | Cannabis sativa | 198 | 7 | | Produces ATP | Energy |
| subunit 4 | | | | | | | from ADP | metabolism |
| ATP synthase | atpA | A0A0C5ARX6 | Cannabis sativa | 507 | 9 | | Produces ATP | Energy |
| subunit alpha | | | | | | | from ADP | metabolism |
| ATP synthase | atpB | F8TR83 | Cannabis sativa | 413 | 1 | 3.6.3.14 | Produces ATP | Energy |
| subunit beta | | | | | | | from ADP | metabolism |
| ATP synthase | atpE | A0A0C5AUH9 | Cannabis sativa | 133 | 1 | | Produces ATP | Energy |
| CF1 epsilon | | | | | | | from ADP | metabolism |
| subunit | | | | | | | | |
| ATP synthase | atpF | A0A0C5AUE9 | Cannabis sativa | 189 | 2 | | Component of | Energy |
| subunit beta, | | | | | | | the F(0) | metabolism |
| chloroplastic | | | | | | | channel | |
| NADH-ubiquinone | nad1 | A0A0M4S8G1 | Cannabis sativa | 324 | 1 | 1.6.5.3 | | Energy |
| oxidoreductase | | | | | | | | metabolism |
| chain 1 | | | | | | | | |
| NADH-ubiquinone | nad5 | A0A0M4RVP1 | Cannabis sativa | 669 | 1 | 1.6.5.3 | | Energy |
| oxidoreductase | | | | | | | | metabolism |
| chain 5 | | | | | | | | |
| NADH dehydrogenase | nad7 | A0A0M4S7M8 | Cannabis sativa | 394 | 1 | | | Energy |
| subunit 7 | | | | | | | | metabolism |
| NADH dehydrogenase | nad9 | A0A0M4R4N3 | Cannabis sativa | 190 | 2 | | | Energy |
| subunit 9 | | | | | | | | metabolism |
| NADH dehydrogenase | nadhd7 | A0A0X8GLG5 | Cannabis sativa | 394 | 1 | | | Energy |
| subunit 7 | | | | | | | | metabolism |
| NADH-quinone | ndhA | A0A0C5APZ2 | Cannabis sativa | 363 | 1 | 1.6.5.11 | NDH-1 shuttles | Energy |
| oxidoreductase | | | | | | | electrons | metabolism |
| subunit H | | | | | | | from NADH to | |
| | | | | | | | quinones | |
| NADH-quinone | ndhB | A0A0C5B2K5 | Cannabis sativa | 510 | 1 | 1.6.5.11 | NDH-1 shuttles | Energy |
| oxidoreductase | | | | | | | electrons | metabolism |
| subunit N | | | | | | | from NADH to | |
| | | | | | | | quinones | |
| NADH-quinone | ndhE | A0A0C5AUJ8 | Cannabis sativa | 101 | 4 | 1.6.5.11 | NDH-1 shuttles | Energy |
| oxidoreductase | | | | | | | electrons | metabolism |
| subunit K | | | | | | | from NADH to | |
| | | | | | | | quinones | |
| NADH-quinone | ndhJ | A0A0C5B2I2 | Cannabis sativa | 158 | 2 | 1.6.5.11 | NDH-1 shuttles | Energy |
| oxidoreductase | | | | | | | electrons | metabolism |
| subunit C | | | | | | | from NADH to | |
| | | | | | | | quinones | |
| 1-deoxy-D- | DXR | A0A1V0QSG8 | Cannabis sativa | 472 | 2 | | Converts 2-C- | Isoprenoid |
| xylulose-5- | | | | | | | methyl-D- | biosynthesis |
| phosphate | | | | | | | erythritol | |
| reductoisomerase | | | | | | | 4P into 1- | |
| | | | | | | | deoxy-D- | |
| | | | | | | | xylulose 5P | |
| Transferase | FPPS1 | A0A1V0QSH0 | Cannabis sativa | 341 | 1 | | | Isoprenoid |
| FPPS1 | | | | | | | | biosynthesis |
| Transferase | FPPS2 | A0A1V0QSH7 | Cannabis sativa | 340 | 3 | | | Isoprenoid |
| FPPS2 | | | | | | | | biosynthesis |
| Transferase | GPPS | A0A1V0QSH4 | Cannabis sativa | 393 | 2 | | | Isoprenoid |
| GPPS large | | | | | | | | biosynthesis |
| subunit | | | | | | | | |
| Transferase | GPPS | A0A1V0QSG9 | Cannabis sativa | 326 | 1 | | | Isoprenoid |
| GPPS small | | | | | | | | biosynthesis |
| subunit | | | | | | | | |
| Transferase | GPPS | A0A1V0QSI1 | Cannabis sativa | 278 | 1 | | | Isoprenoid |
| GPPS small | | | | | | | | biosynthesis |
| subunit2 | | | | | | | | |
| 4-hydroxy-3- | HDR | A0A1V0QSH9 | Cannabis sativa | 408 | 6 | | Converts (E)-4- | Isoprenoid |
| methylbut-2- | | | | | | | hydroxy-3- | biosynthesis |
| en-1-yl diphosphate | | | | | | | methylbut-2- | |
| reductase | | | | | | | en-1-yl-2P | |
| | | | | | | | into | |
| | | | | | | | isopentenyl-2P | |
| Isopentenyl- | IDI | A0A1V0QSG5 | Cannabis sativa | 304 | 7 | | Converts | Isoprenoid |
| diphosphate | | | | | | | isopentenyl | biosynthesis |
| delta-isomerase | | | | | | | diphosphate | |
| | | | | | | | into | |
| | | | | | | | dimethylallyl | |
| | | | | | | | diphosphate | |
| Mevalonate | MK | A0A1V0QSI0 | Cannabis sativa | 416 | 3 | 2.7.1.36 | Converts (R)- | Isoprenoid |
| kinase | | | | | | | mevalonate | biosynthesis |
| | | | | | | | into (R)-5- | |
| | | | | | | | phosphomevalonate | |
| Diphosphomevalonate | MPDC | A0A1V0QSG4 | Cannabis sativa | 455 | 4 | | | Isoprenoid |
| decarboxylase | | | | | | | | biosynthesis |
| Phosphomevalonate | PMK | A0A1V0QSH8 | Cannabis sativa | 486 | 4 | | Converts (R)-5- | Isoprenoid |
| kinase | | | | | | | phosphomevalonate | biosynthesis |
| | | | | | | | into (R)-5- | |
| | | | | | | | diphosphomevalonate | |
| Non-specific | ltp | P86838 | Cannabis sativa | 20 | 3 | | transfer lipids | Lipid |
| lipid-transfer | | | | | | | across | biosynthesis |
| protein | | | | | | | membranes | |
| Non-specific | ltp | W0U0V5 | Cannabis sativa | 91 | 9 | | transfer lipids | Lipid |
| lipid-transfer | | | | | | | across | biosynthesis |
| protein | | | | | | | membranes | |
| 4-coumarate:CoA | 4CL | A0A142EGJ1 | Cannabis sativa | 544 | 1 | 6.2.1.12 | forms 4-coumaroyl- | Phenylpropanoid |
| ligase | | | | | | | CoA from | biosynthesis |
| | | | | | | | 4-coumarate | |
| 4-coumarate:CoA | 4CL | V5KXG5 | Cannabis sativa | 550 | 3 | 6.2.1.12 | forms 4-coumaroyl- | Phenylpropanoid |
| ligase | | | | | | | CoA from | biosynthesis |
| | | | | | | | 4-coumarate | |
| Phenylalanine | PAL | V5KWZ6 | Cannabis sativa | 707 | 4 | 4.3.1.24 | Catalyses L- | Phenylpropanoid |
| ammonia- | | | | | | | phenylalanine = | biosynthesis |
| lyase | | | | | | | trans-cinnamate + | |
| | | | | | | | ammonia | |
| NAD(P)H-quinone | ndhF | A0A0C5AUJ6 | Cannabis sativa | 755 | 1 | 1.6.5.— | NDH shuttles | Photosynthesis |
| oxidoreductase | | | | | | | electrons from | |
| subunit 5, | | | | | | | NAD(P)H:plasto- | |
| chloroplastic | | | | | | | quinone | |
| | | | | | | | to quinones | |
| Photosystem I P700 | pasA | A0A0U2DTB0 | Cannabis sativa | 750 | 2 | 1.97.1.12 | bind P700, | Photosynthesis |
| chlorophyll a | | | | | | | the primary | |
| apoprotein A1 | | | | | | | electron donor | |
| | | | | | | | of PSI | |
| Photosystem I P700 | psaB | A0A0C5APY0 | Cannabis sativa | 734 | 2 | 1.97.1.12 | bind P700, | Photosynthesis |
| chlorophyll a | | | | | | | the primary | |
| apoprotein A2 | | | | | | | electron donor | |
| | | | | | | | of PSI | |
| Photosystem I | psaC | A0A0C5AS17 | Cannabis sativa | 81 | 10 | 1.97.1.12 | assembly of | Photosynthesis |
| iron-sulfur | | | | | | | the PSI | |
| center | | | | | | | complex | |
| Photosystem | psbB | A9XV91 | Cannabis sativa | 488 | 1 | | binds | Photosynthesis |
| II CP47 | | | | | | | chlorophyll | |
| reaction center | | | | | | | in PSH | |
| protein | | | | | | | | |
| Ribulose | rbcL | A0A0B4SX31 | Cannabis sativa | 312 | 15 | 4.1.1.39 | carboxylation | Photosynthesis |
| bisphosphate | | | | | | | of D-ribulose | |
| carboxylase | | | | | | | 1,5-bisphosphate | |
| large chain | | | | | | | | |
| Small | smt3 | Q5TIQ0 | Cannabis sativa | 76 | 2 | | response to | Phytohormone |
| ubiquitin-related | | | | | | | auxin | response |
| modifier | | | | | | | | |
| Cytochrome c | ccmFc | A0A0M4RVN1 | Cannabis sativa | 447 | 1 | | Mitochondrial | Respiration |
| biogenesis FC | | | | | | | electron | |
| | | | | | | | carrier protein | |
| Cytochrome c | ccmFn | A0A0M3UM18 | Cannabis sativa | 575 | 2 | | Mitochondrial | Respiration |
| biogenesis FN | | | | | | | electron | |
| | | | | | | | carrier protein | |
| Cytochrome c | ccsA | A0A0C5B2L0 | Cannabis sativa | 320 | 1 | | biogenesis of | Respiration |
| biogenesis | | | | | | | c-type | |
| protein CcsA | | | | | | | cytochromes | |
| Cytochrome c | cytC | P00053 | Cannabis sativa | 111 | 2 | | Mitochondrial | Respiration |
| | | | | | | | electron | |
| | | | | | | | carrier protein | |
| 7S vicilin- | Cs7S | A0A219D1T7 | Cannabis sativa | 493 | 2 | | nutrient reservoir | Storage |
| like protein | | | | | | | activity | |
| Edestin 1 | ede1D | A0A090CXP5 | Cannabis sativa | 511 | 1 | | Seed storage | Storage |
| | | | | | | | protein | |
| 4-(cytidine | CMK | A0A1V0QSI2 | Cannabis sativa | 408 | 4 | | Adds 2-phosphate | Terpenoid |
| 5′-diphospho)- | | | | | | | to 4-CDP-2-C- | biosynthesis |
| 2-C-methyl- | | | | | | | methyl-D- | |
| D-erythritol | | | | | | | erythritol | |
| kinase | | | | | | | | |
| 1-deoxy-D- | DXPS1 | A0A1V0QSH6 | Cannabis sativa | 730 | 2 | | Converts D- | Terpenoid |
| xylulose-5- | | | | | | | glyceraldehyde | biosynthesis |
| phosphate | | | | | | | 3P into 1-deoxy- | |
| synthase | | | | | | | D-xylulose 5P | |
| 1-deoxy-D- | DXS2 | A0A1V0QSH5 | Cannabis sativa | 606 | 5 | | Converts D- | Terpenoid |
| xylulose-5- | | | | | | | glyceraldehyde | biosynthesis |
| phosphate | | | | | | | 3P into 1-deoxy- | |
| synthase | | | | | | | D-xylulose 5P | |
| 4-hydroxy-3- | HDS | A0A1V0QSG3 | Cannabis sativa | 748 | 3 | | Converts (E)- | Terpenoid |
| methylbut-2-en- | | | | | | | 4-hydroxy-3- | biosynthesis |
| 1-yl diphosphate | | | | | | | methylbut-2-en- | |
| synthase | | | | | | | 1-yl-2P into | |
| | | | | | | | 2-C-methyl-D- | |
| | | | | | | | erythritol | |
| | | | | | | | 2,4-cyclo-2P | |
| 3-hydroxy-3- | hmgR | A0A1V0QSF5 | Cannabis sativa | 588 | 5 | 1.1.1.34 | synthesizes | Terpenoid |
| methylglutaryl | | | | | | | (R)-mevalonate | biosynthesis |
| coenzyme A | | | | | | | from acetyl- | |
| reductase | | | | | | | CoA | |
| 3-hydroxy-3- | hmgR | A0A1V0QSG7 | Cannabis sativa | 572 | 2 | 1.1.1.34 | synthesizes | Terpenoid |
| methylglutaryl | | | | | | | (R)-mevalonate | biosynthesis |
| coenzyme A | | | | | | | from acetyl- | |
| reductase | | | | | | | CoA | |
| Terpene synthase | TPS | A0A1V0QSF2 | Cannabis sativa | 567 | 1 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSF3 | Cannabis sativa | 551 | 3 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSF4 | Cannabis sativa | 613 | 1 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSF6 | Cannabis sativa | 551 | 1 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSF8 | Cannabis sativa | 629 | 2 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSF9 | Cannabis sativa | 624 | 2 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSG0 | Cannabis sativa | 573 | 1 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSG1 | Cannabis sativa | 640 | 1 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSG6 | Cannabis sativa | 556 | 3 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| Terpene synthase | TPS | A0A1V0QSH1 | Cannabis sativa | 594 | 1 | | formation of | Terpenoid |
| | | | | | | | cyclic terpenes | biosynthesis |
| | | | | | | | through the | |
| | | | | | | | cyclization | |
| | | | | | | | of linear | |
| | | | | | | | terpenes | |
| (−)-limonene | TPS1 | A7IZZ1 | Cannabis sativa | 622 | 2 | 4.2.3.16 | monoterpene | Terpenoid |
| synthase, | | | | | | | (C10) olefins | biosynthesis |
| chloroplastic | | | | | | | biosynthesis | |
| Maturase K | matK | A0A1V0IS32 | Cannabis sativa | 509 | 1 | | assists in | Transcription |
| | | | | | | | splicing its | |
| | | | | | | | own and other | |
| | | | | | | | chloroplast | |
| | | | | | | | group II intron | |
| Maturase K | matK | Q95BY0 | Cannabis sativa | 507 | 2 | | assists in | Transcription |
| | | | | | | | splicing its | |
| | | | | | | | own and other | |
| | | | | | | | chloroplast | |
| | | | | | | | group II intron | |
| Maturase R | matR | A0A0M5M254 | Cannabis sativa | 651 | 1 | | assists in | Transcription |
| | | | | | | | splicing introns | |
| DNA-directed | rpoB | A0A0C5ARQ8 | Cannabis sativa | 1070 | 3 | 2.7.7.6 | transcription | Transcription |
| RNA polymerase | | | | | | | of DNA | |
| subunit beta | | | | | | | into RNA | |
| DNA-directed | rpoB | A0A0C5ARX9 | Cannabis sativa | 1393 | 4 | 2.7.7.6 | transcription | Transcription |
| RNA polymerase | | | | | | | of DNA | |
| subunit beta | | | | | | | into RNA | |
| DNA-directed | rpoB | A0A0U2H5U7 | Cannabis sativa | 1070 | 1 | 2.7.7.6 | transcription | Transcription |
| RNA polymerase | | | | | | | of DNA | |
| subunit beta | | | | | | | into RNA | |
| DNA-directed | rpoC1 | A0A0C5AUF5 | Cannabis sativa | 683 | 6 | 2.7.7.6 | transcription | Transcription |
| RNA polymerase | | | | | | | of DNA | |
| subunit beta | | | | | | | into RNA | |
| DNA-directed | rpoC2 | A0A0H3W6G1 | Cannabis sativa | 1389 | 1 | 2.7.7.6 | transcription | Transcription |
| RNA polymerase | | | | | | | of DNA | |
| subunit beta | | | | | | | into RNA | |
| DNA-directed | rpoC2 | A0A0X8GKF1 | Cannabis sativa | 1391 | 1 | 2.7.7.6 | transcription | Transcription |
| RNA polymerase | | | | | | | of DNA | |
| subunit beta | | | | | | | into RNA | |
| DNA-directed | rpoC2 | A0A1V0IS28 | Cannabis sativa | 1393 | 1 | 2.7.7.7 | transcription | Transcription |
| RNA polymerase | | | | | | | of DNA | |
| subunit beta | | | | | | | into RNA | |
| Ribosomal | rpl14 | A0A0C5AS10 | Cannabis sativa | 122 | 2 | | assembly of | Translation |
| protein L14 | | | | | | | the ribosome | |
| 50S ribosomal | rpl16 | A0A0C5AUJ2 | Cannabis sativa | 119 | 2 | | assembly of | Translation |
| protein L16, | | | | | | | the 50S | |
| chloroplastic | | | | | | | ribosomal subunit | |
| Ribosomal | rpl2 | A0A0M3ULW5 | Cannabis sativa | 337 | 2 | | assembly of | Translation |
| protein L2 | | | | | | | the ribosome | |
| 50S ribosomal | rpl20 | A0A0C5B2J3 | Cannabis sativa | 120 | 1 | | Binds directly | Translation |
| protein L20 | | | | | | | to 23S rRNA to | |
| | | | | | | | assemble the 50S | |
| | | | | | | | ribosomal subunit | |
| Ribosomal | rps11 | A0A0C5ART4 | Cannabis sativa | 138 | 1 | | assembly of | Translation |
| protein S11 | | | | | | | the ribosome | |
| 30S ribosomal | rps12 | A0A0C5APY5 | Cannabis sativa | 132 | 1 | | translational | Translation |
| protein S12, | | | | | | | accuracy | |
| chloroplastic | | | | | | | | |
| 30S ribosomal | rps12 | A0A0C5B2L8 | Cannabis sativa | 125 | 1 | | translational | Translation |
| protein S12, | | | | | | | accuracy | |
| chloroplastic | | | | | | | | |
| Ribosomal | rps13 | A0A0M5M201 | Cannabis sativa | 116 | 1 | | assembly of | Translation |
| protein S13 | | | | | | | the ribosome | |
| Ribosomal | rps19 | A0A0M3ULW7 | Cannabis sativa | 94 | 1 | | assembly of | Translation |
| protein S19 | | | | | | | the ribosome | |
| Ribosomal | rps2 | A0A0C5APX8 | Cannabis sativa | 236 | 1 | | assembly of | Translation |
| protein S2 | | | | | | | the ribosome | |
| 30S ribosomal | rps3 | A0A0C5ART6 | Cannabis sativa | 155 | 3 | | assembly of | Translation |
| protein S3, | | | | | | | the 30S | |
| chloroplastic | | | | | | | ribosomal | |
| | | | | | | | subunit | |
| Ribosomal | rps3 | A0A0M3UM22 | Cannabis sativa | 548 | 1 | | assembly of | Translation |
| protein S3 | | | | | | | the ribosome | |
| Ribosomal | rps3 | A0A110BC84 | Cannabis sativa | 548 | 1 | | assembly of | Translation |
| protein S3 | | | | | | | the ribosome | |
| Ribosomal | rps4 | A0A0M4RG21 | Cannabis sativa | 352 | 1 | | assembly of | Translation |
| protein S4 | | | | | | | the ribosome | |
| Ribosomal | rps7 | A0A0C5ARU3 | Cannabis sativa | 155 | 2 | | assembly of | Translation |
| protein S7 | | | | | | | the ribosome | |
| Ribosomal | rps7 | A0A0M4R6T5 | Cannabis sativa | 148 | 1 | | assembly of | Translation |
| protein S7 | | | | | | | the ribosome | |
| Protein | ycf1 | A0A0C5AS14 | Cannabis sativa | 356 | 2 | | protein | Translation |
| TIC 214 | | | | | | | precursor | |
| | | | | | | | import into | |
| | | | | | | | chloroplasts | |
| Protein | ycf1 | A0A0H3W815 | Cannabis sativa | 1878 | 21 | | protein | Translation |
| TIC 214 | | | | | | | precursor | |
| | | | | | | | import into | |
| | | | | | | | chloroplasts | |
| Acyl-activating | aae1 | H9A1V3 | Cannabis sativa | 720 | 1 | | | Unknown |
| enzyme 1 | | | | | | | | |
| Acyl-activating | aae10 | H9A1W2 | Cannabis sativa | 564 | 1 | | | Unknown |
| enzyme 10 | | | | | | | | |
| Acyl-activating | aae12 | H9A8L1 | Cannabis sativa | 757 | 2 | | | Unknown |
| enzyme 12 | | | | | | | | |
| Acyl-activating | aae13 | H9A8L2 | Cannabis sativa | 715 | 3 | | | Unknown |
| enzyme 13 | | | | | | | | |
| Acyl-activating | aae2 | H9A1V4 | Cannabis sativa | 662 | 3 | | | Unknown |
| enzyme 2 | | | | | | | | |
| Acyl-activating | aae3 | H9A1V5 | Cannabis sativa | 543 | 7 | | | Unknown |
| enzyme 3 | | | | | | | | |
| Acyl-activating | aae4 | H9A1V6 | Cannabis sativa | 723 | 3 | | | Unknown |
| enzyme 4 | | | | | | | | |
| Acyl-activating | aae5 | H9A1V7 | Cannabis sativa | 575 | 1 | | | Unknown |
| enzyme 5 | | | | | | | | |
| Acyl-activating | aae6 | H9A1V8 | Cannabis sativa | 569 | 1 | | | Unknown |
| enzyme 6 | | | | | | | | |
| Acyl-activating | aae8 | H9A1W0 | Cannabis sativa | 526 | 3 | | | Unknown |
| enzyme 8 | | | | | | | | |
| Cannabidiolic acid | CBDAS- | A6P6W1 | Cannabis sativa | 545 | 1 | | Has no | Unknown |
| synthase-like 2 | like 2 | | | | | | cannabidiolic | |
| | | | | | | | acid | |
| | | | | | | | synthase | |
| | | | | | | | activity | |
| Putative LOV domain- | LOV | A0A126WVX7 | Cannabis sativa | 664 | 8 | | | Unknown |
| containing protein | | | | | | | | |
| Putative LOV domain- | LOV | A0A126WVX8 | Cannabis sativa | 1063 | 7 | | | Unknown |
| containing protein | | | | | | | | |
| Putative LOV domain- | LOV | A0A126WZD3 | Cannabis sativa | 574 | 1 | | | Unknown |
| containing protein | | | | | | | | |
| Putative LOV domain- | LOV | A0A126X0M1 | Cannabis sativa | 725 | 4 | | | Unknown |
| containing protein | | | | | | | | |
| Putative LOV domain- | LOV | A0A126X1H2 | Cannabis sativa | 910 | 6 | | | Unknown |
| containing protein | | | | | | | | |
| Putative LysM | lyk2 | U6EFF4 | Cannabis sativa | 599 | 1 | | | Unknown |
| domain containing | | | | | | | | |
| receptor kinase | | | | | | | | |
| Uncharacterized | unknown | A0A1V0IS79 | Cannabis sativa | 1525 | 2 | | | Unknown |
| protein | | | | | | | | |
| Uncharacterized | unknown | L0N5C8 | Cannabis sativa | 543 | 1 | | | Unknown |
| protein | | | | | | | | |
| Protein Ycf2 | ycf2 | A0A0C5APZ4 | Cannabis sativa | 2302 | 9 | | ATPase of | Unknown |
| | | | | | | | unknown | |
| | | | | | | | function | |
| Protein | secA | A0A0N9ZJA6 | Cannabis sativa' | 158 | 7 | | Binds ATP | Translation |
| translocase | | | phytoplasma | | | | | |
| subunit | | | | | | | | |
| ATP synthase | atpB | A0A0U2DTF2 | Cannabis sativa | 498 | 20 | 3.6.3.14 | Produces ATP | Energy |
| subunit beta, | | | subsp. sativa | | | | from ADP | metabolism |
| chloroplastic | | | | | | | | |
| Acetyl-coenzyme A | accD | A0A0U2DTG7 | Cannabis sativa | 497 | 3 | 2.1.3.15 | acetyl | Lipid |
| carboxylase | | | subsp. sativa | | | | coenzyme A | biosynthesis |
| carboxyl | | | | | | | carboxylase | |
| transferase | | | | | | | complex | |
| subunit beta, | | | | | | | | |
| chloroplastic | | | | | | | | |
| NAD(P)H-quinone | ndhK | A0A0U2DTF9 | Cannabis sativa | 226 | 1 | 1.6.5.— | NDH shuttles | Photosynthesis |
| oxidoreductase | | | subsp. sativa | | | | electrons | |
| subunit K, | | | | | | | from | |
| chloroplastic | | | | | | | NAD(P)H:plasto- | |
| | | | | | | | quinone | |
| | | | | | | | to quinones | |
| Cytochrome f | petA | A0A0U2DW83 | Cannabis sativa | 320 | 1 | | mediates | Photosynthesis |
| | | | subsp. sativa | | | | electron | |
| | | | | | | | transfer | |
| | | | | | | | between PSII | |
| | | | | | | | and PSI | |
| Photosystem II | psbA | A0A0U2DTE4 | Cannabis sativa | 353 | 2 | 1.10.3.9 | assembly of | Photosynthesis |
| protein D1 | | | subsp. sativa | | | | the PSII | |
| | | | | | | | complex | |
| Photosystem | psbC | A0A0U2DTE2 | Cannabis sativa | 473 | 5 | | core complex | Photosynthesis |
| II CP43 reaction | | | subsp. sativa | | | | of PSII | |
| center protein | | | | | | | | |
| Photosystem | psbD | A0A0U2DVP6 | Cannabis sativa | 353 | 3 | 1.10.3.9 | assembly of | Photosynthesis |
| II D2 protein | | | subsp. sativa | | | | the PSII | |
| | | | | | | | complex | |
| Cytochrome | psbE | A0A0U2DTH9 | Cannabis sativa | 83 | 2 | | reaction center | Photosynthesis |
| b559 subunit | | | subsp. sativa | | | | of PSII | |
| alpha | | | | | | | | |
| Ribulose | rbcL | A0A0U2DW50 | Cannabis sativa | 475 | 13 | 4.1.1.39 | carboxylation | Photosynthesis |
| bisphosphate | | | subsp. sativa | | | | of D-ribulose | |
| carboxylase | | | | | | | 1,5-bisphosphate | |
| large chain | | | | | | | | |
| Photosystem I | ycf4 | A0A0U2DVM4 | Cannabis sativa | 184 | 1 | | assembly of | Photosynthesis |
| assembly | | | subsp. sativa | | | | the PSI | |
| protein Ycf4 | | | | | | | complex | |
| 30S ribosomal | rps14 | A0A0U2DTI4 | Cannabis sativa | 100 | 2 | | Binds 16S rRNA, | Translation |
| protein S14, | | | subsp. sativa | | | | required for | |
| chloroplastic | | | | | | | the assembly of | |
| | | | | | | | 30S particles | |
| 30S ribosomal | rps15 | A0A0U2DW79 | Cannabis sativa | 90 | 1 | | assembly of | Translation |
| protein S15, | | | subsp. sativa | | | | the 30S | |
| chloroplastic | | | | | | | ribosomal | |
| | | | | | | | subunit | |
| ATP synthase | atpB | A0A0U2H0U7 | Humulus lupulus | 498 | 2 | 3.6.3.14 | Produces ATP | Energy |
| subunit beta, | | | | | | | from ADP | metabolism |
| chloroplastic | | | | | | | | |
| ATP synthase | atpB | A0A0U2H587 | Humulus lupulus | 191 | 1 | | Component of | Energy |
| subunit beta, | | | | | | | the F(0) | metabolism |
| chloroplastic | | | | | | | channel | |
| NAD(P)H-quinone | ndhI | A0A0U2GY49 | Humulus lupulus | 171 | 2 | 1.6.5.— | NDH shuttles | Photosynthesis |
| oxidoreductase | | | | | | | electrons from | |
| subunit I, | | | | | | | NAD(P)H:plasto- | |
| chloroplastic | | | | | | | quinone | |
| | | | | | | | to quinones | |
| DNA-directed RNA | rpoC2 | A0A0U2H146 | Humulus lupulus | 1398 | 1 | 2.7.7.6 | transcription | Transcription |
| polymerase | | | | | | | of DNA into | |
| subunit beta | | | | | | | RNA | |
| 50S ribosomal | rpl20 | A0A0U2H0V8 | Humulus lupulus | 120 | 1 | | Binds directly | Translation |
| protein L20, | | | | | | | to 23S rRNA to | |
| chloroplastic | | | | | | | assemble the 50S | |
| | | | | | | | ribosomal subunit | |
| 30S ribosomal | rps4 | A0A0U2H5A0 | Humulus lupulus | 202 | 1 | | binds directly | Translation |
| protein S4, | | | | | | | to 16S rRNA to | |
| chloroplastic | | | | | | | assemble the | |
| | | | | | | | 30S subunit | |
| 30S ribosomal | rps8 | A0A0U2GZU5 | Humulus lupulus | 134 | 2 | | binds directly | Translation |
| protein S8, | | | | | | | to 16S rRNA to | |
| chloroplastic | | | | | | | assemble the | |
| | | | | | | | 30S subunit | |
| Protein Ycf2 | ycf2 | A0A0U2H6B6 | Humulus lupulus | 2287 | 1 | | ATPase of | Unknown |
| | | | | | | | unknown | |
| | | | | | | | function | |

The frequency of protein for each pathway in apical buds and trichomes is illustrated in pie charts (FIG. 4).

For buds, most proteins belong to the cannabis secondary metabolism (24% in apical buds and 27% in trichomes), which encompasses the biosynthesis of phenylpropanoids, lipid, isoprenoids, terpenoids, and cannabinoids. Cannabinoid biosynthesis (5.6% in buds and 7.1% in trichomes) and terpenoid biosynthesis (6.8% in buds and 7.5% in trichomes) are a significant portion of this classification, with many terpene synthases (TPS, Table 4). We have identified two major enzymes involved in monolignol biosynthesis: phenylalanine ammonia-lyase (PAL) and 4-coumarate:CoA ligase (4CL) (Table 4); with three accessions the phenylpropanoid pathway only contributes to 1.9% of the identification results.

The second most prominent category is energy metabolism (28% in buds and 24% in trichomes), comprising photosynthesis and respiration. The third major category is gene expression metabolism (22% in buds and 26% in trichomes) which includes transcriptional and translational mechanisms. A significant portion of protein accessions remain of unknown function (13.4% in apical buds and 12.3% in trichomes). The pattern in the trichomes is very similar to that of apical buds although there is an enrichment of cannabinoid biosynthetic proteins (7.1% compared to 5.6%) and terpenoid biosynthetic proteins (7.5% to 6.8%).

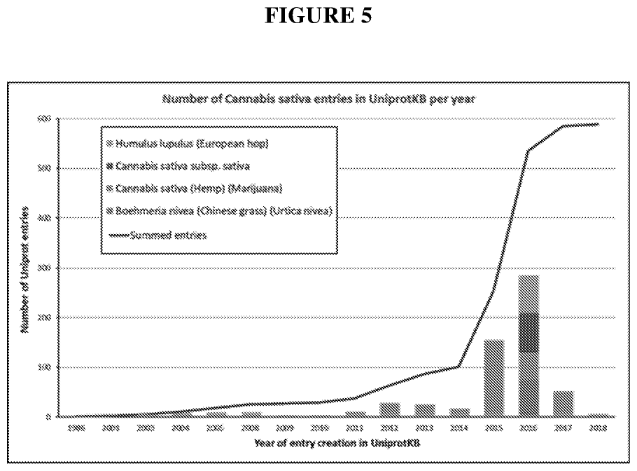

We retrieved all the entries referenced under the keyword “Cannabis sativa” in UniprotKB and produced a histogram of their distribution per year of creation; most entries (81%) were created in 2015-2017, with only 10 created in 2018 (FIG. 5). Therefore, whilst ever-increasing, the number of sequences from C. sativa publicly available in Uniprot is far from sufficient, and the proteomics community still must rely on information from unrelated plants species, such as Arabidopsis, and rice, to identify cannabis proteins.

### Example 4—Enzymes Involved in Phytocannabinoid Pathway

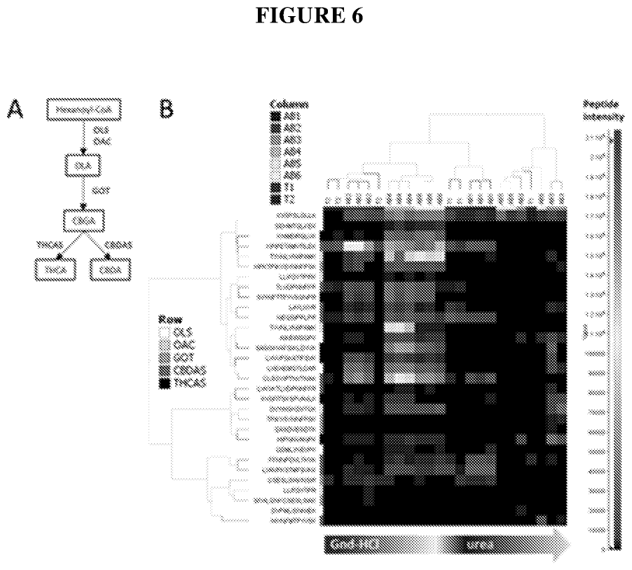

To validate the extraction methods, we focused on the cannabis-specific pathway that attracts most of the interest in the medicinal cannabis industry, namely the biosynthesis of phytocannabinoids. In our bottom-up results, five enzymes involved in phytocannabinoid biosynthesis and whose functions were described in the introduction were identified: 3,5,7-trioxododecanoyl-CoA synthase (OLS) identified with 7 peptides (19% coverage), olivetolic acid cyclase (OAC) identified with 6 peptides (13% coverage), geranyl-pyrophosphate-olivetolic acid geranyltransferase (GOT) identified with 5 peptides (17% coverage), delta9-tetrahydrocannabinolic acid synthase (THCAS) identified with 6 peptides (15% coverage), and cannabidiolic acid synthase (CBDAS) identified with 8 peptides (17% coverage). The steps these enzymes catalyse are summarised in FIG. 6A.

The two-dimensional hierarchical clustering analysis (2-D HCA) presented in FIG. 6B clusters guanidine-HCl-based samples away from the urea-based samples, in particular, methods 3 and 5. Peptides do not cluster based on the protein they belong to. The greatest majority of the peptides (24, 84%) are more abundant in samples prepared using extraction methods 4 and 6. Both methods apply a TCA/solvent precipitation step followed by resuspension in a guanidine-HCl buffer. Consequently, this is the protein extraction method we recommend in order to recover and analyse the phytocannabinoid-related enzymes using a bottom-up proteomics strategy.

As more genomes are released, the identification of additional genes in the biosynthetic pathways is likely. Already THCAS and CBDAS gene clusters have been identified where the genes are highly homologous. The function of all these genes is yet to be confirmed and proteomics methods will be useful to identify which of genes are translated at high efficiency in different cannabis strains. In designing medicinal cannabis strains for specific therapeutic requirements, either by genomic assisted breeding techniques (especially genomic selection) or through genome editing this protein expression information will be critical to optimise cannabinoid and terpene biosynthesis.

### Discussion

Six different extraction methods were assessed to analyse proteins from medicinal cannabis apical buds and trichomes. This is the first-time protein extraction is optimised from cannabis reproductive organs, and the guanidine-HCl buffer employed here has never been used before on C. sativa samples. Based on the number of intact proteins quantified and the number of peptides identified it is evident that guanidine-HCl-based methods (2, 4, and 6) are best suited to recover proteins from medicinal cannabis buds and preceding this with a precipitation step in TCA/acetone (AB4) or TCA/ethanol (AB6), ensures optimum trypsin digestion followed by MS. The method is equally applicable to trichomes and buds and the trichomes display and will be instrumental in the production of designer medicinal cannabis strains.

### Example 5—Optimisation of manual top-down proteomics analysis

The known protein standards tested are myoglobin (Myo), β-lactoglobulin (β-LG), α-S1-casein (α-S1-CN) and bovine serum albumin (BSA) which vary not only in their AA sequence, their MW, but also the number of disulfide bridges and post-translational modifications (PTMs) they present. Only mature AA sequences, i.e. not including initial methionine residues and signal peptides, are used for sequencing annotations. Myoglobin (P68083., 153 AAs) can carry a phosphoserine on its third residue, 3-lactoglobulin (P02754, 162 AAs) has two disulfide bonds, α-S1-casein (P02662, 199 AAs) is constitutively phosphorylated with up to nine phosphoserines, and BSA (P02769, 583 AAs) contains 35 disulfide bonds as well as various PTMs, most of which are phosphorylation sites. Oxidation of methionine residues of protein standards was encountered, possibly resulting from vortexing during the sample preparation. Precursors of oxidized proteoforms is purposefully disregarded in the manual annotation step, however, it is included as a dynamic modification for the Mascot search.

Tandem MS data from infused known protein standards fragmented using SID, ETD, CID and HCD were processed either manually in order to include SID data which are not considered as genuine MS/MS data, or automatically on bona fide MS/MS data only to test whether an automated workflow would successfully reproduce manual searches, and therefore could be applied to unknown proteins from cannabis samples. For manual curation, not all the MS/MS data produced was used, only that corresponding to the major isoforms. For instance, an oxidised proteoform of myoglobin was found but ignored for the manual annotation step which proved very labour-intensive and time-consuming.

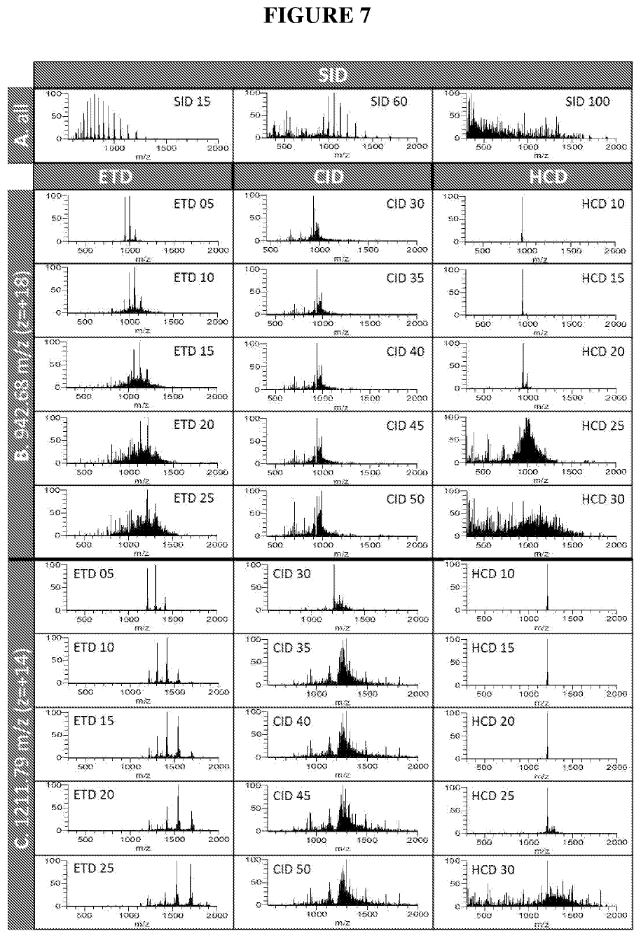

FIG. 7 displays spectra from myoglobin acquired following SID, ETD, CID, and HCD where increased energy was applied. No fragmentation is observed at SID 15V. Fragmentation of the most abundant ions of lower m/z starts to occur at SID 45V (not shown), is evident at SID 60V, and complete at SID 100V (FIG. 7A).

Whilst MS/MS spectra of the most abundant multiply-charged ions were obtained as attested in Table 5, only two charge states, 942.68 m/z (z=+18) and 1211.79 m/z (z=+14), are exemplified in FIGS. 7B and 7C, respectively. Applying ETD for increasingly longer periods, from 5 to 25 ms, results in greater protein dissociations. As ETD fragmentation improves, fragments mass range extends from intermediate to high m/z values (FIG. 7B). Less fragmentation is observed when ETD is applied for 5 ms (356 and 143 deisotoped fragments for 942.68 m/z and 1211.79 m/z, respectively), than when ETD is sustained for longer activation times (Table 5).

Maximum number of fragments are reached with 20 ms for 942.68 m/z (516 deisotoped fragments) and 15 ms from 1211.79 m/z (455 deisotoped fragments) (Table 5).

TABLE 5
Number of spectral MS/MS fragments for each protein standard

| Myoglobin | | m/z | All | 848.51 | 893.22 | 942.68 | 1211.79 | 1304.93 | |
|---|---|---|---|---|---|---|---|---|---|
| | | Z | NA | 20 | 19 | 18 | 14 | 13 | |
| | | RI(%) | NA | 100 | 98 | 96 | 38 | 24 | |
| | MS/MS mode | NCE | | | | | | | Mean |
| | SID | 15 | 171 | | | | | | 171 |
| | SID | 60 | 725 | | | | | | 725 |
| | SID | 100 | 656 | | | | | | 656 |
| | CID | 30 | | 210 | 174 | 194 | 241 | 180 | 200 |
| | CID | 35 | | 255 | 180 | 233 | 369 | 389 | 285 |
| | CID | 40 | | 223 | 176 | 243 | 389 | 411 | 288 |
| | CID | 45 | | 226 | 219 | 227 | 385 | 383 | 288 |
| | CID | 50 | | 233 | 227 | 209 | 402 | 368 | 288 |
| | ETD | 5 | | 220 | 229 | 356 | 143 | 79 | 205 |
| | ETD | 10 | | 66 | 172 | 470 | 392 | 282 | 276 |
| | ETD | 15 | | 120 | 190 | 504 | 455 | 273 | 308 |
| | ETD | 20 | | 135 | 457 | 516 | 411 | 309 | 366 |
| | ETD | 25 | | 89 | 431 | 468 | 365 | 263 | 323 |
| | HCD | 10 | | 102 | 71 | 116 | 60 | 42 | 78 |
| | HCD | 15 | | 146 | 148 | 175 | 105 | 118 | 138 |
| | HCD | 20 | | 250 | 244 | 280 | 252 | 262 | 258 |
| | HCD | 25 | | 253 | 301 | 511 | 529 | 499 | 419 |
| | HCD | 30 | | 303 | 260 | 376 | 462 | 572 | 395 |
| | Min | | 171 | 66 | 71 | 116 | 60 | 42 | |
| | Max | | 656 | 303 | 457 | 516 | 529 | 572 | |
| | Mean | | 517 | 189 | 232 | 325 | 331 | 295 | 274 |
| b-LG | | m/z | All | 972.19 | 1026.15 | 1091.4 | 1232.84 | | |
| | | Z | NA | 19 | 18 | 17 | 15 | | |
| | | RI(%) | NA | 46 | 74 | 80 | 100 | | |
| | SID | 15 | 543 | | | | | | 543 |
| | SID | 60 | 2160 | | | | | | 2160 |
| | SID | 100 | 3882 | | | | | | 3882 |
| | CID | 30 | | 336 | 344 | 397 | 481 | | 390 |
| | CID | 35 | | 392 | 412 | 507 | 529 | | 460 |
| | CID | 40 | | 333 | 397 | 474 | 571 | | 444 |
| | CID | 45 | | 358 | 439 | 511 | 531 | | 460 |
| | CID | 50 | | 343 | 387 | 440 | 544 | | 429 |
| | ETD | 5 | | 379 | 220 | | 160 | | 253 |
| | ETD | 10 | | 375 | 271 | | 456 | | 367 |
| | ETD | 15 | | 325 | 137 | | 433 | | 298 |
| | ETD | 20 | | 412 | 170 | | 431 | | 338 |
| | ETD | 25 | | 242 | 102 | | 443 | | 262 |
| | HCD | 10 | | 155 | 230 | 252 | 119 | | 189 |
| | HCD | 15 | | 395 | 469 | 608 | 517 | | 497 |
| | HCD | 20 | | 504 | 588 | 815 | 664 | | 643 |
| | HCD | 25 | | 310 | 449 | 634 | 737 | | 533 |
| | HCD | 30 | | 298 | 350 | 443 | 419 | | 378 |
| | Min | | 543 | 155 | 102 | 252 | 119 | | |
| | Max | | 3882 | 504 | 588 | 815 | 737 | | |
| | Mean | | 2195 | 344 | 331 | 508 | 469 | | 413 |
| a-S1-CN | | m/z | All | 1139.6 | 1193.38 | 1319.14 | 1480.59 | | |
| | | Z | NA | 21 | 20 | 18 | 17 | 16 | |
| | | RI(%) | NA | 94 | 100 | 70 | 52 | 36 | |
| | SID | 15 | 414 | | | | | | 414 |
| | SID | 60 | 728 | | | | | | 728 |
| | SID | 100 | 891 | | | | | | 891 |
| | CID | 30 | | 159 | 166 | | 51 | | 125 |
| | CID | 35 | | 455 | 460 | | 247 | | 387 |
| | CID | 40 | | 401 | 466 | | 259 | | 375 |
| | CID | 45 | | 455 | 389 | | 254 | | 366 |
| | CID | 50 | | 432 | 375 | | 259 | | 356 |
| | ETD | 5 | | | 111 | 97 | | | 104 |
| | ETD | 10 | | | 424 | 302 | | | 363 |
| | ETD | 15 | | | 352 | 224 | | | 288 |
| | ETD | 20 | | | 292 | 209 | | | 251 |
| | ETD | 25 | | | 193 | 145 | | | 169 |
| | HCD | 10 | | 112 | 120 | 51 | | 46 | 82 |
| | HCD | 15 | | 660 | 702 | 721 | | 472 | 639 |
| | HCD | 20 | | 660 | 651 | 586 | | 464 | 590 |
| | HCD | 25 | | 431 | 519 | 544 | | 459 | 488 |
| | HCD | 30 | | 289 | 301 | 256 | | 251 | 274 |
| | Min | | 414 | 112 | 111 | 51 | 51 | 46 | |
| | Max | | 891 | 660 | 702 | 721 | 259 | 472 | |
| | Mean | | 678 | 406 | 368 | 314 | 214 | 338 | 324 |
| BSA | | m/z | All | 953.93 | 994.98 | 1061.5 | 118.08 | | |
| | | Z | NA | 72 | 69 | 65 | 59 | | |
| | | RI(%) | NA | 72 | 76 | 68 | 44 | | |
| | SID | 15 | | | | | | | |
| | SID | 60 | 84 | | | | | | 84 |
| | SID | 100 | 436 | | | | | | 436 |
| | CID | 30 | | | 0 | 0 | 0 | | 0 |
| | CID | 35 | | | 182 | 203 | 109 | | 165 |
| | CID | 40 | | | 150 | 177 | 96 | | 141 |
| | CID | 45 | | | 153 | 196 | 101 | | 150 |
| | CID | 50 | | | 157 | 223 | 125 | | 168 |
| | ETD | 5 | | 0 | | 0 | | | 0 |
| | ETD | 10 | | 161 | | 359 | | | 260 |
| | ETD | 15 | | 58 | | 409 | | | 234 |
| | ETD | 20 | | 124 | | 352 | | | 238 |
| | ETD | 25 | | 58 | | 277 | | | 168 |
| | HCD | 10 | | 0 | 0 | | | | 0 |
| | HCD | 15 | | 232 | 196 | | | | 214 |
| | HCD | 20 | | 238 | 227 | | | | 233 |
| | HCD | 25 | | 113 | 121 | | | | 117 |
| | HCD | 30 | | 85 | 87 | | | | 86 |
| | Min | | 84 | 0 | 0 | 0 | 0 | | |
| | Max | | 436 | 238 | 227 | 409 | 125 | | |
| | Mean | | 260 | 107 | 127 | 220 | 86 | | 145 |

Increasing the energy of CID mode from 35 to 50 eV has less impact on fragmentation as can be visually assessed on FIGS. 7B and 7C and in Table 5, with more constant numbers of fragments generated, albeit still increasing with the energy levels applied. As CID fragmentation intensifies, more ions of low m/z appear (FIG. 7B). The least number of fragments are obtained at CID 35 eV (194 and 241 deisotoped fragments for 942.68 m/z and 1211.79 m/z, respectively) and maximum numbers are reached at CID 50 eV with 209 and 402 fragments for 942.68 m/z and 1211.79 m/z, respectively (Table 5). Compiling all CID fragment masses together in Prosight Lite program yields a myoglobin sequence coverage of 44%. Similar to ETD, fragmentation resulting from HCD mode is enhanced as more energy is applied, from 10 to 30 eV. This is clearly visible on FIGS. 7B and 7C, with only a handful of fragments observed at HCD 10-15 eV, and fragmentation fully developing at HCD 20 eV and above. As HCD fragmentation improves, the mass range of the ions visibly extends (FIGS. 7B and 7C). Only 116 and 60 deisotoped fragments were detected at HCD 10 eV from 942.68 m/z and 1211.79 m/z, respectively, with number of fragments peaking at HCD 25 eV to 511 and 529 for 942.68 m/z and 1211.79 m/z, respectively (Table 5). Compiling all HCD fragment masses together in Prosight Lite program yielded a myoglobin sequence coverage of 57%. The outcome of fragmentation is much less dependent on a particular collisional value for CID than for HCD. Furthermore, while CID and HCD spectra are very similar, HCD achieves optimal fragmentation at lower energy levels.

Different precursors of the same protein (i.e. different charge states) require different energy level for optimum fragmentation (Table 5). Furthermore, targeting a lower charge state shifts the fragment masses to the right of the mass range, towards high m/z values (FIG. 7C). Row averages of fragments across all five charge states of myoglobin (+20, +19, +18, +14, +13) highlight that a minimum energy level must be reached for any meaningful protein dissociation to occur (Table 5). As far as myglobin is concerned, these values are 60 eV for SID, 25 eV for HCD, 20 ms for ETD, and 40-50 eV for CID, sorted in decreasing order. Column averages of fragments across all MS/MS modes indicate that some precursors are more amenable to fragmentation than others, with charge states +18 (942.68 m/z) and +14 (1211.79 m/z) on average generating most fragments (325 and 331, respectively, Table 5). This suggests that parent ions displaying both high m/z (low charge state) and high intensity should be favoured for top-down sequencing experiments.

All the deconvoluted and deisotoped masses obtained by applying increasing energy levels of SID, CID, HCD and ETD were submitted to ProSight Lite and searched against the AA sequence of myoglobin, without the initial methionine which gets processed out during the maturation step. All the resulting matching b-, c-, y-, and z-type ions are reported into Table 6 and plotted according to their position along the mature AA sequence of myoglobin (153 AA).

TABLE 6
Number of matching ions in Prosight Lite program (tolerance of 50 ppm) for each protein standard

| Myoglobin | | m/z | All | 848.51 | 893.22 | 942.68 | 1211.79 | 1304.93 | |
|---|---|---|---|---|---|---|---|---|---|
| | | Z | NA | 20 | 19 | 18 | 14 | 13 | |
| | | RI(%) | NA | 100 | 98 | 96 | 38 | 24 | |
| | MS/MS mode | NCE | | | | | | | Mean |
| | SID | 15 | 1 | | | | | | 1 |
| | SID | 60 | 19 | | | | | | 19 |
| | SID | 100 | 20 | | | | | | 20 |
| | CID | 30 | | 10 | 4 | 10 | 27 | 13 | 13 |
| | CID | 35 | | 12 | 8 | 12 | 42 | 41 | 23 |
| | CID | 40 | | 11 | 8 | 14 | 44 | 40 | 23 |
| | CID | 45 | | 10 | 9 | 14 | 39 | 44 | 23 |
| | CID | 50 | | 19 | 12 | 14 | 36 | 44 | 25 |
| | ETD | 5 | | 25 | 6 | 17 | 5 | 2 | 11 |
| | ETD | 10 | | 17 | 24 | 36 | 24 | 21 | 24 |
| | ETD | 15 | | 28 | 17 | 45 | 29 | 20 | 28 |
| | ETD | 20 | | 40 | 45 | 57 | 36 | 21 | 40 |
| | ETD | 25 | | 28 | 48 | 53 | 26 | 19 | 35 |
| | HCD | 10 | | 2 | 3 | 2 | 1 | 1 | 2 |
| | HCD | 15 | | 4 | 2 | 5 | 2 | 4 | 3 |
| | HCD | 20 | | 9 | 11 | 22 | 12 | 7 | 12 |
| | HCD | 25 | | 17 | 11 | 33 | 48 | 55 | 33 |
| | HCD | 30 | | 17 | 11 | 22 | 52 | 47 | 30 |
| | Min | | 1 | 2 | 2 | 2 | 1 | 1 | 2 |
| | Max | | 20 | 40 | 48 | 57 | 52 | 55 | 45 |
| | Mean | | 13 | 17 | 15 | 24 | 28 | 25 | 20 |
| | Length of seq (AA) | | 153 | 153 | 153 | 153 | 153 | 153 | 153 |
| | % Max | | 13.1 | 26.1 | 31.4 | 37.3 | 34.0 | 35.9 | 30 |
| b-LG | | m/z | All | 972.19 | 1026.15 | 1091.4 | 1232.84 | | |
| | | Z | NA | 19 | 18 | 17 | 15 | | |
| | | RI(%) | NA | 46 | 74 | 80 | 100 | | |
| | SID | 15 | 2 | | | | | | 2 |
| | SID | 60 | 27 | | | | | | 27 |
| | SID | 100 | 66 | | | | | | 66 |
| | CID | 30 | | 11 | 11 | 11 | 23 | | 14 |
| | CID | 35 | | 17 | 18 | 24 | 23 | | 21 |
| | CID | 40 | | 20 | 19 | 23 | 21 | | 21 |
| | CID | 45 | | 20 | 20 | 26 | 23 | | 22 |
| | CID | 50 | | 21 | 17 | 18 | 22 | | 20 |
| | ETD | 5 | | 8 | 4 | | 4 | | 5 |
| | ETD | 10 | | 20 | 9 | | 8 | | 12 |
| | ETD | 15 | | 14 | 9 | | 12 | | 12 |
| | ETD | 20 | | 20 | 14 | | 13 | | 16 |
| | ETD | 25 | | 20 | 11 | | 19 | | 17 |
| | HCD | 10 | | 1 | 6 | 5 | 3 | | 4 |
| | HCD | 15 | | 14 | 28 | 34 | 17 | | 23 |
| | HCD | 20 | | 19 | 24 | 29 | 27 | | 25 |
| | HCD | 25 | | 15 | 22 | 28 | 27 | | 23 |
| | HCD | 30 | | 21 | 20 | 26 | 21 | | 22 |
| | Min | | 2 | 1 | 4 | 5 | 3 | | 3 |
| | Max | | 66 | 21 | 28 | 29 | 23 | | 33 |
| | Mean | | 32 | 16 | 15 | 22 | 18 | | 21 |
| | Length of seq (AA) | | 162 | 162 | 162 | 162 | 162 | | 162 |
| | % Max | | 40.7 | 13.0 | 17.3 | 17.9 | 14.2 | | 21 |
| a-S1-CN | | m/z | All | 1139.6 | 1193.38 | 1319.14 | 1480.59 | | |
| | | Z | NA | 21 | 20 | 18 | 17 | 16 | |
| | | RI(%) | NA | 94 | 100 | 70 | 52 | 36 | |
| | SID | 15 | 1 | | | | | | 1 |
| | SID | 60 | 3 | | | | | | 3 |
| | SID | 100 | 7 | | | | | | 7 |
| | CID | 30 | | 4 | 2 | | 6 | | 4 |
| | CID | 35 | | 7 | 10 | | 12 | | 10 |
| | CID | 40 | | 8 | 9 | | 12 | | 10 |
| | CID | 45 | | 7 | 10 | | 9 | | 9 |
| | CID | 50 | | 17 | 6 | | 15 | | 13 |
| | ETD | 5 | | | 3 | 0 | | | 2 |
| | ETD | 10 | | | 23 | 13 | | | 18 |
| | ETD | 15 | | | 25 | 15 | | | 20 |
| | ETD | 20 | | | 24 | 19 | | | 22 |
| | ETD | 25 | | | 25 | 18 | | | 22 |
| | HCD | 10 | | 1 | 2 | 1 | | 1 | 1 |
| | HCD | 15 | | 24 | 32 | 30 | | 28 | 29 |
| | HCD | 20 | | 37 | 41 | 35 | | 33 | 37 |
| | HCD | 25 | | 43 | 37 | 39 | | 39 | 40 |
| | HCD | 30 | | 37 | 36 | 38 | | 38 | 37 |
| | Min | | 1 | 1 | 2 | 0 | 6 | 1 | 2 |
| | Max | | 7 | 43 | 41 | 39 | 15 | 39 | 31 |
| | Mean | | 4 | 19 | 19 | 23 | 11 | 28 | 17 |
| | Length of seq (AA) | | 199 | 199 | 199 | 199 | 199 | 199 | 199 |
| | % Max | | 3.5 | 21.6 | 20.6 | 19.6 | 7.5 | 19.6 | 15 |
| BSA | | m/z | All | 953.93 | 994.98 | 1061.5 | 118.08 | | |
| | | Z | NA | 72 | 69 | 65 | 59 | | |
| | | RI(%) | NA | 72 | 76 | 68 | 44 | | |
| | SID | 15 | | | | | | | |
| | SID | 60 | 1 | | | | | | 1 |
| | SID | 100 | 4 | | | | | | 4 |
| | CID | 30 | | | 0 | 0 | 0 | | 0 |
| | CID | 35 | | | 4 | 6 | 4 | | 5 |
| | CID | 40 | | | 5 | 5 | 2 | | 4 |
| | CID | 45 | | | 5 | 5 | 3 | | 4 |
| | CID | 50 | | | 1 | 6 | 7 | | 5 |
| | ETD | 5 | | 0 | | 0 | | | 0 |
| | ETD | 10 | | 6 | | 4 | | | 5 |
| | ETD | 15 | | 4 | | 8 | | | 6 |
| | ETD | 20 | | 8 | | 4 | | | 6 |
| | ETD | 25 | | 7 | | 8 | | | 8 |
| | HCD | 10 | | 0 | 0 | | | | 0 |
| | HCD | 15 | | 9 | 3 | | | | 6 |
| | HCD | 20 | | 13 | 11 | | | | 12 |
| | HCD | 25 | | 11 | 12 | | | | 12 |
| | HCD | 30 | | 9 | 11 | | | | 10 |
| | Min | | 1 | 0 | 0 | 0 | 0 | | 0 |
| | Max | | 4 | 13 | 12 | 8 | 7 | | 9 |
| | Mean | | 2 | 7 | 5 | 5 | 3 | | 4 |
| | Length of seq (AA) | | 583 | 583 | 583 | 583 | 583 | | 583 |
| | % Max | | 0.7 | 2.2 | 2.1 | 1.4 | 1.2 | | 2 |

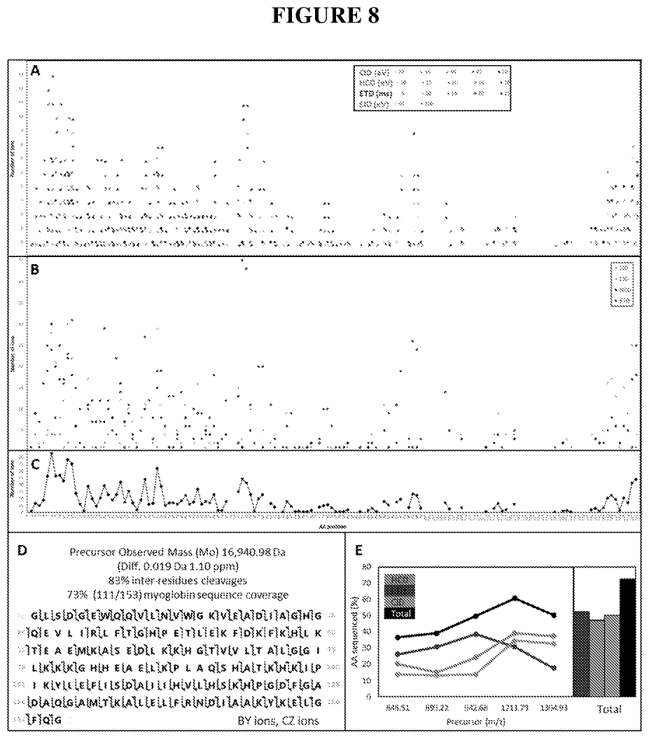

Because different ions of the same protein underwent different types of fragmentation at varying energy levels, the data is quite redundant, with many dots depicted at a particular AA position (FIG. 8A).

Mostly darker colours are represented, confirming that higher energy levels produced meaningful data. FIG. 8B corresponds to the summation of the number of matched ions per MS/MS mode, irrespective of the energy applied. It shows that some parts of the sequence are highly amenable to specific dissociation modes. For instance, ETD is more suited for N-terminus and the central part of the protein, while CID and HCD help sequence the C-terminus. CID generates predominantly low yields N- and C-terminal fragments from intact proteins. SID was only effective on the N-terminus of myoglobin.

FIG. 8C represents a summation of the number of matched ions at each AA position, irrespective of the MS/MS mode or the energy applied. Because less dots are displayed, the areas of myoglobin that resisted fragmentation under our conditions become more visible. Myoglobin N-terminus is well covered up to position 99, albeit with some interruptions, whereas the C-terminus is only covered up to the last 10 AAs. The region spanning AAs 100 to 140 of myoglobin is only partially sequenced

ProSight Lite output confirmed that both N- and C-termini of myoglobin sequence are well covered, with many AAs identified from b-, c-, y-, and z-types of ions (FIG. 8D). Some AAs were could only be fragmented once, either using ETD or HCD. Therefore, resorting to multiple MS/MS modes is essential to maximise top-down sequencing. Overall, 83% inter-residues cleavages were annotated, accounting for 73% (111/153 AAs) sequence coverage of myoglobin (FIG. 8D). FIG. 8C summarizes top-down sequencing efficiency for myoglobin in these experiments. It varies according to the charge state and the dissociation type.

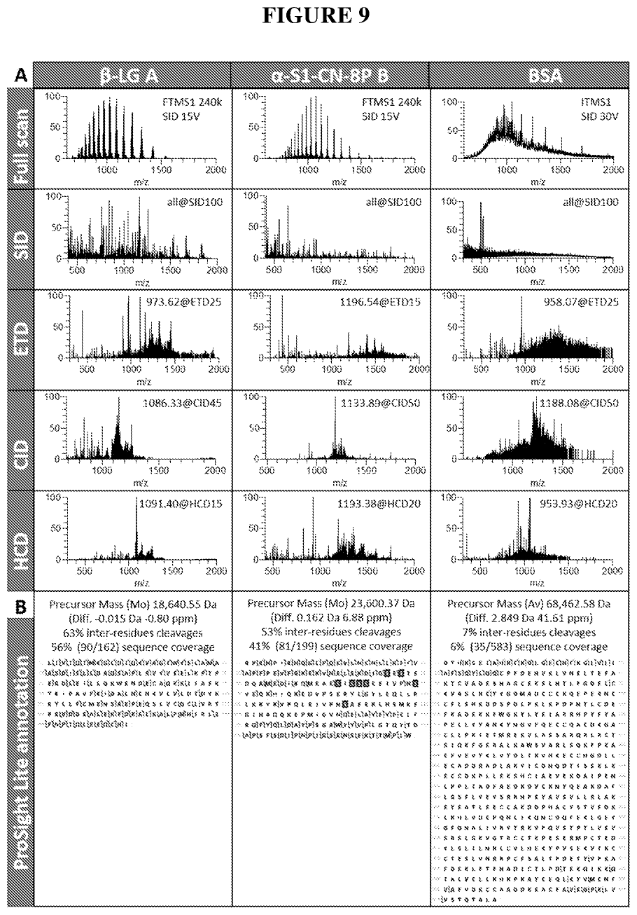

The commercial standards used in this study contain mixtures of protein isoforms. Deconvolution of full scan FTMS1 (FIG. 9A) supplied accurate masses for β-lactoglobulin, α-S1-casein and average masses for BSA with an error <50 ppm, which assisted in the determination of which protein isoforms underwent MS/MS analysis and which sequence to use for ProSight Lite annotation.

Precursors from allelic variant A of β-lactoglobulin and allelic variant B of α-S1-casein with eight phosphorylation were selected for fragmentation. Examples of SID, ETD, CID, and HCD spectra for each protein are shown in FIG. 9A. Theoretical charge state distributions for proteins showed that the absolute number of charges that precursors carry and the relative width of the charge state distribution both increased as protein mass augmented. In this study, high numbers of microscans were used to perform spectral averaging in order to increase S/N but the trade-off is a longer duty cycle and acquisition time, which restricts throughput.

The number of deconvoluted, deisotoped fragments of all protein standards are listed in Table 5. As previously observed for myoglobin, fragmentation efficiency assessed on the number of fragments generated depends on the charge state of the precursor, the MS/MS mode, and the energy applied, albeit in a protein-specific fashion. For instance, abundant parents of lower charge states yielded numerous fragments in the case of β-lactoglobulin (z=+17, 508 fragments on average) and BSA (z=+68, 220 fragments on average), whereas abundant precursor of high charge state yielded numerous fragments in the case of α-S1-casein (z=+21, 406 fragments on average). If we look at which MS/MS mode and which energy level produced the greatest number of fragments on average across all charge states, we find that the ranking for β-lactoglobulin is SID 100 V>HCD 20 eV>CID 35-45 eV>ETD 10 ms. The ranking for α-S1-casein is SID 100 V>HCD 15 eV>CID 35 eV>ETD 10 ms. The ranking for BSA is SID 100 V>ETD 10 ms>HCD 20 eV>CID 50 eV.

A plethora of fragments does not necessary translate into high AA sequence coverage as can be seen when Tables 5 and 6, similarly arranged, are compared. The phenomenon of “overfragmentation” is predicted to result from secondary dissociation of the initial daughter ions when normalized collision energies are enhanced. Whilst noticeable for all MS/MS modes tested, the best evidence of this applied to SID fragmentation with at best only 3% (26/656 for myoglobin) of the fragments being annotated in ProSight Lite. Its efficacy in top-down sequencing varies greatly among the proteins studied here, accounting for as little as 1% coverage of BSA sequence, 4% coverage of α-S1-casein sequence, up to 13% for myoglobin and an impressive 41% for (3-lactoglobulin (Table 6).

When true MS/MS data resulting from ETD, CID, HCD experiments are considered, high number of fragments are a requisite for proper top-down sequencing, yet it is not the MS/MS spectra with the maximum number of peaks that yields the greatest number of matched ions in ProSight Lite (Tables 5 and 6). For instance, in the case of (3-lactoglobulin precursor 1091.4 m/z undergoing HCD fragmentation, 815 fragments were obtained with 20 eV which accounted for 29 matched ions, and 608 fragments were obtained with 15 eV which accounted for 34 matched ions. In another example, looking at α-S1-casein precursor 1139.6 m/z undergoing CID fragmentations, 35 eV created 455 fragments with only 7 being annotated in Prosight Lite, while 435 fragments obtained with 50 eV led to 17 matches. Compiling all fragmentation data obtained for each protein and submitting them to Prosight Lite program gave the maximum sequence coverage achieved in this study: 56% for β-lactoglobulin, 41% for α-S1-casein and 6% for BSA (FIG. 9B).

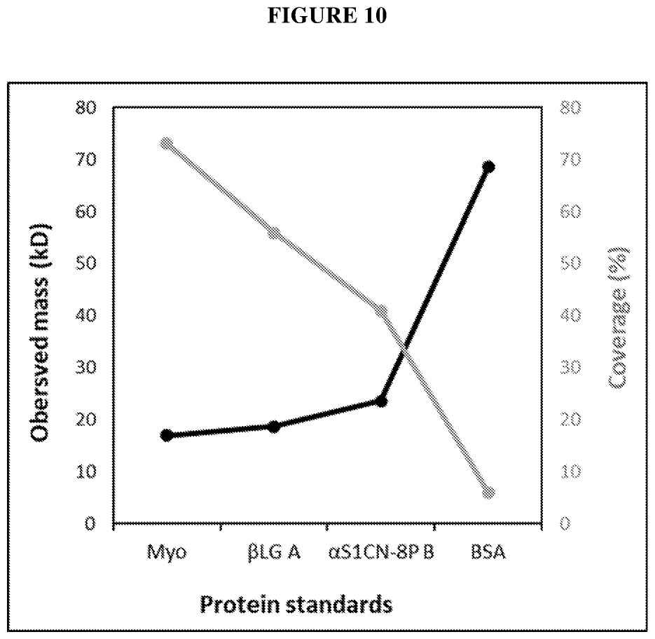

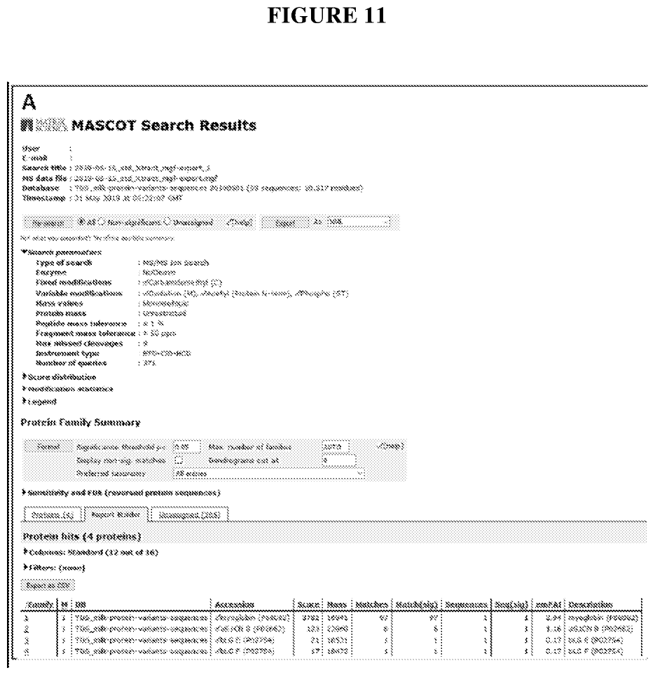

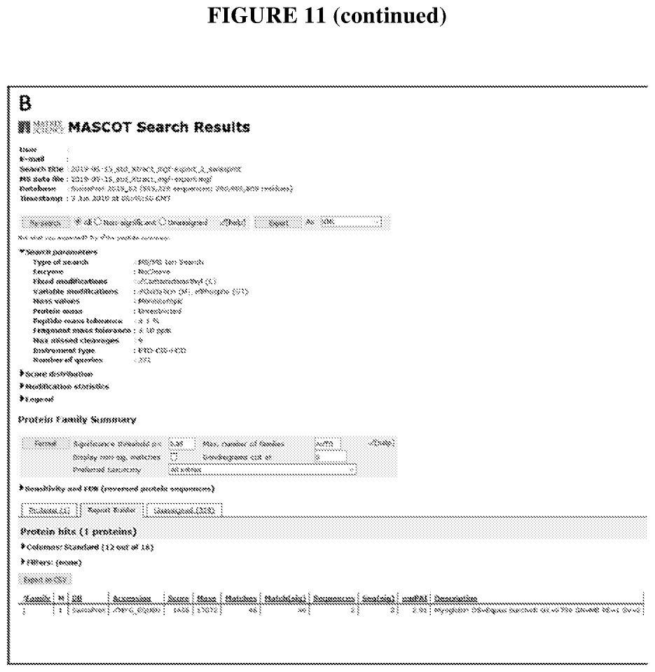

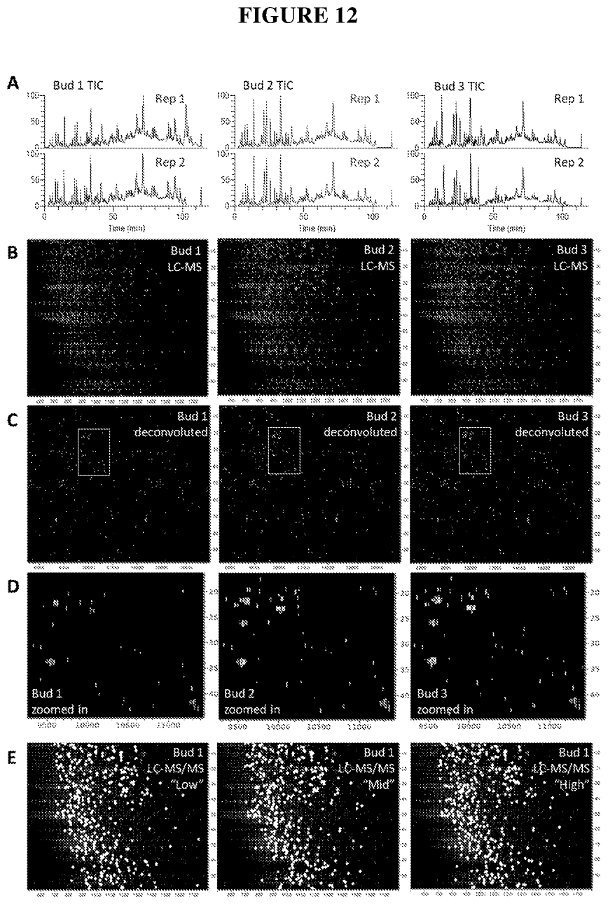

These data demonstrate that for known proteins of different MWs, sequence coverage varies according to the protein itself, its size (FIG. 10) and intrinsic properties, the abundance and charge state of the precursor ion, the MS/MS mode, and the level of energy applied. Therefore, not many general rules can be surmised apart from the fact that the more MS/MS data, the greater the sequence coverage. A key factor though is the signal intensity, the higher S/N the better the fragmentation pattern (data not shown). Generally speaking and under the optimised conditions, medium to high energy levels tend to improve sequence annotation.

### Example 6—Optimisation of Automatic Top-Down Proteomics Analysis

An automated workflow was developed using Proteome Discovered to export a Mascot Generic File (MGF) containing 371 MS/MS peak lists which was submitted to Mascot algorithm. The parameters bearing the greatest impact on the results were tested, namely the database, the type of dynamic modifications and the fragment tolerance. The search results are summarised in Table 7. Mascot outcome was then compared to the manual curation described above. The immediate advantage of automation is the speed at which all the data is processed, not accounting for database search times which can be significant (days if the error-tolerant option is selected in mascot program). Another advantage is that the search runs in the background, freeing up time to perform other tasks. Automation also greatly limits the potential for man-made errors.

TABLE 7
Summary of Mascot results for standards and cannabis samples using
various databases, dynamic modifications, and fragment tolerance.

| Mascot | | | | # | # | Static | Dynamic | Frag. | |
|---|---|---|---|---|---|---|---|---|---|
| job # | Sample | DB | Taxonomy | entries | residues | mods. | mods. | toler. | |
| 19018 | Stand. | HM | all | 59 | 10,517 | carbamidomethyl C | Protein N-term | 50 | ppm |
| | | | | | | | acetyl, | | |
| | | | | | | | oxidation M, | | |
| | | | | | | | phospho ST | | |
| 19037 | Stand. | HM | all | 59 | 10,517 | carbamidomethyl C | Protein N-term | 2 | Da |
| | | | | | | | acetyl, | | |
| | | | | | | | oxidation M, | | |
| | | | | | | | phospho ST | | |
| 19020 | Stand. | SP | all | 559228 | 200,905,869 | carbamidomethyl C | oxidation M, | 50 | ppm |
| | | | | | | | phospho ST | | |
| 19040 | Stand. | SP | all | 559228 | 200,905,869 | carbamidomethyl C | oxidation M, | 2 | Da |
| | | | | | | | phospho ST | | |
| 19052 | Stand. | SP | other | 13186 | | carbamidomethyl C | Protein N-term | 50 | ppm |
| | | | mammalia | | | | acetyl, | | |
| | | | | | | | oxidation M, | | |
| | | | | | | | phospho ST | | |
| 19047 | Stand. | SP | other | 13186 | | carbamidomethyl C | Protein N-term | 2 | Da |
| | | | mammalia | | | | acetyl, | | |
| | | | | | | | oxidation M, | | |
| | | | | | | | phospho ST | | |
| 19031 | Canna. | UP | all | 663 | 221,206 | carbamidomethyl C | Protein N-term | 50 | ppm |
| | | | | | | | acetyl, | | |
| | | | | | | | oxidation M | | |
| 19030 | Canna. | UP | all | 663 | 221,206 | carbamidomethyl C | Protein N-term | 50 | ppm |
| | | | | | | | acetyl, | | |
| | | | | | | | oxidation M | | |
| 19048 | Canna. | UP | all | 663 | 221,206 | carbamidomethyl C | Protein N-term | 2 | Da |
| | | | | | | | acetyl, | | |
| | | | | | | | oxidation M | | |
| 19050 | Canna. | UP | all | 663 | 221,206 | carbamidomethyl C | Protein N-term | 50 | ppm |
| | | | | | | | acetyl, | | |
| | | | | | | | oxidation M, | | |
| | | | | | | | phospho ST | | |
| 19049 | Canna. | UP | all | 663 | 221,206 | carbamidomethyl C | Protein N-term | 2 | Da |
| | | | | | | | acetyl, | | |
| | | | | | | | oxidation M, | | |
| | | | | | | | phospho ST | | |
| 19051 | Canna. | UP | all | 663 | 221,206 | carbamidomethyl C | none | 50 | ppm |
| 19043 | Canna. | UP | all | 663 | 221,206 | carbamidomethyl C | none | 2 | Da |
| 19042 | Canna. | SP | all | 559228 | 200,905,869 | carbamidomethyl C | none | 2 | Da |
| 19044 | Canna. | SP | viridiplantae | 39800 | | carbamidomethyl C | none | 2 | Da |
| 19045 | Canna. | SP | viridiplantae | 39800 | | carbamidomethyl C | Protein N-term | 2 | Da |
| | | | | | | | acetyl, | | |
| | | | | | | | oxidation M | | |
| 19046 | Canna. | SP | viridiplantae | 39800 | | carbamidomethyl C | Protein N-term | 2 | Da |
| | | | | | | | acetyl, | | |
| | | | | | | | oxidation M, | | |
| | | | | | | | phospho ST | | |

| | | | | | | # | | | |
|---|---|---|---|---|---|---|---|---|---|
| | | | | | Total # | unassign | # MS2 | % MS2 | # |
| Mascot | Decoy or | Duration | | | MS2 | MS/MS | spectra | spectra | unique |
| job # | Error | (s) | (min) | (h) | spectra | spectra | matched | matched | proteins |
| 19018 | decoy | 118 | 2.0 | 0.03 | 371 | 266 | 105 | 28 | 4 |
| 19037 | decoy | 189 | 3.2 | 0.05 | 371 | 49 | 322 | 87 | 13 |
| 19020 | decoy | 259236 | 4320.6 | 72.01 | 371 | 325 | 46 | 12 | 1 |
| 19040 | decoy | 145144 | 2419.1 | 40.32 | 371 | 258 | 113 | 30 | 1 |
| 19052 | decoy | 17651 | 294.2 | 4.90 | 371 | 309 | 62 | 17 | 1 |
| 19047 | decoy | 11549 | 192.5 | 3.21 | 371 | 235 | 136 | 37 | 3 |
| 19031 | error | 88377 | 1473.0 | 24.55 | 11250 | 11040 | 210 | 2 | 12 |
| 19030 | decoy | 29 | 0.5 | 0.01 | 11250 | 11037 | 213 | 2 | 20 |
| 19048 | decoy | 150 | 2.5 | 0.04 | 11250 | 10895 | 355 | 3 | 36 |
| 19050 | decoy | 6308 | 105.1 | 1.75 | 11250 | 11063 | 187 | 2 | 21 |
| 19049 | decoy | 6195 | 103.3 | 1.72 | 11250 | 10660 | 590 | 5 | 61 |
| 19051 | decoy | 12 | 0.2 | 0.00 | 11250 | 11036 | 214 | 2 | 20 |
| 19043 | decoy | 18 | 0.3 | 0.01 | 11250 | 10959 | 291 | 3 | 24 |
| 19042 | decoy | 883 | 14.7 | 0.25 | 11250 | 10252 | 998 | 9 | 94 |
| 19044 | decoy | 233 | 3.9 | 0.06 | 11250 | 10069 | 1181 | 10 | 80 |
| 19045 | decoy | 1685 | 28.1 | 0.47 | 11250 | 9898 | 1352 | 12 | 141 |
| 19046 | decoy | 192376 | 3206.3 | 53.44 | 11250 | 9387 | 1863 | 17 | 274 |

A ‘homemade’ database of 59 fasta sequences comprising horse myoglobin, all known allelic variants of bovine caseins, and the most abundant bovine whey proteins (α-lactalbumin, β-lactoglobulin, bovine serum albumin) was searched on our local Mascot server using a ±50 ppm fragment tolerance. The Mascot output is reported in as a list of proteins and proteoforms in Tables 8 and 9, respectively as well as exemplified in FIG. 12A. Four accessions are listed, based on 105 (28%) MS/MS spectra matched, correctly identifying myoglobin, α-S1-casein variant B and β-lactoglobulin, albeit not the correct allelic variant. Based on accurate mass and accounting for carbamidomethylation sites, variant A of β-lactoglobulin was expected and Mascot identified variants E and F instead which differ at five AA positions, due to insufficient sequence coverage. Bovine serum albumin was not identified. Myoglobin achieves the highest score (3782), with 97 MS/MS spectra yielding annotations, 82% of them being redundant, which is expected as our data is on purpose highly repetitive. Unmodified myoglobin was the most frequently identified (41%), as it was the most abundant proteoform in the spectra. Oxidised proteoforms were also identified, in combination or not with phosphorylated and acetylated proteoforms. Six MS/MS spectra led to the correct identification of α-S1-casein B with a score of 123. Several proteoforms are listed, all of them oxidized and bearing from 6 to 13 phosphorylations. Mascot scores for β-lactoglobulin were below the ion score threshold (<27), indicative of low sequence homology. If the fragment tolerance is increased to ±2 Da, 13 proteins are identified from 322 (87%) MS/MS spectra matches (Tables 8 and 9). Search times presented are in the order of minutes.

TABLE 8
List of proteins identified from standard samples using Mascot
algorithm and either a homemade or SwissProt database

| Job no. | DB | Taxonomy | PTM | Frag. tol. | | Family | M | DB |
|---|---|---|---|---|---|---|---|---|
| 19018 | HM | all | AOP | 50 | ppm | 1 | 1 | TDS_milk-protein-variants-sequences |
| 19018 | HM | all | AOP | 50 | ppm | 2 | 1 | TDS_milk-protein-variants-sequences |
| 19018 | HM | all | AOP | 50 | ppm | 3 | 1 | TDS_milk-protein-variants-sequences |
| 19018 | HM | all | AOP | 50 | ppm | 4 | 1 | TDS_milk-protein-variants-sequences |
| 19037 | HM | all | AOP | 2 | Da | 1 | 1 | TDS_milk-protein-variants-sequences |
| 19037 | HM | all | AOP | 2 | Da | 2 | 1 | TDS_milk-protein-variants-sequences |
| 19037 | HM | all | AOP | 2 | Da | 3 | 1 | TDS_milk-protein-variants-sequences |
| 19037 | HM | all | AOP | 2 | Da | 4 | 1 | TDS_milk-protein-variants-sequences |
| 19037 | HM | all | AOP | 2 | Da | 5 | 1 | TDS_milk-protein-variants-sequences |
| 19037 | HM | all | AOP | 2 | Da | 6 | 1 | TDS_milk-protein-variants-sequences |
| 19037 | HM | all | AOP | 2 | Da | 7 | 1 | TDS_milk-protein-variants-sequences |
| 19037 | HM | all | AOP | 2 | Da | 7 | 2 | TDS_milk-protein-variants-sequences |
| 19037 | HM | all | AOP | 2 | Da | 8 | 1 | TDS_milk-protein-variants-sequences |
| 19037 | HM | all | AOP | 2 | Da | 9 | 1 | TDS_milk-protein-variants-sequences |
| 19037 | HM | all | AOP | 2 | Da | 10 | 1 | TDS_milk-protein-variants-sequences |
| 19037 | HM | all | AOP | 2 | Da | 11 | 1 | TDS_milk-protein-variants-sequences |
| 19037 | HM | all | AOP | 2 | Da | 12 | 1 | TDS_milk-protein-variants-sequences |
| 19037 | HM | all | AOP | 2 | Da | 13 | 1 | TDS_milk-protein-variants-sequences |
| 19020 | SP | all | OP | 50 | ppm | 1 | 1 | SwissProt |
| 19040 | SP | all | OP | 2 | Da | 1 | 1 | SwissProt |
| 19052 | SP | other mammalia | AOP | 50 | ppm | 1 | 1 | SwissProt |
| 19047 | SP | other mammalia | AOP | 2 | Da | 1 | 1 | SwissProt |
| 19047 | SP | other mammalia | AOP | 2 | Da | 2 | 1 | SwissProt |
| 19047 | SP | other mammalia | AOP | 2 | Da | 3 | 1 | SwissProt |

| | | | | | Match | | Seq | |
|---|---|---|---|---|---|---|---|---|
| Job no. | Accession | Score | Mass | Matches | (sig) | Seqs | (sig) | emPAI |
| 19018 | P68082 | 3782 | 16941 | 97 | 97 | 1 | 1 | 2.94 |
| 19018 | P02662 | 123 | 22960 | 6 | 6 | 1 | 1 | 1.16 |
| 19018 | P02754 | 21 | 18531 | 1 | 1 | 1 | 1 | 0.17 |
| 19018 | P02754 | 17 | 18472 | 1 | 1 | 1 | 1 | 0.17 |
| 19037 | P68082 | 12740 | 16941 | 131 | 131 | 1 | 1 | 5.59 |
| 19037 | P02662 | 628 | 22960 | 22 | 22 | 1 | 1 | 5 |
| 19037 | P02662 | 407 | 22888 | 13 | 13 | 1 | 1 | 2.18 |
| 19037 | P02754 | 395 | 18482 | 35 | 35 | 1 | 1 | 3.13 |
| 19037 | P02662 | 359 | 22987 | 10 | 10 | 1 | 1 | 1.79 |
| 19037 | P02662 | 332 | 22990 | 18 | 18 | 1 | 1 | 6.76 |
| 19037 | P02754 | 330 | 18472 | 30 | 30 | 1 | 1 | 2.03 |
| 19037 | P02754 | 72 | 18564 | 5 | 5 | 1 | 1 | 0.37 |
| 19037 | P02754 | 292 | 18500 | 25 | 25 | 1 | 1 | 2.01 |
| 19037 | P02754 | 117 | 18554 | 10 | 10 | 1 | 1 | 0.88 |
| 19037 | P02754 | 98 | 18531 | 9 | 9 | 1 | 1 | 0.88 |
| 19037 | P02754 | 75 | 18555 | 7 | 7 | 1 | 1 | 0.88 |
| 19037 | P02754 | 50 | 18641 | 3 | 3 | 1 | 1 | 0.17 |
| 19037 | P02754 | 41 | 18571 | 4 | 4 | 1 | 1 | 0.6 |
| 19020 | MYG_EQUBU | 1456 | 17072 | 46 | 46 | 2 | 2 | 2.91 |
| 19040 | MYG_EQUBU | 8764 | 17072 | 113 | 113 | 2 | 2 | 4.49 |
| 19052 | MYG_EQUBU | 2119 | 17072 | 62 | 62 | 2 | 2 | 6.72 |
| 19047 | MYG_EQUBU | 10298 | 17072 | 134 | 134 | 2 | 2 | 11.87 |
| 19047 | NU6M_TACAC | 46 | 18085 | 1 | 1 | 1 | 1 | 0.18 |
| 19047 | NU6M_HIPAM | 34 | 18642 | 1 | 1 | 1 | 1 | 0.17 |

Legend: HM, homemade database; SP, SwissProt database; A, Protein N-term acetylation; O, oxidation (M); P, phosphorylation.

TABLE 9
List of proteoforms identified from standard samples using Mascot
algorithms and either a homemade or SwissProt database.

| Job no. | Description | Score | Mass | Matches | Seqs | emPAI | Query | Dupes | Observed |
|---|---|---|---|---|---|---|---|---|---|
| 19018 | myoglobin (P68082) | 3782 | 16941 | 97 | 1 | 2.94 | 35 | 3 | 16947.0184 |
| 19018 | myoglobin (P68082) | 3782 | 16941 | 97 | 1 | 2.94 | 48 | 4 | 16948.0746 |
| 19018 | myoglobin (P68082) | 3782 | 16941 | 97 | 1 | 2.94 | 62 | | 16949.0282 |
| 19018 | myoglobin (P68082) | 3782 | 16941 | 97 | 1 | 2.94 | 63 | | 16949.0282 |
| 19018 | myoglobin (P68082) | 3782 | 16941 | 97 | 1 | 2.94 | 64 | | 16949.0395 |
| 19018 | myoglobin (P68082) | 3782 | 16941 | 97 | 1 | 2.94 | 66 | 4 | 16949.0395 |
| 19018 | myoglobin (P68082) | 3782 | 16941 | 97 | 1 | 2.94 | 71 | | 16949.0502 |
| 19018 | myoglobin (P68082) | 3782 | 16941 | 97 | 1 | 2.94 | 72 | | 16949.0502 |
| 19018 | myoglobin (P68082) | 3782 | 16941 | 97 | 1 | 2.94 | 74 | | 16949.0738 |
| 19018 | myoglobin (P68082) | 3782 | 16941 | 97 | 1 | 2.94 | 133 | 17 | 16951.0397 |
| 19018 | myoglobin (P68082) | 3782 | 16941 | 97 | 1 | 2.94 | 143 | 40 | 16951.0512 |
| 19018 | myoglobin (P68082) | 3782 | 16941 | 97 | 1 | 2.94 | 147 | 11 | 16952.0406 |
| 19018 | myoglobin (P68082) | 3782 | 16941 | 97 | 1 | 2.94 | 165 | | 16953.0819 |
| 19018 | myoglobin (P68082) | 3782 | 16941 | 97 | 1 | 2.94 | 188 | 1 | 17008.0223 |
| 19018 | aS1CN B (P02662) | 123 | 22960 | 6 | 1 | 1.16 | 301 | | 23673.3328 |
| 19018 | aS1CN B (P02662) | 123 | 22960 | 6 | 1 | 1.16 | 306 | | 23673.426 |
| 19018 | aS1CN B (P02662) | 123 | 22960 | 6 | 1 | 1.16 | 308 | | 23673.426 |
| 19018 | aS1CN B (P02662) | 123 | 22960 | 6 | 1 | 1.16 | 313 | | 23729.3675 |
| 19018 | aS1CN B (P02662) | 123 | 22960 | 6 | 1 | 1.16 | 348 | | 23846.4878 |
| 19018 | aS1CN B (P02662) | 123 | 22960 | 6 | 1 | 1.16 | 353 | | 23848.4692 |
| 19018 | bLG E (P02754) | 21 | 18531 | 1 | 1 | 0.17 | 236 | | 18452.5792 |
| 19018 | bLG F (P02754) | 17 | 18472 | 1 | 1 | 0.17 | 195 | | 18394.4984 |
| 19037 | myoglobin (P68082) | 12740 | 16941 | 131 | 1 | 5.59 | 47 | 6 | 16948.0746 |
| 19037 | myoglobin (P68082) | 12740 | 16941 | 131 | 1 | 5.59 | 48 | 2 | 16948.0746 |
| 19037 | myoglobin (P68082) | 12740 | 16941 | 131 | 1 | 5.59 | 53 | | 16948.1149 |
| 19037 | myoglobin (P68082) | 12740 | 16941 | 131 | 1 | 5.59 | 57 | | 16949.0234 |
| 19037 | myoglobin (P68082) | 12740 | 16941 | 131 | 1 | 5.59 | 59 | | 16949.0282 |
| 19037 | myoglobin (P68082) | 12740 | 16941 | 131 | 1 | 5.59 | 66 | 2 | 16949.0395 |
| 19037 | myoglobin (P68082) | 12740 | 16941 | 131 | 1 | 5.59 | 69 | | 16949.0502 |
| 19037 | myoglobin (P68082) | 12740 | 16941 | 131 | 1 | 5.59 | 72 | 1 | 16949.0502 |
| 19037 | myoglobin (P68082) | 12740 | 16941 | 131 | 1 | 5.59 | 73 | | 16949.0502 |
| 19037 | myoglobin (P68082) | 12740 | 16941 | 131 | 1 | 5.59 | 76 | | 16949.0738 |
| 19037 | myoglobin (P68082) | 12740 | 16941 | 131 | 1 | 5.59 | 80 | | 16950.0213 |
| 19037 | myoglobin (P68082) | 12740 | 16941 | 131 | 1 | 5.59 | 85 | | 16950.063 |
| 19037 | myoglobin (P68082) | 12740 | 16941 | 131 | 1 | 5.59 | 96 | | 16950.0707 |
| 19037 | myoglobin (P68082) | 12740 | 16941 | 131 | 1 | 5.59 | 97 | | 16950.0707 |
| 19037 | myoglobin (P68082) | 12740 | 16941 | 131 | 1 | 5.59 | 106 | | 16950.1168 |
| 19037 | myoglobin (P68082) | 12740 | 16941 | 131 | 1 | 5.59 | 107 | | 16950.1168 |
| 19037 | myoglobin (P68082) | 12740 | 16941 | 131 | 1 | 5.59 | 113 | 37 | 16950.999 |
| 19037 | myoglobin (P68082) | 12740 | 16941 | 131 | 1 | 5.59 | 116 | | 16951.0228 |
| 19037 | myoglobin (P68082) | 12740 | 16941 | 131 | 1 | 5.59 | 117 | | 16951.0228 |
| 19037 | myoglobin (P68082) | 12740 | 16941 | 131 | 1 | 5.59 | 118 | | 16951.0228 |
| 19037 | myoglobin (P68082) | 12740 | 16941 | 131 | 1 | 5.59 | 120 | | 16951.0229 |
| 19037 | myoglobin (P68082) | 12740 | 16941 | 131 | 1 | 5.59 | 127 | | 16951.0272 |
| 19037 | myoglobin (P68082) | 12740 | 16941 | 131 | 1 | 5.59 | 133 | 2 | 16951.0397 |
| 19037 | myoglobin (P68082) | 12740 | 16941 | 131 | 1 | 5.59 | 138 | | 16951.0491 |
| 19037 | myoglobin (P68082) | 12740 | 16941 | 131 | 1 | 5.59 | 140 | | 16951.0512 |
| 19037 | myoglobin (P68082) | 12740 | 16941 | 131 | 1 | 5.59 | 146 | | 16952.0406 |
| 19037 | myoglobin (P68082) | 12740 | 16941 | 131 | 1 | 5.59 | 148 | 21 | 16952.0406 |
| 19037 | myoglobin (P68082) | 12740 | 16941 | 131 | 1 | 5.59 | 162 | | 16952.0964 |
| 19037 | myoglobin (P68082) | 12740 | 16941 | 131 | 1 | 5.59 | 163 | | 16952.0964 |
| 19037 | myoglobin (P68082) | 12740 | 16941 | 131 | 1 | 5.59 | 187 | 28 | 17008.0223 |
| 19037 | myoglobin (P68082) | 12740 | 16941 | 131 | 1 | 5.59 | 188 | | 17008.0223 |
| 19037 | aS1CN B (P02662) | 628 | 22960 | 22 | 1 | 5 | 296 | | 23672.2825 |
| 19037 | aS1CN B (P02662) | 628 | 22960 | 22 | 1 | 5 | 301 | | 23673.3328 |
| 19037 | aS1CN B (P02662) | 628 | 22960 | 22 | 1 | 5 | 303 | | 23673.3328 |
| 19037 | aS1CN B (P02662) | 628 | 22960 | 22 | 1 | 5 | 306 | | 23673.426 |
| 19037 | aS1CN B (P02662) | 628 | 22960 | 22 | 1 | 5 | 308 | | 23673.426 |
| 19037 | aS1CN B (P02662) | 628 | 22960 | 22 | 1 | 5 | 313 | 2 | 23729.3675 |
| 19037 | aS1CN B (P02662) | 628 | 22960 | 22 | 1 | 5 | 314 | | 23729.3675 |
| 19037 | aS1CN B (P02662) | 628 | 22960 | 22 | 1 | 5 | 316 | | 23729.3675 |
| 19037 | aS1CN B (P02662) | 628 | 22960 | 22 | 1 | 5 | 323 | | 23788.3773 |
| 19037 | aS1CN B (P02662) | 628 | 22960 | 22 | 1 | 5 | 348 | | 23846.4878 |
| 19037 | aS1CN B (P02662) | 628 | 22960 | 22 | 1 | 5 | 350 | | 23846.4878 |
| 19037 | aS1CN B (P02662) | 628 | 22960 | 22 | 1 | 5 | 351 | 1 | 23846.4878 |
| 19037 | aS1CN B (P02662) | 628 | 22960 | 22 | 1 | 5 | 353 | | 23848.4692 |
| 19037 | aS1CN B (P02662) | 628 | 22960 | 22 | 1 | 5 | 355 | | 23848.4692 |
| 19037 | aS1CN B (P02662) | 628 | 22960 | 22 | 1 | 5 | 363 | | 23910.537 |
| 19037 | aS1CN B (P02662) | 628 | 22960 | 22 | 1 | 5 | 364 | | 23910.537 |
| 19037 | aS1CN B (P02662) | 628 | 22960 | 22 | 1 | 5 | 366 | | 23910.537 |
| 19037 | aS1CN B (P02662) | 628 | 22960 | 22 | 1 | 5 | 369 | | 23910.567 |
| 19037 | aS1CN B (P02662) | 628 | 22960 | 22 | 1 | 5 | 370 | | 23910.567 |
| 19037 | aS1CN E (P02662) | 407 | 22888 | 13 | 1 | 2.18 | 306 | | 23673.426 |
| 19037 | aS1CN E (P02662) | 407 | 22888 | 13 | 1 | 2.18 | 313 | | 23729.3675 |
| 19037 | aS1CN E (P02662) | 407 | 22888 | 13 | 1 | 2.18 | 323 | | 23788.3773 |
| 19037 | aS1CN E (P02662) | 407 | 22888 | 13 | 1 | 2.18 | 343 | | 23846.462 |
| 19037 | aS1CN E (P02662) | 407 | 22888 | 13 | 1 | 2.18 | 348 | | 23846.4878 |
| 19037 | aS1CN E (P02662) | 407 | 22888 | 13 | 1 | 2.18 | 350 | | 23846.4878 |
| 19037 | aS1CN E (P02662) | 407 | 22888 | 13 | 1 | 2.18 | 351 | | 23846.4878 |
| 19037 | aS1CN E (P02662) | 407 | 22888 | 13 | 1 | 2.18 | 353 | | 23848.4692 |
| 19037 | aS1CN E (P02662) | 407 | 22888 | 13 | 1 | 2.18 | 356 | | 23848.4692 |
| 19037 | aS1CN E (P02662) | 407 | 22888 | 13 | 1 | 2.18 | 363 | | 23910.537 |
| 19037 | aS1CN E (P02662) | 407 | 22888 | 13 | 1 | 2.18 | 364 | | 23910.537 |
| 19037 | aS1CN E (P02662) | 407 | 22888 | 13 | 1 | 2.18 | 366 | | 23910.537 |
| 19037 | aS1CN E (P02662) | 407 | 22888 | 13 | 1 | 2.18 | 368 | | 23910.567 |
| 19037 | bLG I (P02754) | 395 | 18482 | 35 | 1 | 3.13 | 190 | 2 | 18392.5387 |
| 19037 | bLG I (P02754) | 395 | 18482 | 35 | 1 | 3.13 | 192 | | 18392.5387 |
| 19037 | bLG I (P02754) | 395 | 18482 | 35 | 1 | 3.13 | 193 | | 18392.5387 |
| 19037 | bLG I (P02754) | 395 | 18482 | 35 | 1 | 3.13 | 212 | 1 | 18422.5717 |
| 19037 | bLG I (P02754) | 395 | 18482 | 35 | 1 | 3.13 | 228 | 2 | 18450.559 |
| 19037 | bLG I (P02754) | 395 | 18482 | 35 | 1 | 3.13 | 236 | 1 | 18452.5792 |
| 19037 | bLG I (P02754) | 395 | 18482 | 35 | 1 | 3.13 | 239 | | 18452.5792 |
| 19037 | bLG I (P02754) | 395 | 18482 | 35 | 1 | 3.13 | 242 | | 18475.5423 |
| 19037 | bLG I (P02754) | 395 | 18482 | 35 | 1 | 3.13 | 244 | | 18475.5423 |
| 19037 | bLG I (P02754) | 395 | 18482 | 35 | 1 | 3.13 | 246 | | 18476.5099 |
| 19037 | bLG I (P02754) | 395 | 18482 | 35 | 1 | 3.13 | 248 | | 18476.5099 |
| 19037 | bLG I (P02754) | 395 | 18482 | 35 | 1 | 3.13 | 249 | 1 | 18476.5099 |
| 19037 | bLG I (P02754) | 395 | 18482 | 35 | 1 | 3.13 | 251 | | 18477.6176 |
| 19037 | bLG I (P02754) | 395 | 18482 | 35 | 1 | 3.13 | 254 | | 18477.6176 |
| 19037 | bLG I (P02754) | 395 | 18482 | 35 | 1 | 3.13 | 258 | | 18478.5355 |
| 19037 | bLG I (P02754) | 395 | 18482 | 35 | 1 | 3.13 | 261 | 1 | 18478.5709 |
| 19037 | bLG I (P02754) | 395 | 18482 | 35 | 1 | 3.13 | 266 | | 18478.6278 |
| 19037 | bLG I (P02754) | 395 | 18482 | 35 | 1 | 3.13 | 268 | | 18478.6278 |
| 19037 | bLG I (P02754) | 395 | 18482 | 35 | 1 | 3.13 | 269 | | 18478.6278 |
| 19037 | bLG I (P02754) | 395 | 18482 | 35 | 1 | 3.13 | 274 | | 18479.5647 |
| 19037 | bLG I (P02754) | 395 | 18482 | 35 | 1 | 3.13 | 281 | | 18533.656 |
| 19037 | bLG I (P02754) | 395 | 18482 | 35 | 1 | 3.13 | 282 | | 18533.656 |
| 19037 | bLG I (P02754) | 395 | 18482 | 35 | 1 | 3.13 | 284 | | 18533.656 |
| 19037 | bLG I (P02754) | 395 | 18482 | 35 | 1 | 3.13 | 287 | | 18535.632 |
| 19037 | bLG I (P02754) | 395 | 18482 | 35 | 1 | 3.13 | 293 | | 18536.5494 |
| 19037 | bLG I (P02754) | 395 | 18482 | 35 | 1 | 3.13 | 294 | | 18536.5494 |
| 19037 | aS1CN F (P02662) | 359 | 22987 | 10 | 1 | 1.79 | 296 | | 23672.2825 |
| 19037 | aS1CN F (P02662) | 359 | 22987 | 10 | 1 | 1.79 | 301 | 1 | 23673.3328 |
| 19037 | aS1CN F (P02662) | 359 | 22987 | 10 | 1 | 1.79 | 307 | | 23673.426 |
| 19037 | aS1CN F (P02662) | 359 | 22987 | 10 | 1 | 1.79 | 313 | | 23729.3675 |
| 19037 | aS1CN F (P02662) | 359 | 22987 | 10 | 1 | 1.79 | 323 | | 23788.3773 |
| 19037 | aS1CN F (P02662) | 359 | 22987 | 10 | 1 | 1.79 | 348 | | 23846.4878 |
| 19037 | aS1CN F (P02662) | 359 | 22987 | 10 | 1 | 1.79 | 350 | | 23846.4878 |
| 19037 | aS1CN F (P02662) | 359 | 22987 | 10 | 1 | 1.79 | 353 | | 23848.4692 |
| 19037 | aS1CN F (P02662) | 359 | 22987 | 10 | 1 | 1.79 | 370 | | 23910.567 |
| 19037 | aS1CN D (P02662) | 332 | 22990 | 18 | 1 | 6.76 | 296 | | 23672.2825 |
| 19037 | aS1CN D (P02662) | 332 | 22990 | 18 | 1 | 6.76 | 302 | 1 | 23673.3328 |
| 19037 | aS1CN D (P02662) | 332 | 22990 | 18 | 1 | 6.76 | 307 | | 23673.426 |
| 19037 | aS1CN D (P02662) | 332 | 22990 | 18 | 1 | 6.76 | 308 | | 23673.426 |
| 19037 | aS1CN D (P02662) | 332 | 22990 | 18 | 1 | 6.76 | 309 | | 23673.426 |
| 19037 | aS1CN D (P02662) | 332 | 22990 | 18 | 1 | 6.76 | 316 | | 23729.3675 |
| 19037 | aS1CN D (P02662) | 332 | 22990 | 18 | 1 | 6.76 | 326 | | 23788.3773 |
| 19037 | aS1CN D (P02662) | 332 | 22990 | 18 | 1 | 6.76 | 343 | | 23846.462 |
| 19037 | aS1CN D (P02662) | 332 | 22990 | 18 | 1 | 6.76 | 348 | | 23846.4878 |
| 19037 | aS1CN D (P02662) | 332 | 22990 | 18 | 1 | 6.76 | 350 | | 23846.4878 |
| 19037 | aS1CN D (P02662) | 332 | 22990 | 18 | 1 | 6.76 | 353 | | 23848.4692 |
| 19037 | aS1CN D (P02662) | 332 | 22990 | 18 | 1 | 6.76 | 356 | | 23848.4692 |
| 19037 | aS1CN D (P02662) | 332 | 22990 | 18 | 1 | 6.76 | 363 | | 23910.537 |
| 19037 | aS1CN D (P02662) | 332 | 22990 | 18 | 1 | 6.76 | 364 | | 23910.537 |
| 19037 | aS1CN D (P02662) | 332 | 22990 | 18 | 1 | 6.76 | 365 | | 23910.537 |
| 19037 | aS1CN D (P02662) | 332 | 22990 | 18 | 1 | 6.76 | 369 | | 23910.567 |
| 19037 | aS1CN D (P02662) | 332 | 22990 | 18 | 1 | 6.76 | 370 | | 23910.567 |
| 19037 | bLG F/C (P02754) | 330 | 18472 | 30 | 1 | 2.03 | 190 | | 18392.5387 |
| 19037 | bLG F/C (P02754) | 330 | 18472 | 30 | 1 | 2.03 | 196 | | 18394.4984 |
| 19037 | bLG F/C (P02754) | 330 | 18472 | 30 | 1 | 2.03 | 201 | 1 | 18394.5584 |
| 19037 | bLG F/C (P02754) | 330 | 18472 | 30 | 1 | 2.03 | 206 | | 18416.4322 |
| 19037 | bLG F/C (P02754) | 330 | 18472 | 30 | 1 | 2.03 | 209 | | 18419.4725 |
| 19037 | bLG F/C (P02754) | 330 | 18472 | 30 | 1 | 2.03 | 218 | 2 | 18449.5008 |
| 19037 | bLG F/C (P02754) | 330 | 18472 | 30 | 1 | 2.03 | 231 | | 18451.5042 |
| 19037 | bLG F/C (P02754) | 330 | 18472 | 30 | 1 | 2.03 | 242 | 1 | 18475.5423 |
| 19037 | bLG F/C (P02754) | 330 | 18472 | 30 | 1 | 2.03 | 246 | | 18476.5099 |
| 19037 | bLG F/C (P02754) | 330 | 18472 | 30 | 1 | 2.03 | 248 | | 18476.5099 |
| 19037 | bLG F/C (P02754) | 330 | 18472 | 30 | 1 | 2.03 | 257 | | 18478.5355 |
| 19037 | bLG F/C (P02754) | 330 | 18472 | 30 | 1 | 2.03 | 258 | | 18478.5355 |
| 19037 | bLG F/C (P02754) | 330 | 18472 | 30 | 1 | 2.03 | 262 | | 18478.5709 |
| 19037 | bLG F/C (P02754) | 330 | 18472 | 30 | 1 | 2.03 | 268 | | 18478.6278 |
| 19037 | bLG F/C (P02754) | 330 | 18472 | 30 | 1 | 2.03 | 271 | | 18479.5647 |
| 19037 | bLG F/C (P02754) | 330 | 18472 | 30 | 1 | 2.03 | 274 | | 18479.5647 |
| 19037 | bLG F/C (P02754) | 330 | 18472 | 30 | 1 | 2.03 | 281 | 1 | 18533.656 |
| 19037 | bLG F/C (P02754) | 330 | 18472 | 30 | 1 | 2.03 | 284 | | 18533.656 |
| 19037 | bLG F/C (P02754) | 330 | 18472 | 30 | 1 | 2.03 | 286 | 1 | 18535.632 |
| 19037 | bLG F/C (P02754) | 330 | 18472 | 30 | 1 | 2.03 | 288 | 1 | 18535.632 |
| 19037 | bLG F/C (P02754) | 330 | 18472 | 30 | 1 | 2.03 | 289 | | 18535.632 |
| 19037 | bLG F/C (P02754) | 330 | 18472 | 30 | 1 | 2.03 | 292 | | 18536.5494 |
| 19037 | bLG F/C (P02754) | 330 | 18472 | 30 | 1 | 2.03 | 293 | | 18536.5494 |
| 19037 | bLG F/C (P02754) | 330 | 18472 | 30 | 1 | 2.03 | 294 | 1 | 18536.5494 |
| 19037 | bLG G (P02754) | 292 | 18500 | 25 | 1 | 2.01 | 195 | | 18394.4984 |
| 19037 | bLG G (P02754) | 292 | 18500 | 25 | 1 | 2.01 | 197 | 1 | 18394.4984 |
| 19037 | bLG G (P02754) | 292 | 18500 | 25 | 1 | 2.01 | 206 | | 18416.4322 |
| 19037 | bLG G (P02754) | 292 | 18500 | 25 | 1 | 2.01 | 227 | | 18450.559 |
| 19037 | bLG G (P02754) | 292 | 18500 | 25 | 1 | 2.01 | 236 | | 18452.5792 |
| 19037 | bLG G (P02754) | 292 | 18500 | 25 | 1 | 2.01 | 239 | | 18452.5792 |
| 19037 | bLG G (P02754) | 292 | 18500 | 25 | 1 | 2.01 | 241 | | 18475.5423 |
| 19037 | bLG G (P02754) | 292 | 18500 | 25 | 1 | 2.01 | 245 | | 18476.5099 |
| 19037 | bLG G (P02754) | 292 | 18500 | 25 | 1 | 2.01 | 246 | | 18476.5099 |
| 19037 | bLG G (P02754) | 292 | 18500 | 25 | 1 | 2.01 | 247 | | 18476.5099 |
| 19037 | bLG G (P02754) | 292 | 18500 | 25 | 1 | 2.01 | 248 | | 18476.5099 |
| 19037 | bLG G (P02754) | 292 | 18500 | 25 | 1 | 2.01 | 254 | | 18477.6176 |
| 19037 | bLG G (P02754) | 292 | 18500 | 25 | 1 | 2.01 | 264 | | 18478.5709 |
| 19037 | bLG G (P02754) | 292 | 18500 | 25 | 1 | 2.01 | 271 | | 18479.5647 |
| 19037 | bLG G (P02754) | 292 | 18500 | 25 | 1 | 2.01 | 272 | 1 | 18479.5647 |
| 19037 | bLG G (P02754) | 292 | 18500 | 25 | 1 | 2.01 | 281 | | 18533.656 |
| 19037 | bLG G (P02754) | 292 | 18500 | 25 | 1 | 2.01 | 282 | | 18533.656 |
| 19037 | bLG G (P02754) | 292 | 18500 | 25 | 1 | 2.01 | 284 | | 18533.656 |
| 19037 | bLG G (P02754) | 292 | 18500 | 25 | 1 | 2.01 | 286 | | 18535.632 |
| 19037 | bLG G (P02754) | 292 | 18500 | 25 | 1 | 2.01 | 288 | 1 | 18535.632 |
| 19037 | bLG G (P02754) | 292 | 18500 | 25 | 1 | 2.01 | 289 | | 18535.632 |
| 19037 | bLG G (P02754) | 292 | 18500 | 25 | 1 | 2.01 | 291 | | 18536.5494 |
| 19037 | bLG G (P02754) | 292 | 18500 | 25 | 1 | 2.01 | 292 | | 18536.5494 |
| 19037 | bLG D (P02754) | 117 | 18554 | 10 | 1 | 0.88 | 228 | | 18450.559 |
| 19037 | bLG D (P02754) | 117 | 18554 | 11 | 2 | 1.88 | 236 | | 18452.5792 |
| 19037 | bLG D (P02754) | 117 | 18554 | 12 | 3 | 2.88 | 238 | | 18452.5792 |
| 19037 | bLG D (P02754) | 117 | 18554 | 13 | 4 | 3.88 | 244 | | 18475.5423 |
| 19037 | bLG D (P02754) | 117 | 18554 | 14 | 5 | 4.88 | 251 | | 18477.6176 |
| 19037 | bLG D (P02754) | 117 | 18554 | 15 | 6 | 5.88 | 254 | | 18477.6176 |
| 19037 | bLG D (P02754) | 117 | 18554 | 16 | 7 | 6.88 | 257 | | 18478.5355 |
| 19037 | bLG D (P02754) | 117 | 18554 | 17 | 8 | 7.88 | 258 | | 18478.5355 |
| 19037 | bLG D (P02754) | 117 | 18554 | 18 | 9 | 8.88 | 278 | | 18482.6285 |
| 19037 | bLG D (P02754) | 117 | 18554 | 19 | 10 | 9.88 | 289 | 1 | 18535.632 |
| 19037 | bLG E (P02754) | 98 | 18531 | 9 | 1 | 0.88 | 192 | | 18392.5387 |
| 19037 | bLG E (P02754) | 98 | 18531 | 9 | 1 | 0.88 | 237 | 1 | 18452.5792 |
| 19037 | bLG E (P02754) | 98 | 18531 | 9 | 1 | 0.88 | 239 | 1 | 18452.5792 |
| 19037 | bLG E (P02754) | 98 | 18531 | 9 | 1 | 0.88 | 247 | 1 | 18476.5099 |
| 19037 | bLG E (P02754) | 98 | 18531 | 9 | 1 | 0.88 | 272 | | 18479.5647 |
| 19037 | bLG E (P02754) | 98 | 18531 | 9 | 1 | 0.88 | 287 | | 18535.632 |
| 19037 | bLG B (P02754) | 75 | 18555 | 7 | 1 | 0.88 | 193 | | 18392.5387 |
| 19037 | bLG B (P02754) | 75 | 18555 | 7 | 1 | 0.88 | 228 | | 18450.559 |
| 19037 | bLG B (P02754) | 75 | 18555 | 7 | 1 | 0.88 | 245 | | 18476.5099 |
| 19037 | bLG B (P02754) | 75 | 18555 | 7 | 1 | 0.88 | 258 | | 18478.5355 |
| 19037 | bLG B (P02754) | 75 | 18555 | 7 | 1 | 0.88 | 261 | | 18478.5709 |
| 19037 | bLG B (P02754) | 75 | 18555 | 7 | 1 | 0.88 | 279 | | 18482.6285 |
| 19037 | bLG B (P02754) | 75 | 18555 | 7 | 1 | 0.88 | 293 | | 18536.5494 |
| 19037 | bLG A (P02754) | 50 | 18641 | 3 | 1 | 0.17 | 254 | 1 | 18477.6176 |
| 19037 | bLG A (P02754) | 50 | 18641 | 3 | 1 | 0.17 | 287 | | 18535.632 |
| 19037 | bLG J (P02754) | 41 | 18571 | 4 | 1 | 0.6 | 227 | | 18450.559 |
| 19037 | bLG J (P02754) | 41 | 18571 | 4 | 1 | 0.6 | 284 | | 18533.656 |
| 19037 | bLG J (P02754) | 41 | 18571 | 4 | 1 | 0.6 | 286 | | 18535.632 |
| 19037 | bLG J (P02754) | 41 | 18571 | 4 | 1 | 0.6 | 289 | | 18535.632 |
| 19020 | MYG_EQUBU | 1456 | 17072 | 46 | 2 | 2.91 | 35 | 1 | 16947.0184 |
| 19020 | MYG_EQUBU | 1456 | 17072 | 46 | 2 | 2.91 | 48 | 1 | 16948.0746 |
| 19020 | MYG_EQUBU | 1456 | 17072 | 46 | 2 | 2.91 | 53 | 2 | 16948.1149 |
| 19020 | MYG_EQUBU | 1456 | 17072 | 46 | 2 | 2.91 | 67 | | 16949.0395 |
| 19020 | MYG_EQUBU | 1456 | 17072 | 46 | 2 | 2.91 | 71 | | 16949.0502 |
| 19020 | MYG_EQUBU | 1456 | 17072 | 46 | 2 | 2.91 | 105 | | 16950.1168 |
| 19020 | MYG_EQUBU | 1456 | 17072 | 46 | 2 | 2.91 | 133 | 2 | 16951.0397 |
| 19020 | MYG_EQUBU | 1456 | 17072 | 46 | 2 | 2.91 | 137 | 1 | 16951.0491 |
| 19020 | MYG_EQUBU | 1456 | 17072 | 46 | 2 | 2.91 | 138 | | 16951.0491 |
| 19020 | MYG_EQUBU | 1456 | 17072 | 46 | 2 | 2.91 | 143 | 18 | 16951.0512 |
| 19020 | MYG_EQUBU | 1456 | 17072 | 46 | 2 | 2.91 | 147 | 6 | 16952.0406 |
| 19020 | MYG_EQUBU | 1456 | 17072 | 46 | 2 | 2.91 | 180 | 1 | 16968.0376 |
| 19020 | MYG_EQUBU | 1456 | 17072 | 46 | 2 | 2.91 | 188 | | 17008.0223 |
| 19040 | MYG_EQUBU | 8764 | 17072 | 113 | 2 | 4.49 | 47 | 3 | 16948.0746 |
| 19040 | MYG_EQUBU | 8764 | 17072 | 113 | 2 | 4.49 | 48 | 2 | 16948.0746 |
| 19040 | MYG_EQUBU | 8764 | 17072 | 113 | 2 | 4.49 | 53 | | 16948.1149 |
| 19040 | MYG_EQUBU | 8764 | 17072 | 113 | 2 | 4.49 | 61 | 3 | 16949.0282 |
| 19040 | MYG_EQUBU | 8764 | 17072 | 113 | 2 | 4.49 | 66 | 2 | 16949.0395 |
| 19040 | MYG_EQUBU | 8764 | 17072 | 113 | 2 | 4.49 | 69 | | 16949.0502 |
| 19040 | MYG_EQUBU | 8764 | 17072 | 113 | 2 | 4.49 | 72 | | 16949.0502 |
| 19040 | MYG_EQUBU | 8764 | 17072 | 113 | 2 | 4.49 | 73 | | 16949.0502 |
| 19040 | MYG_EQUBU | 8764 | 17072 | 113 | 2 | 4.49 | 100 | 2 | 16950.078 |
| 19040 | MYG_EQUBU | 8764 | 17072 | 113 | 2 | 4.49 | 113 | 24 | 16950.999 |
| 19040 | MYG_EQUBU | 8764 | 17072 | 113 | 2 | 4.49 | 116 | | 16951.0228 |
| 19040 | MYG_EQUBU | 8764 | 17072 | 113 | 2 | 4.49 | 118 | | 16951.0228 |
| 19040 | MYG_EQUBU | 8764 | 17072 | 113 | 2 | 4.49 | 133 | | 16951.0397 |
| 19040 | MYG_EQUBU | 8764 | 17072 | 113 | 2 | 4.49 | 138 | | 16951.0491 |
| 19040 | MYG_EQUBU | 8764 | 17072 | 113 | 2 | 4.49 | 148 | 14 | 16952.0406 |
| 19040 | MYG_EQUBU | 8764 | 17072 | 113 | 2 | 4.49 | 156 | 3 | 16952.0839 |
| 19040 | MYG_EQUBU | 8764 | 17072 | 113 | 2 | 4.49 | 165 | 1 | 16953.0819 |
| 19040 | MYG_EQUBU | 8764 | 17072 | 113 | 2 | 4.49 | 173 | | 16965.0545 |
| 19040 | MYG_EQUBU | 8764 | 17072 | 113 | 2 | 4.49 | 187 | 20 | 17008.0223 |
| 19040 | MYG_EQUBU | 8764 | 17072 | 113 | 2 | 4.49 | 188 | | 17008.0223 |
| 19052 | MYG_EQUBU | 2119 | 17072 | 62 | 2 | 6.72 | 35 | 1 | 16947.0184 |
| 19052 | MYG_EQUBU | 2119 | 17072 | 62 | 2 | 6.72 | 48 | 1 | 16948.0746 |
| 19052 | MYG_EQUBU | 2119 | 17072 | 62 | 2 | 6.72 | 53 | 1 | 16948.1149 |
| 19052 | MYG_EQUBU | 2119 | 17072 | 62 | 2 | 6.72 | 67 | | 16949.0395 |
| 19052 | MYG_EQUBU | 2119 | 17072 | 62 | 2 | 6.72 | 69 | 2 | 16949.0502 |
| 19052 | MYG_EQUBU | 2119 | 17072 | 62 | 2 | 6.72 | 71 | | 16949.0502 |
| 19052 | MYG_EQUBU | 2119 | 17072 | 62 | 2 | 6.72 | 72 | | 16949.0502 |
| 19052 | MYG_EQUBU | 2119 | 17072 | 62 | 2 | 6.72 | 105 | | 16950.1168 |
| 19052 | MYG_EQUBU | 2119 | 17072 | 62 | 2 | 6.72 | 133 | 5 | 16951.0397 |
| 19052 | MYG_EQUBU | 2119 | 17072 | 62 | 2 | 6.72 | 137 | | 16951.0491 |
| 19052 | MYG_EQUBU | 2119 | 17072 | 62 | 2 | 6.72 | 138 | | 16951.0491 |
| 19052 | MYG_EQUBU | 2119 | 17072 | 62 | 2 | 6.72 | 143 | 22 | 16951.0512 |
| 19052 | MYG_EQUBU | 2119 | 17072 | 62 | 2 | 6.72 | 147 | 6 | 16952.0406 |
| 19052 | MYG_EQUBU | 2119 | 17072 | 62 | 2 | 6.72 | 180 | 1 | 16968.0376 |
| 19052 | MYG_EQUBU | 2119 | 17072 | 62 | 2 | 6.72 | 188 | | 17008.0223 |
| 19047 | MYG_EQUBU | 10298 | 17072 | 134 | 2 | 11.87 | 47 | 4 | 16948.0746 |
| 19047 | MYG_EQUBU | 10298 | 17072 | 134 | 2 | 11.87 | 48 | 2 | 16948.0746 |
| 19047 | MYG_EQUBU | 10298 | 17072 | 134 | 2 | 11.87 | 53 | | 16948.1149 |
| 19047 | MYG_EQUBU | 10298 | 17072 | 134 | 2 | 11.87 | 66 | 2 | 16949.0395 |
| 19047 | MYG_EQUBU | 10298 | 17072 | 134 | 2 | 11.87 | 69 | | 16949.0502 |
| 19047 | MYG_EQUBU | 10298 | 17072 | 134 | 2 | 11.87 | 72 | | 16949.0502 |
| 19047 | MYG_EQUBU | 10298 | 17072 | 134 | 2 | 11.87 | 73 | | 16949.0502 |
| 19047 | MYG_EQUBU | 10298 | 17072 | 134 | 2 | 11.87 | 100 | 3 | 16950.078 |
| 19047 | MYG_EQUBU | 10298 | 17072 | 134 | 2 | 11.87 | 113 | 25 | 16950.999 |
| 19047 | MYG_EQUBU | 10298 | 17072 | 134 | 2 | 11.87 | 116 | | 16951.0228 |
| 19047 | MYG_EQUBU | 10298 | 17072 | 134 | 2 | 11.87 | 118 | | 16951.0228 |
| 19047 | MYG_EQUBU | 10298 | 17072 | 134 | 2 | 11.87 | 133 | 1 | 16951.0397 |
| 19047 | MYG_EQUBU | 10298 | 17072 | 134 | 2 | 11.87 | 137 | | 16951.0491 |
| 19047 | MYG_EQUBU | 10298 | 17072 | 134 | 2 | 11.87 | 138 | | 16951.0491 |
| 19047 | MYG_EQUBU | 10298 | 17072 | 134 | 2 | 11.87 | 148 | 15 | 16952.0406 |
| 19047 | MYG_EQUBU | 10298 | 17072 | 134 | 2 | 11.87 | 156 | 3 | 16952.0839 |
| 19047 | MYG_EQUBU | 10298 | 17072 | 134 | 2 | 11.87 | 165 | 3 | 16953.0819 |
| 19047 | MYG_EQUBU | 10298 | 17072 | 134 | 2 | 11.87 | 166 | 1 | 16953.0819 |
| 19047 | MYG_EQUBU | 10298 | 17072 | 134 | 2 | 11.87 | 173 | | 16965.0545 |
| 19047 | MYG_EQUBU | 10298 | 17072 | 134 | 2 | 11.87 | 187 | 24 | 17008.0223 |
| 19047 | MYG_EQUBU | 10298 | 17072 | 134 | 2 | 11.87 | 188 | | 17008.0223 |
| 19047 | NU6M_TACAC | 46 | 18085 | 1 | 1 | 0.18 | 294 | | 18536.5494 |
| 19047 | NU6M_HIPAM | 34 | 18642 | 1 | 1 | 0.17 | 267 | | 18478.6278 |

| Job no. | Mr(expt) | Mr(calc) | % | M | Score | Expect | Rank | SEQ ID |
|---|---|---|---|---|---|---|---|---|
| 19018 | 16946.0112 | 17036.9261 | −0.5336 | 0 | 66 | 2.60E−07 | 1 | 1 |
| 19018 | 16947.0673 | 17036.9261 | −0.5274 | 0 | 148 | 1.70E−15 | 1 | 2 |
| 19018 | 16948.021 | 17116.8924 | −0.9866 | 0 | 13 | 0.049 | 1 | 3 |
| 19018 | 16948.021 | 17116.8924 | −0.9866 | 0 | 15 | 0.029 | 1 | 4 |
| 19018 | 16948.0322 | 17116.8924 | −0.9865 | 0 | 32 | 0.0007 | 1 | 5 |
| 19018 | 16948.0322 | 17116.8924 | −0.9865 | 0 | 39 | 0.00014 | 1 | 6 |
| 19018 | 16948.0429 | 17036.9261 | −0.5217 | 0 | 103 | 5.00E−11 | 1 | 7 |
| 19018 | 16948.0429 | 17116.8924 | −0.9864 | 0 | 50 | 9.30E−06 | 1 | 8 |
| 19018 | 16948.0665 | 17078.9367 | −0.7663 | 0 | 18 | 0.017 | 1 | 9 |
| 19018 | 16950.0324 | 16956.9598 | −0.0409 | 0 | 122 | 5.80E−13 | 1 | 10 |
| 19018 | 16950.044 | 16940.9649 | 0.0536 | 0 | 143 | 5.30E−15 | 1 | 11 |
| 19018 | 16951.0333 | 16956.9598 | −0.035 | 0 | 92 | 6.60E−10 | 1 | 12 |
| 19018 | 16952.0746 | 16998.9704 | −0.2759 | 0 | 53 | 5.20E−06 | 1 | 13 |
| 19018 | 17007.0151 | 17020.9312 | −0.0818 | 0 | 172 | 6.50E−18 | 1 | 14 |
| 19018 | 23672.3256 | 23456.2738 | 0.9211 | 0 | 59 | 7.00E−05 | 1 | 15 |
| 19018 | 23672.4187 | 23872.1004 | −0.8365 | 0 | 55 | 0.00019 | 1 | 16 |
| 19018 | 23672.4187 | 23616.2065 | 0.238 | 0 | 31 | 0.043 | 1 | 17 |
| 19018 | 23728.3602 | 23936.0718 | −0.8678 | 0 | 47 | 0.0012 | 1 | 18 |
| 19018 | 23845.4805 | 24016.0381 | −0.7102 | 0 | 42 | 0.0051 | 1 | 19 |
| 19018 | 23847.4619 | 23632.2014 | 0.9109 | 0 | 41 | 0.0056 | 2 | 20 |
| 19018 | 18451.5719 | 18610.5071 | −0.854 | 0 | 21 | 0.043 | 1 | 21 |
| 19018 | 18393.4911 | 18488.4786 | −0.5138 | 0 | 17 | 0.046 | 1 | 22 |
| 19037 | 16947.0673 | 17036.9261 | −0.5274 | 0 | 229 | 1.30E−23 | 1 | 23 |
| 19037 | 16947.0673 | 17036.9261 | −0.5274 | 0 | 245 | 3.50E−25 | 1 | 24 |
| 19037 | 16947.1076 | 17062.9418 | −0.6789 | 0 | 243 | 5.00E−25 | 1 | 25 |
| 19037 | 16948.0161 | 17116.8924 | −0.9866 | 0 | 22 | 0.0069 | 1 | 26 |
| 19037 | 16948.021 | 17078.9367 | −0.7665 | 0 | 23 | 0.0051 | 1 | 27 |
| 19037 | 16948.0322 | 17036.9261 | −0.5218 | 0 | 155 | 2.90E−16 | 1 | 28 |
| 19037 | 16948.0429 | 17036.9261 | −0.5217 | 0 | 142 | 6.20E−15 | 1 | 29 |
| 19037 | 16948.0429 | 17036.9261 | −0.5217 | 0 | 168 | 1.60E−17 | 1 | 30 |
| 19037 | 16948.0429 | 17020.9312 | −0.4282 | 0 | 140 | 9.60E−15 | 1 | 31 |
| 19037 | 16948.0665 | 17116.8924 | −0.9863 | 0 | 35 | 0.00033 | 1 | 32 |
| 19037 | 16949.014 | 17078.9367 | −0.7607 | 0 | 67 | 1.80E−07 | 1 | 33 |
| 19037 | 16949.0557 | 17052.921 | −0.6091 | 0 | 23 | 0.0052 | 1 | 34 |
| 19037 | 16949.0635 | 17036.9261 | −0.5157 | 0 | 27 | 0.002 | 1 | 35 |
| 19037 | 16949.0635 | 17036.9261 | −0.5157 | 0 | 30 | 0.0011 | 1 | 36 |
| 19037 | 16949.1095 | 17100.8975 | −0.8876 | 0 | 41 | 7.80E−05 | 1 | 37 |
| 19037 | 16949.1095 | 16998.9704 | −0.2933 | 0 | 66 | 2.30E−07 | 1 | 38 |
| 19037 | 16949.9917 | 16956.9598 | −0.0411 | 0 | 202 | 5.60E−21 | 1 | 39 |
| 19037 | 16950.0155 | 17052.921 | −0.6034 | 0 | 63 | 5.30E−07 | 1 | 40 |
| 19037 | 16950.0155 | 17036.9261 | −0.5101 | 0 | 18 | 0.016 | 1 | 41 |
| 19037 | 16950.0155 | 17094.9316 | −0.8477 | 0 | 68 | 1.70E−07 | 1 | 42 |
| 19037 | 16950.0156 | 17094.9316 | −0.8477 | 0 | 58 | 1.60E−06 | 1 | 43 |
| 19037 | 16950.0199 | 17100.8975 | −0.8823 | 0 | 18 | 0.014 | 1 | 44 |
| 19037 | 16950.0324 | 17020.9312 | −0.4165 | 0 | 212 | 5.90E−22 | 1 | 45 |
| 19037 | 16950.0418 | 17100.8975 | −0.8822 | 0 | 164 | 4.10E−17 | 1 | 46 |
| 19037 | 16950.044 | 17052.921 | −0.6033 | 0 | 14 | 0.044 | 1 | 47 |
| 19037 | 16951.0333 | 17036.9261 | −0.5042 | 0 | 16 | 0.026 | 1 | 48 |
| 19037 | 16951.0333 | 16940.9649 | 0.0594 | 0 | 285 | 3.40E−29 | 1 | 49 |
| 19037 | 16951.0891 | 17062.9418 | −0.6555 | 0 | 40 | 9.00E−05 | 1 | 50 |
| 19037 | 16951.0891 | 17116.8924 | −0.9687 | 0 | 14 | 0.043 | 1 | 51 |
| 19037 | 17007.0151 | 16956.9598 | 0.2952 | 0 | 276 | 2.50E−28 | 1 | 52 |
| 19037 | 17007.0151 | 17116.8924 | −0.6419 | 0 | 253 | 5.60E−26 | 1 | 53 |
| 19037 | 23671.2753 | 23824.1239 | −0.6416 | 0 | 43 | 0.0025 | 3 | 54 |
| 19037 | 23672.3256 | 23472.2688 | 0.8523 | 0 | 107 | 1.10E−09 | 1 | 55 |
| 19037 | 23672.3256 | 23712.1677 | −0.168 | 0 | 36 | 0.015 | 1 | 56 |
| 19037 | 23672.4187 | 23872.1004 | −0.8365 | 0 | 108 | 7.90E−10 | 1 | 57 |
| 19037 | 23672.4187 | 23616.2065 | 0.238 | 0 | 57 | 0.00011 | 3 | 58 |
| 19037 | 23728.3602 | 23856.1055 | −0.5355 | 0 | 102 | 4.20E−09 | 1 | 59 |
| 19037 | 23728.3602 | 23872.1004 | −0.6021 | 0 | 41 | 0.0045 | 4 | 60 |
| 19037 | 23728.3602 | 23712.1677 | 0.0683 | 0 | 46 | 0.0016 | 1 | 61 |
| 19037 | 23787.37 | 23728.1626 | 0.2495 | 0 | 35 | 0.024 | 3 | 62 |
| 19037 | 23845.4805 | 24032.033 | −0.7763 | 0 | 74 | 2.90E−06 | 1 | 63 |
| 19037 | 23845.4805 | 23664.1912 | 0.7661 | 0 | 50 | 0.00077 | 1 | 64 |
| 19037 | 23845.4805 | 23856.1055 | −0.0445 | 0 | 46 | 0.0019 | 1 | 65 |
| 19037 | 23847.4619 | 23808.129 | 0.1652 | 0 | 74 | 2.90E−06 | 7 | 66 |
| 19037 | 23847.4619 | 24032.033 | −0.768 | 0 | 42 | 0.0049 | 1 | 67 |
| 19037 | 23909.5298 | 23824.1239 | 0.3585 | 0 | 40 | 0.0075 | 6 | 68 |
| 19037 | 23909.5298 | 23744.1576 | 0.6965 | 0 | 41 | 0.0065 | 5 | 69 |
| 19037 | 23909.5298 | 24143.9892 | −0.9711 | 0 | 58 | 0.00011 | 3 | 70 |
| 19037 | 23909.5597 | 23904.0902 | 0.0229 | 0 | 56 | 0.0002 | 1 | 71 |
| 19037 | 23909.5597 | 23818.1497 | 0.3838 | 0 | 38 | 0.011 | 2 | 72 |
| 19037 | 23672.4187 | 23736.1442 | −0.2685 | 0 | 104 | 2.40E−09 | 2 | 73 |
| 19037 | 23728.3602 | 23576.2116 | 0.6453 | 0 | 99 | 7.70E−09 | 4 | 74 |
| 19037 | 23787.37 | 23656.1779 | 0.5546 | 0 | 37 | 0.013 | 1 | 75 |
| 19037 | 23845.4547 | 23752.1391 | 0.3929 | 0 | 32 | 0.048 | 3 | 76 |
| 19037 | 23845.4805 | 23752.1391 | 0.393 | 0 | 73 | 3.40E−06 | 2 | 77 |
| 19037 | 23845.4805 | 23624.1881 | 0.9367 | 0 | 48 | 0.0013 | 2 | 78 |
| 19037 | 23845.4805 | 24024.0197 | −0.7432 | 0 | 45 | 0.0021 | 2 | 79 |
| 19037 | 23847.4619 | 23672.1728 | 0.7405 | 0 | 75 | 2.20E−06 | 2 | 80 |
| 19037 | 23847.4619 | 23784.1207 | 0.2663 | 0 | 36 | 0.019 | 7 | 81 |
| 19037 | 23909.5298 | 24119.9809 | −0.8725 | 0 | 42 | 0.0052 | 3 | 82 |
| 19037 | 23909.5298 | 23784.1207 | 0.5273 | 0 | 41 | 0.0058 | 4 | 83 |
| 19037 | 23909.5298 | 23752.1391 | 0.6626 | 0 | 59 | 8.60E−05 | 1 | 84 |
| 19037 | 23909.5597 | 24119.9809 | −0.8724 | 0 | 87 | 1.60E−07 | 3 | 85 |
| 19037 | 18391.5315 | 18498.4994 | −0.5783 | 0 | 32 | 0.0013 | 1 | 86 |
| 19037 | 18391.5315 | 18514.4943 | −0.6641 | 0 | 20 | 0.019 | 2 | 87 |
| 19037 | 18391.5315 | 18498.4994 | −0.5783 | 0 | 18 | 0.033 | 3 | 88 |
| 19037 | 18421.5644 | 18578.4657 | −0.8445 | 0 | 41 | 0.00031 | 1 | 89 |
| 19037 | 18449.5517 | 18514.4943 | −0.3508 | 0 | 48 | 7.80E−05 | 1 | 90 |
| 19037 | 18451.5719 | 18578.4657 | −0.683 | 0 | 35 | 0.0017 | 10 | 91 |
| 19037 | 18451.5719 | 18562.4708 | −0.5974 | 0 | 34 | 0.002 | 9 | 92 |
| 19037 | 18474.535 | 18658.432 | −0.9856 | 0 | 36 | 0.0018 | 3 | 93 |
| 19037 | 18474.535 | 18658.432 | −0.9856 | 0 | 32 | 0.0042 | 1 | 94 |
| 19037 | 18475.5026 | 18578.4657 | −0.5542 | 0 | 39 | 0.00087 | 1 | 95 |
| 19037 | 18475.5026 | 18594.4606 | −0.6397 | 0 | 34 | 0.003 | 6 | 96 |
| 19037 | 18475.5026 | 18578.4657 | −0.5542 | 0 | 42 | 0.0004 | 1 | 97 |
| 19037 | 18476.6103 | 18578.4657 | −0.5482 | 0 | 39 | 0.00093 | 1 | 98 |
| 19037 | 18476.6103 | 18578.4657 | −0.5482 | 0 | 28 | 0.012 | 5 | 99 |
| 19037 | 18477.5282 | 18642.4371 | −0.8846 | 0 | 23 | 0.037 | 6 | 100 |
| 19037 | 18477.5636 | 18594.4606 | −0.6287 | 0 | 30 | 0.0079 | 1 | 101 |
| 19037 | 18477.6205 | 18658.432 | −0.9691 | 0 | 32 | 0.0047 | 1 | 102 |
| 19037 | 18477.6205 | 18658.432 | −0.9691 | 0 | 30 | 0.0066 | 2 | 103 |
| 19037 | 18477.6205 | 18578.4657 | −0.5428 | 0 | 31 | 0.0052 | 1 | 104 |
| 19037 | 18478.5574 | 18594.4606 | −0.6233 | 0 | 34 | 0.0025 | 1 | 105 |
| 19037 | 18532.6488 | 18674.4269 | −0.7592 | 0 | 34 | 0.0041 | 1 | 106 |
| 19037 | 18532.6488 | 18674.4269 | −0.7592 | 0 | 24 | 0.043 | 4 | 107 |
| 19037 | 18532.6488 | 18610.4555 | −0.4181 | 0 | 27 | 0.022 | 5 | 108 |
| 19037 | 18534.6247 | 18610.4555 | −0.4075 | 0 | 26 | 0.029 | 4 | 109 |
| 19037 | 18535.5421 | 18578.4657 | −0.231 | 0 | 33 | 0.005 | 4 | 110 |
| 19037 | 18535.5421 | 18578.4657 | −0.231 | 0 | 30 | 0.01 | 4 | 111 |
| 19037 | 23671.2753 | 23674.2484 | −0.0126 | 0 | 45 | 0.0017 | 1 | 112 |
| 19037 | 23672.3256 | 23802.1912 | −0.5456 | 0 | 102 | 3.80E−09 | 5 | 113 |
| 19037 | 23672.4187 | 23460.365 | 0.9039 | 0 | 39 | 0.0066 | 3 | 114 |
| 19037 | 23728.3602 | 23882.1575 | −0.644 | 0 | 97 | 1.20E−08 | 6 | 115 |
| 19037 | 23787.37 | 24010.1086 | −0.9277 | 0 | 34 | 0.027 | 10 | 116 |
| 19037 | 23845.4805 | 24058.0851 | −0.8837 | 0 | 73 | 3.70E−06 | 3 | 117 |
| 19037 | 23845.4805 | 24026.0952 | −0.7517 | 0 | 47 | 0.0015 | 4 | 118 |
| 19037 | 23847.4619 | 23754.2147 | 0.3926 | 0 | 75 | 2.30E−06 | 4 | 119 |
| 19037 | 23909.5597 | 23754.2147 | 0.654 | 0 | 35 | 0.026 | 7 | 120 |
| 19037 | 23671.2753 | 23678.2069 | −0.0293 | 0 | 42 | 0.0036 | 6 | 121 |
| 19037 | 23672.3256 | 23566.2507 | 0.4501 | 0 | 53 | 0.00025 | 1 | 122 |
| 19037 | 23672.4187 | 23688.2276 | −0.0667 | 0 | 40 | 0.0058 | 1 | 123 |
| 19037 | 23672.4187 | 23598.2406 | 0.3143 | 0 | 61 | 4.30E−05 | 1 | 124 |
| 19037 | 23672.4187 | 23646.2171 | 0.1108 | 0 | 48 | 0.0008 | 1 | 125 |
| 19037 | 23728.3602 | 23582.2457 | 0.6196 | 0 | 42 | 0.0042 | 6 | 126 |
| 19037 | 23787.37 | 23998.0722 | −0.878 | 0 | 38 | 0.01 | 1 | 127 |
| 19037 | 23845.4547 | 23710.1967 | 0.5705 | 0 | 34 | 0.031 | 1 | 128 |
| 19037 | 23845.4805 | 23614.2355 | 0.9793 | 0 | 72 | 4.20E−06 | 4 | 129 |
| 19037 | 23845.4805 | 23630.2304 | 0.9109 | 0 | 43 | 0.0035 | 7 | 130 |
| 19037 | 23847.4619 | 23854.1345 | −0.028 | 0 | 76 | 1.90E−06 | 1 | 131 |
| 19037 | 23847.4619 | 23806.1497 | 0.1735 | 0 | 36 | 0.017 | 6 | 132 |
| 19037 | 23909.5298 | 24094.0334 | −0.7658 | 0 | 45 | 0.0026 | 1 | 133 |
| 19037 | 23909.5298 | 23710.1967 | 0.8407 | 0 | 45 | 0.0021 | 1 | 134 |
| 19037 | 23909.5298 | 24126.015 | −0.8973 | 0 | 37 | 0.015 | 1 | 135 |
| 19037 | 23909.5597 | 23838.1395 | 0.2996 | 0 | 50 | 0.00078 | 4 | 136 |
| 19037 | 23909.5597 | 23934.1008 | −0.1025 | 0 | 40 | 0.0083 | 1 | 137 |
| 19037 | 18391.5315 | 18552.45 | −0.8674 | 0 | 28 | 0.003 | 2 | 138 |
| 19037 | 18393.4911 | 18568.4449 | −0.9422 | 0 | 21 | 0.015 | 5 | 139 |
| 19037 | 18393.5511 | 18568.4449 | −0.9419 | 0 | 36 | 0.00056 | 1 | 140 |
| 19037 | 18415.4249 | 18584.4399 | −0.9094 | 0 | 35 | 0.00099 | 2 | 141 |
| 19037 | 18418.4653 | 18488.4786 | −0.3787 | 0 | 21 | 0.027 | 2 | 142 |
| 19037 | 18448.4935 | 18568.4449 | −0.646 | 0 | 31 | 0.0036 | 1 | 143 |
| 19037 | 18450.4969 | 18600.4348 | −0.8061 | 0 | 22 | 0.032 | 1 | 144 |
| 19037 | 18474.535 | 18568.4449 | −0.5058 | 0 | 37 | 0.0013 | 1 | 145 |
| 19037 | 18475.5026 | 18584.4399 | −0.5862 | 0 | 37 | 0.0014 | 4 | 146 |
| 19037 | 18475.5026 | 18659.4871 | −0.986 | 0 | 39 | 0.00082 | 1 | 147 |
| 19037 | 18477.5282 | 18568.4449 | −0.4896 | 0 | 24 | 0.027 | 1 | 148 |
| 19037 | 18477.5282 | 18579.5208 | −0.549 | 0 | 22 | 0.05 | 8 | 149 |
| 19037 | 18477.5636 | 18648.4113 | −0.9162 | 0 | 26 | 0.017 | 1 | 150 |
| 19037 | 18477.6205 | 18648.4113 | −0.9158 | 0 | 31 | 0.0053 | 1 | 151 |
| 19037 | 18478.5574 | 18584.4399 | −0.5697 | 0 | 46 | 0.00018 | 1 | 152 |
| 19037 | 18478.5574 | 18659.4871 | −0.9696 | 0 | 30 | 0.0071 | 5 | 153 |
| 19037 | 18532.6488 | 18648.4113 | −0.6208 | 0 | 31 | 0.0085 | 5 | 154 |
| 19037 | 18532.6488 | 18648.4113 | −0.6208 | 0 | 31 | 0.0084 | 1 | 155 |
| 19037 | 18534.6247 | 18664.4062 | −0.6953 | 0 | 38 | 0.0019 | 1 | 156 |
| 19037 | 18534.6247 | 18664.4062 | −0.6953 | 0 | 46 | 0.00029 | 1 | 157 |
| 19037 | 18534.6247 | 18664.4062 | −0.6953 | 0 | 30 | 0.012 | 1 | 158 |
| 19037 | 18535.5421 | 18568.4449 | −0.1772 | 0 | 47 | 0.0002 | 1 | 159 |
| 19037 | 18535.5421 | 18664.4062 | −0.6904 | 0 | 35 | 0.0037 | 3 | 160 |
| 19037 | 18535.5421 | 18664.4062 | −0.6904 | 0 | 38 | 0.0017 | 1 | 161 |
| 19037 | 18393.4911 | 18516.4558 | −0.6641 | 0 | 19 | 0.026 | 3 | 162 |
| 19037 | 18393.4911 | 18532.4507 | −0.7498 | 0 | 28 | 0.0036 | 1 | 163 |
| 19037 | 18415.4249 | 18596.4221 | −0.9733 | 0 | 36 | 0.00076 | 1 | 164 |
| 19037 | 18449.5517 | 18612.417 | −0.875 | 0 | 22 | 0.03 | 3 | 165 |
| 19037 | 18451.5719 | 18612.417 | −0.8642 | 0 | 39 | 0.00067 | 1 | 166 |
| 19037 | 18451.5719 | 18596.4221 | −0.7789 | 0 | 37 | 0.001 | 4 | 167 |
| 19037 | 18474.535 | 18628.4119 | −0.826 | 0 | 24 | 0.028 | 1 | 168 |
| 19037 | 18475.5026 | 18612.417 | −0.7356 | 0 | 27 | 0.014 | 3 | 169 |
| 19037 | 18475.5026 | 18580.4272 | −0.5647 | 0 | 37 | 0.0015 | 7 | 170 |
| 19037 | 18475.5026 | 18612.417 | −0.7356 | 0 | 39 | 0.00081 | 1 | 171 |
| 19037 | 18475.5026 | 18612.417 | −0.7356 | 0 | 39 | 0.00087 | 2 | 172 |
| 19037 | 18476.6103 | 18628.4119 | −0.8149 | 0 | 30 | 0.0074 | 4 | 173 |
| 19037 | 18477.5636 | 18612.417 | −0.7245 | 0 | 25 | 0.022 | 4 | 174 |
| 19037 | 18478.5574 | 18628.4119 | −0.8044 | 0 | 42 | 0.00046 | 8 | 175 |
| 19037 | 18478.5574 | 18612.417 | −0.7192 | 0 | 39 | 0.00093 | 1 | 176 |
| 19037 | 18532.6488 | 18676.3884 | −0.7696 | 0 | 34 | 0.0045 | 2 | 177 |
| 19037 | 18532.6488 | 18596.4221 | −0.3429 | 0 | 25 | 0.033 | 1 | 178 |
| 19037 | 18532.6488 | 18628.4119 | −0.5141 | 0 | 28 | 0.016 | 3 | 179 |
| 19037 | 18534.6247 | 18596.4221 | −0.3323 | 0 | 32 | 0.0069 | 3 | 180 |
| 19037 | 18534.6247 | 18612.417 | −0.418 | 0 | 39 | 0.0015 | 7 | 181 |
| 19037 | 18534.6247 | 18596.4221 | −0.3323 | 0 | 25 | 0.031 | 10 | 182 |
| 19037 | 18535.5421 | 18676.3884 | −0.7541 | 0 | 26 | 0.03 | 4 | 183 |
| 19037 | 18535.5421 | 18676.3884 | −0.7541 | 0 | 46 | 0.00025 | 2 | 184 |
| 19037 | 18449.5517 | 18553.5416 | −0.5605 | 0 | 40 | 0.00056 | 8 | 185 |
| 19037 | 18451.5719 | 18633.5079 | −0.9764 | 0 | 39 | 0.00069 | 7 | 186 |
| 19037 | 18451.5719 | 18633.5079 | −0.9764 | 0 | 34 | 0.0021 | 5 | 187 |
| 19037 | 18474.535 | 18649.5028 | −0.9382 | 0 | 26 | 0.016 | 2 | 188 |
| 19037 | 18476.6103 | 18649.5028 | −0.9271 | 0 | 34 | 0.003 | 3 | 189 |
| 19037 | 18476.6103 | 18569.5365 | −0.5004 | 0 | 26 | 0.016 | 6 | 190 |
| 19037 | 18477.5282 | 18649.5028 | −0.9221 | 0 | 24 | 0.027 | 2 | 191 |
| 19037 | 18477.5282 | 18649.5028 | −0.9221 | 0 | 27 | 0.015 | 1 | 192 |
| 19037 | 18481.6212 | 18649.5028 | −0.9002 | 0 | 27 | 0.016 | 1 | 193 |
| 19037 | 18534.6247 | 18633.5079 | −0.5307 | 0 | 29 | 0.014 | 3 | 194 |
| 19037 | 18391.5315 | 18562.5307 | −0.9212 | 0 | 27 | 0.0037 | 1 | 195 |
| 19037 | 18451.5719 | 18546.5357 | −0.512 | 0 | 32 | 0.003 | 5 | 196 |
| 19037 | 18451.5719 | 18562.5307 | −0.5978 | 0 | 39 | 0.00061 | 1 | 197 |
| 19037 | 18475.5026 | 18610.5071 | −0.7254 | 0 | 33 | 0.0036 | 8 | 198 |
| 19037 | 18478.5574 | 18626.5021 | −0.7943 | 0 | 30 | 0.0068 | 10 | 199 |
| 19037 | 18534.6247 | 18626.5021 | −0.4933 | 0 | 25 | 0.036 | 6 | 200 |
| 19037 | 18391.5315 | 18570.5205 | −0.9638 | 0 | 20 | 0.021 | 1 | 201 |
| 19037 | 18449.5517 | 18554.5256 | −0.5658 | 0 | 42 | 0.00036 | 2 | 202 |
| 19037 | 18475.5026 | 18634.4919 | −0.8532 | 0 | 28 | 0.011 | 1 | 203 |
| 19037 | 18477.5282 | 18650.4868 | −0.9274 | 0 | 23 | 0.034 | 4 | 204 |
| 19037 | 18477.5636 | 18650.4868 | −0.9272 | 0 | 23 | 0.035 | 4 | 205 |
| 19037 | 18481.6212 | 18650.4868 | −0.9054 | 0 | 23 | 0.033 | 1 | 206 |
| 19037 | 18535.5421 | 18650.4868 | −0.6163 | 0 | 39 | 0.0015 | 1 | 207 |
| 19037 | 18476.6103 | 18656.5573 | −0.9645 | 0 | 36 | 0.0016 | 1 | 208 |
| 19037 | 18534.6247 | 18656.5573 | −0.6536 | 0 | 24 | 0.039 | 8 | 209 |
| 19037 | 18449.5517 | 18602.5467 | −0.8224 | 0 | 26 | 0.014 | 1 | 210 |
| 19037 | 18532.6488 | 18682.513 | −0.8022 | 0 | 27 | 0.02 | 4 | 211 |
| 19037 | 18534.6247 | 18682.513 | −0.7916 | 0 | 28 | 0.017 | 10 | 212 |
| 19037 | 18534.6247 | 18666.5181 | −0.7066 | 0 | 26 | 0.025 | 8 | 213 |
| 19020 | 16946.0112 | 17036.9261 | −0.5336 | 0 | 66 | 0.0065 | 1 | 214 |
| 19020 | 16947.0673 | 17036.9261 | −0.5274 | 0 | 148 | 4.30E−11 | 1 | 215 |
| 19020 | 16947.1076 | 17088.0003 | −0.8245 | 0 | 151 | 2.00E−11 | 1 | 216 |
| 19020 | 16948.0322 | 17020.9312 | −0.4283 | 0 | 58 | 0.043 | 1 | 217 |
| 19020 | 16948.0429 | 17036.9261 | −0.5217 | 0 | 103 | 1.20E−06 | 1 | 218 |
| 19020 | 16949.1095 | 17072.0054 | −0.7199 | 0 | 22 | 0.017 | 1 | 219 |
| 19020 | 16950.0324 | 16956.9598 | −0.0409 | 0 | 122 | 1.40E−08 | 1 | 220 |
| 19020 | 16950.0418 | 17088.0003 | −0.8073 | 0 | 70 | 0.0025 | 1 | 221 |
| 19020 | 16950.0418 | 17100.8975 | −0.8822 | 0 | 128 | 4.10E−09 | 1 | 222 |
| 19020 | 16950.044 | 16940.9649 | 0.0536 | 0 | 143 | 1.30E−10 | 1 | 223 |
| 19020 | 16951.0333 | 16956.9598 | −0.035 | 0 | 92 | 1.60E−05 | 1 | 224 |
| 19020 | 16967.0303 | 17088.0003 | −0.7079 | 0 | 94 | 2.30E−06 | 1 | 225 |
| 19020 | 17007.0151 | 17020.9312 | −0.0818 | 0 | 172 | 1.60E−13 | 1 | 226 |
| 19040 | 16947.0673 | 17036.9261 | −0.5274 | 0 | 229 | 3.10E−19 | 1 | 227 |
| 19040 | 16947.0673 | 17036.9261 | −0.5274 | 0 | 245 | 8.60E−21 | 1 | 228 |
| 19040 | 16947.1076 | 17036.9261 | −0.5272 | 0 | 236 | 6.00E−20 | 1 | 229 |
| 19040 | 16948.021 | 17103.9952 | −0.9119 | 0 | 67 | 0.0046 | 1 | 230 |
| 19040 | 16948.0322 | 17036.9261 | −0.5218 | 0 | 155 | 7.20E−12 | 1 | 231 |
| 19040 | 16948.0429 | 17036.9261 | −0.5217 | 0 | 142 | 1.50E−10 | 1 | 232 |
| 19040 | 16948.0429 | 17036.9261 | −0.5217 | 0 | 168 | 4.00E−13 | 1 | 233 |
| 19040 | 16948.0429 | 17020.9312 | −0.4282 | 0 | 140 | 2.40E−10 | 1 | 234 |
| 19040 | 16949.0707 | 17088.0003 | −0.813 | 0 | 116 | 6.30E−08 | 1 | 235 |
| 19040 | 16949.9917 | 16956.9598 | −0.0411 | 0 | 202 | 1.40E−16 | 1 | 236 |
| 19040 | 16950.0155 | 17052.921 | −0.6034 | 0 | 63 | 0.013 | 1 | 237 |
| 19040 | 16950.0155 | 17052.921 | −0.6034 | 0 | 61 | 0.019 | 1 | 238 |
| 19040 | 16950.0324 | 17020.9312 | −0.4165 | 0 | 212 | 1.50E−17 | 1 | 239 |
| 19040 | 16950.0418 | 17100.8975 | −0.8822 | 0 | 164 | 1.00E−12 | 1 | 240 |
| 19040 | 16951.0333 | 16940.9649 | 0.0594 | 0 | 285 | 8.40E−25 | 1 | 241 |
| 19040 | 16951.0766 | 17088.0003 | −0.8013 | 0 | 80 | 0.00027 | 1 | 242 |
| 19040 | 16952.0746 | 17088.0003 | −0.7954 | 0 | 165 | 8.30E−13 | 1 | 243 |
| 19040 | 16964.0472 | 17116.8924 | −0.8929 | 0 | 101 | 1.90E−06 | 6 | 244 |
| 19040 | 17007.0151 | 16956.9598 | 0.2952 | 0 | 276 | 6.10E−24 | 1 | 245 |
| 19040 | 17007.0151 | 17116.8924 | −0.6419 | 0 | 253 | 1.40E−21 | 1 | 246 |
| 19052 | 16946.0112 | 17036.9261 | −0.5336 | 0 | 66 | 0.00042 | 1 | 247 |
| 19052 | 16947.0673 | 17036.9261 | −0.5274 | 0 | 148 | 2.80E−12 | 1 | 248 |
| 19052 | 16947.1076 | 17088.0003 | −0.8245 | 0 | 151 | 1.30E−12 | 1 | 249 |
| 19052 | 16948.0322 | 17020.9312 | −0.4283 | 0 | 58 | 0.0027 | 1 | 250 |
| 19052 | 16948.0429 | 17103.9952 | −0.9118 | 0 | 54 | 0.0066 | 1 | 251 |
| 19052 | 16948.0429 | 17036.9261 | −0.5217 | 0 | 103 | 7.90E−08 | 1 | 252 |
| 19052 | 16948.0429 | 17116.8924 | −0.9864 | 0 | 50 | 0.015 | 1 | 253 |
| 19052 | 16949.1095 | 17072.0054 | −0.7199 | 0 | 22 | 0.017 | 1 | 254 |
| 19052 | 16950.0324 | 16956.9598 | −0.0409 | 0 | 122 | 9.10E−10 | 1 | 255 |
| 19052 | 16950.0418 | 17088.0003 | −0.8073 | 0 | 70 | 0.00016 | 1 | 256 |
| 19052 | 16950.0418 | 17100.8975 | −0.8822 | 0 | 128 | 2.60E−10 | 1 | 257 |
| 19052 | 16950.044 | 16940.9649 | 0.0536 | 0 | 143 | 8.30E−12 | 1 | 258 |
| 19052 | 16951.0333 | 16956.9598 | −0.035 | 0 | 92 | 1.00E−06 | 1 | 259 |
| 19052 | 16967.0303 | 17088.0003 | −0.7079 | 0 | 94 | 6.70E−07 | 1 | 260 |
| 19052 | 17007.0151 | 17020.9312 | −0.0818 | 0 | 172 | 1.00E−14 | 1 | 261 |
| 19047 | 16947.0673 | 17036.9261 | −0.5274 | 0 | 229 | 2.00E−20 | 1 | 262 |
| 19047 | 16947.0673 | 17036.9261 | −0.5274 | 0 | 245 | 5.50E−22 | 1 | 263 |
| 19047 | 16947.1076 | 17062.9418 | −0.6789 | 0 | 243 | 7.80E−22 | 1 | 264 |
| 19047 | 16948.0322 | 17036.9261 | −0.5218 | 0 | 155 | 4.60E−13 | 1 | 265 |
| 19047 | 16948.0429 | 17036.9261 | −0.5217 | 0 | 142 | 9.70E−12 | 1 | 266 |
| 19047 | 16948.0429 | 17036.9261 | −0.5217 | 0 | 168 | 2.50E−14 | 1 | 267 |
| 19047 | 16948.0429 | 17020.9312 | −0.4282 | 0 | 140 | 1.50E−11 | 1 | 268 |
| 19047 | 16949.0707 | 17088.0003 | −0.813 | 0 | 116 | 4.00E−09 | 1 | 269 |
| 19047 | 16949.9917 | 16956.9598 | −0.0411 | 0 | 202 | 8.90E−18 | 1 | 270 |
| 19047 | 16950.0155 | 17052.921 | −0.6034 | 0 | 63 | 0.00084 | 1 | 271 |
| 19047 | 16950.0155 | 17094.9316 | −0.8477 | 0 | 68 | 0.00026 | 1 | 272 |
| 19047 | 16950.0324 | 17020.9312 | −0.4165 | 0 | 212 | 9.40E−19 | 1 | 273 |
| 19047 | 16950.0418 | 17114.0159 | −0.9581 | 0 | 141 | 1.30E−11 | 1 | 274 |
| 19047 | 16950.0418 | 17100.8975 | −0.8822 | 0 | 164 | 6.50E−14 | 1 | 275 |
| 19047 | 16951.0333 | 16940.9649 | 0.0594 | 0 | 285 | 5.40E−26 | 1 | 276 |
| 19047 | 16951.0766 | 17088.0003 | −0.8013 | 0 | 80 | 1.70E−05 | 1 | 277 |
| 19047 | 16952.0746 | 17088.0003 | −0.7954 | 0 | 165 | 5.30E−14 | 1 | 278 |
| 19047 | 16952.0746 | 17072.0054 | −0.7025 | 0 | 217 | 3.00E−19 | 1 | 279 |
| 19047 | 16964.0472 | 17116.8924 | −0.8929 | 0 | 101 | 1.20E−07 | 6 | 280 |
| 19047 | 17007.0151 | 16956.9598 | 0.2952 | 0 | 276 | 3.90E−25 | 1 | 281 |
| 19047 | 17007.0151 | 17116.8924 | −0.6419 | 0 | 253 | 8.90E−23 | 1 | 282 |
| 19047 | 18535.5421 | 18577.8376 | −0.2277 | 0 | 46 | 0.042 | 1 | 283 |
| 19047 | 18477.6205 | 18654.5484 | −0.9484 | 0 | 34 | 0.039 | 1 | 284 |

All the entries of Swissprot database (559,228 sequences) were also searched with a ±50 ppm fragment tolerance. The Mascot search result is reported in Table 8 and FIG. 12. Not only was the search much longer than with our smaller more targeted homemade database lasting 3 days, but also only myoglobin could be identified, based on a total of 46 (12%) MS/MS spectra (71% redundancy) yielding a protein score of 1,456. As observed with the ‘homemade’ database described at [0185], above, the unmodified isoform was the most frequently identified (39%), the other proteoforms comprised oxidation and/or phosphorylation sites (Table 9). Raising the MS/MS tolerance to 2 Da did not increase the list of protein identified but adjusted the score to 8,764 with 113 (30%) matches. Limiting Swissprot taxonomy to “other mammalia” adjusted myoglobin scores to 17,072 with 62 (17%) matches and 10,298 with 136 (37%) matches, respectively applying ±50 ppm and ±2 Da fragment tolerance. While this reduces search times to hours, it results in the identification of a protein we do not expect in our known protein samples, NADH-ubiquinone oxidoreductase (Tables 8 and 9). As the commercial standards we used are not pure, it is possible that this protein is genuinely present in the sample. In any case, these data indicated that increasing the search space by choosing a database with more entries and selecting more dynamic modifications lengthens the time needed to complete the search (Table 7), without necessarily yielding more relevant identities (Table 8).

### Example 7—Proteins Identified by Top-Down Proteomics

Protein extracts from cannabis mature buds were concentrated by evaporation to maximise signal intensity. The chromatographic separation of intact denatured proteins was optimised from 15 to 40% of mobile phase B for 87 min. ETD, CID and HCD was applied in succession with three levels of energy so called “Low” (ETD 5 ms, CID 35 eV, HCD 19 eV), “Mid” (ETD 10 ms, CID 42 eV, HCD 23 eV) and “High” (ETD 15 ms, CID 50 eV, HCD 27 eV).

Three cannabis extracts (bud 1 to 3) were run using LC-MS in duplicate and using LC-MS/MS in triplicate with high reproducibility (FIG. 12). Total ion chromatograms (TIC) were very similar across technical replicates, as well as among biological replicates 2 and 3 (FIG. 12A); sample bud 1 differed slightly mostly due to lower signal intensities during the first half of the LC run. LC-MS patterns are very similar, generally differing in peak intensities across biological replicates (FIG. 12B) as the number of protein groups was consistent with small standard deviation (SD) values (470±17 groups) (Table 10).

TABLE 10
Statistics on cannabis proteins analysed by LC-MS and
LC-MS/MS obtained from Genedata Refiner analysis.

| Tech. Rep. | Bud 1 | Bud 2 | Bud 3 | Mean | SD |
|---|---|---|---|---|---|
| Replicate 1 | 442 | 483 | 483 | 469 | 19 |
| Replicate 2 | 474 | 486 | 453 | 471 | 14 |
| Mean | 458 | 485 | 468 | | |
| SD | 16 | 2 | 15 | | |

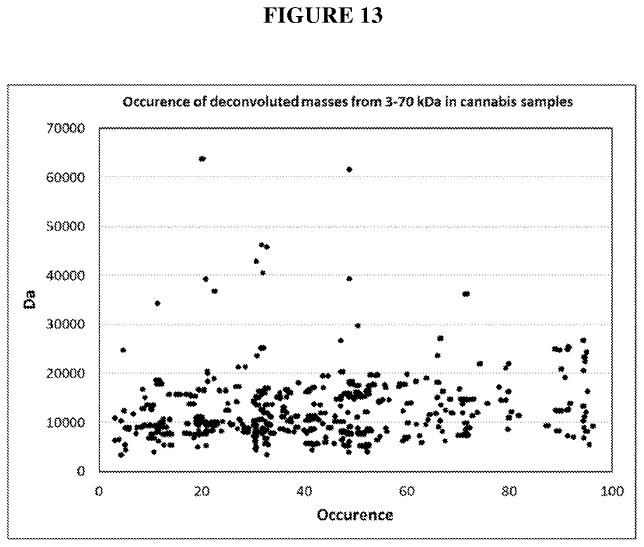

Maps of deconvoluted masses were also highly comparable, with the greatest majority of proteins (93%) being smaller than 20 kD (FIG. 12C and FIG. 13); a zoom-in confirms the lesser intensity of bud 1 pattern (FIG. 12D). Increasing the chromatographic separation from 60 to 120 min and using HPLC column packed with a C4 rather than a C8 stationary phase. This results in better utilisation of the 500-2000 m/z range (503-1799 m/z), enhanced dynamic range (from 10⁴ to 10⁸, i.e. 4 orders of magnitude), increased numbers of multiply-charged ions, and overall superior and more reproducible LC-MS profiles.

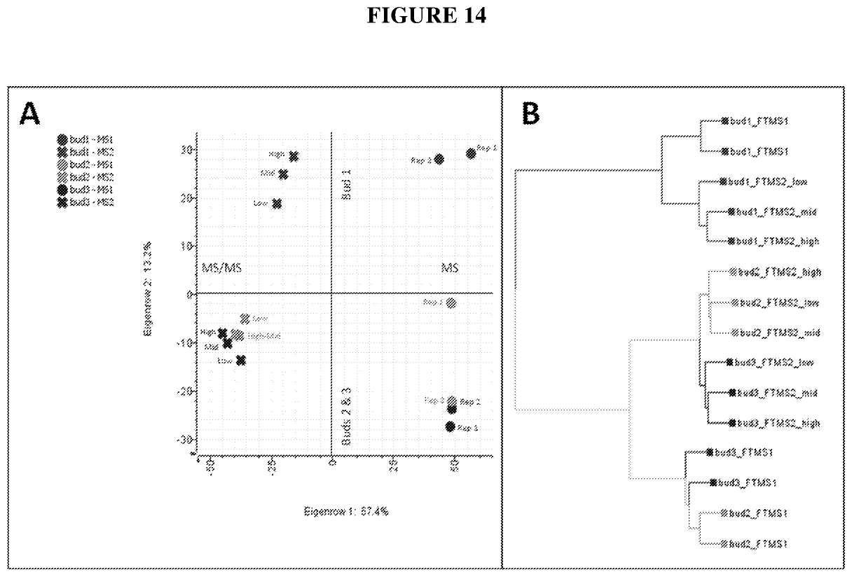

The triplicated LC-MS/MS patterns are also very similar as exemplified in bud 1 (FIG. 12E). Table 11 lists the number of MS/MS spectra per sample (1160 to 1220 MS/MS spectra on average) and method (1178 to 1189 MS/MS spectra on average); SD values were very small and comparable across samples (±8 to 11) and methods (±22 to 31), indicative of high reproducibility. The reproducibility of the LC-MS and LC-MS/MS analyses was statistically assessed (FIG. 14). Both PCA and HCA clearly separate the bud 1 sample from the other two biological samples, and on the LC-MS data from LC-MS/MS data. Technical replicates clustered together.

TABLE 11
Number of MS/MS spectra collected across each “Low, “Mid”, and
“High” MS/MS method.

| Method | Bud 1 | Bud 2 | Bud 3 | Mean | SD |
|---|---|---|---|---|---|
| “Low” | 1157 | 1169 | 1208 | 1178 | 22 |
| “Mid” | 1173 | 1193 | 1226 | 1197 | 22 |
| “High” | 1149 | 1192 | 1225 | 1189 | 31 |
| Mean | 1160 | 1185 | 1220 | | |
| SD | 10 | 11 | 8 | | |

The most abundant multiply charged precursors were selected for MS/MS experiments (Table 12).

TABLE 12
Statistics on parent ions from cannabis
proteins analysed by LC-MS/MS.

| | | | | Min. | Max. | No. of |
|---|---|---|---|---|---|---|
| Charge | No. of | Min. | Max. | Mass | Mass | MS/MS |
| state | precursors | m/z | m/z | (Da) | (Da) | events |
| 2 | 34 | 714.18 | 1500.37 | 1426.36 | 2998.73 | 63 |
| 3 | 8 | 848.75 | 1176.15 | 2543.23 | 3525.44 | 32 |
| 4 | 45 | 714.08 | 1380.06 | 2852.31 | 5516.21 | 143 |
| 5 | 39 | 803.49 | 1325.52 | 4012.42 | 6622.58 | 120 |
| 6 | 43 | 775.62 | 1458.49 | 4647.67 | 8744.89 | 109 |
| 7 | 61 | 747.77 | 1534.29 | 5227.35 | 10732.96 | 222 |
| 8 | 86 | 787.70 | 1429.84 | 6293.52 | 11430.63 | 341 |
| 9 | 69 | 700.41 | 1564.79 | 6294.62 | 14074.01 | 262 |
| 10 | 48 | 756.92 | 1729.69 | 7559.16 | 17286.78 | 195 |
| 11 | 32 | 726.96 | 1338.87 | 7985.51 | 14716.50 | 113 |
| 12 | 30 | 710.98 | 1338.68 | 8519.65 | 16052.07 | 99 |
| 13 | 32 | 762.47 | 1256.51 | 9898.99 | 16321.52 | 114 |
| 14 | 36 | 732.89 | 1318.67 | 10246.31 | 18447.31 | 125 |
| 15 | 32 | 738.60 | 1099.47 | 11063.95 | 16433.03 | 109 |
| 16 | 29 | 708.10 | 1153.96 | 11269.49 | 18447.30 | 105 |
| 17 | 29 | 737.28 | 1129.03 | 12516.63 | 19176.39 | 86 |
| 18 | 27 | 754.89 | 1163.66 | 13569.88 | 20927.81 | 96 |
| 19 | 37 | 715.21 | 1135.96 | 13569.85 | 21564.03 | 124 |
| 20 | 38 | 710.24 | 1240.59 | 14184.59 | 24791.58 | 126 |
| 21 | 34 | 723.89 | 1185.04 | 15180.59 | 24864.66 | 106 |
| 22 | 28 | 701.95 | 1155.10 | 15420.70 | 25390.00 | 92 |
| 23 | 14 | 711.74 | 1104.83 | 16346.79 | 25387.98 | 31 |
| 24 | 8 | 746.08 | 1036.99 | 17881.77 | 24863.64 | 18 |
| 25 | 3 | 745.98 | 992.59 | 18624.23 | 24789.59 | 3 |

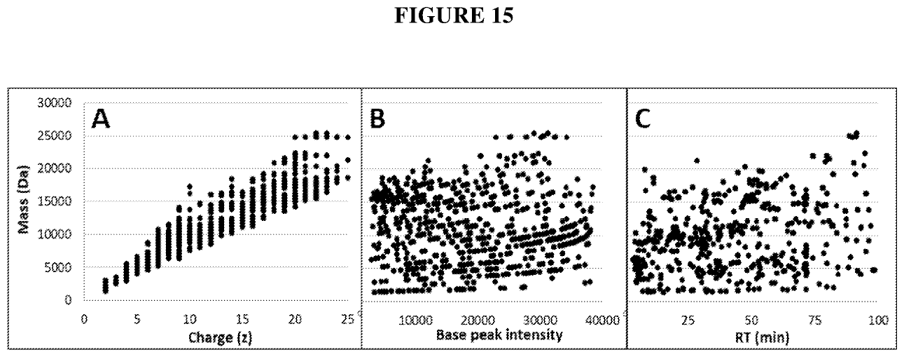

Overall, precursor charge states ranged from +2 to +25, parent ions from 700.4 to 1729.7 m/z, and their accurate masses span 1.4 to 25.4 kDa. Inherent to MS, the greater the charge state, the greater the mass of cannabis proteins (FIG. 15A). The most abundant precursors comprised 4 to 10 charges and their accurate masses range from 2.8 to 17.3 kDa. Therefore, this type of analysis predominantly favours small proteins from cannabis buds. Another factor determining precursor selection pertains to protein abundance, emulated by base peak intensity in the mass spectrometer. In particular, for a proteins larger than 20 kDa to undergo MS/MS, its base peak intensity must exceed 2,000 counts (FIG. 15B).

The last factor determining precursor selection relates to protein hydrophobicity which affects the chromatographic elution. FIG. 15C demonstrates that proteins larger than 20 kD were eluted after 75 min of reverse phase separation, indicating that these proteins were more hydrophobic than proteins of smaller size. Therefore, for highly hydrophobic proteins, the separation method prior to the MS analysis needs to be refined using a different type of stationary phase and/or different mobile phases and gradients.

A total of 11,250 MS/MS peak lists were searched against the UniprotKB C. sativa database (663 entries) using Mascot algorithm, a fragment tolerance of ±50 ppm or ±2 Da, and validating the results using a decoy or an error tolerant method (Table 7). With a ±50 ppm fragment tolerance, Protein N-term acetylation and Met oxidation set as dynamic modifications and an error tolerant method, 12 proteins were identified (210 (2%) matches) with 11,040 (98%) MS/MS spectra remaining unassigned and a search time of over 24 h. Using the same parameters but changing error tolerance to decoy brings the number of accessions identified to 21 from 213 (2%) matched MS/MS spectra and a very fast search time of 29 s (Table 13). Excessive stringency in Mascot algorithm could justify the low number of database hits. Rising the fragment tolerance to ±2 Da, listed 36 proteins based on 355 (3%) assigned MS/MS spectra with a search time of 2.5 min. With a ±50 ppm fragment tolerance, Protein N-term acetylation, Met oxidation, phosphorylations of Ser and Tyr residues set as dynamic modifications and a decoy method, the number of unique protein identified was 21 (187 matches) after almost 2 h search. Lifting the fragment tolerance to ±2 Da as well as the number of hits (61 proteins, 590 (5%) MS/MS spectra assigned). Forsaking dynamic modifications reduced search times and yielded 20 and 24 identities using ±50 ppm and ±2 Da fragment tolerance, respectively (Tables 7 and 14).

TABLE 13
List of cannabis proteins identified by top-down proteomics using Mascot
algorithm, C. sativa UniprotKB database and ±50 ppm fragment tolerance.

| | | | Mass | No. of | No. of | | |
|---|---|---|---|---|---|---|---|
| Member | Accession | Score | (Da) | matches | sequences | emPAI | Description |
| 1 | A0A0C5ARS8 | 2265 | 9367 | 37 | 1 | 0.83 | Cytochrome b559 subunit alpha |
| 1 | A0A0C5AS17 | 1664 | 9545 | 39 | 1 | 1.43 | Photosystem I iron-sulfur center |
| 1 | A0A0U2DTK8 | 1555 | 3815 | 25 | 1 | 13.87 | Photosystem II reaction center protein T |
| 1 | A0A0C5B2J7 | 1348 | 7645 | 12 | 1 | 1.06 | Photosystem II reaction center protein H |
| 1 | A0A0U2GZT5 | 902 | 9381 | 21 | 1 | 0.35 | Cytochrome b559 subunit alpha |
| 1 | A0A0C5APX7 | 292 | 4165 | 9 | 1 | 5.31 | Photosystem II reaction center protein I |
| 1 | A0A0C5ARQ5 | 272 | 7985 | 12 | 1 | 1.84 | ATP synthase CF0 C subunit |
| 1 | A0A0U2H3S7 | 182 | 11833 | 5 | 1 | 0.62 | 30S ribosomal protein S14, chloroplastic |
| 1 | A0A0C5AUI2 | 182 | 4421 | 17 | 1 | 0.8 | Cytochrome b559 subunit beta |
| 1 | I6WU39 | 162 | 11994 | 9 | 1 | 0.61 | Olivetolic acid cyclase |
| 1 | A0A0H3W6G0 | 123 | 10414 | 5 | 1 | 0.72 | Ribosomal protein S16 |
| 1 | I6XT51 | 113 | 17597 | 7 | 2 | 1.28 | Betv1-like protein |
| 2 | A0A0U2DTC8 | 111 | 10380 | 4 | 1 | 0.72 | 30S ribosomal protein S16, chloroplastic |
| 1 | A0A0C5APY3 | 79 | 4128 | 2 | 1 | 0.87 | Photosystem II reaction center protein J |
| 1 | A0A0C5AUI5 | 72 | 7910 | 1 | 1 | 0.42 | Ribosomal protein L33 |
| 1 | A0A0C5AUH9 | 62 | 14696 | 1 | 1 | 0.22 | ATP synthase CF1 epsilon subunit |
| 1 | A0A0C5APY4 | 27 | 4167 | 1 | 1 | 0.85 | Cytochrome b6-f complex subunit 5 |
| 1 | W0U0V5 | 26 | 9489 | 2 | 1 | 0.35 | Non-specific lipid-transfer protein |
| 1 | A0A0H3W8G1 | 25 | 4494 | 2 | 1 | 0.8 | Photosystem II reaction center protein L |
| 1 | A0A0H3W844 | 24 | 17504 | 1 | 1 | 0.18 | Cytochrome b6-f complex subunit 4 |
| 1 | A0A0C5AS04 | 15 | 4770 | 1 | 1 | 0.74 | Photosystem I reaction center subunit IX |

| Member | Species | Proteoforms | BUP¹ |
|---|---|---|---|
| 1 | Cannabis sativa | Unmodified, Acetyl | yes |
| 1 | Cannabis sativa | Unmodified, 1 and 2 Oxidations | yes |
| 1 | C. sativa subsp. sativa | Unmodified | no |
| 1 | Cannabis sativa | Unmodified, Oxidation | no |
| 1 | Humulus lupulus | Unmodified | yes |
| 1 | Cannabis sativa | Unmodified, Acetyl, Oxidation | no |
| 1 | Cannabis sativa | Unmodified, Oxidation | no |
| 1 | Humulus lupulus | Unmodified, Oxidation | yes |
| 1 | Cannabis sativa | Unmodified | no |
| 1 | Cannabis sativa | Unmodified, Acetyl | yes |
| 1 | Cannabis sativa | Unmodified, Oxidation | no |
| 1 | Cannabis sativa | Unmodified, Acetyl, Oxidation | yes |
| 2 | C. sativa subsp. sativa | Unmodified | no |
| 1 | Cannabis sativa | Acetyl | no |
| 1 | Cannabis sativa | Unmodified | no |
| 1 | Cannabis sativa | Acetyl | yes |
| 1 | Cannabis sativa | Unmodified | no |
| 1 | Cannabis sativa | Unmodified | yes |
| 1 | Cannabis sativa | Unmodified | no |
| 1 | Cannabis sativa | Unmodified | no |
| 1 | Cannabis sativa | Acetyl, Oxidation | no |

¹BUP, protein identified by bottom-up proteomics in Table 4.

TABLE 14
List of proteins identified from medicinal cannabis protein samples using
Mascot algorithm and UniProtKB and SwissProt C. sativa databases

| Job | | | fragment | | decoy/ | | | | |
|---|---|---|---|---|---|---|---|---|---|
| no. | Taxonomy | PTMs | tolerance | | error | Family | M | Accession | Score |
| 19031 | C. sativa and | AO | 50 | ppm | error | 1 | 1 | tr\|A0A0C5ARS8\|A0A0C5ARS8_CANSA | 2174 |
| | relatives | | | | | | | | |
| 19031 | C. sativa and | AO | 50 | ppm | error | 2 | 1 | tr\|A0A0C5AS17\|A0A0C5AS17_CANSA | 1649 |
| | relatives | | | | | | | | |
| 19031 | C. sativa and | AO | 50 | ppm | error | 3 | 1 | tr\|A0A0C5B2J7\|A0A0C5B2J7_CANSA | 1348 |
| | relatives | | | | | | | | |
| 19031 | C. sativa and | AO | 50 | ppm | error | 4 | 1 | tr\|A0A0U2GZT5\|A0A0U2GZT5_HUMLU | 902 |
| | relatives | | | | | | | | |
| 19031 | C. sativa and | AO | 50 | ppm | error | 5 | 1 | tr\|A0A0U2DTK8\|A0A0U2DTK8_CANSA | 448 |
| | relatives | | | | | | | | |
| 19031 | C. sativa and | AO | 50 | ppm | error | 6 | 1 | tr\|A0A0C5ARQ5\|A0A0C5ARQ5_CANSA | 167 |
| | relatives | | | | | | | | |
| 19031 | C. sativa and | AO | 50 | ppm | error | 7 | 1 | sp\|I6WU39\|OLIAC_CANSA | 162 |
| | relatives | | | | | | | | |
| 19031 | C. sativa and | AO | 50 | ppm | error | 8 | 1 | tr\|A0A0C5APX7\|A0A0C5APX7_CANSA | 127 |
| | relatives | | | | | | | | |
| 19031 | C. sativa and | AO | 50 | ppm | error | 9 | 1 | tr\|A0A0U2DTC8\|A0A0U2DTC8_CANSA | 111 |
| | relatives | | | | | | | | |
| 19031 | C. sativa and | AO | 50 | ppm | error | 10 | 1 | tr\|A0A0C5APY3\|A0A0C5APY3_CANSA | 79 |
| | relatives | | | | | | | | |
| 19031 | C. sativa and | AO | 50 | ppm | error | 11 | 1 | tr\|A0A0U2H159\|A0A0U2H159_HUMLU | 54 |
| | relatives | | | | | | | | |
| 19031 | C. sativa and | AO | 50 | ppm | error | 12 | 1 | tr\|A0A0H3W8G1\|A0A0H3W8G1_CANSA | 25 |
| | relatives | | | | | | | | |
| 19030 | C. sativa and | AO | 50 | ppm | decoy | 1 | 1 | tr\|A0A0C5ARS8\|A0A0C5ARS8_CANSA | 2265 |
| | relatives | | | | | | | | |
| 19030 | C. sativa and | AO | 50 | ppm | decoy | 2 | 1 | tr\|A0A0C5AS17\|A0A0C5AS17_CANSA | 1664 |
| | relatives | | | | | | | | |
| 19030 | C. sativa and | AO | 50 | ppm | decoy | 3 | 1 | tr\|A0A0U2DTK8\|A0A0U2DTK8_CANSA | 1555 |
| | relatives | | | | | | | | |
| 19030 | C. sativa and | AO | 50 | ppm | decoy | 4 | 1 | tr\|A0A0C5B2J7\|A0A0C5B2J7_CANSA | 1348 |
| | relatives | | | | | | | | |
| 19030 | C. sativa and | AO | 50 | ppm | decoy | 5 | 1 | tr\|A0A0U2GZT5\|A0A0U2GZT5_HUMLU | 902 |
| | relatives | | | | | | | | |
| 19030 | C. sativa and | AO | 50 | ppm | decoy | 6 | 1 | tr\|A0A0C5APX7\|A0A0C5APX7_CANSA | 292 |
| | relatives | | | | | | | | |
| 19030 | C. sativa and | AO | 50 | ppm | decoy | 7 | 1 | tr\|A0A0C5ARQ5\|A0A0C5ARQ5_CANSA | 272 |
| | relatives | | | | | | | | |
| 19030 | C. sativa and | AO | 50 | ppm | decoy | 8 | 1 | tr\|A0A0U2H3S7\|A0A0U2H3S7_HUMLU | 182 |
| | relatives | | | | | | | | |
| 19030 | C. sativa and | AO | 50 | ppm | decoy | 9 | 1 | tr\|A0A0C5AUI2\|A0A0C5AUI2_CANSA | 182 |
| | relatives | | | | | | | | |
| 19030 | C. sativa and | AO | 50 | ppm | decoy | 10 | 1 | sp\|I6WU39\|OLIAC_CANSA | 162 |
| | relatives | | | | | | | | |
| 19030 | C. sativa and | AO | 50 | ppm | decoy | 11 | 1 | tr\|A0A0H3W6G0\|A0A0H3W6G0_CANSA | 123 |
| | relatives | | | | | | | | |
| 19030 | C. sativa and | AO | 50 | ppm | decoy | 11 | 2 | tr\|A0A0U2DTC8\|A0A0U2DTC8_CANSA | 111 |
| | relatives | | | | | | | | |
| 19030 | C. sativa and | AO | 50 | ppm | decoy | 12 | 1 | tr\|I6XT51\|I6XT51_CANSA | 113 |
| | relatives | | | | | | | | |
| 19030 | C. sativa and | AO | 50 | ppm | decoy | 13 | 1 | tr\|A0A0C5APY3\|A0A0C5APY3_CANSA | 79 |
| | relatives | | | | | | | | |
| 19030 | C. sativa and | AO | 50 | ppm | decoy | 14 | 1 | tr\|A0A0C5AUI5\|A0A0C5AUI5_CANSA | 72 |
| | relatives | | | | | | | | |
| 19030 | C. sativa and | AO | 50 | ppm | decoy | 15 | 1 | tr\|A0A0C5AUH9\|A0A0C5AUH9_CANSA | 62 |
| | relatives | | | | | | | | |
| 19030 | C. sativa and | AO | 50 | ppm | decoy | 16 | 1 | tr\|A0A0C5APY4\|A0A0C5APY4_CANSA | 27 |
| | relatives | | | | | | | | |
| 19030 | C. sativa and | AO | 50 | ppm | decoy | 17 | 1 | tr\|W0U0V5\|W0U0V5_CANSA | 26 |
| | relatives | | | | | | | | |
| 19030 | C. sativa and | AO | 50 | ppm | decoy | 18 | 1 | tr\|A0A0H3W8G1\|A0A0H3W8G1_CANSA | 25 |
| | relatives | | | | | | | | |
| 19030 | C. sativa and | AO | 50 | ppm | decoy | 19 | 1 | tr\|A0A0H3W844\|A0A0H3W844_CANSA | 24 |
| | relatives | | | | | | | | |
| 19030 | C. sativa and | AO | 50 | ppm | decoy | 20 | 1 | tr\|A0A0C5AS04\|A0A0C5AS04_CANSA | 15 |
| | relatives | | | | | | | | |
| 19048 | C. sativa and | AO | 2 | Da | decoy | 1 | 1 | tr\|A0A0C5AS17\|A0A0C5AS17_CANSA | 3341 |
| | relatives | | | | | | | | |
| 19048 | C. sativa and | AO | 2 | Da | decoy | 2 | 1 | tr\|A0A0C5ARS8\|A0A0C5ARS8_CANSA | 3243 |
| | relatives | | | | | | | | |
| 19048 | C. sativa and | AO | 2 | Da | decoy | 3 | 1 | tr\|A0A0C5B2J7\|A0A0C5B2J7_CANSA | 2046 |
| | relatives | | | | | | | | |
| 19048 | C. sativa and | AO | 2 | Da | decoy | 4 | 1 | tr\|A0A0U2DTK8\|A0A0U2DTK8_CANSA | 1983 |
| | relatives | | | | | | | | |
| 19048 | C. sativa and | AO | 2 | Da | decoy | 5 | 1 | tr\|I6XT51\|I6XT51_CANSA | 1227 |
| | relatives | | | | | | | | |
| 19048 | C. sativa and | AO | 2 | Da | decoy | 6 | 1 | tr\|A0A0C5ARQ5\|A0A0C5ARQ5_CANSA | 618 |
| | relatives | | | | | | | | |
| 19048 | C. sativa and | AO | 2 | Da | decoy | 7 | 1 | tr\|W0U0V5\|W0U0V5_CANSA | 477 |
| | relatives | | | | | | | | |
| 19048 | C. sativa and | AO | 2 | Da | decoy | 8 | 1 | sp\|I6WU39\|OLIAC_CANSA | 445 |
| | relatives | | | | | | | | |
| 19048 | C. sativa and | AO | 2 | Da | decoy | 9 | 1 | tr\|A0A0U2H3S7\|A0A0U2H3S7_HUMLU | 418 |
| | relatives | | | | | | | | |
| 19048 | C. sativa and | AO | 2 | Da | decoy | 10 | 1 | tr\|A0A0C5APX7\|A0A0C5APX7_CANSA | 333 |
| | relatives | | | | | | | | |
| 19048 | C. sativa and | AO | 2 | Da | decoy | 11 | 1 | tr\|A0A0U2H3Q7\|A0A0U2H3Q7_HUMLU | 293 |
| | relatives | | | | | | | | |
| 19048 | C. sativa and | AO | 2 | Da | decoy | 12 | 1 | tr\|A0A0H3W6G0\|A0A0H3W6G0_CANSA | 272 |
| | relatives | | | | | | | | |
| 19048 | C. sativa and | AO | 2 | Da | decoy | 13 | 1 | tr\|A0A0C5B2H7\|A0A0C5B2H7_CANSA | 266 |
| | relatives | | | | | | | | |
| 19048 | C. sativa and | AO | 2 | Da | decoy | 14 | 1 | tr\|A0A0C5AUI2\|A0A0C5AUI2_CANSA | 262 |
| | relatives | | | | | | | | |
| 19048 | C. sativa and | AO | 2 | Da | decoy | 15 | 1 | tr\|A0A0C5AUH9\|A0A0C5AUH9_CANSA | 240 |
| | relatives | | | | | | | | |
| 19048 | C. sativa and | AO | 2 | Da | decoy | 16 | 1 | tr\|A0A0U2DTC8\|A0A0U2DTC8_CANSA | 239 |
| | relatives | | | | | | | | |
| 19048 | C. sativa and | AO | 2 | Da | decoy | 17 | 1 | tr\|A0A0C5AUI5\|A0A0C5AUI5_CANSA | 137 |
| | relatives | | | | | | | | |
| 19048 | C. sativa and | AO | 2 | Da | decoy | 18 | 1 | tr\|A0A0C5APY3\|A0A0C5APY3_CANSA | 114 |
| | relatives | | | | | | | | |
| 19048 | C. sativa and | AO | 2 | Da | decoy | 19 | 1 | tr\|A0A172J205\|A0A172J205_BOENI | 86 |
| | relatives | | | | | | | | |
| 19048 | C. sativa and | AO | 2 | Da | decoy | 20 | 1 | tr\|A0A0H3W844\|A0A0H3W844_CANSA | 57 |
| | relatives | | | | | | | | |
| 19048 | C. sativa and | AO | 2 | Da | decoy | 21 | 1 | tr\|A0A0C5AS04\|A0A0C5AS04_CANSA | 54 |
| | relatives | | | | | | | | |
| 19048 | C. sativa and | AO | 2 | Da | decoy | 22 | 1 | tr\|A0A0C5APY7\|A0A0C5APY7_CANSA | 45 |
| | relatives | | | | | | | | |
| 19048 | C. sativa and | AO | 2 | Da | decoy | 23 | 1 | tr\|A0A0H3W8G1\|A0A0H3W8G1_CANSA | 33 |
| | relatives | | | | | | | | |
| 19048 | C. sativa and | AO | 2 | Da | decoy | 24 | 1 | tr\|A0A172J223\|A0A172J223_BOENI | 31 |
| | relatives | | | | | | | | |
| 19048 | C. sativa and | AO | 2 | Da | decoy | 25 | 1 | tr\|A0A3G3NDF5\|A0A3G3NDF5_CANSA | 29 |
| | relatives | | | | | | | | |
| 19048 | C. sativa and | AO | 2 | Da | decoy | 26 | 1 | tr\|A0A0C5APY4\|A0A0C5APY4_CANSA | 28 |
| | relatives | | | | | | | | |
| 19048 | C. sativa and | AO | 2 | Da | decoy | 27 | 1 | tr\|A0A172J276\|A0A172J276_BOENI | 27 |
| | relatives | | | | | | | | |
| 19048 | C. sativa and | AO | 2 | Da | decoy | 28 | 1 | tr\|A0A172J254\|A0A172J254_BOENI | 27 |
| | relatives | | | | | | | | |
| 19048 | C. sativa and | AO | 2 | Da | decoy | 29 | 1 | tr\|A0A0U2H2X0\|A0A0U2H2X0_HUMLU | 22 |
| | relatives | | | | | | | | |
| 19048 | C. sativa and | AO | 2 | Da | decoy | 30 | 1 | tr\|A0A172J266\|A0A172J266_BOENI | 22 |
| | relatives | | | | | | | | |
| 19048 | C. sativa and | AO | 2 | Da | decoy | 31 | 1 | tr\|A0A0Y0UZ03\|A0A0Y0UZ03_CANSA | 19 |
| | relatives | | | | | | | | |
| 19048 | C. sativa and | AO | 2 | Da | decoy | 32 | 1 | tr\|Q5TIQ0\|Q5TIQ0_CANSA | 16 |
| | relatives | | | | | | | | |
| 19048 | C. sativa and | AO | 2 | Da | decoy | 33 | 1 | tr\|A0A172J200\|A0A172J200_BOENI | 16 |
| | relatives | | | | | | | | |
| 19048 | C. sativa and | AO | 2 | Da | decoy | 34 | 1 | tr\|A0A0C5B2J2\|A0A0C5B2J2_CANSA | 15 |
| | relatives | | | | | | | | |
| 19048 | C. sativa and | AO | 2 | Da | decoy | 35 | 1 | tr\|A0A1W2KS31\|A0A1W2KS31_CANSA | 15 |
| | relatives | | | | | | | | |
| 19048 | C. sativa and | AO | 2 | Da | decoy | 36 | 1 | tr\|A0A1U9VXL5\|A0A1U9VXL5_CANSA | 14 |
| | relatives | | | | | | | | |
| 19050 | C. sativa and | AOP | 50 | ppm | decoy | 1 | 1 | tr\|A0A0C5ARS8\|A0A0C5ARS8_CANSA | 2166 |
| | relatives | | | | | | | | |
| 19050 | C. sativa and | AOP | 50 | ppm | decoy | 2 | 1 | tr\|A0A0C5B2J7\|A0A0C5B2J7_CANSA | 1547 |
| | relatives | | | | | | | | |
| 19050 | C. sativa and | AOP | 50 | ppm | decoy | 3 | 1 | tr\|A0A0C5AS17\|A0A0C5AS17_CANSA | 1499 |
| | relatives | | | | | | | | |
| 19050 | C. sativa and | AOP | 50 | ppm | decoy | 4 | 1 | tr\|A0A0U2DTK8\|A0A0U2DTK8_CANSA | 1459 |
| | relatives | | | | | | | | |
| 19050 | C. sativa and | AOP | 50 | ppm | decoy | 5 | 1 | tr\|A0A0C5AUI2\|A0A0C5AUI2_CANSA | 676 |
| | relatives | | | | | | | | |
| 19050 | C. sativa and | AOP | 50 | ppm | decoy | 6 | 1 | tr\|A0A0C5APX7\|A0A0C5APX7_CANSA | 279 |
| | relatives | | | | | | | | |
| 19050 | C. sativa and | AOP | 50 | ppm | decoy | 7 | 1 | tr\|A0A0C5ARQ5\|A0A0C5ARQ5_CANSA | 223 |
| | relatives | | | | | | | | |
| 19050 | C. sativa and | AOP | 50 | ppm | decoy | 8 | 1 | sp\|I6WU39\|OLIAC_CANSA | 156 |
| | relatives | | | | | | | | |
| 19050 | C. sativa and | AOP | 50 | ppm | decoy | 9 | 1 | tr\|A0A0U2H3S7\|A0A0U2H3S7_HUMLU | 140 |
| | relatives | | | | | | | | |
| 19050 | C. sativa and | AOP | 50 | ppm | decoy | 10 | 1 | tr\|A0A0H3W6G0\|A0A0H3W6G0_CANSA | 112 |
| | relatives | | | | | | | | |
| 19050 | C. sativa and | AOP | 50 | ppm | decoy | 11 | 1 | tr\|A0A0U2DTC8\|A0A0U2DTC8_CANSA | 111 |
| | relatives | | | | | | | | |
| 19050 | C. sativa and | AOP | 50 | ppm | decoy | 12 | 1 | tr\|A0A0C5APY3\|A0A0C5APY3_CANSA | 74 |
| | relatives | | | | | | | | |
| 19050 | C. sativa and | AOP | 50 | ppm | decoy | 13 | 1 | tr\|A0A0C5AUI5\|A0A0C5AUI5_CANSA | 72 |
| | relatives | | | | | | | | |
| 19050 | C. sativa and | AOP | 50 | ppm | decoy | 14 | 1 | tr\|I6XT51\|I6XT51_CANSA | 68 |
| | relatives | | | | | | | | |
| 19050 | C. sativa and | AOP | 50 | ppm | decoy | 15 | 1 | tr\|A0A0C5AUH9\|A0A0C5AUH9_CANSA | 62 |
| | relatives | | | | | | | | |
| 19050 | C. sativa and | AOP | 50 | ppm | decoy | 16 | 1 | tr\|W0U0V5\|W0U0V5_CANSA | 34 |
| | relatives | | | | | | | | |
| 19050 | C. sativa and | AOP | 50 | ppm | decoy | 17 | 1 | tr\|A0A0C5AS00\|A0A0C5AS00_CANSA | 30 |
| | relatives | | | | | | | | |
| 19050 | C. sativa and | AOP | 50 | ppm | decoy | 18 | 1 | tr\|A0A0C5APY4\|A0A0C5APY4_CANSA | 27 |
| | relatives | | | | | | | | |
| 19050 | C. sativa and | AOP | 50 | ppm | decoy | 19 | 1 | tr\|A0A0H3W8G1\|A0A0H3W8G1_CANSA | 25 |
| | relatives | | | | | | | | |
| 19050 | C. sativa and | AOP | 50 | ppm | decoy | 20 | 1 | tr\|A0A0H3W844\|A0A0H3W844_CANSA | 24 |
| | relatives | | | | | | | | |
| 19050 | C. sativa and | AOP | 50 | ppm | decoy | 21 | 1 | tr\|A0A0C5AS04\|A0A0C5AS04_CANSA | 15 |
| | relatives | | | | | | | | |
| 19049 | C. sativa and | AOP | 2 | Da | decoy | 1 | 1 | tr\|A0A0C5ARS8\|A0A0C5ARS8_CANSA | 3186 |
| | relatives | | | | | | | | |
| 19049 | C. sativa and | AOP | 2 | Da | decoy | 2 | 1 | tr\|A0A0C5AS17\|A0A0C5AS17_CANSA | 3158 |
| | relatives | | | | | | | | |
| 19049 | C. sativa and | AOP | 2 | Da | decoy | 3 | 1 | tr\|A0A0C5B2J7\|A0A0C5B2J7_CANSA | 2468 |
| | relatives | | | | | | | | |
| 19049 | C. sativa and | AOP | 2 | Da | decoy | 4 | 1 | tr\|A0A0U2DTK8\|A0A0U2DTK8_CANSA | 2057 |
| | relatives | | | | | | | | |
| 19049 | C. sativa and | AOP | 2 | Da | decoy | 5 | 1 | tr\|A0A0C5ARQ5\|A0A0C5ARQ5_CANSA | 1902 |
| | relatives | | | | | | | | |
| 19049 | C. sativa and | AOP | 2 | Da | decoy | 6 | 1 | tr\|A0A0U2GZT5\|A0A0U2GZT5_HUMLU | 1831 |
| | relatives | | | | | | | | |
| 19049 | C. sativa and | AOP | 2 | Da | decoy | 7 | 1 | tr\|A0A0C5AUI2\|A0A0C5AUI2_CANSA | 1314 |
| | relatives | | | | | | | | |
| 19049 | C. sativa and | AOP | 2 | Da | decoy | 8 | 1 | tr\|I6XT51\|I6XT51_CANSA | 986 |
| | relatives | | | | | | | | |
| 19049 | C. sativa and | AOP | 2 | Da | decoy | 9 | 1 | tr\|W0U0V5\|W0U0V5_CANSA | 896 |
| | relatives | | | | | | | | |
| 19049 | C. sativa and | AOP | 2 | Da | decoy | 10 | 1 | tr\|A0A0C5APX7\|A0A0C5APX7_CANSA | 691 |
| | relatives | | | | | | | | |
| 19049 | C. sativa and | AOP | 2 | Da | decoy | 11 | 1 | tr\|A0A0U2DTC8\|A0A0U2DTC8_CANSA | 382 |
| | relatives | | | | | | | | |
| 19049 | C. sativa and | AOP | 2 | Da | decoy | 12 | 1 | sp\|I6WU39\|OLIAC_CANSA | 379 |
| | relatives | | | | | | | | |
| 19049 | C. sativa and | AOP | 2 | Da | decoy | 13 | 1 | tr\|A0A0C5AS04\|A0A0C5AS04_CANSA | 285 |
| | relatives | | | | | | | | |
| 19049 | C. sativa and | AOP | 2 | Da | decoy | 14 | 1 | tr\|A0A0U2H3S7\|A0A0U2H3S7_HUMLU | 278 |
| | relatives | | | | | | | | |
| 19049 | C. sativa and | AOP | 2 | Da | decoy | 15 | 1 | tr\|A0A0C5AUH9\|A0A0C5AUH9_CANSA | 229 |
| | relatives | | | | | | | | |
| 19049 | C. sativa and | AOP | 2 | Da | decoy | 16 | 1 | tr\|A0A0C5B2H7\|A0A0C5B2H7_CANSA | 224 |
| | relatives | | | | | | | | |
| 19049 | C. sativa and | AOP | 2 | Da | decoy | 17 | 1 | tr\|A0A0C5AS00\|A0A0C5AS00_CANSA | 217 |
| | relatives | | | | | | | | |
| 19049 | C. sativa and | AOP | 2 | Da | decoy | 18 | 1 | tr\|A0A0C5APY3\|A0A0C5APY3_CANSA | 195 |
| | relatives | | | | | | | | |
| 19049 | C. sativa and | AOP | 2 | Da | decoy | 19 | 1 | tr\|A0A0U2H159\|A0A0U2H159_HUMLU | 167 |
| | relatives | | | | | | | | |
| 19049 | C. sativa and | AOP | 2 | Da | decoy | 20 | 1 | tr\|A0A0U2H3Q7\|A0A0U2H3Q7_HUMLU | 161 |
| | relatives | | | | | | | | |
| 19049 | C. sativa and | AOP | 2 | Da | decoy | 21 | 1 | tr\|A0A172J1Y7\|A0A172J1Y7_BOENI | 160 |
| | relatives | | | | | | | | |
| 19049 | C. sativa and | AOP | 2 | Da | decoy | 22 | 1 | tr\|A0A0C5AUI5\|A0A0C5AUI5_CANSA | 137 |
| | relatives | | | | | | | | |
| 19049 | C. sativa and | AOP | 2 | Da | decoy | 23 | 1 | tr\|A0A0M4QYI4\|A0A0M4QYI4_CANSA | 88 |
| | relatives | | | | | | | | |
| 19049 | C. sativa and | AOP | 2 | Da | decoy | 24 | 1 | tr\|A0A0H3W8G1\|A0A0H3W8G1_CANSA | 78 |
| | relatives | | | | | | | | |
| 19049 | C. sativa and | AOP | 2 | Da | decoy | 25 | 1 | tr\|A0A0H3W8B6\|A0A0H3W8B6_CANSA | 78 |
| | relatives | | | | | | | | |
| 19049 | C. sativa and | AOP | 2 | Da | decoy | 26 | 1 | tr\|A0A0H3W844\|A0A0H3W844_CANSA | 77 |
| | relatives | | | | | | | | |
| 19049 | C. sativa and | AOP | 2 | Da | decoy | 27 | 1 | tr\|A0A172J205\|A0A172J205_BOENI | 73 |
| | relatives | | | | | | | | |
| 19049 | C. sativa and | AOP | 2 | Da | decoy | 28 | 1 | tr\|R4I7F6\|R4I7F6_CANSA | 63 |
| | relatives | | | | | | | | |
| 19049 | C. sativa and | AOP | 2 | Da | decoy | 29 | 1 | tr\|A0A3G3NDF5\|A0A3G3NDF5_CANSA | 60 |
| | relatives | | | | | | | | |
| 19049 | C. sativa and | AOP | 2 | Da | decoy | 30 | 1 | tr\|A0A0M3ULW1\|A0A0M3ULW1_CANSA | 60 |
| | relatives | | | | | | | | |
| 19049 | C. sativa and | AOP | 2 | Da | decoy | 31 | 1 | tr\|A0A0C5AS02\|A0A0C5AS02_CANSA | 53 |
| | relatives | | | | | | | | |
| 19049 | C. sativa and | AOP | 2 | Da | decoy | 32 | 1 | tr\|A0A0C5ARS1\|A0A0C5ARS1_CANSA | 46 |
| | relatives | | | | | | | | |
| 19049 | C. sativa and | AOP | 2 | Da | decoy | 33 | 1 | tr\|A0A0C5APY7\|A0A0C5APY7_CANSA | 45 |
| | relatives | | | | | | | | |
| 19049 | C. sativa and | AOP | 2 | Da | decoy | 34 | 1 | tr\|A0A172J1X8\|A0A172J1X8_BOENI | 42 |
| | relatives | | | | | | | | |
| 19049 | C. sativa and | AOP | 2 | Da | decoy | 35 | 1 | tr\|A0A172J290\|A0A172J290_BOENI | 41 |
| | relatives | | | | | | | | |
| 19049 | C. sativa and | AOP | 2 | Da | decoy | 36 | 1 | tr\|A0A172J266\|A0A172J266_BOENI | 41 |
| | relatives | | | | | | | | |
| 19049 | C. sativa and | AOP | 2 | Da | decoy | 37 | 1 | tr\|A0A172J222\|A0A172J222_BOENI | 40 |
| | relatives | | | | | | | | |
| 19049 | C. sativa and | AOP | 2 | Da | decoy | 38 | 1 | tr\|A0A172J232\|A0A172J232_BOENI | 39 |
| | relatives | | | | | | | | |
| 19049 | C. sativa and | AOP | 2 | Da | decoy | 39 | 1 | tr\|A0A0Y0UZ03\|A0A0Y0UZ03_CANSA | 39 |
| | relatives | | | | | | | | |
| 19049 | C. sativa and | AOP | 2 | Da | decoy | 40 | 1 | tr\|A0A3G3NDF7\|A0A3G3NDF7_CANSA | 37 |
| | relatives | | | | | | | | |
| 19049 | C. sativa and | AOP | 2 | Da | decoy | 41 | 1 | tr\|A0A172J230\|A0A172J230_BOENI | 36 |
| | relatives | | | | | | | | |
| 19049 | C. sativa and | AOP | 2 | Da | decoy | 42 | 1 | tr\|A0A172J220\|A0A172J220_BOENI | 34 |
| | relatives | | | | | | | | |
| 19049 | C. sativa and | AOP | 2 | Da | decoy | 43 | 1 | tr\|A0A172J239\|A0A172J239_BOENI | 34 |
| | relatives | | | | | | | | |
| 19049 | C. sativa and | AOP | 2 | Da | decoy | 44 | 1 | tr\|A0A0C5ART4\|A0A0C5ART4_CANSA | 34 |
| | relatives | | | | | | | | |
| 19049 | C. sativa and | AOP | 2 | Da | decoy | 45 | 1 | tr\|A0A3R5T0F7\|A0A3R5T0F7_CANSA | 33 |
| | relatives | | | | | | | | |
| 19049 | C. sativa and | AOP | 2 | Da | decoy | 46 | 1 | tr\|A0A172J1X4\|A0A172J1X4_BOENI | 33 |
| | relatives | | | | | | | | |
| 19049 | C. sativa and | AOP | 2 | Da | decoy | 47 | 1 | tr\|A0A0C5APY8\|A0A0C5APY8_CANSA | 32 |
| | relatives | | | | | | | | |
| 19049 | C. sativa and | AOP | 2 | Da | decoy | 48 | 1 | tr\|A0A0C5AUJ2\|A0A0C5AUJ2_CANSA | 31 |
| | relatives | | | | | | | | |
| 19049 | C. sativa and | AOP | 2 | Da | decoy | 49 | 1 | tr\|A0A172J1Y0\|A0A172J1Y0_BOENI | 31 |
| | relatives | | | | | | | | |
| 19049 | C. sativa and | AOP | 2 | Da | decoy | 50 | 1 | tr\|A0A172J237\|A0A172J237_BOENI | 30 |
| | relatives | | | | | | | | |
| 19049 | C. sativa and | AOP | 2 | Da | decoy | 51 | 1 | tr\|A0A172J213\|A0A172J213_BOENI | 30 |
| | relatives | | | | | | | | |
| 19049 | C. sativa and | AOP | 2 | Da | decoy | 52 | 1 | tr\|A0A0C5APY4\|A0A0C5APY4_CANSA | 28 |
| | relatives | | | | | | | | |
| 19049 | C. sativa and | AOP | 2 | Da | decoy | 53 | 1 | tr\|A0A0U2DTJ2\|A0A0U2DTJ2_CANSA | 28 |
| | relatives | | | | | | | | |
| 19049 | C. sativa and | AOP | 2 | Da | decoy | 54 | 1 | tr\|Q5TIQ0\|Q5TIQ0_CANSA | 28 |
| | relatives | | | | | | | | |
| 19049 | C. sativa and | AOP | 2 | Da | decoy | 55 | 1 | tr\|B5AFH3\|B5AFH3_CANSA | 27 |
| | relatives | | | | | | | | |
| 19049 | C. sativa and | AOP | 2 | Da | decoy | 56 | 1 | tr\|Q5TIP7\|Q5TIP7_CANSA | 27 |
| | relatives | | | | | | | | |
| 19049 | C. sativa and | AOP | 2 | Da | decoy | 57 | 1 | tr\|A0A1U9VXK6\|A0A1U9VXK6_CANSA | 23 |
| | relatives | | | | | | | | |
| 19049 | C. sativa and | AOP | 2 | Da | decoy | 58 | 1 | tr\|A9XV94\|A9XV94_CANSA | 20 |
| | relatives | | | | | | | | |
| 19049 | C. sativa and | AOP | 2 | Da | decoy | 59 | 1 | tr\|A0A0C5B2J2\|A0A0C5B2J2_CANSA | 19 |
| | relatives | | | | | | | | |
| 19049 | C. sativa and | AOP | 2 | Da | decoy | 60 | 1 | tr\|A0A0C5B2G1\|A0A0C5B2G1_CANSA | 19 |
| | relatives | | | | | | | | |
| 19049 | C. sativa and | AOP | 2 | Da | decoy | 61 | 1 | tr\|Q5TIP6\|Q5TIP6_CANSA | 18 |
| | relatives | | | | | | | | |
| 19051 | C. sativa and | none | 50 | ppm | decoy | 1 | 1 | tr\|A0A0C5ARS8\|A0A0C5ARS8_CANSA | 2260 |
| | relatives | | | | | | | | |
| 19051 | C. sativa and | none | 50 | ppm | decoy | 2 | 1 | tr\|A0A0C5AS17\|A0A0C5AS17_CANSA | 1696 |
| | relatives | | | | | | | | |
| 19051 | C. sativa and | none | 50 | ppm | decoy | 3 | 1 | tr\|A0A0U2DTK8\|A0A0U2DTK8_CANSA | 1326 |
| | relatives | | | | | | | | |
| 19051 | C. sativa and | none | 50 | ppm | decoy | 4 | 1 | tr\|A0A0C5B2J7\|A0A0C5B2J7_CANSA | 1285 |
| | relatives | | | | | | | | |
| 19051 | C. sativa and | none | 50 | ppm | decoy | 5 | 1 | tr\|A0A0U2GZT5\|A0A0U2GZT5_HUMLU | 905 |
| | relatives | | | | | | | | |
| 19051 | C. sativa and | none | 50 | ppm | decoy | 6 | 1 | tr\|A0A0C5APX7\|A0A0C5APX7_CANSA | 291 |
| | relatives | | | | | | | | |
| 19051 | C. sativa and | none | 50 | ppm | decoy | 7 | 1 | tr\|A0A0C5ARQ5\|A0A0C5ARQ5_CANSA | 250 |
| | relatives | | | | | | | | |
| 19051 | C. sativa and | none | 50 | ppm | decoy | 8 | 1 | sp\|I6WU39\|OLIAC_CANSA | 191 |
| | relatives | | | | | | | | |
| 19051 | C. sativa and | none | 50 | ppm | decoy | 9 | 1 | tr\|A0A0C5AUI2\|A0A0C5AUI2_CANSA | 182 |
| | relatives | | | | | | | | |
| 19051 | C. sativa and | none | 50 | ppm | decoy | 10 | 1 | tr\|A0A0H3W6G0\|A0A0H3W6G0_CANSA | 152 |
| | relatives | | | | | | | | |
| 19051 | C. sativa and | none | 50 | ppm | decoy | 11 | 1 | tr\|A0A0U2H3S7\|A0A0U2H3S7_HUMLU | 144 |
| | relatives | | | | | | | | |
| 19051 | C. sativa and | none | 50 | ppm | decoy | 12 | 1 | tr\|A0A0U2DTC8\|A0A0U2DTC8_CANSA | 132 |
| | relatives | | | | | | | | |
| 19051 | C. sativa and | none | 50 | ppm | decoy | 13 | 1 | tr\|I6XT51\|I6XT51_CANSA | 125 |
| | relatives | | | | | | | | |
| 19051 | C. sativa and | none | 50 | ppm | decoy | 14 | 1 | tr\|A0A0C5AUI5\|A0A0C5AUI5_CANSA | 72 |
| | relatives | | | | | | | | |
| 19051 | C. sativa and | none | 50 | ppm | decoy | 15 | 1 | tr\|A0A0C5AUH9\|A0A0C5AUH9_CANSA | 51 |
| | relatives | | | | | | | | |
| 19051 | C. sativa and | none | 50 | ppm | decoy | 16 | 1 | tr\|W0U0V5\|W0U0V5_CANSA | 29 |
| | relatives | | | | | | | | |
| 19051 | C. sativa and | none | 50 | ppm | decoy | 17 | 1 | tr\|A0A0C5APY4\|A0A0C5APY4_CANSA | 27 |
| | relatives | | | | | | | | |
| 19051 | C. sativa and | none | 50 | ppm | decoy | 18 | 1 | tr\|A0A0H3W8G1\|A0A0H3W8G1_CANSA | 25 |
| | relatives | | | | | | | | |
| 19051 | C. sativa and | none | 50 | ppm | decoy | 19 | 1 | tr\|A0A0H3W844\|A0A0H3W844_CANSA | 24 |
| | relatives | | | | | | | | |
| 19051 | C. sativa and | none | 50 | ppm | decoy | 20 | 1 | tr\|A0A0C5AS04\|A0A0C5AS04_CANSA | 14 |
| | relatives | | | | | | | | |
| 19043 | C. sativa and | none | 2 | Da | decoy | 1 | 1 | tr\|A0A0C5AS17\|A0A0C5AS17_CANSA | 3384 |
| | relatives | | | | | | | | |
| 19043 | C. sativa and | none | 2 | Da | decoy | 2 | 1 | tr\|A0A0C5ARS8\|A0A0C5ARS8_CANSA | 3236 |
| | relatives | | | | | | | | |
| 19043 | C. sativa and | none | 2 | Da | decoy | 3 | 1 | tr\|A0A0C5B2J7\|A0A0C5B2J7_CANSA | 1996 |
| | relatives | | | | | | | | |
| 19043 | C. sativa and | none | 2 | Da | decoy | 4 | 1 | tr\|A0A0U2DTK8\|A0A0U2DTK8_CANSA | 1606 |
| | relatives | | | | | | | | |
| 19043 | C. sativa and | none | 2 | Da | decoy | 5 | 1 | tr\|I6XT51\|I6XT51_CANSA | 959 |
| | relatives | | | | | | | | |
| 19043 | C. sativa and | none | 2 | Da | decoy | 6 | 1 | tr\|W0U0V5\|W0U0V5_CANSA | 521 |
| | relatives | | | | | | | | |
| 19043 | C. sativa and | none | 2 | Da | decoy | 7 | 1 | sp\|I6WU39\|OLIAC_CANSA | 464 |
| | relatives | | | | | | | | |
| 19043 | C. sativa and | none | 2 | Da | decoy | 8 | 1 | tr\|A0A0C5ARQ5\|A0A0C5ARQ5_CANSA | 449 |
| | relatives | | | | | | | | |
| 19043 | C. sativa and | none | 2 | Da | decoy | 9 | 1 | tr\|A0A0U2H3S7\|A0A0U2H3S7_HUMLU | 344 |
| | relatives | | | | | | | | |
| 19043 | C. sativa and | none | 2 | Da | decoy | 10 | 1 | tr\|A0A0H3W6G0\|A0A0H3W6G0_CANSA | 310 |
| | relatives | | | | | | | | |
| 19043 | C. sativa and | none | 2 | Da | decoy | 11 | 1 | tr\|A0A0C5APX7\|A0A0C5APX7_CANSA | 294 |
| | relatives | | | | | | | | |
| 19043 | C. sativa and | none | 2 | Da | decoy | 12 | 1 | tr\|A0A0C5AUI2\|A0A0C5AUI2_CANSA | 262 |
| | relatives | | | | | | | | |
| 19043 | C. sativa and | none | 2 | Da | decoy | 13 | 1 | tr\|A0A0U2DTC8\|A0A0U2DTC8_CANSA | 243 |
| | relatives | | | | | | | | |
| 19043 | C. sativa and | none | 2 | Da | decoy | 14 | 1 | tr\|A0A0C5B2H7\|A0A0C5B2H7_CANSA | 208 |
| | relatives | | | | | | | | |
| 19043 | C. sativa and | none | 2 | Da | decoy | 15 | 1 | tr\|A0A0C5AUH9\|A0A0C5AUH9_CANSA | 149 |
| | relatives | | | | | | | | |
| 19043 | C. sativa and | none | 2 | Da | decoy | 16 | 1 | tr\|A0A0C5AUI5\|A0A0C5AUI5_CANSA | 137 |
| | relatives | | | | | | | | |
| 19043 | C. sativa and | none | 2 | Da | decoy | 17 | 1 | tr\|A0A0H3W844\|A0A0H3W844_CANSA | 62 |
| | relatives | | | | | | | | |
| 19043 | C. sativa and | none | 2 | Da | decoy | 18 | 1 | tr\|A0A0H3W8G1\|A0A0H3W8G1_CANSA | 33 |
| | relatives | | | | | | | | |
| 19043 | C. sativa and | none | 2 | Da | decoy | 19 | 1 | tr\|A0A0C5APY7\|A0A0C5APY7_CANSA | 32 |
| | relatives | | | | | | | | |
| 19043 | C. sativa and | none | 2 | Da | decoy | 20 | 1 | tr\|A0A0C5APY4\|A0A0C5APY4_CANSA | 28 |
| | relatives | | | | | | | | |
| 19043 | C. sativa and | none | 2 | Da | decoy | 21 | 1 | tr\|A0A0C5AS04\|A0A0C5AS04_CANSA | 18 |
| | relatives | | | | | | | | |
| 19043 | C. sativa and | none | 2 | Da | decoy | 22 | 1 | tr\|A0A172J269\|A0A172J269_BOENI | 17 |
| | relatives | | | | | | | | |
| 19043 | C. sativa and | none | 2 | Da | decoy | 23 | 1 | tr\|A0A172J229\|A0A172J229_BOENI | 15 |
| | relatives | | | | | | | | |
| 19043 | C. sativa and | none | 2 | Da | decoy | 24 | 1 | tr\|A0A1U9VXP2\|A0A1U9VXP2_CANSA | 14 |
| | relatives | | | | | | | | |
| 19042 | all | none | 2 | Da | decoy | 1 | 1 | H42_WHEAT | 21948 |
| 19042 | all | none | 2 | Da | decoy | 2 | 1 | H4_CAPAN | 4176 |
| 19042 | all | none | 2 | Da | decoy | 3 | 1 | UBIQ_AVESA | 2508 |
| 19042 | all | none | 2 | Da | decoy | 4 | 1 | PSAC_AETCO | 2359 |
| 19042 | all | none | 2 | Da | decoy | 5 | 1 | PSBF_EPHSI | 2249 |
| 19042 | all | none | 2 | Da | decoy | 6 | 1 | PSAC_PHAAO | 1938 |
| 19042 | all | none | 2 | Da | decoy | 7 | 1 | ATPH_CYCTA | 1710 |
| 19042 | all | none | 2 | Da | decoy | 8 | 1 | PSBE_AMBTC | 1608 |
| 19042 | all | none | 2 | Da | decoy | 9 | 1 | PSBT_PELHO | 1460 |
| 19042 | all | none | 2 | Da | decoy | 10 | 1 | UBIQ_COPCO | 1421 |
| 19042 | all | none | 2 | Da | decoy | 11 | 1 | PSBT_ALLTE | 1419 |
| 19042 | all | none | 2 | Da | decoy | 12 | 1 | H32_ENCAL | 1364 |
| 19042 | all | none | 2 | Da | decoy | 13 | 1 | PSBT_PIPCE | 1249 |
| 19042 | all | none | 2 | Da | decoy | 14 | 1 | PSBE_CITSI | 979 |
| 19042 | all | none | 2 | Da | decoy | 14 | 2 | PSBE_MESCR | 673 |
| 19042 | all | none | 2 | Da | decoy | 15 | 1 | H33_TRIPS | 862 |
| 19042 | all | none | 2 | Da | decoy | 16 | 1 | PSBE_AGRST | 742 |
| 19042 | all | none | 2 | Da | decoy | 17 | 1 | H3_VOLCA | 740 |
| 19042 | all | none | 2 | Da | decoy | 18 | 1 | PSAC_SPIOL | 695 |
| 19042 | all | none | 2 | Da | decoy | 19 | 1 | RL23_ARATH | 588 |
| 19042 | all | none | 2 | Da | decoy | 20 | 1 | PSBF_AGARO | 546 |
| 19042 | all | none | 2 | Da | decoy | 21 | 1 | RL371_ORYSJ | 415 |
| 19042 | all | none | 2 | Da | decoy | 22 | 1 | H31_CHLRE | 397 |
| 19042 | all | none | 2 | Da | decoy | 23 | 1 | RL37A_GOSHI | 360 |
| 19042 | all | none | 2 | Da | decoy | 24 | 1 | RL391_ARATH | 353 |
| 19042 | all | none | 2 | Da | decoy | 25 | 1 | RR14_NICSY | 348 |
| 19042 | all | none | 2 | Da | decoy | 26 | 1 | OLIAC_CANSA | 299 |
| 19042 | all | none | 2 | Da | decoy | 27 | 1 | PSBI_CRYJA | 234 |
| 19042 | all | none | 2 | Da | decoy | 28 | 1 | RS28_OSTOS | 220 |
| 19042 | all | none | 2 | Da | decoy | 29 | 1 | PSAC_DRIGR | 217 |
| 19042 | all | none | 2 | Da | decoy | 30 | 1 | RR14_SOLBU | 203 |
| 19042 | all | none | 2 | Da | decoy | 31 | 1 | H332_CAEEL | 173 |
| 19042 | all | none | 2 | Da | decoy | 32 | 1 | RL38_SOLLC | 162 |
| 19042 | all | none | 2 | Da | decoy | 33 | 1 | H32_CICIN | 153 |
| 19042 | all | none | 2 | Da | decoy | 34 | 1 | H32_MEDSA | 150 |
| 19042 | all | none | 2 | Da | decoy | 35 | 1 | H3L1_ARATH | 143 |
| 19042 | all | none | 2 | Da | decoy | 36 | 1 | PLAS_MERPE | 123 |
| 19042 | all | none | 2 | Da | decoy | 37 | 1 | RS30_ARATH | 122 |
| 19042 | all | none | 2 | Da | decoy | 38 | 1 | PSBI_LEPVR | 101 |
| 19042 | all | none | 2 | Da | decoy | 39 | 1 | PSAJ_LEMMI | 94 |
| 19042 | all | none | 2 | Da | decoy | 40 | 1 | H2A3_ORYSI | 74 |
| 19042 | all | none | 2 | Da | decoy | 41 | 1 | PETD_ATRBE | 57 |
| 19042 | all | none | 2 | Da | decoy | 42 | 1 | H2B8_ARATH | 57 |
| 19042 | all | none | 2 | Da | decoy | 43 | 1 | GRP1_ARATH | 50 |
| 19042 | all | none | 2 | Da | decoy | 44 | 1 | EX7S_BEUC1 | 47 |
| 19042 | all | none | 2 | Da | decoy | 45 | 1 | TATAO_HALVD | 46 |
| 19042 | all | none | 2 | Da | decoy | 46 | 1 | H3C_CAIMO | 45 |
| 19042 | all | none | 2 | Da | decoy | 47 | 1 | RR16_MORIN | 45 |
| 19042 | all | none | 2 | Da | decoy | 48 | 1 | PLAS_LACSA | 43 |
| 19042 | all | none | 2 | Da | decoy | 49 | 1 | HSL32_DICDI | 41 |
| 19042 | all | none | 2 | Da | decoy | 50 | 1 | H2A2_ORYSI | 40 |
| 19042 | all | none | 2 | Da | decoy | 51 | 1 | RL342_ARATH | 40 |
| 19042 | all | none | 2 | Da | decoy | 52 | 1 | ATPL_LACPL | 40 |
| 19042 | all | none | 2 | Da | decoy | 53 | 1 | ATPL_ILYTA | 39 |
| 19042 | all | none | 2 | Da | decoy | 54 | 1 | CX6B3_ARATH | 37 |
| 19042 | all | none | 2 | Da | decoy | 55 | 1 | CRCB1_CORDI | 37 |
| 19042 | all | none | 2 | Da | decoy | 56 | 1 | ACYP_MANSM | 36 |
| 19042 | all | none | 2 | Da | decoy | 57 | 1 | UBIQ_HELAN | 36 |
| 19042 | all | none | 2 | Da | decoy | 58 | 1 | RL30_LUPLU | 35 |
| 19042 | all | none | 2 | Da | decoy | 59 | 1 | RL13_PSEHT | 34 |
| 19042 | all | none | 2 | Da | decoy | 60 | 1 | GRP2_ORYSI | 33 |
| 19042 | all | none | 2 | Da | decoy | 61 | 1 | Y2513_ANAVT | 33 |
| 19042 | all | none | 2 | Da | decoy | 62 | 1 | MOAC_SALAR | 33 |
| 19042 | all | none | 2 | Da | decoy | 63 | 1 | PSAJ_OSTTA | 33 |
| 19042 | all | none | 2 | Da | decoy | 64 | 1 | HSL39_DICDI | 32 |
| 19042 | all | none | 2 | Da | decoy | 65 | 1 | RBR1_CANAL | 32 |
| 19042 | all | none | 2 | Da | decoy | 66 | 1 | GBG_YARLI | 32 |
| 19042 | all | none | 2 | Da | decoy | 67 | 1 | OLF9_APILI | 32 |
| 19042 | all | none | 2 | Da | decoy | 68 | 1 | UBL1_SCHPO | 31 |
| 19042 | all | none | 2 | Da | decoy | 69 | 1 | CWP2_YEAST | 29 |
| 19042 | all | none | 2 | Da | decoy | 70 | 1 | HEM3_DICCH | 29 |
| 19042 | all | none | 2 | Da | decoy | 71 | 1 | PSBX_GUITH | 29 |
| 19042 | all | none | 2 | Da | decoy | 72 | 1 | COCA_CONCL | 28 |
| 19042 | all | none | 2 | Da | decoy | 73 | 1 | PETG_CUSEX | 28 |
| 19042 | all | none | 2 | Da | decoy | 74 | 1 | R15A1_ARATH | 27 |
| 19042 | all | none | 2 | Da | decoy | 75 | 1 | PSAJ_AMBTC | 27 |
| 19042 | all | none | 2 | Da | decoy | 76 | 1 | H2B10_ARATH | 27 |
| 19042 | all | none | 2 | Da | decoy | 77 | 1 | PSBJ_AGRST | 27 |
| 19042 | all | none | 2 | Da | decoy | 78 | 1 | ANP4_PSEAM | 26 |
| 19042 | all | none | 2 | Da | decoy | 79 | 1 | R35A3_ARATH | 26 |
| 19042 | all | none | 2 | Da | decoy | 80 | 1 | H2B1_ARATH | 26 |
| 19042 | all | none | 2 | Da | decoy | 81 | 1 | RS12_ACTPL | 25 |
| 19042 | all | none | 2 | Da | decoy | 82 | 1 | RL34_LEUCK | 25 |
| 19042 | all | none | 2 | Da | decoy | 83 | 1 | U512A_DICDI | 25 |
| 19042 | all | none | 2 | Da | decoy | 84 | 1 | PPNP_AERHH | 25 |
| 19042 | all | none | 2 | Da | decoy | 85 | 1 | ANFB_TAKRU | 25 |
| 19042 | all | none | 2 | Da | decoy | 86 | 1 | YWZA_BACSU | 24 |
| 19042 | all | none | 2 | Da | decoy | 87 | 1 | RL15_SHEFN | 24 |
| 19042 | all | none | 2 | Da | decoy | 88 | 1 | HIS2_METMJ | 24 |
| 19042 | all | none | 2 | Da | decoy | 89 | 1 | MOAC_SHEB2 | 23 |
| 19042 | all | none | 2 | Da | decoy | 90 | 1 | RL35_EUPES | 22 |
| 19042 | all | none | 2 | Da | decoy | 91 | 1 | NLTP3_VITSX | 22 |
| 19042 | all | none | 2 | Da | decoy | 92 | 1 | SLYX_NITWN | 20 |
| 19042 | all | none | 2 | Da | decoy | 93 | 1 | RL13_AERS4 | 20 |
| 19042 | all | none | 2 | Da | decoy | 94 | 1 | NUOK_FRASN | 20 |
| 19044 | viridiplantae | none | 2 | Da | decoy | 1 | 1 | H42_WHEAT | 24087 |
| 19044 | viridiplantae | none | 2 | Da | decoy | 1 | 2 | H4_CAPAN | 5384 |
| 19044 | viridiplantae | none | 2 | Da | decoy | 2 | 1 | UBIQ_AVESA | 2884 |
| 19044 | viridiplantae | none | 2 | Da | decoy | 3 | 1 | PSAC_AETCO | 2788 |
| 19044 | viridiplantae | none | 2 | Da | decoy | 4 | 1 | PSBF_EPHSI | 2335 |
| 19044 | viridiplantae | none | 2 | Da | decoy | 5 | 1 | PSAC_PHAAO | 2286 |
| 19044 | viridiplantae | none | 2 | Da | decoy | 6 | 1 | H32_ENCAL | 2015 |
| 19044 | viridiplantae | none | 2 | Da | decoy | 7 | 1 | ATPH_CYCTA | 1880 |
| 19044 | viridiplantae | none | 2 | Da | decoy | 8 | 1 | PSBE_AMBTC | 1858 |
| 19044 | viridiplantae | none | 2 | Da | decoy | 8 | 2 | PSBE_MESCR | 903 |
| 19044 | viridiplantae | none | 2 | Da | decoy | 9 | 1 | PSBT_PELHO | 1571 |
| 19044 | viridiplantae | none | 2 | Da | decoy | 10 | 1 | PSBT_ALLTE | 1487 |
| 19044 | viridiplantae | none | 2 | Da | decoy | 11 | 1 | PSBT_PIPCE | 1352 |
| 19044 | viridiplantae | none | 2 | Da | decoy | 12 | 1 | H3_VOLCA | 1314 |
| 19044 | viridiplantae | none | 2 | Da | decoy | 12 | 2 | H31_CHLRE | 875 |
| 19044 | viridiplantae | none | 2 | Da | decoy | 12 | 3 | H32_MEDSA | 517 |
| 19044 | viridiplantae | none | 2 | Da | decoy | 13 | 1 | PSBE_AGRST | 950 |
| 19044 | viridiplantae | none | 2 | Da | decoy | 14 | 1 | PSAC_SPIOL | 932 |
| 19044 | viridiplantae | none | 2 | Da | decoy | 15 | 1 | PSAC_CUSRE | 764 |
| 19044 | viridiplantae | none | 2 | Da | decoy | 16 | 1 | RL23_ARATH | 657 |
| 19044 | viridiplantae | none | 2 | Da | decoy | 17 | 1 | PSBF_AGARO | 636 |
| 19044 | viridiplantae | none | 2 | Da | decoy | 18 | 1 | H33_ARATH | 295 |
| 19044 | viridiplantae | none | 2 | Da | decoy | 19 | 1 | H32_CICIN | 495 |
| 19044 | viridiplantae | none | 2 | Da | decoy | 20 | 1 | RL371_ORYSJ | 480 |
| 19044 | viridiplantae | none | 2 | Da | decoy | 21 | 1 | RL391_ARATH | 430 |
| 19044 | viridiplantae | none | 2 | Da | decoy | 22 | 1 | RL37A_GOSHI | 425 |
| 19044 | viridiplantae | none | 2 | Da | decoy | 23 | 1 | RR14_NICSY | 404 |
| 19044 | viridiplantae | none | 2 | Da | decoy | 24 | 1 | OLIAC_CANSA | 370 |
| 19044 | viridiplantae | none | 2 | Da | decoy | 25 | 1 | PSAC_DRIGR | 348 |
| 19044 | viridiplantae | none | 2 | Da | decoy | 26 | 1 | RL38_SOLLC | 285 |
| 19044 | viridiplantae | none | 2 | Da | decoy | 27 | 1 | PSBI_CYCTA | 251 |
| 19044 | viridiplantae | none | 2 | Da | decoy | 28 | 1 | RR14_SOLBU | 245 |
| 19044 | viridiplantae | none | 2 | Da | decoy | 29 | 1 | ATPH_CRYJA | 229 |
| 19044 | viridiplantae | none | 2 | Da | decoy | 30 | 1 | PLAS_MERPE | 219 |
| 19044 | viridiplantae | none | 2 | Da | decoy | 31 | 1 | RS30_ARATH | 133 |
| 19044 | viridiplantae | none | 2 | Da | decoy | 32 | 1 | PSAJ_LEMMI | 122 |
| 19044 | viridiplantae | none | 2 | Da | decoy | 33 | 1 | PSBI_LEPVR | 113 |
| 19044 | viridiplantae | none | 2 | Da | decoy | 34 | 1 | H2A3_ORYSI | 104 |
| 19044 | viridiplantae | none | 2 | Da | decoy | 35 | 1 | PLAS_LACSA | 89 |
| 19044 | viridiplantae | none | 2 | Da | decoy | 36 | 1 | H2B8_ARATH | 77 |
| 19044 | viridiplantae | none | 2 | Da | decoy | 37 | 1 | GRP2_ORYSI | 71 |
| 19044 | viridiplantae | none | 2 | Da | decoy | 38 | 1 | GRP1_ARATH | 65 |
| 19044 | viridiplantae | none | 2 | Da | decoy | 39 | 1 | RR16_MORIN | 64 |
| 19044 | viridiplantae | none | 2 | Da | decoy | 40 | 1 | H2A2_ORYSI | 58 |
| 19044 | viridiplantae | none | 2 | Da | decoy | 41 | 1 | PETD_ATRBE | 57 |
| 19044 | viridiplantae | none | 2 | Da | decoy | 42 | 1 | RL30_LUPLU | 51 |
| 19044 | viridiplantae | none | 2 | Da | decoy | 43 | 1 | PSAJ_OSTTA | 44 |
| 19044 | viridiplantae | none | 2 | Da | decoy | 44 | 1 | UBIQ_HELAN | 42 |
| 19044 | viridiplantae | none | 2 | Da | decoy | 45 | 1 | RL342_ARATH | 40 |
| 19044 | viridiplantae | none | 2 | Da | decoy | 46 | 1 | R35A3_ARATH | 39 |
| 19044 | viridiplantae | none | 2 | Da | decoy | 47 | 1 | PLAS2_TOBAC | 38 |
| 19044 | viridiplantae | none | 2 | Da | decoy | 48 | 1 | CX6B3_ARATH | 37 |
| 19044 | viridiplantae | none | 2 | Da | decoy | 49 | 1 | BCP1_ARATH | 33 |
| 19044 | viridiplantae | none | 2 | Da | decoy | 50 | 1 | RK33_MORIN | 31 |
| 19044 | viridiplantae | none | 2 | Da | decoy | 51 | 1 | RL35_EUPES | 29 |
| 19044 | viridiplantae | none | 2 | Da | decoy | 52 | 1 | RL271_ARATH | 29 |
| 19044 | viridiplantae | none | 2 | Da | decoy | 53 | 1 | PETG_CUSEX | 28 |
| 19044 | viridiplantae | none | 2 | Da | decoy | 54 | 1 | R15A1_ARATH | 27 |
| 19044 | viridiplantae | none | 2 | Da | decoy | 55 | 1 | PSAJ_AMBTC | 27 |
| 19044 | viridiplantae | none | 2 | Da | decoy | 56 | 1 | H2B10_ARATH | 27 |
| 19044 | viridiplantae | none | 2 | Da | decoy | 57 | 1 | PSBJ_AGRST | 27 |
| 19044 | viridiplantae | none | 2 | Da | decoy | 58 | 1 | PEP7_ARATH | 26 |
| 19044 | viridiplantae | none | 2 | Da | decoy | 59 | 1 | PSAM_ZYGCR | 26 |
| 19044 | viridiplantae | none | 2 | Da | decoy | 60 | 1 | H2B1_ARATH | 26 |
| 19044 | viridiplantae | none | 2 | Da | decoy | 61 | 1 | H2B_GOSHI | 25 |
| 19044 | viridiplantae | none | 2 | Da | decoy | 62 | 1 | PSBJ_AMBTC | 25 |
| 19044 | viridiplantae | none | 2 | Da | decoy | 63 | 1 | PSBL_MARPO | 25 |
| 19044 | viridiplantae | none | 2 | Da | decoy | 64 | 1 | NDUA5_SOLTU | 25 |
| 19044 | viridiplantae | none | 2 | Da | decoy | 65 | 1 | PSBL_ACOCL | 25 |
| 19044 | viridiplantae | none | 2 | Da | decoy | 66 | 1 | PSBE_PANGI | 24 |
| 19044 | viridiplantae | none | 2 | Da | decoy | 67 | 1 | NLTP3_VITSX | 22 |
| 19044 | viridiplantae | none | 2 | Da | decoy | 68 | 1 | DPM2_ARATH | 22 |
| 19044 | viridiplantae | none | 2 | Da | decoy | 69 | 1 | RLF17_ARATH | 22 |
| 19044 | viridiplantae | none | 2 | Da | decoy | 70 | 1 | RS252_ARATH | 21 |
| 19044 | viridiplantae | none | 2 | Da | decoy | 71 | 1 | M1210_ARATH | 20 |
| 19044 | viridiplantae | none | 2 | Da | decoy | 72 | 1 | DPM3_ARATH | 20 |
| 19044 | viridiplantae | none | 2 | Da | decoy | 73 | 1 | ACBP1_ORYSJ | 19 |
| 19044 | viridiplantae | none | 2 | Da | decoy | 74 | 1 | PSBH_LACSA | 19 |
| 19044 | viridiplantae | none | 2 | Da | decoy | 75 | 1 | GASA7_ARATH | 18 |
| 19044 | viridiplantae | none | 2 | Da | decoy | 76 | 1 | M7_LILHE | 18 |
| 19044 | viridiplantae | none | 2 | Da | decoy | 77 | 1 | PSBK_VITVI | 17 |
| 19044 | viridiplantae | none | 2 | Da | decoy | 78 | 1 | ATP9_ARATH | 16 |
| 19044 | viridiplantae | none | 2 | Da | decoy | 79 | 1 | EA1_MAIZE | 16 |
| 19044 | viridiplantae | none | 2 | Da | decoy | 80 | 1 | H2A2_PEA | 16 |
| 19045 | viridiplantae | AO | 2 | Da | decoy | 1 | 1 | H4_ARATH | 31819 |
| 19045 | viridiplantae | AO | 2 | Da | decoy | 2 | 1 | H4_CHLRE | 12691 |
| 19045 | viridiplantae | AO | 2 | Da | decoy | 3 | 1 | PSBF_AGARO | 3132 |
| 19045 | viridiplantae | AO | 2 | Da | decoy | 4 | 1 | PSBF_PINKO | 2822 |
| 19045 | viridiplantae | AO | 2 | Da | decoy | 5 | 1 | UBIQ_AVESA | 2738 |
| 19045 | viridiplantae | AO | 2 | Da | decoy | 6 | 1 | PSBF_MARPO | 2603 |
| 19045 | viridiplantae | AO | 2 | Da | decoy | 7 | 1 | PSAC_AETCO | 2538 |
| 19045 | viridiplantae | AO | 2 | Da | decoy | 8 | 1 | H32_ENCAL | 2507 |
| 19045 | viridiplantae | AO | 2 | Da | decoy | 9 | 1 | PSAC_SPIOL | 2084 |
| 19045 | viridiplantae | AO | 2 | Da | decoy | 10 | 1 | H3_VOLCA | 1969 |
| 19045 | viridiplantae | AO | 2 | Da | decoy | 11 | 1 | ATPH_ARAHI | 1906 |
| 19045 | viridiplantae | AO | 2 | Da | decoy | 12 | 1 | ATPH_CYCTA | 1760 |
| 19045 | viridiplantae | AO | 2 | Da | decoy | 13 | 1 | PSBE_AMBTC | 1694 |
| 19045 | viridiplantae | AO | 2 | Da | decoy | 14 | 1 | ATPH_CERDE | 1670 |
| 19045 | viridiplantae | AO | 2 | Da | decoy | 15 | 1 | PSBT_ALLTE | 1651 |
| 19045 | viridiplantae | AO | 2 | Da | decoy | 16 | 1 | PSBT_PELHO | 1434 |
| 19045 | viridiplantae | AO | 2 | Da | decoy | 17 | 1 | PSAC_DRIGR | 1381 |
| 19045 | viridiplantae | AO | 2 | Da | decoy | 18 | 1 | PSBT_PIPCE | 1263 |
| 19045 | viridiplantae | AO | 2 | Da | decoy | 19 | 1 | H31_CHLRE | 1184 |
| 19045 | viridiplantae | AO | 2 | Da | decoy | 20 | 1 | RL391_ARATH | 1124 |
| 19045 | viridiplantae | AO | 2 | Da | decoy | 21 | 1 | H32_ARATH | 880 |
| 19045 | viridiplantae | AO | 2 | Da | decoy | 22 | 1 | PSBE_AGRST | 756 |
| 19045 | viridiplantae | AO | 2 | Da | decoy | 23 | 1 | RL23_ARATH | 736 |
| 19045 | viridiplantae | AO | 2 | Da | decoy | 24 | 1 | H32_MEDSA | 697 |
| 19045 | viridiplantae | AO | 2 | Da | decoy | 25 | 1 | ATPH_AGRST | 688 |
| 19045 | viridiplantae | AO | 2 | Da | decoy | 26 | 1 | PSBE_MESCR | 612 |
| 19045 | viridiplantae | AO | 2 | Da | decoy | 27 | 1 | RL371_ORYSJ | 473 |
| 19045 | viridiplantae | AO | 2 | Da | decoy | 28 | 1 | RL37A_GOSHI | 390 |
| 19045 | viridiplantae | AO | 2 | Da | decoy | 29 | 1 | PLAS_MERPE | 387 |
| 19045 | viridiplantae | AO | 2 | Da | decoy | 30 | 1 | RR14_NICSY | 366 |
| 19045 | viridiplantae | AO | 2 | Da | decoy | 31 | 1 | OLIAC_CANSA | 334 |
| 19045 | viridiplantae | AO | 2 | Da | decoy | 32 | 1 | RS28_MAIZE | 332 |
| 19045 | viridiplantae | AO | 2 | Da | decoy | 33 | 1 | H3L1_ARATH | 321 |
| 19045 | viridiplantae | AO | 2 | Da | decoy | 34 | 1 | PSBI_CRYJA | 248 |
| 19045 | viridiplantae | AO | 2 | Da | decoy | 35 | 1 | PSBI_CYCTA | 245 |
| 19045 | viridiplantae | AO | 2 | Da | decoy | 36 | 1 | RR14_SOLBU | 221 |
| 19045 | viridiplantae | AO | 2 | Da | decoy | 37 | 1 | RL38_SOLLC | 216 |
| 19045 | viridiplantae | AO | 2 | Da | decoy | 38 | 1 | PSBI_PINKO | 195 |
| 19045 | viridiplantae | AO | 2 | Da | decoy | 39 | 1 | H33_ARATH | 182 |
| 19045 | viridiplantae | AO | 2 | Da | decoy | 40 | 1 | RS30_ARATH | 124 |
| 19045 | viridiplantae | AO | 2 | Da | decoy | 41 | 1 | RL30_EUPES | 116 |
| 19045 | viridiplantae | AO | 2 | Da | decoy | 42 | 1 | ATPH_PEA | 113 |
| 19045 | viridiplantae | AO | 2 | Da | decoy | 43 | 1 | H32_LILLO | 109 |
| 19045 | viridiplantae | AO | 2 | Da | decoy | 44 | 1 | PSBJ_AETCO | 99 |
| 19045 | viridiplantae | AO | 2 | Da | decoy | 45 | 1 | PSAJ_LEMMI | 98 |
| 19045 | viridiplantae | AO | 2 | Da | decoy | 46 | 1 | H2A3_ORYSI | 93 |
| 19045 | viridiplantae | AO | 2 | Da | decoy | 47 | 1 | PSBJ_ARATH | 91 |
| 19045 | viridiplantae | AO | 2 | Da | decoy | 48 | 1 | RL373_ARATH | 87 |
| 19045 | viridiplantae | AO | 2 | Da | decoy | 49 | 1 | H32_CICIN | 77 |
| 19045 | viridiplantae | AO | 2 | Da | decoy | 50 | 1 | GRP1_ARATH | 74 |
| 19045 | viridiplantae | AO | 2 | Da | decoy | 51 | 1 | PSK2_ARATH | 73 |
| 19045 | viridiplantae | AO | 2 | Da | decoy | 52 | 1 | RR16_MORIN | 68 |
| 19045 | viridiplantae | AO | 2 | Da | decoy | 53 | 1 | RS242_ARATH | 67 |
| 19045 | viridiplantae | AO | 2 | Da | decoy | 54 | 1 | H2B8_ARATH | 66 |
| 19045 | viridiplantae | AO | 2 | Da | decoy | 55 | 1 | PSAC_PINTH | 66 |
| 19045 | viridiplantae | AO | 2 | Da | decoy | 56 | 1 | PSAJ_CHLAT | 59 |
| 19045 | viridiplantae | AO | 2 | Da | decoy | 57 | 1 | GRP2_ORYSI | 58 |
| 19045 | viridiplantae | AO | 2 | Da | decoy | 58 | 1 | PSBH_COFAR | 58 |
| 19045 | viridiplantae | AO | 2 | Da | decoy | 59 | 1 | PETD_ATRBE | 57 |
| 19045 | viridiplantae | AO | 2 | Da | decoy | 60 | 1 | PLAS_CAPBU | 55 |
| 19045 | viridiplantae | AO | 2 | Da | decoy | 61 | 1 | RL30_LUPLU | 54 |
| 19045 | viridiplantae | AO | 2 | Da | decoy | 62 | 1 | EA1_MAIZE | 54 |
| 19045 | viridiplantae | AO | 2 | Da | decoy | 63 | 1 | KRP6_ORYSJ | 54 |
| 19045 | viridiplantae | AO | 2 | Da | decoy | 64 | 1 | H2A2_ORYSI | 52 |
| 19045 | viridiplantae | AO | 2 | Da | decoy | 65 | 1 | RTS_ORYSJ | 48 |
| 19045 | viridiplantae | AO | 2 | Da | decoy | 66 | 1 | ATP9_OENBI | 48 |
| 19045 | viridiplantae | AO | 2 | Da | decoy | 67 | 1 | H3L3_ARATH | 47 |
| 19045 | viridiplantae | AO | 2 | Da | decoy | 68 | 1 | EMP1_ORYSJ | 45 |
| 19045 | viridiplantae | AO | 2 | Da | decoy | 69 | 1 | PSBH_NYMAL | 45 |
| 19045 | viridiplantae | AO | 2 | Da | decoy | 70 | 1 | RS142_MAIZE | 44 |
| 19045 | viridiplantae | AO | 2 | Da | decoy | 71 | 1 | RLF36_ARATH | 44 |
| 19045 | viridiplantae | AO | 2 | Da | decoy | 72 | 1 | PSAI_HORVU | 44 |
| 19045 | viridiplantae | AO | 2 | Da | decoy | 73 | 1 | PSBI_ANTAG | 42 |
| 19045 | viridiplantae | AO | 2 | Da | decoy | 74 | 1 | ATP9_MARPO | 41 |
| 19045 | viridiplantae | AO | 2 | Da | decoy | 75 | 1 | ACBP1_ORYSJ | 41 |
| 19045 | viridiplantae | AO | 2 | Da | decoy | 76 | 1 | RR8_MESVI | 41 |
| 19045 | viridiplantae | AO | 2 | Da | decoy | 77 | 1 | PROFW_OLEEU | 40 |
| 19045 | viridiplantae | AO | 2 | Da | decoy | 78 | 1 | RL342_ARATH | 40 |
| 19045 | viridiplantae | AO | 2 | Da | decoy | 79 | 1 | GRC14_ORYSJ | 39 |
| 19045 | viridiplantae | AO | 2 | Da | decoy | 80 | 1 | PROF4_ARATH | 39 |
| 19045 | viridiplantae | AO | 2 | Da | decoy | 81 | 1 | GRXS3_ORYSJ | 38 |
| 19045 | viridiplantae | AO | 2 | Da | decoy | 82 | 1 | ACBP_BRANA | 38 |
| 19045 | viridiplantae | AO | 2 | Da | decoy | 83 | 1 | TIM13_ARATH | 38 |
| 19045 | viridiplantae | AO | 2 | Da | decoy | 84 | 1 | RLF28_ARATH | 38 |
| 19045 | viridiplantae | AO | 2 | Da | decoy | 85 | 1 | PSBH_HORVU | 38 |
| 19045 | viridiplantae | AO | 2 | Da | decoy | 86 | 1 | PETG_PLAOC | 38 |
| 19045 | viridiplantae | AO | 2 | Da | decoy | 87 | 1 | PST2_PETHY | 38 |
| 19045 | viridiplantae | AO | 2 | Da | decoy | 88 | 1 | H2B10_ARATH | 38 |
| 19045 | viridiplantae | AO | 2 | Da | decoy | 89 | 1 | H2B1_ARATH | 37 |
| 19045 | viridiplantae | AO | 2 | Da | decoy | 90 | 1 | ATP9_PEA | 37 |
| 19045 | viridiplantae | AO | 2 | Da | decoy | 91 | 1 | CX6B3_ARATH | 37 |
| 19045 | viridiplantae | AO | 2 | Da | decoy | 92 | 1 | PST2_ARATH | 37 |
| 19045 | viridiplantae | AO | 2 | Da | decoy | 93 | 1 | PFD5_ARATH | 37 |
| 19045 | viridiplantae | AO | 2 | Da | decoy | 94 | 1 | RR11_PHAVU | 37 |
| 19045 | viridiplantae | AO | 2 | Da | decoy | 95 | 1 | H2B9_ARATH | 36 |
| 19045 | viridiplantae | AO | 2 | Da | decoy | 96 | 1 | RK16_OENAM | 36 |
| 19045 | viridiplantae | AO | 2 | Da | decoy | 97 | 1 | COPT3_ARATH | 36 |
| 19045 | viridiplantae | AO | 2 | Da | decoy | 98 | 1 | PLAS_PHYPA | 35 |
| 19045 | viridiplantae | AO | 2 | Da | decoy | 99 | 1 | PSBK_CHLVU | 35 |
| 19045 | viridiplantae | AO | 2 | Da | decoy | 100 | 1 | NLTP3_HORVU | 35 |
| 19045 | viridiplantae | AO | 2 | Da | decoy | 101 | 1 | PSBH_PHAAO | 34 |
| 19045 | viridiplantae | AO | 2 | Da | decoy | 102 | 1 | AGP12_ARATH | 34 |
| 19045 | viridiplantae | AO | 2 | Da | decoy | 103 | 1 | PSAI_MARPO | 34 |
| 19045 | viridiplantae | AO | 2 | Da | decoy | 104 | 1 | GRC10_ORYSJ | 34 |
| 19045 | viridiplantae | AO | 2 | Da | decoy | 105 | 1 | EM3_WHEAT | 34 |
| 19045 | viridiplantae | AO | 2 | Da | decoy | 106 | 1 | ACBP_RICCO | 34 |
| 19045 | viridiplantae | AO | 2 | Da | decoy | 107 | 1 | LGB2_MEDTR | 33 |
| 19045 | viridiplantae | AO | 2 | Da | decoy | 108 | 1 | DEF97_ARATH | 33 |
| 19045 | viridiplantae | AO | 2 | Da | decoy | 109 | 1 | PSAI_WELMI | 32 |
| 19045 | viridiplantae | AO | 2 | Da | decoy | 110 | 1 | TOM91_ARATH | 32 |
| 19045 | viridiplantae | AO | 2 | Da | decoy | 111 | 1 | RK33_MORIN | 32 |
| 19045 | viridiplantae | AO | 2 | Da | decoy | 112 | 1 | R35A3_ARATH | 31 |
| 19045 | viridiplantae | AO | 2 | Da | decoy | 113 | 1 | POLC3_CHEAL | 31 |
| 19045 | viridiplantae | AO | 2 | Da | decoy | 114 | 1 | RR19_OEDCA | 31 |
| 19045 | viridiplantae | AO | 2 | Da | decoy | 115 | 1 | POLC4_BETPN | 31 |
| 19045 | viridiplantae | AO | 2 | Da | decoy | 116 | 1 | CML4_ORYSJ | 30 |
| 19045 | viridiplantae | AO | 2 | Da | decoy | 117 | 1 | ICI2_HORVU | 30 |
| 19045 | viridiplantae | AO | 2 | Da | decoy | 118 | 1 | MT2_MUSAC | 29 |
| 19045 | viridiplantae | AO | 2 | Da | decoy | 119 | 1 | APEP2_ORYSJ | 29 |
| 19045 | viridiplantae | AO | 2 | Da | decoy | 120 | 1 | UBIQ_HELAN | 29 |
| 19045 | viridiplantae | AO | 2 | Da | decoy | 121 | 1 | CH60_SOLTU | 29 |
| 19045 | viridiplantae | AO | 2 | Da | decoy | 122 | 1 | PSBH_PIPCE | 29 |
| 19045 | viridiplantae | AO | 2 | Da | decoy | 123 | 1 | PSBH_MAIZE | 29 |
| 19045 | viridiplantae | AO | 2 | Da | decoy | 124 | 1 | GRS13_ARATH | 29 |
| 19045 | viridiplantae | AO | 2 | Da | decoy | 125 | 1 | ATP9_PETHY | 29 |
| 19045 | viridiplantae | AO | 2 | Da | decoy | 126 | 1 | CYCK_PETHY | 28 |
| 19045 | viridiplantae | AO | 2 | Da | decoy | 127 | 1 | PSBK_STIHE | 28 |
| 19045 | viridiplantae | AO | 2 | Da | decoy | 128 | 1 | PSAJ_AMBTC | 27 |
| 19045 | viridiplantae | AO | 2 | Da | decoy | 129 | 1 | RK16_GOSHI | 27 |
| 19045 | viridiplantae | AO | 2 | Da | decoy | 130 | 1 | RS192_ARATH | 27 |
| 19045 | viridiplantae | AO | 2 | Da | decoy | 131 | 1 | ICIA_HORVU | 27 |
| 19045 | viridiplantae | AO | 2 | Da | decoy | 132 | 1 | PS5_PINST | 25 |
| 19045 | viridiplantae | AO | 2 | Da | decoy | 133 | 1 | DEF84_ARATH | 25 |
| 19045 | viridiplantae | AO | 2 | Da | decoy | 134 | 1 | RK14_VIGUN | 23 |
| 19045 | viridiplantae | AO | 2 | Da | decoy | 135 | 1 | GRP3_POPEU | 22 |
| 19045 | viridiplantae | AO | 2 | Da | decoy | 136 | 1 | SMAP1_ARATH | 22 |
| 19045 | viridiplantae | AO | 2 | Da | decoy | 137 | 1 | DPM2_ARATH | 22 |
| 19045 | viridiplantae | AO | 2 | Da | decoy | 138 | 1 | PSBJ_WHEAT | 21 |
| 19045 | viridiplantae | AO | 2 | Da | decoy | 139 | 1 | LSM5_ARATH | 21 |
| 19045 | viridiplantae | AO | 2 | Da | decoy | 140 | 1 | AGP15_ARATH | 20 |
| 19045 | viridiplantae | AO | 2 | Da | decoy | 141 | 1 | ALFC_PINST | 20 |
| 19046 | viridiplantae | AOP | 2 | Da | decoy | 1 | 1 | H4_ARATH | 28165 |
| 19046 | viridiplantae | AOP | 2 | Da | decoy | 2 | 1 | H42_WHEAT | 21440 |
| 19046 | viridiplantae | AOP | 2 | Da | decoy | 3 | 1 | H4_CAPAN | 8894 |
| 19046 | viridiplantae | AOP | 2 | Da | decoy | 4 | 1 | H4_CHLRE | 6116 |
| 19046 | viridiplantae | AOP | 2 | Da | decoy | 5 | 1 | UBIQ_AVESA | 2941 |
| 19046 | viridiplantae | AOP | 2 | Da | decoy | 6 | 1 | PSBF_AGARO | 2936 |
| 19046 | viridiplantae | AOP | 2 | Da | decoy | 7 | 1 | PSBF_PINKO | 2628 |
| 19046 | viridiplantae | AOP | 2 | Da | decoy | 8 | 1 | PSBF_MARPO | 2434 |
| 19046 | viridiplantae | AOP | 2 | Da | decoy | 9 | 1 | PSAC_HELAN | 2191 |
| 19046 | viridiplantae | AOP | 2 | Da | decoy | 10 | 1 | H32_ENCAL | 1905 |
| 19046 | viridiplantae | AOP | 2 | Da | decoy | 11 | 1 | ATPH_ARAHI | 1777 |
| 19046 | viridiplantae | AOP | 2 | Da | decoy | 12 | 1 | ATPH_CYCTA | 1633 |
| 19046 | viridiplantae | AOP | 2 | Da | decoy | 13 | 1 | PSAC_SPIOL | 1620 |
| 19046 | viridiplantae | AOP | 2 | Da | decoy | 14 | 1 | PSBT_ALLTE | 1557 |
| 19046 | viridiplantae | AOP | 2 | Da | decoy | 15 | 1 | ATPH_ACOAM | 1550 |
| 19046 | viridiplantae | AOP | 2 | Da | decoy | 16 | 1 | ATPH_CERDE | 1530 |
| 19046 | viridiplantae | AOP | 2 | Da | decoy | 17 | 1 | PSBE_AMBTC | 1512 |
| 19046 | viridiplantae | AOP | 2 | Da | decoy | 18 | 1 | PSBT_PIPCE | 1352 |
| 19046 | viridiplantae | AOP | 2 | Da | decoy | 19 | 1 | H3_VOLCA | 1342 |
| 19046 | viridiplantae | AOP | 2 | Da | decoy | 20 | 1 | ATPH_IPOPU | 1157 |
| 19046 | viridiplantae | AOP | 2 | Da | decoy | 21 | 1 | PSBT_PELHO | 1141 |
| 19046 | viridiplantae | AOP | 2 | Da | decoy | 22 | 1 | RL391_ARATH | 1025 |
| 19046 | viridiplantae | AOP | 2 | Da | decoy | 23 | 1 | PSBE_CITSI | 797 |
| 19046 | viridiplantae | AOP | 2 | Da | decoy | 24 | 1 | RS28_MAIZE | 705 |
| 19046 | viridiplantae | AOP | 2 | Da | decoy | 25 | 1 | UBIQ_WHEAT | 602 |
| 19046 | viridiplantae | AOP | 2 | Da | decoy | 26 | 1 | UBIQ_HELAN | 582 |
| 19046 | viridiplantae | AOP | 2 | Da | decoy | 27 | 1 | H32_MEDSA | 513 |
| 19046 | viridiplantae | AOP | 2 | Da | decoy | 28 | 1 | PSBI_ACOAM | 497 |
| 19046 | viridiplantae | AOP | 2 | Da | decoy | 29 | 1 | RL23_ARATH | 466 |
| 19046 | viridiplantae | AOP | 2 | Da | decoy | 30 | 1 | RL371_ORYSJ | 461 |
| 19046 | viridiplantae | AOP | 2 | Da | decoy | 31 | 1 | PSAC_DRIGR | 428 |
| 19046 | viridiplantae | AOP | 2 | Da | decoy | 32 | 1 | GRP2_ORYSI | 424 |
| 19046 | viridiplantae | AOP | 2 | Da | decoy | 33 | 1 | RS281_ARATH | 404 |
| 19046 | viridiplantae | AOP | 2 | Da | decoy | 34 | 1 | ATPH_AGRST | 385 |
| 19046 | viridiplantae | AOP | 2 | Da | decoy | 35 | 1 | RR14_SOLBU | 380 |
| 19046 | viridiplantae | AOP | 2 | Da | decoy | 36 | 1 | RTS_ORYSI | 345 |
| 19046 | viridiplantae | AOP | 2 | Da | decoy | 37 | 1 | H32_ARATH | 272 |
| 19046 | viridiplantae | AOP | 2 | Da | decoy | 38 | 1 | PSAC_ACOCL | 269 |
| 19046 | viridiplantae | AOP | 2 | Da | decoy | 39 | 1 | PLAS_SOLTU | 254 |
| 19046 | viridiplantae | AOP | 2 | Da | decoy | 40 | 1 | RTS_ORYSJ | 250 |
| 19046 | viridiplantae | AOP | 2 | Da | decoy | 41 | 1 | OLIAC_CANSA | 250 |
| 19046 | viridiplantae | AOP | 2 | Da | decoy | 42 | 1 | ATPH_ATRBE | 241 |
| 19046 | viridiplantae | AOP | 2 | Da | decoy | 43 | 1 | RL30_LUPLU | 233 |
| 19046 | viridiplantae | AOP | 2 | Da | decoy | 44 | 1 | PSAI_ZYGCR | 230 |
| 19046 | viridiplantae | AOP | 2 | Da | decoy | 45 | 1 | LE25_SOLLC | 230 |
| 19046 | viridiplantae | AOP | 2 | Da | decoy | 46 | 1 | PSAI_LOTJA | 216 |
| 19046 | viridiplantae | AOP | 2 | Da | decoy | 47 | 1 | TGD5_ARATH | 210 |
| 19046 | viridiplantae | AOP | 2 | Da | decoy | 48 | 1 | RL37A_GOSHI | 194 |
| 19046 | viridiplantae | AOP | 2 | Da | decoy | 49 | 1 | H3L1_ARATH | 190 |
| 19046 | viridiplantae | AOP | 2 | Da | decoy | 50 | 1 | PSBE_MESCR | 189 |
| 19046 | viridiplantae | AOP | 2 | Da | decoy | 51 | 1 | PLAS_MERPE | 186 |
| 19046 | viridiplantae | AOP | 2 | Da | decoy | 52 | 1 | PSBE_OSTTA | 159 |
| 19046 | viridiplantae | AOP | 2 | Da | decoy | 53 | 1 | RL38_SOLLC | 140 |
| 19046 | viridiplantae | AOP | 2 | Da | decoy | 54 | 1 | SC61B_CHLRE | 138 |
| 19046 | viridiplantae | AOP | 2 | Da | decoy | 55 | 1 | EA1_MAIZE | 128 |
| 19046 | viridiplantae | AOP | 2 | Da | decoy | 56 | 1 | DEF97_ARATH | 124 |
| 19046 | viridiplantae | AOP | 2 | Da | decoy | 57 | 1 | RS30_ARATH | 115 |
| 19046 | viridiplantae | AOP | 2 | Da | decoy | 58 | 1 | SC61B_ARATH | 114 |
| 19046 | viridiplantae | AOP | 2 | Da | decoy | 59 | 1 | IF5A_SENVE | 109 |
| 19046 | viridiplantae | AOP | 2 | Da | decoy | 60 | 1 | ATP9_BETVU | 105 |
| 19046 | viridiplantae | AOP | 2 | Da | decoy | 61 | 1 | ALFC_PINST | 103 |
| 19046 | viridiplantae | AOP | 2 | Da | decoy | 62 | 1 | H2A3_ORYSI | 102 |
| 19046 | viridiplantae | AOP | 2 | Da | decoy | 63 | 1 | PSBI_LEPVR | 98 |
| 19046 | viridiplantae | AOP | 2 | Da | decoy | 64 | 1 | PSAK_CHLRE | 98 |
| 19046 | viridiplantae | AOP | 2 | Da | decoy | 65 | 1 | H2B11_ORYSI | 96 |
| 19046 | viridiplantae | AOP | 2 | Da | decoy | 66 | 1 | ACBP_RICCO | 95 |
| 19046 | viridiplantae | AOP | 2 | Da | decoy | 67 | 1 | PSBJ_AETCO | 93 |
| 19046 | viridiplantae | AOP | 2 | Da | decoy | 68 | 1 | SP1L2_ARATH | 93 |
| 19046 | viridiplantae | AOP | 2 | Da | decoy | 69 | 1 | ACBP2_ORYSJ | 91 |
| 19046 | viridiplantae | AOP | 2 | Da | decoy | 70 | 1 | AMP_AMARE | 89 |
| 19046 | viridiplantae | AOP | 2 | Da | decoy | 71 | 1 | PSBJ_GNEPA | 88 |
| 19046 | viridiplantae | AOP | 2 | Da | decoy | 72 | 1 | MT2C_ORYSI | 87 |
| 19046 | viridiplantae | AOP | 2 | Da | decoy | 73 | 1 | H32_LILLO | 86 |
| 19046 | viridiplantae | AOP | 2 | Da | decoy | 74 | 1 | MFS18_MAIZE | 86 |
| 19046 | viridiplantae | AOP | 2 | Da | decoy | 75 | 1 | H2A2_ORYSI | 85 |
| 19046 | viridiplantae | AOP | 2 | Da | decoy | 76 | 1 | PSBJ_ARATH | 85 |
| 19046 | viridiplantae | AOP | 2 | Da | decoy | 77 | 1 | ATPH_CHLAT | 84 |
| 19046 | viridiplantae | AOP | 2 | Da | decoy | 78 | 1 | HSBP_ARATH | 84 |
| 19046 | viridiplantae | AOP | 2 | Da | decoy | 79 | 1 | MT4A_ARATH | 83 |
| 19046 | viridiplantae | AOP | 2 | Da | decoy | 80 | 1 | ATP5E_IPOBA | 81 |
| 19046 | viridiplantae | AOP | 2 | Da | decoy | 81 | 1 | GRP1_ORYSJ | 79 |
| 19046 | viridiplantae | AOP | 2 | Da | decoy | 82 | 1 | PLAS_CAPBU | 79 |
| 19046 | viridiplantae | AOP | 2 | Da | decoy | 83 | 1 | SAU19_ARATH | 74 |
| 19046 | viridiplantae | AOP | 2 | Da | decoy | 84 | 1 | DLDH_SOLTU | 74 |
| 19046 | viridiplantae | AOP | 2 | Da | decoy | 85 | 1 | PSBI_JASNU | 73 |
| 19046 | viridiplantae | AOP | 2 | Da | decoy | 86 | 1 | PSK2_ARATH | 73 |
| 19046 | viridiplantae | AOP | 2 | Da | decoy | 87 | 1 | H2B9_ARATH | 73 |
| 19046 | viridiplantae | AOP | 2 | Da | decoy | 88 | 1 | RS242_ARATH | 73 |
| 19046 | viridiplantae | AOP | 2 | Da | decoy | 89 | 1 | RL272_ARATH | 72 |
| 19046 | viridiplantae | AOP | 2 | Da | decoy | 90 | 1 | PSAJ_LEMMI | 71 |
| 19046 | viridiplantae | AOP | 2 | Da | decoy | 91 | 1 | RUXG_MEDSA | 71 |
| 19046 | viridiplantae | AOP | 2 | Da | decoy | 92 | 1 | PSAI_MORIN | 71 |
| 19046 | viridiplantae | AOP | 2 | Da | decoy | 93 | 1 | GRP1_ORYSI | 70 |
| 19046 | viridiplantae | AOP | 2 | Da | decoy | 94 | 1 | PROCK_OLEEU | 70 |
| 19046 | viridiplantae | AOP | 2 | Da | decoy | 95 | 1 | PSAI_CALFG | 70 |
| 19046 | viridiplantae | AOP | 2 | Da | decoy | 96 | 1 | DIRL1_ARATH | 70 |
| 19046 | viridiplantae | AOP | 2 | Da | decoy | 97 | 1 | PSAI_ACOGR | 69 |
| 19046 | viridiplantae | AOP | 2 | Da | decoy | 98 | 1 | FER_SOLLY | 69 |
| 19046 | viridiplantae | AOP | 2 | Da | decoy | 99 | 1 | GRXS1_ARATH | 68 |
| 19046 | viridiplantae | AOP | 2 | Da | decoy | 100 | 1 | MT2A_ARATH | 67 |
| 19046 | viridiplantae | AOP | 2 | Da | decoy | 101 | 1 | PSK5_ORYSJ | 67 |
| 19046 | viridiplantae | AOP | 2 | Da | decoy | 102 | 1 | PSAI_PHAAO | 67 |
| 19046 | viridiplantae | AOP | 2 | Da | decoy | 103 | 1 | NLTPA_RICCO | 66 |
| 19046 | viridiplantae | AOP | 2 | Da | decoy | 104 | 1 | PETD_GOSBA | 66 |
| 19046 | viridiplantae | AOP | 2 | Da | decoy | 105 | 1 | GLRX_VERFO | 65 |
| 19046 | viridiplantae | AOP | 2 | Da | decoy | 106 | 1 | ATPH_STIHE | 65 |
| 19046 | viridiplantae | AOP | 2 | Da | decoy | 107 | 1 | RS241_ARATH | 65 |
| 19046 | viridiplantae | AOP | 2 | Da | decoy | 108 | 1 | PSAI_HORVU | 64 |
| 19046 | viridiplantae | AOP | 2 | Da | decoy | 109 | 1 | DEF85_ARATH | 64 |
| 19046 | viridiplantae | AOP | 2 | Da | decoy | 110 | 1 | RL30_EUPES | 63 |
| 19046 | viridiplantae | AOP | 2 | Da | decoy | 111 | 1 | ATPH_ANEMR | 63 |
| 19046 | viridiplantae | AOP | 2 | Da | decoy | 112 | 1 | WIR1A_WHEAT | 62 |
| 19046 | viridiplantae | AOP | 2 | Da | decoy | 113 | 1 | BCP1_BRACM | 62 |
| 19046 | viridiplantae | AOP | 2 | Da | decoy | 114 | 1 | LEA2_ARATH | 61 |
| 19046 | viridiplantae | AOP | 2 | Da | decoy | 115 | 1 | AGP1_ARATH | 61 |
| 19046 | viridiplantae | AOP | 2 | Da | decoy | 116 | 1 | GRP5_ARATH | 61 |
| 19046 | viridiplantae | AOP | 2 | Da | decoy | 117 | 1 | RR16_MORIN | 60 |
| 19046 | viridiplantae | AOP | 2 | Da | decoy | 118 | 1 | ATP9_PEA | 60 |
| 19046 | viridiplantae | AOP | 2 | Da | decoy | 119 | 1 | ATP9_HELAN | 60 |
| 19046 | viridiplantae | AOP | 2 | Da | decoy | 120 | 1 | NU4LC_CHLAT | 59 |
| 19046 | viridiplantae | AOP | 2 | Da | decoy | 121 | 1 | MT2B_SOLLC | 59 |
| 19046 | viridiplantae | AOP | 2 | Da | decoy | 122 | 1 | AGP4_ARATH | 59 |
| 19046 | viridiplantae | AOP | 2 | Da | decoy | 123 | 1 | PSBH_STIHE | 59 |
| 19046 | viridiplantae | AOP | 2 | Da | decoy | 124 | 1 | GRS10_ARATH | 59 |
| 19046 | viridiplantae | AOP | 2 | Da | decoy | 125 | 1 | RL271_ARATH | 59 |
| 19046 | viridiplantae | AOP | 2 | Da | decoy | 126 | 1 | PSAJ_ACOCL | 59 |
| 19046 | viridiplantae | AOP | 2 | Da | decoy | 127 | 1 | RLA2A_MAIZE | 58 |
| 19046 | viridiplantae | AOP | 2 | Da | decoy | 128 | 1 | NO93_SOYBN | 57 |
| 19046 | viridiplantae | AOP | 2 | Da | decoy | 129 | 1 | H2B8_ARATH | 57 |
| 19046 | viridiplantae | AOP | 2 | Da | decoy | 130 | 1 | IF5A2_MEDSA | 57 |
| 19046 | viridiplantae | AOP | 2 | Da | decoy | 131 | 1 | PLAS_LACSA | 57 |
| 19046 | viridiplantae | AOP | 2 | Da | decoy | 132 | 1 | AGP15_ARATH | 56 |
| 19046 | viridiplantae | AOP | 2 | Da | decoy | 133 | 1 | PCEP6_ARATH | 56 |
| 19046 | viridiplantae | AOP | 2 | Da | decoy | 134 | 1 | PSAC_PINTH | 55 |
| 19046 | viridiplantae | AOP | 2 | Da | decoy | 135 | 1 | NDUA2_ARATH | 55 |
| 19046 | viridiplantae | AOP | 2 | Da | decoy | 136 | 1 | PROFE_OLEEU | 55 |
| 19046 | viridiplantae | AOP | 2 | Da | decoy | 137 | 1 | PSAJ_CHLSC | 55 |
| 19046 | viridiplantae | AOP | 2 | Da | decoy | 138 | 1 | PSBH_ARATH | 55 |
| 19046 | viridiplantae | AOP | 2 | Da | decoy | 139 | 1 | LIRP1_ORYSJ | 55 |
| 19046 | viridiplantae | AOP | 2 | Da | decoy | 140 | 1 | MOC2A_MAIZE | 55 |
| 19046 | viridiplantae | AOP | 2 | Da | decoy | 141 | 1 | CB21_PEA | 55 |
| 19046 | viridiplantae | AOP | 2 | Da | decoy | 142 | 1 | H2B7_ARATH | 54 |
| 19046 | viridiplantae | AOP | 2 | Da | decoy | 143 | 1 | PSBH_TETOB | 54 |
| 19046 | viridiplantae | AOP | 2 | Da | decoy | 144 | 1 | ILI3_ORYSI | 54 |
| 19046 | viridiplantae | AOP | 2 | Da | decoy | 145 | 1 | RS142_MAIZE | 54 |
| 19046 | viridiplantae | AOP | 2 | Da | decoy | 146 | 1 | PSBH_DAUCA | 54 |
| 19046 | viridiplantae | AOP | 2 | Da | decoy | 147 | 1 | MT2_BRARP | 54 |
| 19046 | viridiplantae | AOP | 2 | Da | decoy | 148 | 1 | PROF9_PHLPR | 53 |
| 19046 | viridiplantae | AOP | 2 | Da | decoy | 149 | 1 | CSPL8_ORYSI | 53 |
| 19046 | viridiplantae | AOP | 2 | Da | decoy | 150 | 1 | SDH32_ORYSJ | 53 |
| 19046 | viridiplantae | AOP | 2 | Da | decoy | 151 | 1 | FER_GLEJA | 53 |
| 19046 | viridiplantae | AOP | 2 | Da | decoy | 152 | 1 | EM1_WHEAT | 52 |
| 19046 | viridiplantae | AOP | 2 | Da | decoy | 153 | 1 | SAU21_ARATH | 52 |
| 19046 | viridiplantae | AOP | 2 | Da | decoy | 154 | 1 | ATP9_MARPO | 52 |
| 19046 | viridiplantae | AOP | 2 | Da | decoy | 155 | 1 | PROCJ_OLEEU | 52 |
| 19046 | viridiplantae | AOP | 2 | Da | decoy | 156 | 1 | PSBL_CEDDE | 52 |
| 19046 | viridiplantae | AOP | 2 | Da | decoy | 157 | 1 | PROF2_CORAV | 52 |
| 19046 | viridiplantae | AOP | 2 | Da | decoy | 158 | 1 | RL36_DAUCA | 51 |
| 19046 | viridiplantae | AOP | 2 | Da | decoy | 159 | 1 | POLC7_CYNDA | 51 |
| 19046 | viridiplantae | AOP | 2 | Da | decoy | 160 | 1 | OP164_ARATH | 51 |
| 19046 | viridiplantae | AOP | 2 | Da | decoy | 161 | 1 | PSBI_TUPAK | 51 |
| 19046 | viridiplantae | AOP | 2 | Da | decoy | 162 | 1 | PSBW_ARATH | 51 |
| 19046 | viridiplantae | AOP | 2 | Da | decoy | 163 | 1 | HRD11_ARATH | 51 |
| 19046 | viridiplantae | AOP | 2 | Da | decoy | 164 | 1 | EPFL2_ARATH | 51 |
| 19046 | viridiplantae | AOP | 2 | Da | decoy | 165 | 1 | CML29_ARATH | 50 |
| 19046 | viridiplantae | AOP | 2 | Da | decoy | 166 | 1 | ICIA_HORVU | 50 |
| 19046 | viridiplantae | AOP | 2 | Da | decoy | 167 | 1 | PSBH_COFAR | 50 |
| 19046 | viridiplantae | AOP | 2 | Da | decoy | 168 | 1 | LE19_GOSHI | 50 |
| 19046 | viridiplantae | AOP | 2 | Da | decoy | 169 | 1 | PST2_ARATH | 50 |
| 19046 | viridiplantae | AOP | 2 | Da | decoy | 170 | 1 | PROF3_PHLPR | 50 |
| 19046 | viridiplantae | AOP | 2 | Da | decoy | 171 | 1 | KIC_ARATH | 50 |
| 19046 | viridiplantae | AOP | 2 | Da | decoy | 172 | 1 | PETD_ATRBE | 50 |
| 19046 | viridiplantae | AOP | 2 | Da | decoy | 173 | 1 | PROF1_LILLO | 50 |
| 19046 | viridiplantae | AOP | 2 | Da | decoy | 174 | 1 | PROCB_OLEEU | 50 |
| 19046 | viridiplantae | AOP | 2 | Da | decoy | 175 | 1 | ATPE_LACSA | 50 |
| 19046 | viridiplantae | AOP | 2 | Da | decoy | 176 | 1 | TOM92_ARATH | 50 |
| 19046 | viridiplantae | AOP | 2 | Da | decoy | 177 | 1 | PSBJ_AMBTC | 50 |
| 19046 | viridiplantae | AOP | 2 | Da | decoy | 178 | 1 | GRP10_BRANA | 49 |
| 19046 | viridiplantae | AOP | 2 | Da | decoy | 179 | 1 | PETM_CHLRE | 49 |
| 19046 | viridiplantae | AOP | 2 | Da | decoy | 180 | 1 | ACP1_CASGL | 49 |
| 19046 | viridiplantae | AOP | 2 | Da | decoy | 181 | 1 | PSBL_HUPLU | 49 |
| 19046 | viridiplantae | AOP | 2 | Da | decoy | 182 | 1 | PROAW_OLEEU | 49 |
| 19046 | viridiplantae | AOP | 2 | Da | decoy | 183 | 1 | PSBJ_OENEH | 49 |
| 19046 | viridiplantae | AOP | 2 | Da | decoy | 184 | 1 | PSBH_TUPAK | 49 |
| 19046 | viridiplantae | AOP | 2 | Da | decoy | 185 | 1 | RLA25_ARATH | 49 |
| 19046 | viridiplantae | AOP | 2 | Da | decoy | 186 | 1 | SODC_BRAOC | 49 |
| 19046 | viridiplantae | AOP | 2 | Da | decoy | 187 | 1 | PROCE_OLEEU | 48 |
| 19046 | viridiplantae | AOP | 2 | Da | decoy | 188 | 1 | NLT22_PARJU | 48 |
| 19046 | viridiplantae | AOP | 2 | Da | decoy | 189 | 1 | PIP2_ARATH | 48 |
| 19046 | viridiplantae | AOP | 2 | Da | decoy | 190 | 1 | ACBP_FRIAG | 48 |
| 19046 | viridiplantae | AOP | 2 | Da | decoy | 191 | 1 | RL373_ARATH | 48 |
| 19046 | viridiplantae | AOP | 2 | Da | decoy | 192 | 1 | MT2_MUSAC | 48 |
| 19046 | viridiplantae | AOP | 2 | Da | decoy | 193 | 1 | TIM8_ARATH | 48 |
| 19046 | viridiplantae | AOP | 2 | Da | decoy | 194 | 1 | FB41_ARATH | 48 |
| 19046 | viridiplantae | AOP | 2 | Da | decoy | 195 | 1 | MT21A_ORYSJ | 47 |
| 19046 | viridiplantae | AOP | 2 | Da | decoy | 196 | 1 | PROF_PYRCO | 47 |
| 19046 | viridiplantae | AOP | 2 | Da | decoy | 197 | 1 | TI141_ARATH | 47 |
| 19046 | viridiplantae | AOP | 2 | Da | decoy | 198 | 1 | PSAK_SPIOL | 47 |
| 19046 | viridiplantae | AOP | 2 | Da | decoy | 199 | 1 | PSBJ_MESVI | 47 |
| 19046 | viridiplantae | AOP | 2 | Da | decoy | 200 | 1 | CYC6_BRYMA | 46 |
| 19046 | viridiplantae | AOP | 2 | Da | decoy | 201 | 1 | CYC4_CHACT | 46 |
| 19046 | viridiplantae | AOP | 2 | Da | decoy | 202 | 1 | DEF10_ARATH | 46 |
| 19046 | viridiplantae | AOP | 2 | Da | decoy | 203 | 1 | LSM5_ARATH | 46 |
| 19046 | viridiplantae | AOP | 2 | Da | decoy | 204 | 1 | PSBJ_EUCGG | 46 |
| 19046 | viridiplantae | AOP | 2 | Da | decoy | 205 | 1 | FER_SCEQU | 46 |
| 19046 | viridiplantae | AOP | 2 | Da | decoy | 206 | 1 | ATP9_PETSP | 46 |
| 19046 | viridiplantae | AOP | 2 | Da | decoy | 207 | 1 | BOLA2_ARATH | 45 |
| 19046 | viridiplantae | AOP | 2 | Da | decoy | 208 | 1 | GRC13_ORYSJ | 45 |
| 19046 | viridiplantae | AOP | 2 | Da | decoy | 209 | 1 | PSK6_ARATH | 45 |
| 19046 | viridiplantae | AOP | 2 | Da | decoy | 210 | 1 | ATPH_PEA | 45 |
| 19046 | viridiplantae | AOP | 2 | Da | decoy | 211 | 1 | TOM72_ARATH | 45 |
| 19046 | viridiplantae | AOP | 2 | Da | decoy | 212 | 1 | PSAC_TUPAK | 45 |
| 19046 | viridiplantae | AOP | 2 | Da | decoy | 213 | 1 | EMP1_ORYSJ | 45 |
| 19046 | viridiplantae | AOP | 2 | Da | decoy | 214 | 1 | POLC7_PHLPR | 45 |
| 19046 | viridiplantae | AOP | 2 | Da | decoy | 215 | 1 | PSBH_MARPO | 44 |
| 19046 | viridiplantae | AOP | 2 | Da | decoy | 216 | 1 | DEF73_ARATH | 44 |
| 19046 | viridiplantae | AOP | 2 | Da | decoy | 217 | 1 | LSM6B_ARATH | 44 |
| 19046 | viridiplantae | AOP | 2 | Da | decoy | 218 | 1 | DEF83_ARATH | 44 |
| 19046 | viridiplantae | AOP | 2 | Da | decoy | 219 | 1 | TI143_ARATH | 44 |
| 19046 | viridiplantae | AOP | 2 | Da | decoy | 220 | 1 | PSBH_PHAAO | 44 |
| 19046 | viridiplantae | AOP | 2 | Da | decoy | 221 | 1 | PSBH_SPIMX | 44 |
| 19046 | viridiplantae | AOP | 2 | Da | decoy | 222 | 1 | RK14_OENAM | 44 |
| 19046 | viridiplantae | AOP | 2 | Da | decoy | 223 | 1 | PAFP_PHYAM | 44 |
| 19046 | viridiplantae | AOP | 2 | Da | decoy | 224 | 1 | PSAC_ZYGCR | 43 |
| 19046 | viridiplantae | AOP | 2 | Da | decoy | 225 | 1 | PSBH_CALFG | 43 |
| 19046 | viridiplantae | AOP | 2 | Da | decoy | 226 | 1 | PSBJ_CHLRE | 43 |
| 19046 | viridiplantae | AOP | 2 | Da | decoy | 227 | 1 | PSAK_CUCSA | 43 |
| 19046 | viridiplantae | AOP | 2 | Da | decoy | 228 | 1 | TIM13_ORYSJ | 43 |
| 19046 | viridiplantae | AOP | 2 | Da | decoy | 229 | 1 | ATPH_CICAR | 43 |
| 19046 | viridiplantae | AOP | 2 | Da | decoy | 230 | 1 | NU5C_PSEMZ | 42 |
| 19046 | viridiplantae | AOP | 2 | Da | decoy | 231 | 1 | ATP9_PETHY | 42 |
| 19046 | viridiplantae | AOP | 2 | Da | decoy | 232 | 1 | PSBJ_AETGR | 42 |
| 19046 | viridiplantae | AOP | 2 | Da | decoy | 233 | 1 | DF208_ARATH | 42 |
| 19046 | viridiplantae | AOP | 2 | Da | decoy | 234 | 1 | PSBH_DRIGR | 42 |
| 19046 | viridiplantae | AOP | 2 | Da | decoy | 235 | 1 | PSBH_CHAVU | 42 |
| 19046 | viridiplantae | AOP | 2 | Da | decoy | 236 | 1 | PSBH_HELAN | 42 |
| 19046 | viridiplantae | AOP | 2 | Da | decoy | 237 | 1 | R35A1_ARATH | 42 |
| 19046 | viridiplantae | AOP | 2 | Da | decoy | 238 | 1 | DF117_ARATH | 42 |
| 19046 | viridiplantae | AOP | 2 | Da | decoy | 239 | 1 | PSBM_PINTH | 41 |
| 19046 | viridiplantae | AOP | 2 | Da | decoy | 240 | 1 | AGP14_ARATH | 41 |
| 19046 | viridiplantae | AOP | 2 | Da | decoy | 241 | 1 | MT2A_ORYSJ | 41 |
| 19046 | viridiplantae | AOP | 2 | Da | decoy | 242 | 1 | PSBL_ADICA | 41 |
| 19046 | viridiplantae | AOP | 2 | Da | decoy | 243 | 1 | EC1_WHEAT | 41 |
| 19046 | viridiplantae | AOP | 2 | Da | decoy | 244 | 1 | PSBJ_CYCTA | 40 |
| 19046 | viridiplantae | AOP | 2 | Da | decoy | 245 | 1 | ATPH_OEDCA | 39 |
| 19046 | viridiplantae | AOP | 2 | Da | decoy | 246 | 1 | AGP24_ARATH | 39 |
| 19046 | viridiplantae | AOP | 2 | Da | decoy | 247 | 1 | PSBH_PSINU | 39 |
| 19046 | viridiplantae | AOP | 2 | Da | decoy | 248 | 1 | ATP9 BRANA | 39 |
| 19046 | viridiplantae | AOP | 2 | Da | decoy | 249 | 1 | PSBJ_AGRST | 39 |
| 19046 | viridiplantae | AOP | 2 | Da | decoy | 250 | 1 | PSBL_ANTMA | 39 |
| 19046 | viridiplantae | AOP | 2 | Da | decoy | 251 | 1 | AGP41_ARATH | 39 |
| 19046 | viridiplantae | AOP | 2 | Da | decoy | 252 | 1 | PSBJ_HORJU | 38 |
| 19046 | viridiplantae | AOP | 2 | Da | decoy | 253 | 1 | PSBJ_WHEAT | 38 |
| 19046 | viridiplantae | AOP | 2 | Da | decoy | 254 | 1 | PSBZ_ACOGR | 38 |
| 19046 | viridiplantae | AOP | 2 | Da | decoy | 255 | 1 | PSBJ_PSINU | 38 |
| 19046 | viridiplantae | AOP | 2 | Da | decoy | 256 | 1 | NDUA5_SOLTU | 38 |
| 19046 | viridiplantae | AOP | 2 | Da | decoy | 257 | 1 | PETG_PLAOC | 38 |
| 19046 | viridiplantae | AOP | 2 | Da | decoy | 258 | 1 | PSAI_CHLVU | 38 |
| 19046 | viridiplantae | AOP | 2 | Da | decoy | 259 | 1 | PSBJ_CUSEX | 37 |
| 19046 | viridiplantae | AOP | 2 | Da | decoy | 260 | 1 | PSBZ_PINTH | 37 |
| 19046 | viridiplantae | AOP | 2 | Da | decoy | 261 | 1 | NFD6_ARATH | 37 |
| 19046 | viridiplantae | AOP | 2 | Da | decoy | 262 | 1 | PETN_CHLRE | 36 |
| 19046 | viridiplantae | AOP | 2 | Da | decoy | 263 | 1 | ACBP1_ORYSJ | 35 |
| 19046 | viridiplantae | AOP | 2 | Da | decoy | 264 | 1 | GRP1_PETHY | 34 |
| 19046 | viridiplantae | AOP | 2 | Da | decoy | 265 | 1 | PSBN_CALFL | 34 |
| 19046 | viridiplantae | AOP | 2 | Da | decoy | 266 | 1 | AGP12_ARATH | 34 |
| 19046 | viridiplantae | AOP | 2 | Da | decoy | 267 | 1 | PSAC_PHYPA | 33 |
| 19046 | viridiplantae | AOP | 2 | Da | decoy | 268 | 1 | NLTP3_VITSX | 31 |
| 19046 | viridiplantae | AOP | 2 | Da | decoy | 269 | 1 | Y3974_ARATH | 31 |
| 19046 | viridiplantae | AOP | 2 | Da | decoy | 270 | 1 | F26G_SOLTO | 31 |
| 19046 | viridiplantae | AOP | 2 | Da | decoy | 271 | 1 | DEF43_ARATH | 30 |
| 19046 | viridiplantae | AOP | 2 | Da | decoy | 272 | 1 | APEP2_ORYSJ | 29 |
| 19046 | viridiplantae | AOP | 2 | Da | decoy | 273 | 1 | NLTP_RAPSA | 26 |
| 19046 | viridiplantae | AOP | 2 | Da | decoy | 274 | 1 | HSP90_POPEU | 25 |

| Job | | | Match | | Seq | | |
|---|---|---|---|---|---|---|---|
| no. | Mass | Matches | (sig) | Seqs | (sig) | emPAI | Species |
| 19031 | 9367 | 39 | 16 | 2 | 2 | n.a. | Cannabis sativa |
| 19031 | 9545 | 43 | 4 | 2 | 1 | n.a. | Cannabis sativa |
| 19031 | 7645 | 16 | 5 | 1 | 1 | n.a. | Cannabis sativa |
| 19031 | 9381 | 31 | 5 | 1 | 1 | n.a. | Humulus lupulus |
| 19031 | 3815 | 33 | 2 | 2 | 1 | n.a. | Cannabis sativa subsp. |
| | | | | | | | sativa |
| 19031 | 7985 | 32 | 2 | 2 | 1 | n.a. | Cannabis sativa |
| 19031 | 11994 | 26 | 1 | 2 | 1 | n.a. | Cannabis sativa |
| 19031 | 4165 | 15 | 1 | 2 | 1 | n.a. | Cannabis sativa |
| 19031 | 10380 | 7 | 1 | 1 | 1 | n.a. | Cannabis sativa subsp. |
| | | | | | | | sativa |
| 19031 | 4128 | 2 | 1 | 1 | 1 | n.a. | Cannabis sativa |
| 19031 | 14695 | 3 | 1 | 2 | 1 | n.a. | Humulus lupulus |
| 19031 | 4494 | 2 | 1 | 1 | 1 | n.a. | Cannabis sativa |
| 19030 | 9367 | 37 | 37 | 1 | 1 | 0.83 | Cannabis sativa |
| 19030 | 9545 | 39 | 39 | 1 | 1 | 1.43 | Cannabis sativa |
| 19030 | 3815 | 25 | 25 | 1 | 1 | 13.87 | Cannabis sativa subsp. |
| | | | | | | | sativa |
| 19030 | 7645 | 12 | 12 | 1 | 1 | 1.06 | Cannabis sativa |
| 19030 | 9381 | 21 | 21 | 1 | 1 | 0.35 | Humulus lupulus |
| 19030 | 4165 | 9 | 9 | 1 | 1 | 5.31 | Cannabis sativa |
| 19030 | 7985 | 12 | 12 | 1 | 1 | 1.84 | Cannabis sativa |
| 19030 | 11833 | 5 | 5 | 1 | 1 | 0.62 | Humulus lupulus |
| 19030 | 4421 | 17 | 17 | 1 | 1 | 0.8 | Cannabis sativa |
| 19030 | 11994 | 9 | 9 | 1 | 1 | 0.61 | Cannabis sativa |
| 19030 | 10414 | 5 | 5 | 1 | 1 | 0.72 | Cannabis sativa |
| 19030 | 10380 | 4 | 4 | 1 | 1 | 0.72 | Cannabis sativa subsp. |
| | | | | | | | sativa |
| 19030 | 17597 | 7 | 7 | 2 | 2 | 1.28 | Cannabis sativa |
| 19030 | 4128 | 2 | 2 | 1 | 1 | 0.87 | Cannabis sativa |
| 19030 | 7910 | 1 | 1 | 1 | 1 | 0.42 | Cannabis sativa |
| 19030 | 14696 | 1 | 1 | 1 | 1 | 0.22 | Cannabis sativa |
| 19030 | 4167 | 1 | 1 | 1 | 1 | 0.85 | Cannabis sativa |
| 19030 | 9489 | 2 | 2 | 1 | 1 | 0.35 | Cannabis sativa |
| 19030 | 4494 | 2 | 2 | 1 | 1 | 0.8 | Cannabis sativa |
| 19030 | 17504 | 1 | 1 | 1 | 1 | 0.18 | Cannabis sativa |
| 19030 | 4770 | 1 | 1 | 1 | 1 | 0.74 | Cannabis sativa |
| 19048 | 9545 | 53 | 53 | 1 | 1 | 1.43 | Cannabis sativa |
| 19048 | 9367 | 43 | 43 | 2 | 2 | 1.47 | Cannabis sativa |
| 19048 | 7645 | 23 | 23 | 2 | 2 | 11.61 | Cannabis sativa |
| 19048 | 3815 | 29 | 29 | 1 | 1 | 13.87 | Cannabis sativa subsp. |
| | | | | | | | sativa |
| 19048 | 17597 | 46 | 46 | 2 | 2 | 3.42 | Cannabis sativa |
| 19048 | 7985 | 17 | 17 | 1 | 1 | 4.7 | Cannabis sativa |
| 19048 | 9489 | 17 | 17 | 1 | 1 | 0.82 | Cannabis sativa |
| 19048 | 11994 | 19 | 19 | 1 | 1 | 1.05 | Cannabis sativa |
| 19048 | 11833 | 10 | 10 | 2 | 2 | 1.06 | Humulus lupulus |
| 19048 | 4165 | 9 | 9 | 1 | 1 | 0.85 | Cannabis sativa |
| 19048 | 10464 | 5 | 5 | 2 | 2 | 0.72 | Humulus lupulus |
| 19048 | 10414 | 7 | 7 | 1 | 1 | 0.72 | Cannabis sativa |
| 19048 | 11823 | 4 | 4 | 1 | 1 | 0.62 | Cannabis sativa |
| 19048 | 4421 | 19 | 19 | 1 | 1 | 0.8 | Cannabis sativa |
| 19048 | 14696 | 6 | 6 | 2 | 2 | 1.68 | Cannabis sativa |
| 19048 | 10380 | 7 | 7 | 1 | 1 | 0.72 | Cannabis sativa subsp. |
| | | | | | | | sativa |
| 19048 | 7910 | 1 | 1 | 1 | 1 | 0.42 | Cannabis sativa |
| 19048 | 4128 | 2 | 2 | 1 | 1 | 0.87 | Cannabis sativa |
| 19048 | 10012 | 11 | 11 | 2 | 2 | 6.26 | Boehmeria nivea |
| 19048 | 17504 | 1 | 1 | 1 | 1 | 0.18 | Cannabis sativa |
| 19048 | 4770 | 5 | 5 | 1 | 1 | 2.02 | Cannabis sativa |
| 19048 | 15516 | 1 | 1 | 1 | 1 | 0.21 | Cannabis sativa |
| 19048 | 4494 | 3 | 3 | 1 | 1 | 0.8 | Cannabis sativa |
| 19048 | 11327 | 2 | 2 | 1 | 1 | 0.66 | Boehmeria nivea |
| 19048 | 9475 | 2 | 2 | 1 | 1 | 0.35 | Cannabis sativa |
| 19048 | 4167 | 1 | 1 | 1 | 1 | 0.85 | Cannabis sativa |
| 19048 | 17456 | 1 | 1 | 1 | 1 | 0.18 | Boehmeria nivea |
| 19048 | 12135 | 1 | 1 | 1 | 1 | 0.27 | Boehmeria nivea |
| 19048 | 15282 | 1 | 1 | 1 | 1 | 0.21 | Humulus lupulus |
| 19048 | 9630 | 1 | 1 | 1 | 1 | 0.34 | Boehmeria nivea |
| 19048 | 3386 | 3 | 3 | 1 | 1 | 3.3 | Cannabis sativa |
| 19048 | 8785 | 1 | 1 | 1 | 1 | 0.38 | Cannabis sativa |
| 19048 | 16123 | 1 | 1 | 1 | 1 | 0.2 | Boehmeria nivea |
| 19048 | 3299 | 1 | 1 | 1 | 1 | 1.11 | Cannabis sativa |
| 19048 | 8525 | 1 | 1 | 1 | 1 | 0.39 | Cannabis sativa |
| 19048 | 4711 | 1 | 1 | 1 | 1 | 0.76 | Cannabis sativa |
| 19050 | 9367 | 35 | 35 | 1 | 1 | 2.35 | Cannabis sativa |
| 19050 | 7645 | 14 | 14 | 1 | 1 | 3.26 | Cannabis sativa |
| 19050 | 9545 | 37 | 37 | 1 | 1 | 1.43 | Cannabis sativa |
| 19050 | 3815 | 25 | 25 | 1 | 1 | 13.87 | Cannabis sativa subsp. |
| | | | | | | | sativa |
| 19050 | 4421 | 20 | 20 | 1 | 1 | 2.24 | Cannabis sativa |
| 19050 | 4165 | 8 | 8 | 2 | 2 | 20.57 | Cannabis sativa |
| 19050 | 7985 | 10 | 10 | 2 | 2 | 4.7 | Cannabis sativa |
| 19050 | 11994 | 10 | 10 | 1 | 1 | 1.6 | Cannabis sativa |
| 19050 | 11833 | 5 | 5 | 1 | 1 | 0.62 | Humulus lupulus |
| 19050 | 10414 | 3 | 3 | 1 | 1 | 0.72 | Cannabis sativa |
| 19050 | 10380 | 3 | 3 | 1 | 1 | 0.72 | Cannabis sativa subsp. |
| | | | | | | | sativa |
| 19050 | 4128 | 2 | 2 | 1 | 1 | 0.87 | Cannabis sativa |
| 19050 | 7910 | 1 | 1 | 1 | 1 | 0.42 | Cannabis sativa |
| 19050 | 17597 | 3 | 3 | 1 | 1 | 0.39 | Cannabis sativa |
| 19050 | 14696 | 1 | 1 | 1 | 1 | 0.22 | Cannabis sativa |
| 19050 | 9489 | 3 | 3 | 1 | 1 | 0.82 | Cannabis sativa |
| 19050 | 4008 | 2 | 2 | 1 | 1 | 2.62 | Cannabis sativa |
| 19050 | 4167 | 1 | 1 | 1 | 1 | 0.85 | Cannabis sativa |
| 19050 | 4494 | 2 | 2 | 1 | 1 | 0.8 | Cannabis sativa |
| 19050 | 17504 | 1 | 1 | 1 | 1 | 0.18 | Cannabis sativa |
| 19050 | 4770 | 1 | 1 | 1 | 1 | 0.74 | Cannabis sativa |
| 19049 | 9367 | 44 | 44 | 2 | 2 | 3.53 | Cannabis sativa |
| 19049 | 9545 | 53 | 53 | 1 | 1 | 2.26 | Cannabis sativa |
| 19049 | 7645 | 43 | 43 | 2 | 2 | 5937.4 | Cannabis sativa |
| 19049 | 3815 | 33 | 33 | 2 | 2 | 111.64 | Cannabis sativa subsp. |
| | | | | | | | sativa |
| 19049 | 7985 | 34 | 34 | 2 | 2 | 91.46 | Cannabis sativa |
| 19049 | 9381 | 29 | 29 | 2 | 2 | 9.91 | Humulus lupulus |
| 19049 | 4421 | 23 | 23 | 1 | 1 | 2.24 | Cannabis sativa |
| 19049 | 17597 | 36 | 36 | 2 | 2 | 5.15 | Cannabis sativa |
| 19049 | 9489 | 39 | 39 | 1 | 1 | 3.45 | Cannabis sativa |
| 19049 | 4165 | 16 | 16 | 1 | 1 | 5.31 | Cannabis sativa |
| 19049 | 10380 | 7 | 7 | 1 | 1 | 0.31 | Cannabis sativa subsp. |
| | | | | | | | sativa |
| 19049 | 11994 | 13 | 13 | 1 | 1 | 1.6 | Cannabis sativa |
| 19049 | 4770 | 10 | 10 | 2 | 2 | 2.02 | Cannabis sativa |
| 19049 | 11833 | 5 | 5 | 1 | 1 | 1.06 | Humulus lupulus |
| 19049 | 14696 | 7 | 7 | 2 | 2 | 2.27 | Cannabis sativa |
| 19049 | 11823 | 4 | 4 | 1 | 1 | 0.62 | Cannabis sativa |
| 19049 | 4008 | 17 | 17 | 2 | 2 | 46.41 | Cannabis sativa |
| 19049 | 4128 | 18 | 18 | 1 | 1 | 11.35 | Cannabis sativa |
| 19049 | 14695 | 4 | 4 | 2 | 2 | 0.81 | Humulus lupulus |
| 19049 | 10464 | 2 | 2 | 1 | 1 | 0.31 | Humulus lupulus |
| 19049 | 9893 | 28 | 28 | 2 | 2 | 406.84 | Boehmeria nivea |
| 19049 | 7910 | 1 | 1 | 1 | 1 | 0.42 | Cannabis sativa |
| 19049 | 11151 | 9 | 9 | 2 | 2 | 5.03 | Cannabis sativa |
| 19049 | 4494 | 13 | 13 | 2 | 2 | 4.83 | Cannabis sativa |
| 19049 | 15404 | 2 | 2 | 1 | 1 | 0.46 | Cannabis sativa |
| 19049 | 17504 | 2 | 2 | 2 | 2 | 0.39 | Cannabis sativa |
| 19049 | 10012 | 8 | 8 | 2 | 2 | 6.26 | Boehmeria nivea |
| 19049 | 13263 | 4 | 4 | 1 | 1 | 0.55 | Cannabis sativa |
| 19049 | 9475 | 3 | 3 | 1 | 1 | 0.82 | Cannabis sativa |
| 19049 | 13819 | 9 | 9 | 2 | 2 | 5.59 | Cannabis sativa |
| 19049 | 4464 | 5 | 5 | 1 | 1 | 0.8 | Cannabis sativa |
| 19049 | 6493 | 8 | 8 | 2 | 2 | 4.45 | Cannabis sativa |
| 19049 | 15516 | 1 | 1 | 1 | 1 | 0.21 | Cannabis sativa |
| 19049 | 10484 | 1 | 1 | 1 | 1 | 0.31 | Boehmeria nivea |
| 19049 | 10804 | 1 | 1 | 1 | 1 | 0.3 | Boehmeria nivea |
| 19049 | 9630 | 6 | 6 | 2 | 2 | 3.31 | Boehmeria nivea |
| 19049 | 10864 | 2 | 2 | 1 | 1 | 0.69 | Boehmeria nivea |
| 19049 | 10863 | 1 | 1 | 1 | 1 | 0.3 | Boehmeria nivea |
| 19049 | 3386 | 10 | 10 | 2 | 2 | 339.69 | Cannabis sativa |
| 19049 | 9406 | 2 | 2 | 1 | 1 | 0.82 | Cannabis sativa |
| 19049 | 11172 | 1 | 1 | 1 | 1 | 0.29 | Boehmeria nivea |
| 19049 | 10824 | 1 | 1 | 1 | 1 | 0.3 | Boehmeria nivea |
| 19049 | 11040 | 1 | 1 | 1 | 1 | 0.3 | Boehmeria nivea |
| 19049 | 15045 | 1 | 1 | 1 | 1 | 0.21 | Cannabis sativa |
| 19049 | 13331 | 1 | 1 | 1 | 1 | 0.24 | Cannabis sativa |
| 19049 | 10628 | 2 | 2 | 1 | 1 | 0.31 | Boehmeria nivea |
| 19049 | 10505 | 1 | 1 | 1 | 1 | 0.31 | Cannabis sativa |
| 19049 | 13360 | 2 | 2 | 1 | 1 | 0.54 | Cannabis sativa |
| 19049 | 14563 | 1 | 1 | 1 | 1 | 0.22 | Boehmeria nivea |
| 19049 | 13683 | 1 | 1 | 1 | 1 | 0.24 | Boehmeria nivea |
| 19049 | 12422 | 1 | 1 | 1 | 1 | 0.26 | Boehmeria nivea |
| 19049 | 4167 | 1 | 1 | 1 | 1 | 0.85 | Cannabis sativa |
| 19049 | 4719 | 3 | 3 | 2 | 2 | 4.24 | Cannabis sativa subsp. |
| | | | | | | | sativa |
| 19049 | 8785 | 3 | 3 | 1 | 1 | 1.61 | Cannabis sativa |
| 19049 | 5014 | 7 | 7 | 1 | 1 | 13.21 | Cannabis sativa |
| 19049 | 7198 | 2 | 2 | 2 | 2 | 1.15 | Cannabis sativa |
| 19049 | 4162 | 2 | 2 | 1 | 1 | 2.51 | Cannabis sativa |
| 19049 | 2760 | 1 | 1 | 1 | 1 | 1.38 | Cannabis sativa |
| 19049 | 3299 | 2 | 2 | 1 | 1 | 3.47 | Cannabis sativa |
| 19049 | 3168 | 2 | 2 | 1 | 1 | 3.66 | Cannabis sativa |
| 19049 | 8111 | 1 | 1 | 1 | 1 | 0.41 | Cannabis sativa |
| 19051 | 9367 | 37 | 37 | 2 | 2 | 0.83 | Cannabis sativa |
| 19051 | 9545 | 42 | 42 | 1 | 1 | 0.34 | Cannabis sativa |
| 19051 | 3815 | 18 | 18 | 1 | 1 | 0.96 | Cannabis sativa subsp. |
| | | | | | | | sativa |
| 19051 | 7645 | 12 | 12 | 1 | 1 | 0.44 | Cannabis sativa |
| 19051 | 9381 | 21 | 21 | 1 | 1 | 0.35 | Humulus lupulus |
| 19051 | 4165 | 8 | 8 | 1 | 1 | 0.85 | Cannabis sativa |
| 19051 | 7985 | 11 | 11 | 1 | 1 | 0.42 | Cannabis sativa |
| 19051 | 11994 | 13 | 13 | 1 | 1 | 0.27 | Cannabis sativa |
| 19051 | 4421 | 17 | 17 | 1 | 1 | 0.8 | Cannabis sativa |
| 19051 | 10414 | 5 | 5 | 1 | 1 | 0.31 | Cannabis sativa |
| 19051 | 11833 | 4 | 4 | 1 | 1 | 0.27 | Humulus lupulus |
| 19051 | 10380 | 5 | 5 | 1 | 1 | 0.31 | Cannabis sativa subsp. |
| | | | | | | | sativa |
| 19051 | 17597 | 10 | 10 | 2 | 2 | 0.39 | Cannabis sativa |
| 19051 | 7910 | 1 | 1 | 1 | 1 | 0.42 | Cannabis sativa |
| 19051 | 14696 | 3 | 3 | 2 | 2 | 0.48 | Cannabis sativa |
| 19051 | 9489 | 2 | 2 | 1 | 1 | 0.35 | Cannabis sativa |
| 19051 | 4167 | 1 | 1 | 1 | 1 | 0.85 | Cannabis sativa |
| 19051 | 4494 | 2 | 2 | 1 | 1 | 0.8 | Cannabis sativa |
| 19051 | 17504 | 1 | 1 | 1 | 1 | 0.18 | Cannabis sativa |
| 19051 | 4770 | 1 | 1 | 1 | 1 | 0.74 | Cannabis sativa |
| 19043 | 9545 | 53 | 53 | 1 | 1 | 0.34 | Cannabis sativa |
| 19043 | 9367 | 43 | 43 | 2 | 2 | 0.83 | Cannabis sativa |
| 19043 | 7645 | 16 | 16 | 1 | 1 | 0.44 | Cannabis sativa |
| 19043 | 3815 | 18 | 18 | 1 | 1 | 0.96 | Cannabis sativa subsp. |
| | | | | | | | sativa |
| 19043 | 17597 | 36 | 36 | 2 | 2 | 0.39 | Cannabis sativa |
| 19043 | 9489 | 20 | 20 | 1 | 1 | 0.35 | Cannabis sativa |
| 19043 | 11994 | 18 | 18 | 2 | 2 | 0.61 | Cannabis sativa |
| 19043 | 7985 | 15 | 15 | 1 | 1 | 0.42 | Cannabis sativa |
| 19043 | 11833 | 8 | 8 | 2 | 2 | 0.62 | Humulus lupulus |
| 19043 | 10414 | 8 | 8 | 1 | 1 | 0.31 | Cannabis sativa |
| 19043 | 4165 | 8 | 8 | 1 | 1 | 0.85 | Cannabis sativa |
| 19043 | 4421 | 19 | 19 | 1 | 1 | 0.8 | Cannabis sativa |
| 19043 | 10380 | 7 | 7 | 1 | 1 | 0.31 | Cannabis sativa subsp. |
| | | | | | | | sativa |
| 19043 | 11823 | 4 | 4 | 1 | 1 | 0.27 | Cannabis sativa |
| 19043 | 14696 | 4 | 4 | 2 | 2 | 0.48 | Cannabis sativa |
| 19043 | 7910 | 1 | 1 | 1 | 1 | 0.42 | Cannabis sativa |
| 19043 | 17504 | 2 | 2 | 1 | 1 | 0.18 | Cannabis sativa |
| 19043 | 4494 | 3 | 3 | 1 | 1 | 0.8 | Cannabis sativa |
| 19043 | 15516 | 1 | 1 | 1 | 1 | 0.21 | Cannabis sativa |
| 19043 | 4167 | 1 | 1 | 1 | 1 | 0.85 | Cannabis sativa |
| 19043 | 4770 | 3 | 3 | 1 | 1 | 0.74 | Cannabis sativa |
| 19043 | 11509 | 1 | 1 | 1 | 1 | 0.28 | Boehmeria nivea |
| 19043 | 10743 | 1 | 1 | 1 | 1 | 0.3 | Boehmeria nivea |
| 19043 | 13969 | 1 | 1 | 1 | 1 | 0.23 | Cannabis sativa |
| 19042 | 11460 | 159 | 159 | 2 | | 0.65 | Triticum aestivum |
| 19042 | 11418 | 77 | 77 | 2 | 2 | 0.65 | Capsicum annuum |
| 19042 | 8520 | 26 | 26 | 1 | 1 | 0.39 | Avena sativa |
| 19042 | 9545 | 42 | 42 | 1 | 1 | 0.34 | Aethionema cordifolium |
| 19042 | 4507 | 23 | 23 | 1 | 1 | 0.78 | Ephedra sinica |
| 19042 | 9561 | 34 | 34 | 1 | 1 | 0.34 | Phalaenopsis aphrodite subsp. |
| | | | | | | | formosana |
| 19042 | 7995 | 20 | 20 | 1 | 1 | 0.42 | Cycas taitungensis |
| 19042 | 9381 | 21 | 21 | 1 | 1 | 0.35 | Amborella trichopoda |
| 19042 | 3831 | 25 | 25 | 1 | 1 | 0.93 | Pelargonium hortorum |
| 19042 | 8536 | 25 | 25 | 1 | 1 | 0.39 | Coprinellus congregatus |
| 19042 | 3815 | 18 | 18 | 1 | 1 | 0.96 | Allium textile |
| 19042 | 15344 | 55 | 55 | 1 | 1 | 0.21 | Encephalartos altensteinii |
| 19042 | 3833 | 25 | 25 | 1 | 1 | 0.93 | Piper cenocladum |
| 19042 | 9380 | 18 | 18 | 1 | 1 | 0.35 | Citrus sinensis |
| 19042 | 9353 | 22 | 22 | 1 | 1 | 0.35 | Mesembryanthemum crystallinum |
| 19042 | 15360 | 37 | 37 | 1 | 1 | 0.21 | Trichinella pseudospiralis |
| 19042 | 9439 | 19 | 19 | 1 | 1 | 0.35 | Agrostis stolonifera |
| 19042 | 15358 | 43 | 43 | 2 | 2 | 0.46 | Volvox carteri |
| 19042 | 9531 | 21 | 21 | 1 | 1 | 0.34 | Spinacia oleracea |
| 19042 | 15188 | 14 | 14 | 2 | 2 | 0.46 | Arabidopsis thaliana |
| 19042 | 4481 | 24 | 24 | 1 | 1 | 0.8 | Agathis robusta |
| 19042 | 10464 | 6 | 6 | 1 | 1 | 0.31 | Oryza sativa subsp. japonica |
| 19042 | 15344 | 26 | 26 | 1 | 1 | 0.21 | Chlamydomonas reinhardtii |
| 19042 | 10435 | 6 | 6 | 1 | 1 | 0.31 | Gossypium hirsutum |
| 19042 | 6412 | 7 | 7 | 1 | 1 | 0.53 | Arabidopsis thaliana |
| 19042 | 11850 | 5 | 5 | 1 | 1 | 0.27 | Nicotiana sylvestris |
| 19042 | 11994 | 12 | 12 | 2 | 2 | 0.61 | Cannabis sativa |
| 19042 | 4164 | 5 | 5 | 1 | 1 | 0.85 | Cryptomeria japonica |
| 19042 | 7500 | 7 | 7 | 2 | 2 | 1.08 | Ostertagia ostertagi |
| 19042 | 9529 | 12 | 12 | 1 | 1 | 0.34 | Drimys granadensis |
| 19042 | 11866 | 4 | 4 | 1 | 1 | 0.27 | Solanum bulbocastanum |
| 19042 | 15408 | 15 | 15 | 1 | 1 | 0.21 | Caenorhabditis elegans |
| 19042 | 8192 | 10 | 10 | 1 | 1 | 0.4 | Solanum lycopersicum |
| 19042 | 15425 | 15 | 15 | 1 | 1 | 0.21 | Cichorium intybus |
| 19042 | 15332 | 15 | 15 | 2 | 2 | 0.46 | Medicago sativa |
| 19042 | 15406 | 13 | 13 | 1 | 1 | 0.21 | Arabidopsis thaliana |
| 19042 | 10536 | 6 | 6 | 1 | 1 | 0.31 | Mercurialis perennis |
| 19042 | 6883 | 2 | 2 | 1 | 1 | 0.49 | Arabidopsis thaliana |
| 19042 | 4180 | 5 | 5 | 1 | 1 | 0.85 | Lepidium virginicum |
| 19042 | 4782 | 4 | 4 | 1 | 1 | 0.74 | Lemna minor |
| 19042 | 13909 | 4 | 4 | 1 | 1 | 0.23 | Oryza sativa subsp. indica |
| 19042 | 17504 | 1 | 1 | 1 | 1 | 0.18 | Atropa belladonna |
| 19042 | 15215 | 3 | 3 | 1 | 1 | 0.21 | Arabidopsis thaliana |
| 19042 | 25070 | 3 | 3 | 1 | 1 | 0.12 | Arabidopsis thaliana |
| 19042 | 9351 | 1 | 1 | 1 | 1 | 0.35 | Beutenbergia cavernae (strain ATCC |
| | | | | | | | BAA-8/DSM 12333/NBRC 16432) |
| 19042 | 9577 | 2 | 2 | 1 | 1 | 0.34 | Haloferax volcanii (strain ATCC |
| | | | | | | | 29605/DSM 3757/JCM 8879/ |
| | | | | | | | NBRC 14742/NCIMB 2012/VKM |
| | | | | | | | B-1768/DS2) |
| 19042 | 15535 | 1 | 1 | 1 | 1 | 0.21 | Cairina moschata |
| 19042 | 10496 | 3 | 3 | 1 | 1 | 0.31 | Morus indica |
| 19042 | 10410 | 2 | 2 | 1 | 1 | 0.31 | Lactuca sativa |
| 19042 | 8984 | 1 | 1 | 1 | 1 | 0.37 | Dictyostelium discoideum |
| 19042 | 13968 | 2 | 2 | 1 | 1 | 0.23 | Oryza sativa subsp. indica |
| 19042 | 13699 | 1 | 1 | 1 | 1 | 0.24 | Arabidopsis thaliana |
| 19042 | 7163 | 1 | 1 | 1 | 1 | 0.47 | Lactobacillus plantarum (strain ATCC |
| | | | | | | | BAA-793/NCIMB 8826/WCFS1) |
| 19042 | 8790 | 1 | 1 | 1 | 1 | 0.38 | Ilyobacter tartaricus |
| 19042 | 9474 | 1 | 1 | 1 | 1 | 0.35 | Arabidopsis thaliana |
| 19042 | 9997 | 1 | 1 | 1 | 1 | 0.33 | Corynebacterium diphtheriae (strain |
| | | | | | | | ATCC 700971/NCTC 13129/ |
| | | | | | | | Biotype gravis) |
| 19042 | 10120 | 1 | 1 | 1 | 1 | 0.32 | Mannheimia succiniciproducens |
| | | | | | | | (strain MBEL55E) |
| 19042 | 8667 | 3 | 3 | 1 | 1 | 0.38 | Helianthus annuus |
| 19042 | 12553 | 1 | 1 | 1 | 1 | 0.26 | Lupinus luteus |
| 19042 | 15934 | 3 | 3 | 1 | 1 | 0.2 | Pseudoalteromonas haloplanktis |
| | | | | | | | (strain TAC 125) |
| 19042 | 14873 | 3 | 3 | 1 | 1 | 0.21 | Oryza sativa subsp. indica |
| 19042 | 9665 | 1 | 1 | 1 | 1 | 0.34 | Anabaena variabilis (strain ATCC |
| | | | | | | | 29413/PCC 7937) |
| 19042 | 17590 | 1 | 1 | 1 | 1 | 0.18 | Salmonella arizonae (strain ATCC |
| | | | | | | | BAA-731/CDC346-86/RSK2980) |
| 19042 | 4727 | 2 | 2 | 1 | 1 | 0.74 | Ostreococcus tauri |
| 19042 | 9177 | 2 | 2 | 1 | 1 | 0.36 | Dictyostelium discoideum |
| 19042 | 9524 | 1 | 1 | 1 | 1 | 0.34 | Candida albicans (strain SC5314/ |
| | | | | | | | ATCC MYA-2876) |
| 19042 | 12673 | 1 | 1 | 1 | 1 | 0.25 | Yarrowia lipolytica (strain CLIB 122/ |
| | | | | | | | E 150) |
| 19042 | 9346 | 1 | 1 | 1 | 1 | 0.35 | Apis mellifera ligustica |
| 19042 | 8713 | 1 | 1 | 1 | 1 | 0.38 | Schizosaccharomyces pombe (strain |
| | | | | | | | 972/ATCC 24843) |
| 19042 | 8905 | 1 | 1 | 1 | 1 | 0.37 | Saccharomyces cerevisiae (strain |
| | | | | | | | ATCC 204508/S288c) |
| 19042 | 9973 | 1 | 1 | 1 | 1 | 0.33 | Dickeya chrysanthemi |
| 19042 | 4168 | 1 | 1 | 1 | 1 | 0.85 | Guillardia theta |
| 19042 | 9556 | 1 | 1 | 1 | 1 | 0.34 | Californiconus californicus |
| 19042 | 4181 | 1 | 1 | 1 | 1 | 0.85 | Cuscuta exaltata |
| 19042 | 14852 | 1 | 1 | 1 | 1 | 0.21 | Arabidopsis thaliana |
| 19042 | 4774 | 1 | 1 | 1 | 1 | 0.74 | Amborella trichopoda |
| 19042 | 15723 | 1 | 1 | 1 | 1 | 0.2 | Arabidopsis thaliana |
| 19042 | 4114 | 1 | 1 | 1 | 1 | 0.87 | Agrostis stolonifera |
| 19042 | 7211 | 1 | 1 | 1 | 1 | 0.47 | Pseudopleuronectes americanus |
| 19042 | 12965 | 1 | 1 | 1 | 1 | 0.25 | Arabidopsis thaliana |
| 19042 | 16392 | 1 | 1 | 1 | 1 | 0.19 | Arabidopsis thaliana |
| 19042 | 9242 | 1 | 1 | 1 | 1 | 0.36 | Actinobacillus pleuropneumoniae |
| 19042 | 5317 | 1 | 1 | 1 | 1 | 0.65 | Leuconostoc citreum (strain KM20) |
| 19042 | 9492 | 1 | 1 | 1 | 1 | 0.34 | Dictyostelium discoideum |
| 19042 | 10561 | 1 | 1 | 1 | 1 | 0.31 | Aeromonas hydrophila subsp. |
| | | | | | | | hydrophila (strain ATCC 7966/DSM |
| | | | | | | | 30187/JCM 1027/KCTC 2358/ |
| | | | | | | | NCIMB 9240) |
| 19042 | 14907 | 1 | 1 | 1 | 1 | 0.21 | Takifugu rubripes |
| 19042 | 8289 | 1 | 1 | 1 | 1 | 0.4 | Bacillus subtilis (strain 168) |
| 19042 | 14989 | 1 | 1 | 1 | 1 | 0.21 | Shewanella frigidimarina (strain |
| | | | | | | | NCIMB 400) |
| 19042 | 10776 | 1 | 1 | 1 | 1 | 0.3 | Methanoculleus marisnigri (strain |
| | | | | | | | ATCC 35101/DSM 1498/JR1) |
| 19042 | 17353 | 1 | 1 | 1 | 1 | 0.18 | Shewanella baltica (strain OS223) |
| 19042 | 14405 | 1 | 1 | 1 | 1 | 0.22 | Euphorbia esula |
| 19042 | 9733 | 1 | 1 | 1 | 1 | 0.34 | Vitis sp. |
| 19042 | 8037 | 1 | 1 | 1 | 1 | 0.42 | Nitrobacter winogradskyi (strain |
| | | | | | | | ATCC 25391/DSM 10237/CIP |
| | | | | | | | 104748/NCIMB 11846/Nb-255) |
| 19042 | 15799 | 1 | 1 | 1 | 1 | 0.2 | Aeromonas salmonicida (strain A449) |
| 19042 | 10763 | 1 | 1 | 1 | 1 | 0.3 | Frankia sp. (strain EAN1pec) |
| 19044 | 11460 | 182 | 182 | 2 | 2 | 0.65 | Triticum aestivum |
| 19044 | 11418 | 93 | 93 | 2 | 2 | 0.65 | Capsicum annuum |
| 19044 | 8520 | 27 | 27 | 1 | 1 | 0.39 | Avena sativa |
| 19044 | 9545 | 46 | 46 | 1 | 1 | 0.34 | Aethionema cordifolium |
| 19044 | 4507 | 23 | 23 | 1 | 1 | 0.78 | Ephedra sinica |
| 19044 | 9561 | 38 | 38 | 1 | 1 | 0.34 | Phalaenopsis aphrodite subsp. |
| | | | | | | | formosana |
| 19044 | 15344 | 63 | 63 | 1 | 1 | 0.21 | Encephalartos altensteinii |
| 19044 | 7995 | 23 | 23 | 1 | 1 | 0.42 | Cycas taitungensis |
| 19044 | 9381 | 27 | 27 | 1 | 1 | 0.35 | Amborella trichopoda |
| 19044 | 9353 | 24 | 24 | 1 | 1 | 0.35 | Mesembryanthemum crystallinum |
| 19044 | 3831 | 27 | 27 | 1 | 1 | 0.93 | Pelargonium hortorum |
| 19044 | 3815 | 18 | 18 | 1 | 1 | 0.96 | Allium textile |
| 19044 | 3833 | 25 | 25 | 1 | 1 | 0.93 | Piper cenocladum |
| 19044 | 15358 | 61 | 61 | 2 | 2 | 0.46 | Volvox carteri |
| 19044 | 15344 | 51 | 51 | 1 | 1 | 0.21 | Chlamydomonas reinhardtii |
| 19044 | 15332 | 45 | 45 | 2 | 2 | 0.46 | Medicago sativa |
| 19044 | 9439 | 20 | 20 | 1 | 1 | 0.35 | Agrostis stolonifera |
| 19044 | 9531 | 29 | 29 | 1 | 1 | 0.34 | Spinacia oleracea |
| 19044 | 9545 | 31 | 31 | 1 | 1 | 0.34 | Cuscuta reflexa |
| 19044 | 15188 | 15 | 15 | 2 | 2 | 0.46 | Arabidopsis thaliana |
| 19044 | 4481 | 24 | 24 | 1 | 1 | 0.8 | Agathis robusta |
| 19044 | 15454 | 26 | 26 | 2 | 2 | 0.46 | Arabidopsis thaliana |
| 19044 | 15425 | 38 | 38 | 1 | 1 | 0.21 | Cichorium intybus |
| 19044 | 10464 | 6 | 6 | 1 | 1 | 0.31 | Oryza sativa subsp. japonica |
| 19044 | 6412 | 8 | 8 | 1 | 1 | 0.53 | Arabidopsis thaliana |
| 19044 | 10435 | 6 | 6 | 1 | 1 | 0.31 | Gossypium hirsutum |
| 19044 | 11850 | 6 | 6 | 1 | 1 | 0.27 | Nicotiana sylvestris |
| 19044 | 11994 | 14 | 14 | 2 | 2 | 0.61 | Cannabis sativa |
| 19044 | 9529 | 17 | 17 | 1 | 1 | 0.34 | Drimys granadensis |
| 19044 | 8192 | 14 | 14 | 1 | 1 | 0.4 | Solanum lycopersicum |
| 19044 | 4198 | 7 | 7 | 1 | 1 | 0.85 | Cycas taitungensis |
| 19044 | 11866 | 4 | 4 | 1 | 1 | 0.27 | Solanum bulbocastanum |
| 19044 | 8015 | 7 | 7 | 1 | 1 | 0.42 | Cryptomeria japonica |
| 19044 | 10536 | 21 | 21 | 1 | 1 | 0.31 | Mercurialis perennis |
| 19044 | 6883 | 3 | 3 | 1 | 1 | 0.49 | Arabidopsis thaliana |
| 19044 | 4782 | 7 | 7 | 1 | 1 | 0.74 | Lemna minor |
| 19044 | 4180 | 5 | 5 | 1 | 1 | 0.85 | Lepidium virginicum |
| 19044 | 13909 | 5 | 5 | 1 | 1 | 0.23 | Oryza sativa subsp. indica |
| 19044 | 10410 | 11 | 11 | 1 | 1 | 0.31 | Lactuca sativa |
| 19044 | 15215 | 3 | 3 | 1 | 1 | 0.21 | Arabidopsis thaliana |
| 19044 | 14873 | 8 | 8 | 2 | 2 | 0.48 | Oryza sativa subsp. indica |
| 19044 | 25070 | 8 | 8 | 1 | 1 | 0.12 | Arabidopsis thaliana |
| 19044 | 10496 | 5 | 5 | 1 | 1 | 0.31 | Morus indica |
| 19044 | 13968 | 3 | 3 | 1 | 1 | 0.23 | Oryza sativa subsp. indica |
| 19044 | 17504 | 1 | 1 | 1 | 1 | 0.18 | Atropa belladonna |
| 19044 | 12553 | 3 | 3 | 1 | 1 | 0.26 | Lupinus luteus |
| 19044 | 4727 | 4 | 4 | 1 | 1 | 0.74 | Ostreococcus tauri |
| 19044 | 8667 | 3 | 3 | 1 | 1 | 0.38 | Helianthus annuus |
| 19044 | 13699 | 1 | 1 | 1 | 1 | 0.24 | Arabidopsis thaliana |
| 19044 | 12965 | 3 | 3 | 1 | 1 | 0.25 | Arabidopsis thaliana |
| 19044 | 10409 | 5 | 5 | 1 | 1 | 0.31 | Nicotiana tabacum |
| 19044 | 9474 | 1 | 1 | 1 | 1 | 0.35 | Arabidopsis thaliana |
| 19044 | 11329 | 1 | 1 | 1 | 1 | 0.29 | Arabidopsis thaliana |
| 19044 | 7939 | 1 | 1 | 1 | 1 | 0.42 | Morus indica |
| 19044 | 14405 | 2 | 2 | 2 | 2 | 0.49 | Euphorbia esula |
| 19044 | 15632 | 1 | 1 | 1 | 1 | 0.2 | Arabidopsis thaliana |
| 19044 | 4181 | 1 | 1 | 1 | 1 | 0.85 | Cuscuta exaltata |
| 19044 | 14852 | 1 | 1 | 1 | 1 | 0.21 | Arabidopsis thaliana |
| 19044 | 4774 | 1 | 1 | 1 | 1 | 0.74 | Amborella trichopoda |
| 19044 | 15723 | 1 | 1 | 1 | 1 | 0.2 | Arabidopsis thaliana |
| 19044 | 4114 | 1 | 1 | 1 | 1 | 0.87 | Agrostis stolonifera |
| 19044 | 9395 | 1 | 1 | 1 | 1 | 0.35 | Arabidopsis thaliana |
| 19044 | 3484 | 2 | 2 | 1 | 1 | 1.07 | Zygnema circumcarinatum |
| 19044 | 16392 | 1 | 1 | 1 | 1 | 0.19 | Arabidopsis thaliana |
| 19044 | 16077 | 1 | 1 | 1 | 1 | 0.2 | Gossypium hirsutum |
| 19044 | 4134 | 1 | 1 | 1 | 1 | 0.87 | Amborella trichopoda |
| 19044 | 4476 | 1 | 1 | 1 | 1 | 0.8 | Marchantia polymorpha |
| 19044 | 4071 | 2 | 2 | 1 | 1 | 0.87 | Solanum tuberosum |
| 19044 | 4494 | 1 | 1 | 1 | 1 | 0.8 | Acorus calamus |
| 19044 | 9445 | 1 | 1 | 1 | 1 | 0.35 | Panax ginseng |
| 19044 | 9733 | 1 | 1 | 1 | 1 | 0.34 | Vitis sp. |
| 19044 | 9050 | 1 | 1 | 1 | 1 | 0.36 | Arabidopsis thaliana |
| 19044 | 8657 | 1 | 1 | 1 | 1 | 0.38 | Arabidopsis thaliana |
| 19044 | 12062 | 1 | 1 | 1 | 1 | 0.27 | Arabidopsis thaliana |
| 19044 | 11580 | 1 | 1 | 1 | 1 | 0.28 | Arabidopsis thaliana |
| 19044 | 9918 | 1 | 1 | 1 | 1 | 0.33 | Arabidopsis thaliana |
| 19044 | 10137 | 2 | 2 | 1 | 1 | 0.32 | Oryza sativa subsp. japonica |
| 19044 | 7738 | 1 | 1 | 1 | 1 | 0.43 | Lactuca sativa |
| 19044 | 12058 | 1 | 1 | 1 | 1 | 0.27 | Arabidopsis thaliana |
| 19044 | 9576 | 1 | 1 | 1 | 1 | 0.34 | Lilium henryi |
| 19044 | 7095 | 1 | 1 | 1 | 1 | 0.47 | Vitis vinifera |
| 19044 | 8930 | 1 | 1 | 1 | 1 | 0.37 | Arabidopsis thaliana |
| 19044 | 9635 | 1 | 1 | 1 | 1 | 0.34 | Zea mays |
| 19044 | 15695 | 1 | 1 | 1 | 1 | 0.2 | Pisum sativum |
| 19045 | 11402 | 239 | 239 | 2 | 2 | 8.46 | Arabidopsis thaliana |
| 19045 | 11450 | 113 | 113 | 2 | 2 | 0.65 | Chlamydomonas reinhardtii |
| 19045 | 4481 | 29 | 29 | 1 | 1 | 0.8 | Agathis robusta |
| 19045 | 4465 | 25 | 25 | 1 | 1 | 2.24 | Pinus koraiensis |
| 19045 | 8520 | 27 | 27 | 1 | 1 | 0.92 | Avena sativa |
| 19045 | 4465 | 26 | 26 | 1 | 1 | 0.8 | Marchantia polymorpha |
| 19045 | 9545 | 43 | 43 | 1 | 1 | 0.81 | Aethionema cordifolium |
| 19045 | 15344 | 61 | 61 | 1 | 1 | 0.46 | Encephalartos altensteinii |
| 19045 | 9531 | 40 | 40 | 1 | 1 | 1.43 | Spinacia oleracea |
| 19045 | 15358 | 55 | 55 | 2 | 2 | 0.76 | Volvox carteri |
| 19045 | 7971 | 20 | 20 | 1 | 1 | 0.42 | Arabis hirsuta |
| 19045 | 7995 | 20 | 20 | 1 | 1 | 1.01 | Cycas taitungensis |
| 19045 | 9381 | 24 | 24 | 1 | 1 | 0.82 | Amborella trichopoda |
| 19045 | 8001 | 19 | 19 | 1 | 1 | 0.42 | Ceratophyllum demersum |
| 19045 | 3815 | 25 | 25 | 1 | 1 | 13.87 | Allium textile |
| 19045 | 3831 | 26 | 26 | 1 | 1 | 12.94 | Pelargonium hortorum |
| 19045 | 9529 | 32 | 32 | 1 | 1 | 0.81 | Drimys granadensis |
| 19045 | 3833 | 25 | 25 | 1 | 1 | 12.94 | Piper cenocladum |
| 19045 | 15344 | 41 | 41 | 1 | 1 | 0.46 | Chlamydomonas reinhardtii |
| 19045 | 6412 | 13 | 13 | 1 | 1 | 1.33 | Arabidopsis thaliana |
| 19045 | 15316 | 36 | 36 | 2 | 2 | 1.13 | Arabidopsis thaliana |
| 19045 | 9439 | 18 | 18 | 1 | 1 | 0.35 | Agrostis stolonifera |
| 19045 | 15188 | 29 | 29 | 2 | 2 | 2.79 | Arabidopsis thaliana |
| 19045 | 15332 | 32 | 32 | 2 | 2 | 3.54 | Medicago sativa |
| 19045 | 7969 | 13 | 13 | 1 | 1 | 0.42 | Agrostis stolonifera |
| 19045 | 9353 | 18 | 18 | 1 | 1 | 0.35 | Mesembryanthemum crystallinum |
| 19045 | 10464 | 6 | 6 | 1 | 1 | 0.72 | Oryza sativa subsp. japonica |
| 19045 | 10435 | 6 | 6 | 1 | 1 | 0.31 | Gossypium hirsutum |
| 19045 | 10536 | 23 | 23 | 1 | 1 | 1.94 | Mercurialis perennis |
| 19045 | 11850 | 5 | 5 | 1 | 1 | 0.27 | Nicotiana sylvestris |
| 19045 | 11994 | 11 | 11 | 1 | 1 | 0.61 | Cannabis sativa |
| 19045 | 7463 | 10 | 10 | 1 | 1 | 3.43 | Zea mays |
| 19045 | 15406 | 16 | 16 | 2 | 2 | 1.12 | Arabidopsis thaliana |
| 19045 | 4164 | 5 | 5 | 1 | 1 | 0.85 | Cryptomeria japonica |
| 19045 | 4198 | 7 | 7 | 1 | 1 | 5.31 | Cycas taitungensis |
| 19045 | 11866 | 4 | 4 | 1 | 1 | 0.27 | Solanum bulbocastanum |
| 19045 | 8192 | 12 | 12 | 1 | 1 | 0.97 | Solanum lycopersicum |
| 19045 | 4134 | 2 | 2 | 1 | 1 | 0.87 | Pinus koraiensis |
| 19045 | 15454 | 10 | 10 | 1 | 1 | 0.46 | Arabidopsis thaliana |
| 19045 | 6883 | 2 | 2 | 1 | 1 | 0.49 | Arabidopsis thaliana |
| 19045 | 12505 | 8 | 8 | 1 | 1 | 0.26 | Euphorbia esula |
| 19045 | 8027 | 6 | 6 | 1 | 1 | 1.01 | Pisum sativum |
| 19045 | 15318 | 5 | 5 | 1 | 1 | 0.21 | Lilium longiflorum |
| 19045 | 4128 | 2 | 2 | 1 | 1 | 0.87 | Aethionema cordifolium |
| 19045 | 4782 | 6 | 6 | 1 | 1 | 2.02 | Lemna minor |
| 19045 | 13909 | 4 | 4 | 1 | 1 | 0.52 | Oryza sativa subsp. indica |
| 19045 | 4114 | 2 | 2 | 1 | 1 | 0.87 | Arabidopsis thaliana |
| 19045 | 10993 | 4 | 4 | 1 | 1 | 0.3 | Arabidopsis thaliana |
| 19045 | 15425 | 3 | 3 | 1 | 1 | 0.46 | Cichorium intybus |
| 19045 | 25070 | 9 | 9 | 2 | 2 | 0.42 | Arabidopsis thaliana |
| 19045 | 9906 | 1 | 1 | 1 | 1 | 0.33 | Arabidopsis thaliana |
| 19045 | 10496 | 3 | 3 | 1 | 1 | 0.31 | Morus indica |
| 19045 | 15467 | 4 | 4 | 1 | 1 | 0.46 | Arabidopsis thaliana |
| 19045 | 15215 | 2 | 2 | 1 | 1 | 0.21 | Arabidopsis thaliana |
| 19045 | 9515 | 4 | 4 | 1 | 1 | 0.81 | Pinus thunbergii |
| 19045 | 4746 | 5 | 5 | 1 | 1 | 0.74 | Chlorokybus atmophyticus |
| 19045 | 14873 | 4 | 4 | 2 | 2 | 0.79 | Oryza sativa subsp. indica |
| 19045 | 7742 | 2 | 2 | 1 | 1 | 0.43 | Coffea arabica |
| 19045 | 17504 | 1 | 1 | 1 | 1 | 0.18 | Atropa belladonna |
| 19045 | 10434 | 1 | 1 | 1 | 1 | 0.31 | Capsella bursa-pastoris |
| 19045 | 12553 | 2 | 2 | 1 | 1 | 0.26 | Lupinus luteus |
| 19045 | 9635 | 2 | 2 | 1 | 1 | 0.79 | Zea mays |
| 19045 | 9383 | 4 | 4 | 1 | 1 | 0.82 | Oryza sativa subsp. japonica |
| 19045 | 13968 | 3 | 3 | 1 | 1 | 0.51 | Oryza sativa subsp. indica |
| 19045 | 8851 | 5 | 5 | 1 | 1 | 0.88 | Oryza sativa subsp. japonica |
| 19045 | 7584 | 2 | 2 | 1 | 1 | 1.08 | Oenothera biennis |
| 19045 | 15450 | 1 | 1 | 1 | 1 | 0.21 | Arabidopsis thaliana |
| 19045 | 10159 | 1 | 1 | 1 | 1 | 0.32 | Oryza sativa subsp. japonica |
| 19045 | 7708 | 1 | 1 | 1 | 1 | 0.44 | Nymphaea alba |
| 19045 | 16310 | 1 | 1 | 1 | 1 | 0.19 | Zea mays |
| 19045 | 7637 | 3 | 3 | 2 | 2 | 1.06 | Arabidopsis thaliana |
| 19045 | 4005 | 2 | 2 | 1 | 1 | 0.9 | Hordeum vulgare |
| 19045 | 4221 | 1 | 1 | 1 | 1 | 0.85 | Anthoceros angustus |
| 19045 | 7529 | 2 | 2 | 1 | 1 | 1.08 | Marchantia polymorpha |
| 19045 | 10137 | 2 | 2 | 1 | 1 | 0.32 | Oryza sativa subsp. japonica |
| 19045 | 14869 | 2 | 2 | 1 | 1 | 0.21 | Mesostigma viride |
| 19045 | 14590 | 1 | 1 | 1 | 1 | 0.22 | Olea europaea |
| 19045 | 13699 | 1 | 1 | 1 | 1 | 0.24 | Arabidopsis thaliana |
| 19045 | 11420 | 1 | 1 | 1 | 1 | 0.28 | Oryza sativa subsp. japonica |
| 19045 | 14654 | 1 | 1 | 1 | 1 | 0.22 | Arabidopsis thaliana |
| 19045 | 13912 | 1 | 1 | 1 | 1 | 0.23 | Oryza sativa subsp. japonica |
| 19045 | 10165 | 2 | 2 | 2 | 2 | 0.74 | Brassica napus |
| 19045 | 9634 | 1 | 1 | 1 | 1 | 0.34 | Arabidopsis thaliana |
| 19045 | 9669 | 2 | 2 | 1 | 1 | 0.79 | Arabidopsis thaliana |
| 19045 | 7796 | 1 | 1 | 1 | 1 | 0.43 | Hordeum vulgare |
| 19045 | 4153 | 1 | 1 | 1 | 1 | 0.87 | Platanus occidentalis |
| 19045 | 11481 | 1 | 1 | 1 | 1 | 0.28 | Petunia hybrida |
| 19045 | 15723 | 2 | 2 | 2 | 2 | 0.45 | Arabidopsis thaliana |
| 19045 | 16392 | 1 | 1 | 1 | 1 | 0.19 | Arabidopsis thaliana |
| 19045 | 7500 | 3 | 3 | 1 | 1 | 1.08 | Pisum sativum |
| 19045 | 9474 | 1 | 1 | 1 | 1 | 0.35 | Arabidopsis thaliana |
| 19045 | 11192 | 1 | 1 | 1 | 1 | 0.29 | Arabidopsis thaliana |
| 19045 | 16457 | 1 | 1 | 1 | 1 | 0.19 | Arabidopsis thaliana |
| 19045 | 15183 | 1 | 1 | 1 | 1 | 0.21 | Phaseolus vulgaris |
| 19045 | 14535 | 1 | 1 | 1 | 1 | 0.22 | Arabidopsis thaliana |
| 19045 | 9935 | 1 | 1 | 1 | 1 | 0.33 | Oenothera ammophila |
| 19045 | 16387 | 1 | 1 | 1 | 1 | 0.19 | Arabidopsis thaliana |
| 19045 | 17205 | 1 | 1 | 1 | 1 | 0.18 | Physcomitrella patens subsp. patens |
| 19045 | 4677 | 1 | 1 | 1 | 1 | 0.76 | Chlorella vulgaris |
| 19045 | 12189 | 1 | 1 | 1 | 1 | 0.26 | Hordeum vulgare |
| 19045 | 7695 | 1 | 1 | 1 | 1 | 0.44 | Phalaenopsis aphrodite subsp. |
| | | | | | | | formosana |
| 19045 | 6085 | 1 | 1 | 1 | 1 | 0.56 | Arabidopsis thaliana |
| 19045 | 4015 | 2 | 2 | 1 | 1 | 0.9 | Marchantia polymorpha |
| 19045 | 11339 | 1 | 1 | 1 | 1 | 0.29 | Oryza sativa subsp. japonica |
| 19045 | 9981 | 1 | 1 | 1 | 1 | 0.33 | Triticum aestivum |
| 19045 | 10045 | 1 | 1 | 1 | 1 | 0.33 | Ricinus communis |
| 19045 | 15742 | 1 | 1 | 1 | 1 | 0.2 | Medicago truncatula |
| 19045 | 9593 | 1 | 1 | 1 | 1 | 0.34 | Arabidopsis thaliana |
| 19045 | 4081 | 1 | 1 | 1 | 1 | 0.87 | Welwitschia mirabilis |
| 19045 | 9990 | 1 | 1 | 1 | 1 | 0.33 | Arabidopsis thaliana |
| 19045 | 7939 | 1 | 1 | 1 | 1 | 0.42 | Morus indica |
| 19045 | 12965 | 1 | 1 | 1 | 1 | 0.25 | Arabidopsis thaliana |
| 19045 | 9546 | 1 | 1 | 1 | 1 | 0.34 | Chenopodium album |
| 19045 | 10462 | 1 | 1 | 1 | 1 | 0.31 | Oedogonium cardiacum |
| 19045 | 9442 | 1 | 1 | 1 | 1 | 0.35 | Betula pendula |
| 19045 | 17379 | 1 | 1 | 1 | 1 | 0.18 | Oryza sativa subsp. japonica |
| 19045 | 9375 | 1 | 1 | 1 | 1 | 0.35 | Hordeum vulgare |
| 19045 | 8525 | 1 | 1 | 1 | 1 | 0.39 | Musa acuminata |
| 19045 | 5798 | 1 | 1 | 1 | 1 | 0.6 | Oryza sativa subsp. japonica |
| 19045 | 8667 | 1 | 1 | 1 | 1 | 0.38 | Helianthus annuus |
| 19045 | 4237 | 1 | 1 | 1 | 1 | 0.85 | Solanum tuberosum |
| 19045 | 7750 | 1 | 1 | 1 | 1 | 0.43 | Piper cenocladum |
| 19045 | 7782 | 1 | 1 | 1 | 1 | 0.43 | Zea mays |
| 19045 | 16469 | 1 | 1 | 1 | 1 | 0.19 | Arabidopsis thaliana |
| 19045 | 7558 | 3 | 3 | 2 | 2 | 2.01 | Petunia hybrida |
| 19045 | 8620 | 1 | 1 | 1 | 1 | 0.38 | Petunia hybrida |
| 19045 | 5189 | 1 | 1 | 1 | 1 | 0.67 | Stigeoclonium helveticum |
| 19045 | 4774 | 1 | 1 | 1 | 1 | 0.74 | Amborella trichopoda |
| 19045 | 15408 | 1 | 1 | 1 | 1 | 0.21 | Gossypium hirsutum |
| 19045 | 15864 | 1 | 1 | 1 | 1 | 0.2 | Arabidopsis thaliana |
| 19045 | 8877 | 1 | 1 | 1 | 1 | 0.37 | Hordeum vulgare |
| 19045 | 4312 | 1 | 1 | 1 | 1 | 0.82 | Pinus strobus |
| 19045 | 9899 | 1 | 1 | 1 | 1 | 0.33 | Arabidopsis thaliana |
| 19045 | 5224 | 1 | 1 | 1 | 1 | 0.67 | Vigna unguiculata |
| 19045 | 5214 | 2 | 2 | 1 | 1 | 0.67 | Populus euphratica |
| 19045 | 6937 | 1 | 1 | 1 | 1 | 0.49 | Arabidopsis thaliana |
| 19045 | 9050 | 1 | 1 | 1 | 1 | 0.36 | Arabidopsis thaliana |
| 19045 | 4048 | 1 | 1 | 1 | 1 | 0.9 | Triticum aestivum |
| 19045 | 9709 | 1 | 1 | 1 | 1 | 0.34 | Arabidopsis thaliana |
| 19045 | 5845 | 1 | 1 | 1 | 1 | 0.58 | Arabidopsis thaliana |
| 19045 | 7251 | 1 | 1 | 1 | 1 | 0.47 | Pinus strobus |
| 19046 | 11402 | 208 | 208 | 2 | 2 | 8.46 | Arabidopsis thaliana |
| 19046 | 11460 | 143 | 143 | 1 | 1 | 6.37 | Triticum aestivum |
| 19046 | 11418 | 86 | 86 | 2 | 2 | 2.48 | Capsicum annuum |
| 19046 | 11450 | 49 | 49 | 1 | 1 | 0.28 | Chlamydomonas reinhardtii |
| 19046 | 8520 | 37 | 37 | 2 | 2 | 12.72 | Avena sativa |
| 19046 | 4481 | 29 | 29 | 1 | 1 | 0.8 | Agathis robusta |
| 19046 | 4465 | 22 | 22 | 1 | 1 | 0.8 | Pinus koraiensis |
| 19046 | 4465 | 24 | 24 | 1 | 1 | 0.8 | Marchantia polymorpha |
| 19046 | 9545 | 39 | 39 | 1 | 1 | 1.43 | Helianthus annuus |
| 19046 | 15344 | 53 | 53 | 1 | 1 | 1.13 | Encephalartos altensteinii |
| 19046 | 7971 | 22 | 22 | 1 | 1 | 3.03 | Arabis hirsuta |
| 19046 | 7995 | 19 | 19 | 1 | 1 | 1.84 | Cycas taitungensis |
| 19046 | 9531 | 33 | 33 | 1 | 1 | 2.26 | Spinacia oleracea |
| 19046 | 3815 | 26 | 26 | 2 | 2 | 56.36 | Allium textile |
| 19046 | 7985 | 16 | 16 | 1 | 1 | 0.42 | Acorus americanus |
| 19046 | 8001 | 17 | 17 | 1 | 1 | 1.84 | Ceratophyllum demersum |
| 19046 | 9381 | 19 | 19 | 1 | 1 | 0.82 | Amborella trichopoda |
| 19046 | 3833 | 26 | 26 | 2 | 2 | 25.93 | Piper cenocladum |
| 19046 | 15358 | 37 | 37 | 2 | 2 | 1.13 | Volvox carteri |
| 19046 | 7986 | 13 | 13 | 2 | 2 | 1.01 | Ipomoea purpurea |
| 19046 | 3831 | 24 | 24 | 2 | 2 | 25.93 | Pelargonium hortorum |
| 19046 | 6412 | 12 | 12 | 1 | 1 | 1.33 | Arabidopsis thaliana |
| 19046 | 9380 | 15 | 15 | 1 | 1 | 0.82 | Citrus sinensis |
| 19046 | 7463 | 11 | 11 | 1 | 1 | 0.45 | Zea mays |
| 19046 | 8648 | 10 | 10 | 1 | 1 | 0.91 | Triticum aestivum |
| 19046 | 8667 | 10 | 10 | 1 | 1 | 2.65 | Helianthus annuus |
| 19046 | 15332 | 21 | 21 | 2 | 2 | 1.58 | Medicago sativa |
| 19046 | 4165 | 10 | 10 | 1 | 1 | 5.31 | Acorus americanus |
| 19046 | 15188 | 16 | 16 | 2 | 2 | 1.59 | Arabidopsis thaliana |
| 19046 | 10464 | 6 | 6 | 1 | 1 | 1.97 | Oryza sativa subsp. japonica |
| 19046 | 9529 | 11 | 11 | 1 | 1 | 1.43 | Drimys granadensis |
| 19046 | 14873 | 52 | 52 | 2 | 2 | 613.3 | Oryza sativa subsp. indica |
| 19046 | 7366 | 10 | 10 | 1 | 1 | 2.1 | Arabidopsis thaliana |
| 19046 | 7969 | 10 | 10 | 1 | 1 | 1.84 | Agrostis stolonifera |
| 19046 | 11866 | 4 | 4 | 1 | 1 | 0.27 | Solanum bulbocastanum |
| 19046 | 9078 | 38 | 38 | 2 | 2 | 655.08 | Oryza sativa subsp. indica |
| 19046 | 15316 | 10 | 10 | 1 | 1 | 0.76 | Arabidopsis thaliana |
| 19046 | 9419 | 7 | 7 | 1 | 1 | 0.35 | Acorus calamus |
| 19046 | 10381 | 13 | 13 | 1 | 1 | 1.26 | Solanum tuberosum |
| 19046 | 8851 | 28 | 28 | 2 | 2 | 761.23 | Oryza sativa subsp. japonica |
| 19046 | 11994 | 9 | 9 | 1 | 1 | 1.05 | Cannabis sativa |
| 19046 | 8031 | 7 | 7 | 2 | 2 | 3.03 | Atropa belladonna |
| 19046 | 12553 | 5 | 5 | 1 | 1 | 0.58 | Lupinus luteus |
| 19046 | 3967 | 11 | 11 | 2 | 2 | 12.1 | Zygnema circumcarinatum |
| 19046 | 9253 | 26 | 26 | 2 | 2 | 178.85 | Solanum lycopersicum |
| 19046 | 3813 | 9 | 9 | 1 | 1 | 13.87 | Lotus japonicus |
| 19046 | 9282 | 20 | 20 | 2 | 2 | 91.82 | Arabidopsis thaliana |
| 19046 | 10435 | 3 | 3 | 1 | 1 | 0.31 | Gossypium hirsutum |
| 19046 | 15406 | 7 | 7 | 1 | 1 | 0.21 | Arabidopsis thaliana |
| 19046 | 9353 | 6 | 6 | 1 | 1 | 0.83 | Mesembryanthemum crystallinum |
| 19046 | 10536 | 9 | 9 | 1 | 1 | 0.71 | Mercurialis perennis |
| 19046 | 9220 | 4 | 4 | 1 | 1 | 0.84 | Ostreococcus tauri |
| 19046 | 8192 | 8 | 8 | 1 | 1 | 0.97 | Solanum lycopersicum |
| 19046 | 9183 | 14 | 14 | 2 | 2 | 52.01 | Chlamydomonas reinhardtii |
| 19046 | 9635 | 10 | 10 | 2 | 2 | 17.61 | Zea mays |
| 19046 | 9593 | 7 | 7 | 2 | 2 | 3.38 | Arabidopsis thaliana |
| 19046 | 6883 | 3 | 3 | 1 | 1 | 1.22 | Arabidopsis thaliana |
| 19046 | 8211 | 12 | 12 | 2 | 2 | 57.84 | Arabidopsis thaliana |
| 19046 | 17483 | 1 | 1 | 1 | 1 | 0.18 | Senecio vernalis |
| 19046 | 9001 | 9 | 9 | 2 | 2 | 5.52 | Beta vulgaris |
| 19046 | 7251 | 9 | 9 | 6 | 6 | 30.21 | Pinus strobus |
| 19046 | 13909 | 3 | 3 | 1 | 1 | 0.52 | Oryza sativa subsp. indica |
| 19046 | 4180 | 4 | 4 | 1 | 1 | 2.42 | Lepidium virginicum |
| 19046 | 11194 | 4 | 4 | 1 | 1 | 1.14 | Chlamydomonas reinhardtii |
| 19046 | 15357 | 5 | 5 | 2 | 2 | 1.13 | Oryza sativa subsp. indica |
| 19046 | 10045 | 9 | 9 | 1 | 1 | 4.47 | Ricinus communis |
| 19046 | 4128 | 2 | 2 | 1 | 1 | 0.87 | Aethionema cordifolium |
| 19046 | 10875 | 6 | 6 | 2 | 2 | 1.85 | Arabidopsis thaliana |
| 19046 | 10242 | 4 | 4 | 1 | 1 | 0.32 | Oryza sativa subsp. japonica |
| 19046 | 9374 | 8 | 8 | 2 | 2 | 10.21 | Amaranthus retroflexus |
| 19046 | 4142 | 2 | 2 | 2 | 2 | 2.51 | Gnetum parvifolium |
| 19046 | 8932 | 8 | 8 | 2 | 2 | 8.13 | Oryza sativa subsp. indica |
| 19046 | 15318 | 2 | 2 | 1 | 1 | 0.21 | Lilium longiflorum |
| 19046 | 12527 | 4 | 4 | 2 | 2 | 1.5 | Zea mays |
| 19046 | 13968 | 3 | 3 | 1 | 1 | 0.51 | Oryza sativa subsp. indica |
| 19046 | 4114 | 2 | 2 | 1 | 1 | 0.87 | Arabidopsis thaliana |
| 19046 | 8059 | 3 | 3 | 1 | 1 | 1.81 | Chlorokybus atmophyticus |
| 19046 | 9341 | 7 | 7 | 2 | 2 | 7.28 | Arabidopsis thaliana |
| 19046 | 9254 | 3 | 3 | 2 | 2 | 1.5 | Arabidopsis thaliana |
| 19046 | 8037 | 4 | 4 | 1 | 1 | 1.01 | Ipomoea batatas |
| 19046 | 13830 | 6 | 6 | 1 | 1 | 1.83 | Oryza sativa subsp. japonica |
| 19046 | 10434 | 3 | 3 | 1 | 1 | 0.31 | Capsella bursa-pastoris |
| 19046 | 9789 | 3 | 3 | 2 | 2 | 0.78 | Arabidopsis thaliana |
| 19046 | 3910 | 10 | 10 | 7 | 7 | 193.23 | Solanum tuberosum |
| 19046 | 4293 | 2 | 2 | 1 | 1 | 0.82 | Jasminum nudiflorum |
| 19046 | 9906 | 1 | 1 | 1 | 1 | 0.33 | Arabidopsis thaliana |
| 19046 | 14535 | 3 | 3 | 2 | 2 | 0.82 | Arabidopsis thaliana |
| 19046 | 15467 | 4 | 4 | 1 | 1 | 0.76 | Arabidopsis thaliana |
| 19046 | 15719 | 1 | 1 | 1 | 1 | 0.2 | Arabidopsis thaliana |
| 19046 | 4782 | 2 | 2 | 1 | 1 | 2.02 | Lemna minor |
| 19046 | 8912 | 4 | 4 | 2 | 2 | 2.54 | Medicago sativa |
| 19046 | 4008 | 4 | 4 | 2 | 2 | 5.89 | Morus indica |
| 19046 | 13528 | 5 | 5 | 2 | 2 | 1.9 | Oryza sativa subsp. indica |
| 19046 | 14182 | 3 | 3 | 1 | 1 | 0.5 | Olea europaea |
| 19046 | 3935 | 6 | 6 | 1 | 1 | 12.94 | Calycanthus floridus var. glaucus |
| 19046 | 11150 | 3 | 3 | 2 | 2 | 1.16 | Arabidopsis thaliana |
| 19046 | 3931 | 3 | 3 | 1 | 1 | 0.93 | Acorus gramineus |
| 19046 | 10668 | 2 | 2 | 1 | 1 | 0.31 | Solanum lyratum |
| 19046 | 11232 | 5 | 5 | 2 | 2 | 2.57 | Arabidopsis thaliana |
| 19046 | 8955 | 5 | 5 | 1 | 1 | 3.77 | Arabidopsis thaliana |
| 19046 | 11150 | 5 | 5 | 2 | 2 | 1.16 | Oryza sativa subsp. japonica |
| 19046 | 3975 | 6 | 6 | 1 | 1 | 5.89 | Phalaenopsis aphrodite subsp. |
| | | | | | | | formosana |
| 19046 | 9763 | 3 | 3 | 1 | 1 | 0.78 | Ricinus communis |
| 19046 | 17538 | 1 | 1 | 1 | 1 | 0.18 | Gossypium barbadense |
| 19046 | 11292 | 4 | 4 | 2 | 2 | 1.74 | Vernicia fordii |
| 19046 | 8172 | 5 | 5 | 1 | 1 | 4.46 | Stigeoclonium helveticum |
| 19046 | 15363 | 2 | 2 | 1 | 1 | 0.21 | Arabidopsis thaliana |
| 19046 | 4005 | 2 | 2 | 1 | 1 | 2.62 | Hordeum vulgare |
| 19046 | 9014 | 2 | 2 | 1 | 1 | 0.87 | Arabidopsis thaliana |
| 19046 | 12505 | 2 | 2 | 1 | 1 | 0.58 | Euphorbia esula |
| 19046 | 7895 | 2 | 2 | 1 | 1 | 1.02 | Aneura mirabilis |
| 19046 | 8679 | 3 | 3 | 2 | 2 | 1.64 | Triticum aestivum |
| 19046 | 11283 | 2 | 2 | 1 | 1 | 0.66 | Brassica campestris |
| 19046 | 9821 | 2 | 2 | 1 | 1 | 0.34 | Arabidopsis thaliana |
| 19046 | 12630 | 2 | 2 | 1 | 1 | 0.57 | Arabidopsis thaliana |
| 19046 | 13709 | 3 | 3 | 2 | 2 | 0.87 | Arabidopsis thaliana |
| 19046 | 10496 | 1 | 1 | 1 | 1 | 0.31 | Morus indica |
| 19046 | 7500 | 3 | 3 | 1 | 1 | 1.08 | Pisum sativum |
| 19046 | 8262 | 4 | 4 | 2 | 2 | 2.89 | Helianthus annuus |
| 19046 | 11139 | 1 | 1 | 1 | 1 | 0.29 | Chlorokybus atmophyticus |
| 19046 | 9046 | 2 | 2 | 1 | 1 | 0.87 | Solanum lycopersicum |
| 19046 | 12795 | 3 | 3 | 2 | 2 | 0.96 | Arabidopsis thaliana |
| 19046 | 8853 | 5 | 5 | 2 | 2 | 3.86 | Stigeoclonium helveticum |
| 19046 | 11220 | 2 | 2 | 2 | 2 | 0.66 | Arabidopsis thaliana |
| 19046 | 15632 | 2 | 2 | 2 | 2 | 0.45 | Arabidopsis thaliana |
| 19046 | 4744 | 2 | 2 | 1 | 1 | 0.74 | Acorus calamus |
| 19046 | 11470 | 1 | 1 | 1 | 1 | 0.28 | Zea mays |
| 19046 | 10941 | 1 | 1 | 1 | 1 | 0.3 | Glycine max |
| 19046 | 15215 | 1 | 1 | 1 | 1 | 0.21 | Arabidopsis thaliana |
| 19046 | 17502 | 1 | 1 | 1 | 1 | 0.18 | Medicago sativa |
| 19046 | 10410 | 3 | 3 | 1 | 1 | 0.72 | Lactuca sativa |
| 19046 | 5845 | 3 | 3 | 2 | 2 | 2.97 | Arabidopsis thaliana |
| 19046 | 11215 | 1 | 1 | 1 | 1 | 0.29 | Arabidopsis thaliana |
| 19046 | 9515 | 2 | 2 | 1 | 1 | 0.81 | Pinus thunbergii |
| 19046 | 11015 | 1 | 1 | 1 | 1 | 0.3 | Arabidopsis thaliana |
| 19046 | 14558 | 1 | 1 | 1 | 1 | 0.22 | Olea europaea |
| 19046 | 4726 | 3 | 3 | 2 | 2 | 2.02 | Chloranthus spicatus |
| 19046 | 7697 | 2 | 2 | 1 | 1 | 0.44 | Arabidopsis thaliana |
| 19046 | 13537 | 1 | 1 | 1 | 1 | 0.24 | Oryza sativa subsp. japonica |
| 19046 | 9444 | 3 | 3 | 1 | 1 | 0.82 | Zea mays |
| 19046 | 24369 | 2 | 2 | 1 | 1 | 0.27 | Pisum sativum |
| 19046 | 15902 | 1 | 1 | 1 | 1 | 0.2 | Arabidopsis thaliana |
| 19046 | 9136 | 7 | 7 | 2 | 2 | 5.38 | Tetradesmus obliquus |
| 19046 | 10002 | 2 | 2 | 1 | 1 | 0.76 | Oryza sativa subsp. indica |
| 19046 | 16310 | 1 | 1 | 1 | 1 | 0.19 | Zea mays |
| 19046 | 7734 | 2 | 2 | 1 | 1 | 1.04 | Daucus carota |
| 19046 | 8901 | 1 | 1 | 1 | 1 | 0.37 | Brassica rapa subsp. pekinensis |
| 19046 | 14208 | 1 | 1 | 1 | 1 | 0.23 | Phleum pratense |
| 19046 | 17105 | 1 | 1 | 1 | 1 | 0.19 | Oryza sativa subsp. indica |
| 19046 | 13854 | 1 | 1 | 1 | 1 | 0.23 | Oryza sativa subsp. japonica |
| 19046 | 10511 | 1 | 1 | 1 | 1 | 0.31 | Gleichenia japonica |
| 19046 | 9957 | 3 | 3 | 1 | 1 | 1.34 | Triticum aestivum |
| 19046 | 9671 | 1 | 1 | 1 | 1 | 0.34 | Arabidopsis thaliana |
| 19046 | 7529 | 2 | 2 | 1 | 1 | 1.08 | Marchantia polymorpha |
| 19046 | 14300 | 1 | 1 | 1 | 1 | 0.22 | Olea europaea |
| 19046 | 4464 | 2 | 2 | 1 | 1 | 0.8 | Cedrus deodara |
| 19046 | 14266 | 1 | 1 | 1 | 1 | 0.22 | Corylus avellana |
| 19046 | 12300 | 1 | 1 | 1 | 1 | 0.26 | Daucus carota |
| 19046 | 8852 | 1 | 1 | 1 | 1 | 0.37 | Cynodon dactylon |
| 19046 | 14347 | 1 | 1 | 1 | 1 | 0.22 | Arabidopsis thaliana |
| 19046 | 4080 | 1 | 1 | 1 | 1 | 0.87 | Tupiella akineta |
| 19046 | 13726 | 1 | 1 | 1 | 1 | 0.23 | Arabidopsis thaliana |
| 19046 | 10789 | 1 | 1 | 1 | 1 | 0.3 | Arabidopsis thaliana |
| 19046 | 14651 | 1 | 1 | 1 | 1 | 0.22 | Arabidopsis thaliana |
| 19046 | 9042 | 1 | 1 | 1 | 1 | 0.37 | Arabidopsis thaliana |
| 19046 | 8877 | 1 | 1 | 1 | 1 | 0.37 | Hordeum vulgare |
| 19046 | 7742 | 1 | 1 | 1 | 1 | 0.43 | Coffea arabica |
| 19046 | 11065 | 2 | 2 | 1 | 1 | 0.67 | Gossypium hirsutum |
| 19046 | 11192 | 2 | 2 | 2 | 2 | 0.66 | Arabidopsis thaliana |
| 19046 | 14269 | 1 | 1 | 1 | 1 | 0.22 | Phleum pratense |
| 19046 | 15329 | 1 | 1 | 1 | 1 | 0.21 | Arabidopsis thaliana |
| 19046 | 17504 | 1 | 1 | 1 | 1 | 0.18 | Atropa belladonna |
| 19046 | 14176 | 2 | 2 | 1 | 1 | 0.5 | Lilium longiflorum |
| 19046 | 14143 | 1 | 1 | 1 | 1 | 0.23 | Olea europaea |
| 19046 | 14604 | 1 | 1 | 1 | 1 | 0.22 | Lactuca sativa |
| 19046 | 10372 | 2 | 2 | 1 | 1 | 0.73 | Arabidopsis thaliana |
| 19046 | 4134 | 2 | 2 | 1 | 1 | 2.51 | Amborella trichopoda |
| 19046 | 16351 | 1 | 1 | 1 | 1 | 0.19 | Brassica napus |
| 19046 | 10105 | 2 | 2 | 2 | 2 | 0.75 | Chlamydomonas reinhardtii |
| 19046 | 14514 | 1 | 1 | 1 | 1 | 0.22 | Casuarina glauca |
| 19046 | 4476 | 3 | 3 | 1 | 1 | 2.24 | Huperzia lucidula |
| 19046 | 14608 | 1 | 1 | 1 | 1 | 0.22 | Olea europaea |
| 19046 | 4112 | 3 | 3 | 1 | 1 | 2.51 | Oenothera elata subsp. hookeri |
| 19046 | 8425 | 3 | 3 | 2 | 2 | 1.7 | Tupiella akineta |
| 19046 | 11752 | 2 | 2 | 2 | 2 | 0.63 | Arabidopsis thaliana |
| 19046 | 15276 | 1 | 1 | 1 | 1 | 0.21 | Brassica oleracea var. capitata |
| 19046 | 14199 | 1 | 1 | 1 | 1 | 0.23 | Olea europaea |
| 19046 | 14553 | 1 | 1 | 1 | 1 | 0.22 | Parietaria judaica |
| 19046 | 9027 | 2 | 2 | 2 | 2 | 0.87 | Arabidopsis thaliana |
| 19046 | 9798 | 2 | 2 | 1 | 1 | 0.34 | Fritillaria agrestis |
| 19046 | 10993 | 2 | 2 | 1 | 1 | 0.3 | Arabidopsis thaliana |
| 19046 | 8525 | 1 | 1 | 1 | 1 | 0.39 | Musa acuminata |
| 19046 | 8972 | 3 | 3 | 1 | 1 | 0.87 | Arabidopsis thaliana |
| 19046 | 7337 | 1 | 1 | 1 | 1 | 0.46 | Arabidopsis thaliana |
| 19046 | 9457 | 1 | 1 | 1 | 1 | 0.35 | Oryza sativa subsp. japonica |
| 19046 | 14169 | 2 | 2 | 1 | 1 | 0.5 | Pyrus communis |
| 19046 | 11989 | 1 | 1 | 1 | 1 | 0.27 | Arabidopsis thaliana |
| 19046 | 3056 | 3 | 3 | 3 | 3 | 9.77 | Spinacia oleracea |
| 19046 | 4301 | 1 | 1 | 1 | 1 | 0.82 | Mesostigma viride |
| 19046 | 9395 | 1 | 1 | 1 | 1 | 0.35 | Bryopsis maxima |
| 19046 | 8653 | 1 | 1 | 1 | 1 | 0.38 | Chassalia chartacea |
| 19046 | 8169 | 1 | 1 | 1 | 1 | 0.4 | Arabidopsis thaliana |
| 19046 | 9709 | 1 | 1 | 1 | 1 | 0.34 | Arabidopsis thaliana |
| 19046 | 4158 | 2 | 2 | 1 | 1 | 0.87 | Eucalyptus globulus subsp. globulus |
| 19046 | 10506 | 1 | 1 | 1 | 1 | 0.31 | Scenedesmus quadricauda |
| 19046 | 7789 | 3 | 3 | 1 | 1 | 1.92 | Petunia sp. |
| 19046 | 10425 | 1 | 1 | 1 | 1 | 0.31 | Arabidopsis thaliana |
| 19046 | 11580 | 1 | 1 | 1 | 1 | 0.28 | Oryza sativa subsp. japonica |
| 19046 | 9457 | 1 | 1 | 1 | 1 | 0.35 | Arabidopsis thaliana |
| 19046 | 8027 | 1 | 1 | 1 | 1 | 0.42 | Pisum sativum |
| 19046 | 8357 | 2 | 2 | 1 | 1 | 0.96 | Arabidopsis thaliana |
| 19046 | 9239 | 1 | 1 | 1 | 1 | 0.36 | Tupiella akineta |
| 19046 | 10159 | 1 | 1 | 1 | 1 | 0.32 | Oryza sativa subsp. japonica |
| 19046 | 8728 | 1 | 1 | 1 | 1 | 0.38 | Phleum pratense |
| 19046 | 7923 | 1 | 1 | 1 | 1 | 0.42 | Marchantia polymorpha |
| 19046 | 8321 | 1 | 1 | 1 | 1 | 0.4 | Arabidopsis thaliana |
| 19046 | 9779 | 1 | 1 | 1 | 1 | 0.34 | Arabidopsis thaliana |
| 19046 | 9953 | 1 | 1 | 1 | 1 | 0.33 | Arabidopsis thaliana |
| 19046 | 12056 | 1 | 1 | 1 | 1 | 0.27 | Arabidopsis thaliana |
| 19046 | 7695 | 1 | 1 | 1 | 1 | 0.44 | Phalaenopsis aphrodite subsp. |
| | | | | | | | formosana |
| 19046 | 8337 | 1 | 1 | 1 | 1 | 0.4 | Spirogyra maxima |
| 19046 | 8278 | 1 | 1 | 1 | 1 | 0.4 | Oenothera ammophila |
| 19046 | 7141 | 2 | 2 | 1 | 1 | 1.17 | Phytolacca americana |
| 19046 | 9319 | 1 | 1 | 1 | 1 | 0.35 | Zygnema circumcarinatum |
| 19046 | 7732 | 1 | 1 | 1 | 1 | 0.43 | Calycanthus floridus var. glaucus |
| 19046 | 4287 | 4 | 4 | 1 | 1 | 2.32 | Chlamydomonas reinhardtii |
| 19046 | 3584 | 1 | 1 | 1 | 1 | 1.03 | Cucumis sativus |
| 19046 | 9158 | 2 | 2 | 1 | 1 | 0.84 | Oryza sativa subsp. japonica |
| 19046 | 8057 | 1 | 1 | 1 | 1 | 0.41 | Cicer arietinum |
| 19046 | 3049 | 2 | 2 | 1 | 1 | 4.11 | Pseudotsuga menziesii |
| 19046 | 7558 | 3 | 3 | 2 | 2 | 2.01 | Petunia hybrida |
| 19046 | 4086 | 2 | 2 | 1 | 1 | 2.51 | Aethionema grandiflorum |
| 19046 | 8874 | 1 | 1 | 1 | 1 | 0.37 | Arabidopsis thaliana |
| 19046 | 7814 | 1 | 1 | 1 | 1 | 0.43 | Drimys granadensis |
| 19046 | 8440 | 1 | 1 | 1 | 1 | 0.39 | Chara vulgaris |
| 19046 | 7725 | 1 | 1 | 1 | 1 | 0.43 | Helianthus annuus |
| 19046 | 12897 | 1 | 1 | 1 | 1 | 0.25 | Arabidopsis thaliana |
| 19046 | 8957 | 1 | 1 | 1 | 1 | 0.37 | Arabidopsis thaliana |
| 19046 | 3868 | 1 | 1 | 1 | 1 | 0.93 | Pinus thunbergii |
| 19046 | 6358 | 1 | 1 | 1 | 1 | 0.54 | Arabidopsis thaliana |
| 19046 | 8644 | 1 | 1 | 1 | 1 | 0.38 | Oryza sativa subsp. japonica |
| 19046 | 4460 | 1 | 1 | 1 | 1 | 0.8 | Adiantum capillus-veneris |
| 19046 | 8676 | 1 | 1 | 1 | 1 | 0.38 | Triticum aestivum |
| 19046 | 4146 | 1 | 1 | 1 | 1 | 0.87 | Cycas taitungensis |
| 19046 | 8175 | 1 | 1 | 1 | 1 | 0.4 | Oedogonium cardiacum |
| 19046 | 7104 | 2 | 2 | 1 | 1 | 1.17 | Arabidopsis thaliana |
| 19046 | 8133 | 1 | 1 | 1 | 1 | 0.41 | Psilotum nudum |
| 19046 | 7472 | 2 | 2 | 1 | 1 | 1.1 | Brassica napus |
| 19046 | 4114 | 1 | 1 | 1 | 1 | 0.87 | Agrostis stolonifera |
| 19046 | 4467 | 1 | 1 | 1 | 1 | 0.8 | Antirrhinum majus |
| 19046 | 6570 | 1 | 1 | 1 | 1 | 0.52 | Arabidopsis thaliana |
| 19046 | 4084 | 2 | 2 | 1 | 1 | 0.87 | Hordeum jubatum |
| 19046 | 4048 | 1 | 1 | 1 | 1 | 0.9 | Triticum aestivum |
| 19046 | 6537 | 1 | 1 | 1 | 1 | 0.52 | Acorus gramineus |
| 19046 | 4133 | 1 | 1 | 1 | 1 | 0.87 | Psilotum nudum |
| 19046 | 4071 | 1 | 1 | 1 | 1 | 0.87 | Solanum tuberosum |
| 19046 | 4153 | 1 | 1 | 1 | 1 | 0.87 | Platanus occidentalis |
| 19046 | 3947 | 2 | 2 | 2 | 2 | 2.62 | Chlorella vulgaris |
| 19046 | 4172 | 1 | 1 | 1 | 1 | 0.85 | Cuscuta exaltata |
| 19046 | 6442 | 1 | 1 | 1 | 1 | 0.53 | Pinus thunbergii |
| 19046 | 10558 | 1 | 1 | 1 | 1 | 0.31 | Arabidopsis thaliana |
| 19046 | 3782 | 1 | 1 | 1 | 1 | 0.96 | Chlamydomonas reinhardtii |
| 19046 | 10137 | 1 | 1 | 1 | 1 | 0.32 | Oryza sativa subsp. japonica |
| 19046 | 28873 | 1 | 1 | 1 | 1 | 0.11 | Petunia hybrida |
| 19046 | 4673 | 1 | 1 | 1 | 1 | 0.76 | Calycanthus floridus |
| 19046 | 6085 | 1 | 1 | 1 | 1 | 0.56 | Arabidopsis thaliana |
| 19046 | 9279 | 1 | 1 | 1 | 1 | 0.35 | Physcomitrella patens subsp. patens |
| 19046 | 9733 | 1 | 1 | 1 | 1 | 0.34 | Vitis sp. |
| 19046 | 9603 | 1 | 1 | 1 | 1 | 0.34 | Arabidopsis thaliana |
| 19046 | 6762 | 1 | 1 | 1 | 1 | 0.5 | Solanum torvum |
| 19046 | 9112 | 1 | 1 | 1 | 1 | 0.36 | Arabidopsis thaliana |
| 19046 | 5798 | 1 | 1 | 1 | 1 | 0.6 | Oryza sativa subsp. japonica |
| 19046 | 4537 | 1 | 1 | 1 | 1 | 0.78 | Raphanus sativus |
| 19046 | 5122 | 1 | 1 | 1 | 1 | 0.68 | Populus euphratica |

Swissprot was also searched using the least stringent fragment tolerance (±2 Da) and a decoy method. Without any dynamic modification set, searching the whole taxonomy yielded 94 accessions with 998 (9%) MS/MS matches, while searching only viridiplantae taxonomy (39,800 entries) yielded 80 hits (1181 (10%) matches). Searching viridiplantae taxonomy and setting Protein N-term acetylation and Met oxidation as dynamic modifications listed 141 accessions (1352 (12%) matches). Finally, by searching viridiplantae taxonomy but adding phosphorylations of Ser and Tyr residues as dynamic modification generated 274 accessions (1863 (17%) matches). The latter search lasted the longest (53 h) (Tables 7 and 14). Therefore, while the list of proteins extended when using a bigger database in conjunction with more relaxed mass tolerances, confidence in the identified proteins was relatively low. Accordingly, the search results obtain from the uniprotKB data, with a stringent fragment tolerance (±50 ppm) (Table 13), was selected to continue this study.

The masses of the 21 identified proteins range from 4.1 kD to 17.6 kD. Thirteen accessions had a Mascot score above 100, and 16 accessions were identified using more than one MS/MS spectrum (Tables 13 and 15). No missed cleavage was found (M>0), possibly explaining the low number of identified proteins.

TABLE 15
List of proteoforms identified from protein standards samples using Mascot algorithm
with 50 ppm fragment tolerance and UniProtKB C. sativa database

| Job | | | | | | | | | |
|---|---|---|---|---|---|---|---|---|---|
| no. | Description | Accession | Score | Mass | Matches | Seqs | emPAI | Query | Dupes |
| 19030 | Cytochrome b559 | A0A0C5ARS8_CANSA | 2265 | 9367 | 37 | 1 | 0.83 | 3456 | 34 |
| | subunit alpha | | | | | | | | |
| 19030 | Cytochrome b559 | A0A0C5ARS8_CANSA | 2265 | 9367 | 37 | 1 | 0.83 | 3543 | 1 |
| | subunit alpha | | | | | | | | |
| 19030 | Photosystem I | A0A0C5AS17_CANSA | 1664 | 9545 | 39 | 1 | 1.43 | 3918 | |
| | iron-sulfur center | | | | | | | | |
| 19030 | Photosystem I | A0A0C5AS17_CANSA | 1664 | 9545 | 39 | 1 | 1.43 | 3925 | 26 |
| | iron-sulfur center | | | | | | | | |
| 19030 | Photosystem I | A0A0C5AS17_CANSA | 1664 | 9545 | 39 | 1 | 1.43 | 3970 | 10 |
| | iron-sulfur center | | | | | | | | |
| 19030 | Photosystem II | A0A0U2DTK8_CANSA | 1555 | 3815 | 25 | 1 | 13.87 | 198 | 10 |
| | reaction center | | | | | | | | |
| | protein T | | | | | | | | |
| 19030 | Photosystem II | A0A0C5B2J7_CANSA | 1348 | 7645 | 12 | 1 | 1.06 | 1878 | 8 |
| | reaction center | | | | | | | | |
| | protein H | | | | | | | | |
| 19030 | Photosystem II | A0A0C5B2J7_CANSA | 1348 | 7645 | 12 | 1 | 1.06 | 1886 | 2 |
| | reaction center | | | | | | | | |
| | protein H | | | | | | | | |
| 19030 | Cytochrome b559 | A0A0U2GZT5_HUMLU | 902 | 9381 | 21 | 1 | 0.35 | 3456 | 20 |
| | subunit alpha | | | | | | | | |
| 19030 | Photosystem II | A0A0C5APX7_CANSA | 292 | 4165 | 9 | 1 | 5.31 | 547 | 2 |
| | reaction center | | | | | | | | |
| | protein I | | | | | | | | |
| 19030 | Photosystem II | A0A0C5APX7_CANSA | 292 | 4165 | 9 | 1 | 5.31 | 550 | 4 |
| | reaction center | | | | | | | | |
| | protein I | | | | | | | | |
| 19030 | ATP synthase | A0A0C5ARQ5_CANSA | 272 | 7985 | 12 | 1 | 1.84 | 2264 | 5 |
| | CF0 C subunit | | | | | | | | |
| 19030 | ATP synthase | A0A0C5ARQ5_CANSA | 272 | 7985 | 12 | 1 | 1.84 | 2273 | 3 |
| | CF0 C subunit | | | | | | | | |
| 19030 | ATP synthase | A0A0C5ARQ5_CANSA | 272 | 7985 | 12 | 1 | 1.84 | 2332 | 1 |
| | CF0 C subunit | | | | | | | | |
| 19030 | 30S ribosomal | A0A0U2H3A0A0U2H3S7_HUMLU | 182 | 11833 | 5 | 1 | 0.62 | 6673 | 2 |
| | protein S14, | | | | | | | | |
| | chloroplastic | | | | | | | | |
| 19030 | 30S ribosomal | A0A0U2H3S7_HUMLU | 182 | 11833 | 5 | 1 | 0.62 | 6681 | 1 |
| | protein S14, | | | | | | | | |
| | chloroplastic | | | | | | | | |
| 19030 | Cytochrome b559 | A0A0C5AUI2_CANSA | 182 | 4421 | 17 | 1 | 0.8 | 740 | 16 |
| | subunit beta | | | | | | | | |
| 19030 | Olivetolic acid | OLIAC_CANSA | 162 | 11994 | 9 | 1 | 0.61 | 6725 | 7 |
| | cyclase | | | | | | | | |
| 19030 | Olivetolic acid | OLIAC_CANSA | 162 | 11994 | 9 | 1 | 0.61 | 6795 | |
| | cyclase | | | | | | | | |
| 19030 | Ribosomal | A0A0H3W6G0_CANSA | 123 | 10414 | 5 | 1 | 0.72 | 5400 | 1 |
| | protein S16 | | | | | | | | |
| 19030 | Ribosomal | A0A0H3W6G0_CANSA | 123 | 10414 | 5 | 1 | 0.72 | 5402 | |
| | protein S16 | | | | | | | | |
| 19030 | Ribosomal | A0A0H3W6G0_CANSA | 123 | 10414 | 5 | 1 | 0.72 | 5405 | 3 |
| | protein S16 | | | | | | | | |
| 19030 | Betv1-like | I6XT51_CANSA | 113 | 17597 | 7 | 2 | 1.28 | 10077 | 1 |
| | protein | | | | | | | | |
| 19030 | Betv1-like | I6XT51_CANSA | 113 | 17597 | 7 | 2 | 1.28 | 10081 | |
| | protein | | | | | | | | |
| 19030 | Betv1-like | I6XT51_CANSA | 113 | 17597 | 7 | 2 | 1.28 | 10082 | |
| | protein | | | | | | | | |
| 19030 | Betv1-like | I6XT51_CANSA | 113 | 17597 | 7 | 2 | 1.28 | 10100 | 1 |
| | protein | | | | | | | | |
| 19030 | Photosystem II | A0A0C5APY3_CANSA | 79 | 4128 | 2 | 1 | 0.87 | 553 | 1 |
| | reaction center | | | | | | | | |
| | protein J | | | | | | | | |
| 19030 | Ribosomal | A0A0C5AUI5_CANSA | 72 | 7910 | 1 | 1 | 0.42 | 2163 | |
| | protein L33 | | | | | | | | |
| 19030 | ATP synthase | A0A0C5AUH9_CANSA | 62 | 14696 | 1 | 1 | 0.22 | 8145 | |
| | CF1 epsilon | | | | | | | | |
| | subunit | | | | | | | | |
| 19030 | Cytochrome b6-f | A0A0C5APY4_CANSA | 27 | 4167 | 1 | 1 | 0.85 | 559 | |
| | complex | | | | | | | | |
| | subunit 5 | | | | | | | | |
| 19030 | Non-specific | W0U0V5_CANSA | 26 | 9489 | 2 | 1 | 0.35 | 4269 | 1 |
| | lipid-transfer | | | | | | | | |
| | protein | | | | | | | | |
| 19030 | Photosystem II | A0A0H3W8G1_CANSA | 25 | 4494 | 2 | 1 | 0.8 | 686 | 1 |
| | reaction center | | | | | | | | |
| | protein L | | | | | | | | |
| 19030 | Cytochrome b6-f | A0A0H3W844_CANSA | 24 | 17504 | 1 | 1 | 0.18 | 10025 | |
| | complex | | | | | | | | |
| | subunit 4 | | | | | | | | |
| 19030 | Photosystem I | A0A0C5AS04_CANSA | 15 | 4770 | 1 | 1 | 0.74 | 1002 | |
| | reaction center | | | | | | | | |
| | subunit IX | | | | | | | | |

| Job | | Mr | Mr | | | | | | | SEQ |
|---|---|---|---|---|---|---|---|---|---|---|
| no. | Observed | (expt) | (calc) | % | M | Score | Expect | Rank | U | ID: |
| 19030 | 9237.666 | 9236.658 | 9235.647 | 0.011 | 0 | 197 | 1.90E−20 | 1 | U | 285 |
| 19030 | 9278.672 | 9277.665 | 9277.657 | 0.000 | 0 | 31 | 0.00072 | 1 | U | 286 |
| 19030 | 9416.363 | 9415.356 | 9446.328 | −0.328 | 0 | 20 | 0.018 | 1 | U | 287 |
| 19030 | 9416.378 | 9415.371 | 9414.338 | 0.011 | 0 | 170 | 1.80E−17 | 1 | U | 288 |
| 19030 | 9416.458 | 9415.451 | 9430.333 | −0.158 | 0 | 150 | 2.10E−15 | 1 | U | 289 |
| 19030 | 3844.163 | 3843.156 | 3815.150 | 0.734 | 0 | 138 | 1.70E−14 | 1 | U | 290 |
| 19030 | 7515.975 | 7514.968 | 7529.904 | −0.198 | 0 | 188 | 1.70E−19 | 1 | U | 291 |
| 19030 | 7516.017 | 7515.010 | 7513.909 | 0.015 | 0 | 239 | 1.30E−24 | 1 | U | 292 |
| 19030 | 9237.666 | 9236.658 | 9249.662 | −0.141 | 0 | 91 | 7.70E−10 | 3 | U | 293 |
| 19030 | 4194.221 | 4193.214 | 4165.212 | 0.672 | 0 | 89 | 2.20E−09 | 1 | U | 294 |
| 19030 | 4194.248 | 4193.240 | 4223.217 | −0.710 | 0 | 79 | 2.30E−08 | 1 | U | 295 |
| 19030 | 8015.408 | 8014.400 | 8043.399 | −0.361 | 0 | 49 | 1.40E−05 | 1 | U | 296 |
| 19030 | 8015.472 | 8014.464 | 7985.393 | 0.364 | 0 | 54 | 5.00E−06 | 1 | U | 297 |
| 19030 | 8031.495 | 8030.488 | 8001.388 | 0.364 | 0 | 53 | 6.00E−06 | 1 | U | 298 |
| 19030 | 11721.470 | 11720.463 | 11702.389 | 0.154 | 0 | 68 | 4.10E−07 | 1 | U | 299 |
| 19030 | 11721.561 | 11720.554 | 11718.384 | 0.019 | 0 | 55 | 8.20E−06 | 1 | U | 300 |
| 19030 | 4393.373 | 4392.365 | 4421.355 | −0.656 | 0 | 31 | 0.00073 | 1 | U | 301 |
| 19030 | 11869.288 | 11868.280 | 11863.163 | 0.043 | 0 | 54 | 1.90E−05 | 1 | U | 302 |
| 19030 | 11910.306 | 11909.299 | 11905.174 | 0.035 | 0 | 54 | 1.90E−05 | 1 | U | 303 |
| 19030 | 10442.950 | 10441.942 | 10379.805 | 0.599 | 0 | 70 | 6.10E−07 | 1 | U | 304 |
| 19030 | 10442.953 | 10441.946 | 10429.784 | 0.117 | 0 | 29 | 0.0084 | 1 | U | 305 |
| 19030 | 10444.951 | 10443.943 | 10413.789 | 0.290 | 0 | 63 | 3.30E−06 | 1 | U | 306 |
| 19030 | 17491.194 | 17490.187 | 17466.018 | 0.138 | 0 | 46 | 0.00017 | 1 | U | 307 |
| 19030 | 17491.212 | 17490.205 | 17613.053 | −0.698 | 0 | 29 | 0.0017 | 1 | U | 308 |
| 19030 | 17491.212 | 17490.205 | 17597.058 | −0.607 | 0 | 29 | 0.0021 | 1 | U | 309 |
| 19030 | 17492.208 | 17491.201 | 17508.028 | −0.096 | 0 | 27 | 0.0032 | 4 | U | 310 |
| 19030 | 4194.259 | 4193.252 | 4170.248 | 0.552 | 0 | 66 | 4.30E−07 | 1 | U | 311 |
| 19030 | 7781.137 | 7780.129 | 7779.095 | 0.013 | 0 | 72 | 7.20E−08 | 1 | U | 312 |
| 19030 | 14615.867 | 14614.860 | 14622.683 | −0.054 | 0 | 62 | 3.20E−06 | 1 | U | 313 |
| 19030 | 4196.345 | 4195.338 | 4167.321 | 0.672 | 0 | 27 | 0.0034 | 1 | U | 314 |
| 19030 | 9563.825 | 9562.817 | 9488.689 | 0.781 | 0 | 25 | 0.0078 | 1 | U | 315 |
| 19030 | 4364.282 | 4363.275 | 4363.232 | 0.001 | 0 | 24 | 0.0044 | 1 | U | 316 |
| 19030 | 17382.498 | 17381.491 | 17373.464 | 0.046 | 0 | 24 | 0.0067 | 1 | U | 317 |
| 19030 | 4814.619 | 4813.612 | 4827.612 | −0.290 | 0 | 15 | 0.035 | 1 | U | 318 |

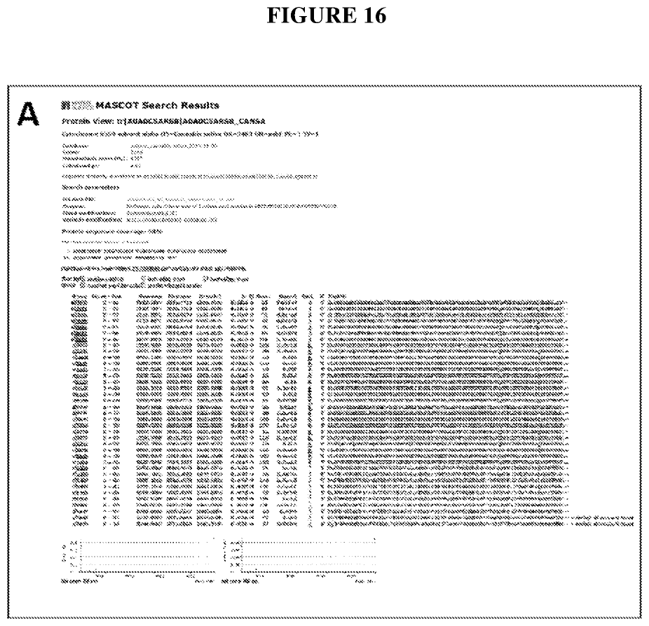

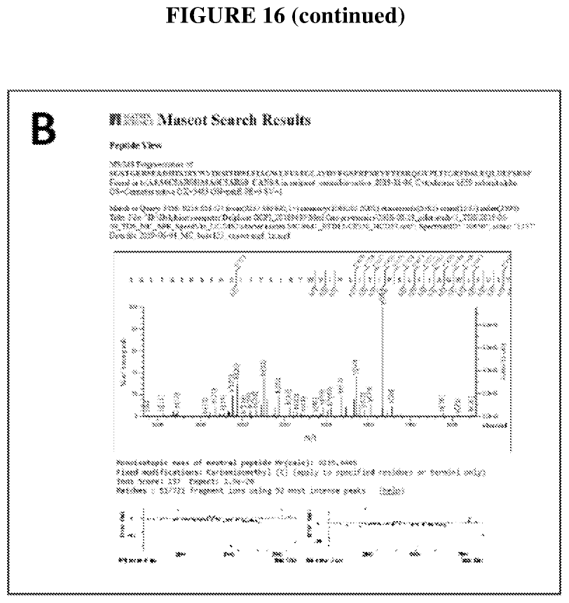

Two of the 20 proteins match hits from hop (Humulus lupulus), with one hit (cytochrome b559 subunit alpha) identified in both C. sativa (accession A0A0C5ARS8, highest score of 2265, FIG. 16) and H. lupulus species (accession A0A0U2GZT5, score of 902). The other protein from H. lupulus was chloroplastic 30S ribosomal protein S14. Overall, 18 accessions were unmodified proteoforms, six with one oxidation, one with 2 oxidations, and seven that display a N-terminus acetylation.

Comparing the list of cannabis intact proteins identified by a top-down approach to that of trypsin-digested proteins identified by bottom-up proteomics described above, 7 proteins overlap and 13 proteins are novel (Table 13).

Most identified proteins (12/20, 60%) are involved in photosynthesis (subunits of cytochromes and photosystems I and II), then in protein translation (4 ribosomal proteins, 20%). Also identified are two ATP synthases, a non-specific lipid-transfer protein, and Betv1-like protein. Only one protein belongs to the phytocannabinoid biosynthesis, olivetolic acid cyclase (I6WU39, OAC), also identified by bottom-up proteomics (Table 4). With a Mascot score of 162, OAC is identified both as an unmodified proteoform and an acetylated proteoform (Table 13).

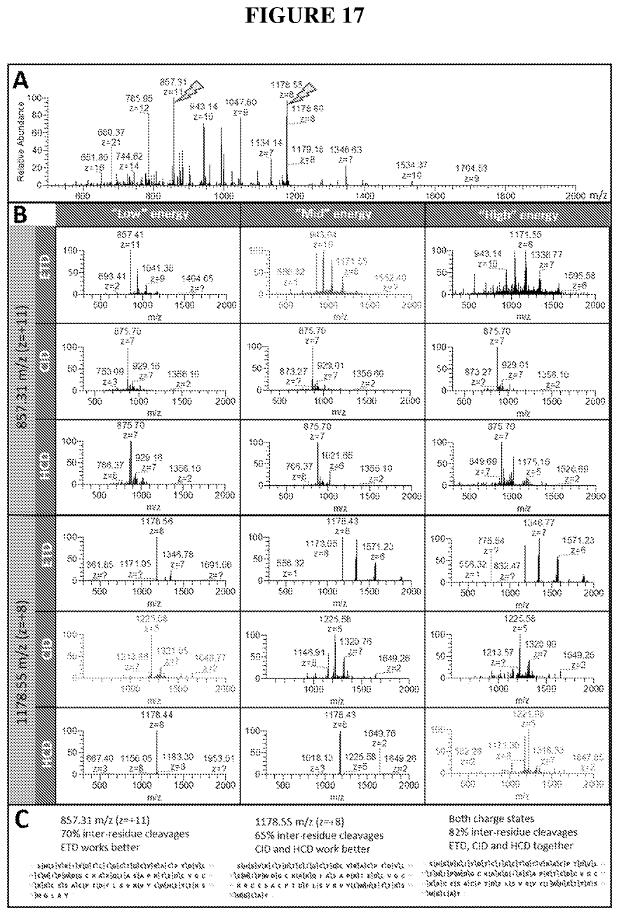

Consistent with the data obtained from the protein standards, fragmentation efficiency of cannabis intact proteins depends on the charge state of the parent ion, on the type of MS/MS mode, and on the level of energy applied. We are illustrating this using the protein exhibiting the second highest Mascot score (1664), Photosystem I iron-sulfur center (PS I Fe—S center, accession A0A0C5AS17) identified with 39 MS/MS spectra. Fragmentation efficiency is assessed using ProSight Lite program by the percentage of inter-residue cleavages achieved. MS/MS spectra differ in the number of peaks and their distribution along the mass range (FIGS. 17A and B).

The optimum dissociation of a precursor ion with high charge state (857.31 m/z, z=+11)) is achieved with ETD at “Mid” energy, whereas a precursor ion of comparable intensity but with lower charge state (1178.55 m/z, z=+8) responds better to CID and HCD at “Low” and “High” energy levels, respectively. All MS/MS data considered, fragmenting 857.31 m/z and 1178.55 m/z parent ions yields 70% and 65% inter-residue cleavages, respectively, and 82% all together (FIG. 17C). In order to maximise AA sequence coverage, it is essential to multiply the MS/MS conditions on as many precursor ions as possible. This of course limits the total number of different proteins analysed in a top-down approach. Coupling this strategy with an extended separation run should alleviate this drawback.

### Example 8—Optimisation of Multiple Protease Strategy for the Preparation of Samples for Bottom-Up and Middle-Down Proteomics

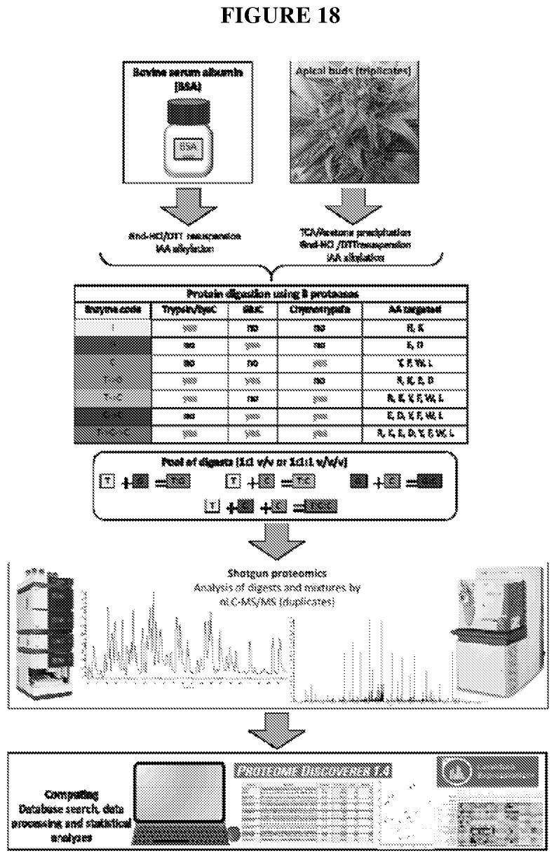

In this experiment, a trypsin/LysC mixture, GluC and chymotrypsin were applied on their own or in combination, either sequentially in a serial digestion fashion, or by pooling individual digests together. The analytical method was first tested on BSA and then applied to complex plant samples. The experimental design is schematised in FIG. 18.

BSA was used as a positive control in the experiment as it is often used as the gold standard for shotgun proteomics. BSA is a monomeric protein particularly amenable to trypsin digestion. Many laboratories determine the sequence coverage of BSA tryptic digest in order to rapidly evaluate instrument performance because it is sensitive to method settings in both MS1 and MS2 acquisition modes. Beside the trypsin/LysC mixture (T), we tested two other proteases, GluC (G) and chymotrypsin (C), either independently or applied sequentially (denoted by an arrow or →) as follows: trypsin/LysC followed by GluC (T→G), trypsin/LysC followed by chymotrypsin (T→C), GluC followed by chymotrypsin (G→C), and trypsin/LysC followed by GluC followed by chymotrypsin (T→G→C). We also pooled equal volumes of the individual digests (denoted by a colon or :) as follows: trypsin/LysC with GluC (T:G), trypsin/LysC with chymotrypsin (T:C), GluC with chymotrypsin (G:C), and trypsin/LysC with GluC and chymotrypsin (T:G:C).

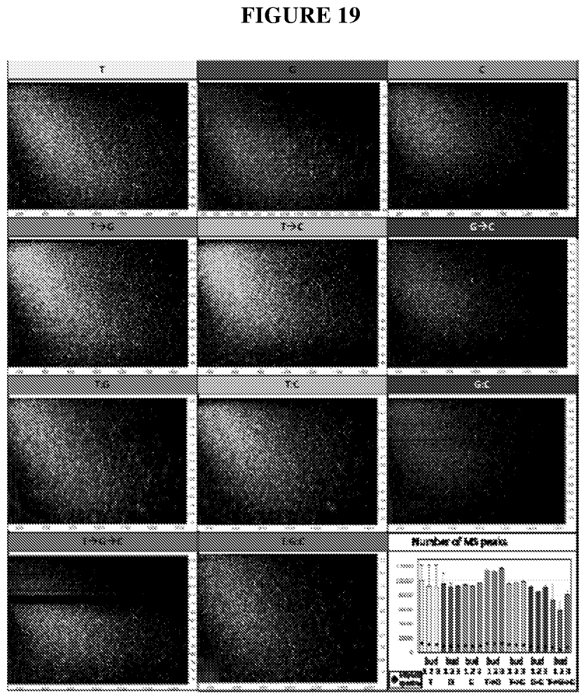

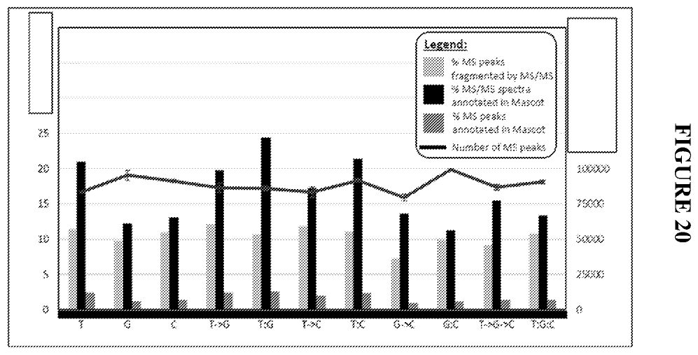

Each BSA digest underwent nLC-MS/MS analysis in which each duty cycle comprised a full MS scan was followed by CID MS/MS events of the 20 most abundant parent ions above a 10,000 counts threshold. FIG. 19 displays the LC-MS profiles corresponding to one replicate of each BSA digest.

The peptides elute from 9 to 39 min corresponding to 9-39% ACN gradient, respectively and span m/z values from 300 to 1600. Visually, LC-MS patterns from samples subject to digestion with trypsin/LysC (T) and GluC followed by chymotrypsin (G->C) are relatively less complex than the other digests. Technical duplicates of the BSA digests yield MS and MS/MS spectra of high reproducibility as can be seen in Table 16.

TABLE 16
Number of MS peaks, MS/MS spectra and MS/MS spectra
annotated with SEQUEST for each BSA digest.

| | Protease | 1. MS | | | | | 2. all MS/MS | | | |
|---|---|---|---|---|---|---|---|---|---|---|
| Sample | mix | Rep 1 | Rep 2 | Mean | SD | % CV | Rep 1 | Rep 2 | Mean | SD |
| BSA | T | 83678 | 83056 | 83367 | 440 | 0.5 | 9769 | 9325 | 9547 | 314 |
| BSA | G | 91922 | 98895 | 95409 | 3487 | 3.7 | 9081 | 9628 | 9355 | 387 |
| BSA | C | 92116 | 90303 | 91210 | 907 | 1.0 | 10327 | 9792 | 10060 | 378 |
| BSA | T−>G | 89648 | 83107 | 86378 | 3271 | 3.8 | 11311 | 9698 | 10505 | 1141 |
| BSA | T:G | 84347 | 87462 | 85905 | 1558 | 1.8 | 8605 | 9720 | 9163 | 788 |
| BSA | T−>C | 87203 | 79616 | 83410 | 3794 | 4.5 | 10944 | 8810 | 9877 | 1509 |
| BSA | T:C | 90847 | 92736 | 91792 | 945 | 1.0 | 10245 | 10115 | 10180 | 92 |
| BSA | G−>C | 77085 | 82055 | 79570 | 2485 | 3.1 | 6450 | 5163 | 5807 | 910 |
| BSA | G:C | 99001 | 100001 | 99501 | 500 | 0.5 | 9980 | 9847 | 9914 | 94 |
| BSA | T−>G−>C | 88919 | 84798 | 86859 | 2061 | 2.4 | 9880 | 6137 | 8009 | 2647 |
| BSA | T:G:C | 91975 | 89420 | 90698 | 1278 | 1.4 | 10201 | 9503 | 9852 | 494 |
| BSA | mean | 88795 | 88314 | 88554 | 1884 | 2 | 9708 | 8885 | 9297 | 796 |
| BSA | SD | 5707 | 6752 | 5811 | 1218 | 1 | 1317 | 1648 | 1333 | 756 |
| | min | 77085 | 79616 | 79570 | 440 | 1 | 6450 | 5163 | 5807 | 92 |
| | max | 99001 | 100001 | 99501 | 3794 | 5 | 11311 | 10115 | 10505 | 2647 |

| | | | 3. SEQUEST | | | | % MS/MS | % MS |
|---|---|---|---|---|---|---|---|---|
| | Protease | % MS/MS^{a} | annotated MS/MS | | | | annotated^{b} | annotated^{c} |
| Sample | mix | Percent | Rep 1 | Rep 2 | Mean | SD | % | % |
| BSA | T | 11 | 2133 | 1875 | 2004 | 182 | 21 | 2.4 |
| BSA | G | 10 | 929 | 1363 | 1146 | 307 | 12 | 1.2 |
| BSA | C | 11 | 1358 | 1267 | 1313 | 64 | 13 | 1.4 |
| BSA | T−>G | 12 | 2178 | 1978 | 2078 | 141 | 20 | 2.4 |
| BSA | T:G | 11 | 2141 | 2332 | 2237 | 135 | 24 | 2.6 |
| BSA | T−>C | 12 | 1864 | 1549 | 1707 | 223 | 17 | 2.0 |
| BSA | T:C | 11 | 2428 | 1931 | 2180 | 351 | 21 | 2.4 |
| BSA | G−>C | 7 | 1103 | 475 | 789 | 444 | 14 | 1.0 |
| BSA | G:C | 10 | 1169 | 1065 | 1117 | 74 | 11 | 1.1 |
| BSA | T−>G−>C | 9 | 1485 | 1005 | 1245 | 339 | 16 | 1.4 |
| BSA | T:G:C | 11 | 1015 | 1616 | 1316 | 425 | 13 | 1.5 |
| BSA | mean | 10 | 1618 | 1496 | 1557 | 244 | 17 | 2 |
| BSA | SD | 1 | 544 | 531 | 501 | 136 | 4 | 1 |
| | min | 7 | 929 | 475 | 789 | 64 | 11 | 1 |
| | max | 12 | 2428 | 2332 | 2237 | 444 | 24 | 3 |

^{a}these percentages were obtained by dividing the mean of the number of MS/MS events by the mean of the number of MS peaks;
^{b}these percentages were obtained by dividing the mean of the number of annotated MS/MS spectra by the mean of the number of MS/MS event;
^{c}these percentages were obtained by dividing the mean of the number of annotated MS/MS spectra by the mean of the number of MS peaks.

All LC-MS patterns are highly complex. The number of MS peaks vary from 77,085 (G→C rep 1) to 100,001 (G:C rep 2) across all patterns and SDs range from 440 (T) to 3,794 (T→C) with coefficient of variations (% CVs) always lower than 5%, even though a full set of eleven digest combinations (FIG. 18) was run first (technical replicate 1), and then fully repeated in the same order (technical replicate 2) with no randomisation applied. The number of MS/MS events ranges from 5,163 (6%, G→C rep 2) to 11,311 (13% T→G rep 1), which amounts to 10% of all the MS peaks on average (Table 16). The number of MS/MS events per sample is determined by the duration of the run (50 min) and the duty cycle (3 sec) which in turn is controlled by the resolution (60,000), number of microscans (2) and number of MS/MS per cycle (20). In our experiment, a 50 min run allows for 1,000 cycles and 20,000 MS/MS events. Proteotypic peptides elute for 30 min, thus allowing for a maximum of 12,000 MS/MS scans. With an average number of 9,297 MS/MS spectra obtained (Table 16), 77% of the potential is thus achieved. Duty cycles can be shortened by lowering the resolving power of the instrument, minimising the number of microscans and diminishing the number of MS/MS events. The MS/MS data was searched against a database containing the BSA sequence using SEQUEST algorithm for protein identification purpose. Of all the MS/MS spectra generated in this study, between 475 (9%, G→C rep 2) and 2,428 (24%, T:C rep 1) are successfully annotated as BSA peptides (Table 16). On average, 17% of the MS/MS spectra yield positive database hits, which amounts to an average of 1.8% of MS peaks. Trypsin/LysC yields 68 unique BSA peptides, GluC yields 79 unique BSA peptides, and chymotrypsin yields 104 unique BSA peptides. BSA was identified with 51 unique peptides obtained using trypsin on its own; therefore, the mixture trypsin/LysC further enhances the digestion of BSA. The percentages of Table 16 are presented as a histogram in FIG. 20. The proportion of MS peaks fragmented by MS/MS remains constant across BSA digests, oscillating around 10±3% (light grey bars). The proportions of MS/MS spectra annotated in SEQUEST (i.e. successful hits) however show more variation across proteases (black bars). Higher percentages are reached when trypsin/LysC is employed on its own or in combination with GluC and/or chymotrypsin (FIG. 20). This is expected as BSA is amenable to trypsin digestion and often used as shotgun proteomics standard.

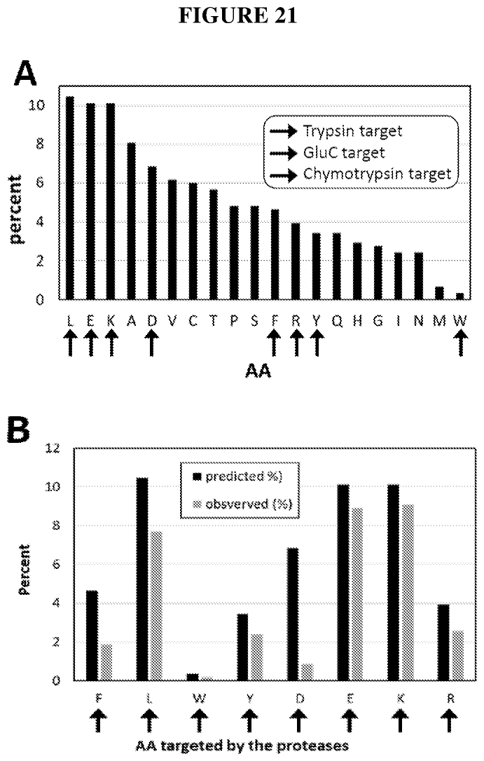

BSA (P02769) mature primary sequence contains 583 amino acids (AA), from position 25 to 607; the signal peptide (position 1 to 18) and propeptide (position 19 to 24) are excised during processing. In theory, BSA should favourably respond to each protease as it contains plethora of the AAs targeted during the digestion step. FIG. 20A indicates the AA composition of BSA. Targets of chymotrypsin (L, F, Y, and W) account for 19% of BSA sequence, targets of GluC (E and D) represent 17% of the sequence, and targets of trypsin/LysC (K, R) make 14% of the total AA composition of BSA. As these percentages are similar, the difference in the numbers of MS/MS spectra successfully matched by SEQUEST from one protease to another cannot be attributed to digestion site predominance. When we compare these predicted percentages to those observed in our study based on unique peptides (FIG. 21B), all the targeted AAs indeed undergo cleavage. The predicted rate always exceeds the observed one, but only moderately for W, Y, E, K, and R residues (less than 1.5% difference). However, F, L, and in particular D residues present an observed cleavage rate that is much lower than the predicted one (FIG. 21B). GluC efficiently cleaves E residues, but misses most of D residues, even though the digestion step is performed under slightly alkaline conditions (pH=7.8) optimal for GluC activity as recommended by the manufacturer.

The number of successfully annotated MS/MS events to that of MS peaks, fluctuated from 1.0% (G->C) to 2.6% (T:C) (Table 16 and dark grey bars in FIG. 19).

Together, these data demonstrate that LC-MS/MS data from BSA digests are very reproducible.

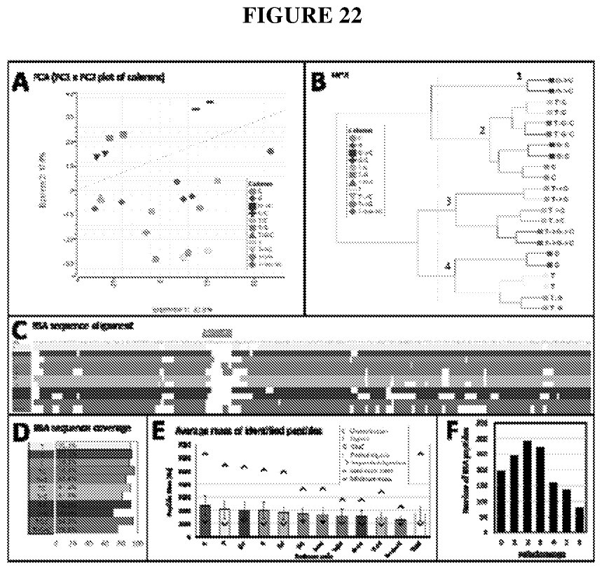

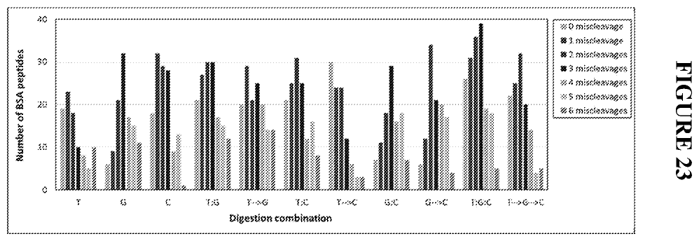

The statistical tests performed and the BSA sequence information as well as a visual assessment of BSA sequencing success for each combination of enzymes is provided by FIG. 22.

PCA shows that technical duplicates group together (FIG. 22A). BSA samples arising from enzymatic digestion using chymotrypsin in combination or not with GluC separate from the rest, particularly tryptic digests, along PC 2 explaining 17.5% of the variance. HCA confirms PCA results and further indicates that samples treated with trypsin/LysC (T) and GluC (G) on their own or pooled (T:C) form one cluster (cluster 4, FIG. 21B). The closest cluster (cluster 3) comprises all the samples subject to sequential digestions (represented by an arrow →), except for digests resulting from the consecutive actions of GluC and chymotrypsin (G→C) which constitute a cluster on their own (cluster 1). The last cluster (cluster 2) groups chymotryptic samples with the remaining pooled digests (represented by a colon). The fact that clusters 1-3 contains samples treated with chymotrypsin (except for T→G) suggests that this protease produces peptides with unique properties, which affect the down-stream analytical process. These data confirm that chymotrypsin acts in an orthogonal fashion to trypsin.

Based on the 589 unique peptides identified in this study, we generated a BSA sequence alignment map (FIG. 22C) and coverage histogram (FIG. 22D). All digests considered, BSA sequence is at least 70% covered (G->C), up to 97% (T:G) (FIG. 22D), with an average of 87% coverage. Despite this almost complete coverage, the seven AA-long area positioned between residues 214 and 220 (ASSARQR) resist digestion, even though R residues targeted by trypsin/LysC are present (FIG. 22C). Other areas resisting cleavage were common across different digests (e.g., position 162-171, LYEIARRHPY, shared between C, T→C, G→C, and T→G→C) or unique to a particular digest (e.g., position 268-275, CCHGDLLE, in G:C) (FIG. 22C). Comparison of digests obtained using a unique enzyme demonstrate excellent BSA sequence coverage: 91.3% for trypsin/LysC, 93.1% for GluC, and 90.2% for chymotrypsin (FIG. 22D).

We compared digests obtained using multiple enzymes and compare sequential digestions (→) with pooled digests (:), and observed better alignment and coverage when individual digests are combined than when proteases are added. For instance, T→C digests covers 81% of the BSA sequence while T:C digest reach 91% coverage (FIG. 22D); the 10% difference represents 56 AAs. This is better exemplified when the three proteases are used together, with a 75% coverage in T→G→C samples and 94% coverage in T:G:C samples (FIG. 22D); the 19% difference representing 111 AAs.

The masses of identified peptides ranged from 688 to 6,412 Da, with an average of 1,758±753 Da (FIG. 22E), containing 5-54 AA residues. GluC is the enzyme that generates the longest peptides with an average of 2,342±1052 Da, followed by trypsin/LysC (2053±1000 Da), the mixture GluC/chymotrypsin (G:C, 2008±765), and chymotrypsin (1989±901 Da). GluC on its own produces peptides large enough to undertake MDP analyses. The smallest peptides result from the sequential actions of GluC and chymotrypsin (G→C, 1541±511 Da), trypsin/LysC and chymotrypsin (T→C, 1481±567 Da), and all three proteases (T→G→C, 1295±348 Da). This confirms that adding multiple proteases to a sample enhances protein cleavage. BSA peptides contain up to six miscleavages, with the majority (59%) presenting 1-3 miscleavages (FIG. 22F). The different digestion conditions peak at different miscleavages as can be seen in FIG. 23. For instance, the greatest number of tryptic and chymotryptic peptides exhibit one miscleavage while GluC-released peptides containing three miscleavages are the most numerous. The longest peptide (VSRSLGKVGTRCCTKPESERMPCTEDYLSLILNRLCVLHEKTPVSEKVTKCCTE, 6.4 kDa) released from the action of GluC contains eight charges, and six miscleavages; it has a SpScore of 1,572 and a Xcorr of 4.14. Where trypsin is used to perform the enzymatic digestion of the protein extracts, the maximum number of missed cleavages is typically set to two. However, these data demonstrate that a significant proportion of BSA peptides (47%) contain more than two miscleavages (35% of BSA tryptic peptides).

Together, these data demonstrate that BSA is highly amenable to enzymatic digestion by trypsin/LysC, GluC and chymotrypsin. Pooling the individual digests does not affect the LC-MS/MS analysis as attested by the high sequencing coverage. Using multiple proteases consecutively yields relatively lower sequence coverage of BSA.

### Example 9—Application of a Multiple Protease Strategy for the Preparation of Medicinal Cannabis Samples for Shotgun Proteomics

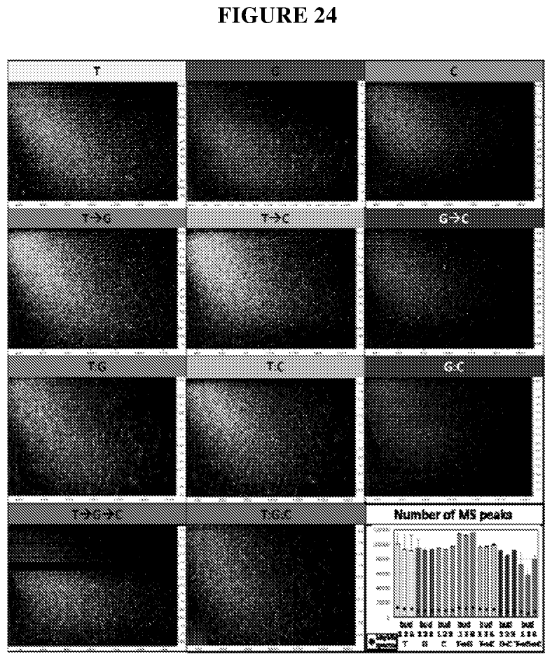

LC-MS patterns are very complex with cannabis peptides eluting from 9-39 min (9-39% ACN gradient) exhibiting m/z values spanning from 300 to 1,700 (FIG. 24).

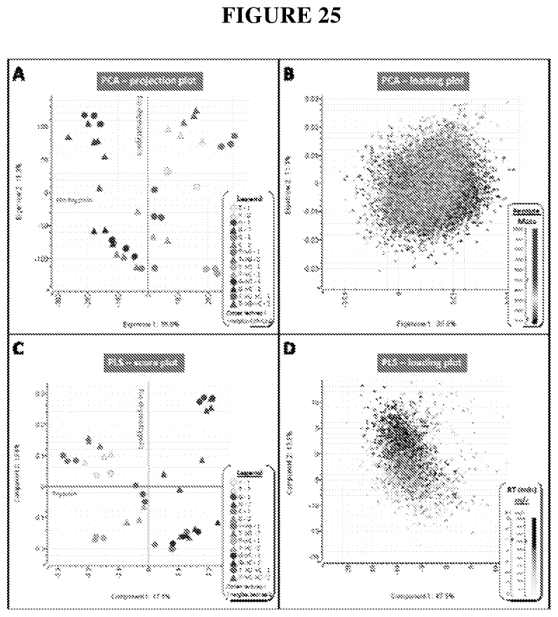

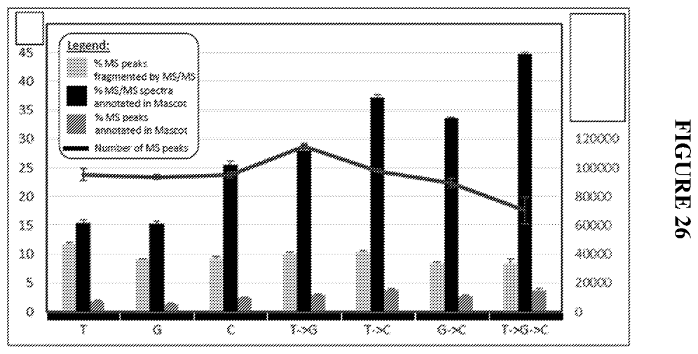

Statistical analyses were carried out on volumes of the 27,635 peptides identified in this study. Multivariate analyses (PCA, PLS, HCA) were performed as well as a linear model which isolated 3,349 peptides significantly responding to the digestion type. The PCA projection plot of PC1 and PC2 using all identified peptides shows that samples are grouped by digestion type, with biological triplicates closely clustering together but technical duplicates separating out as they were run at two independent times (FIG. 25A), which can be resolved by randomizing the LC injection order.

PC1 explains 35% of the total variance and separates samples that include digestion with trypsin/LysC on the right-hand side away from the samples which do not on the left-hand side. PC2 explains 11.3% of the variance and discriminates samples on the basis of their treatment with or without chymotrypsin (FIG. 25A). Peptide mass is the determining factor behind the sample grouping across PC1×PC2 as can be seen on the PCA loading plot which illustrates that samples treated with GluC generate the longest peptides (>5 kDa, FIG. 25B). A PLS analysis was performed using the 3,349 peptides that were most significantly differentially expressed across the seven digestion types. This supervised statistical process defined groups according to a particular experimental design, in this instance the digestion type. The score plot of the first two components indeed achieve better separation of the different digestion types, with samples treated with GluC away from all the other types (FIG. 25C). One group is composed of the samples treated with trypsin/LysC on its own and combined to GluC. Another group comprises samples treated with chymotrypsin on its own and with GluC. The last group positioned in between contains samples treated with trypsin/LysC and chymotrypsin, as well as with GluC. The main peptide characteristics behind such grouping is the m/z value as illustrated on the PLS loading plot (FIG. 25D). These data confirm the orthogonality of the proteases used in this experiment.

The number of MS peaks varies from 49,316 (Bud 2 T→G→C rep 2) to 118,020 (Bud 3 T→G rep 1), with an average value of 93,771±15,426 (Table 17).

TABLE 17
Number of MD peaks, MS/MS spectra and MS/MS spectra annotated
in SEQUEST for each medicinal cannabis digest

| Biol | Protease | 1. MS | | | | | 2. all MS/MS | | | |
|---|---|---|---|---|---|---|---|---|---|---|
| rep | mix | Rep 1 | Rep 2 | Mean | SD | % CV | Rep 1 | Rep 2 | Mean | SD |
| Bud 1 | T | 86458 | 115577 | 101018 | 20590 | 20.4 | 12827 | 11731 | 12279 | 775 |
| Bud 2 | T | 72907 | 113303 | 93105 | 28564 | 30.7 | 10775 | 11160 | 10968 | 272 |
| Bud 3 | T | 70473 | 112818 | 91646 | 29942 | 32.7 | 10541 | 10585 | 10563 | 31 |
| Bud 1 | G | 106622 | 84761 | 95692 | 15458 | 16.2 | 9035 | 8501 | 8768 | 378 |
| Bud 2 | G | 95761 | 88387 | 92074 | 5214 | 5.7 | 8032 | 7906 | 7969 | 89 |
| Bud 3 | G | 93760 | 91846 | 92803 | 1353 | 1.5 | 8810 | 8115 | 8463 | 491 |
| Bud 1 | C | 93117 | 95399 | 94258 | 1614 | 1.7 | 9486 | 8644 | 9065 | 595 |
| Bud 2 | C | 93778 | 92536 | 93157 | 878 | 0.9 | 8433 | 7788 | 8111 | 456 |
| Bud 3 | C | 97359 | 97813 | 97586 | 321 | 0.3 | 9508 | 8341 | 8925 | 825 |
| Bud 1 | T−>G | 116131 | 113352 | 114742 | 1965 | 1.7 | 11909 | 11406 | 11658 | 356 |
| Bud 2 | T−>G | 113690 | 111601 | 112646 | 1477 | 1.3 | 11511 | 10857 | 11184 | 462 |
| Bud 3 | T−>G | 118020 | 115958 | 116989 | 1458 | 1.2 | 12362 | 11811 | 12087 | 390 |
| Bud 1 | T−>C | 98125 | 94395 | 96260 | 2638 | 2.7 | 10963 | 9568 | 10266 | 986 |
| Bud 2 | T−>C | 98455 | 97615 | 98035 | 594 | 0.6 | 10622 | 9090 | 9856 | 1083 |
| Bud 3 | T−>C | 100667 | 97679 | 99173 | 2113 | 2.1 | 11238 | 8873 | 10056 | 1672 |
| Bud 1 | G−>C | 92277 | 90930 | 91604 | 952 | 1.0 | 8219 | 7625 | 7922 | 420 |
| Bud 2 | G−>C | 86056 | 83949 | 85003 | 1490 | 1.8 | 7160 | 6390 | 6775 | 544 |
| Bud 3 | G−>C | 93847 | 89624 | 91736 | 2986 | 3.3 | 8158 | 7398 | 7778 | 537 |
| Bud 1 | T−>G−>C | 88886 | 56861 | 72874 | 22645 | 31.1 | 9479 | 4279 | 6879 | 3677 |
| Bud 2 | T−>G−>C | 67123 | 49316 | 58220 | 12591 | 21.6 | 6835 | 1770 | 4303 | 3581 |
| Bud 3 | T−>G−>C | 84077 | 77062 | 80570 | 4960 | 6.2 | 7685 | 5570 | 6628 | 1496 |
| | Mean | 13559 | 17773 | 13095 | 9797 | 11 | 1743 | 2526 | 2047 | 992 |
| | SD | 13232 | 17345 | 12779 | 9561 | 11 | 1701 | 2465 | 1997 | 968 |
| | Min | 67123 | 49316 | 58220 | 321 | 0.33 | 6835 | 1770 | 4303 | 31.1 |
| | Max | 118020 | 115958 | 116989 | 29942 | 32.7 | 12827 | 11811 | 12279 | 3677 |

| | | | 3. SEQUEST | | | | % MS/MS | % MS |
|---|---|---|---|---|---|---|---|---|
| Biol | Protease | % MS/MS^{a} | annotated MS/MS | | | | annotated^{b} | annotated^{c} |
| rep | mix | Percent | Rep 1 | Rep 2 | Mean | SD | % | % |
| Bud 1 | T | 12 | 2042 | 1929 | 1986 | 80 | 16 | 2.0 |
| Bud 2 | T | 12 | 1606 | 1740 | 1673 | 95 | 15 | 1.8 |
| Bud 3 | T | 12 | 1513 | 1643 | 1578 | 92 | 15 | 1.7 |
| Bud 1 | G | 9 | 1388 | 1376 | 1382 | 8 | 16 | 1.4 |
| Bud 2 | G | 9 | 1200 | 1146 | 1173 | 38 | 15 | 1.3 |
| Bud 3 | G | 9 | 1326 | 1290 | 1308 | 25 | 15 | 1.4 |
| Bud 1 | C | 10 | 2589 | 2200 | 2395 | 275 | 26 | 2.5 |
| Bud 2 | C | 9 | 2232 | 1857 | 2045 | 265 | 25 | 2.2 |
| Bud 3 | C | 9 | 2382 | 2098 | 2240 | 201 | 25 | 2.3 |
| Bud 1 | T−>G | 10 | 3416 | 3163 | 3290 | 179 | 28 | 2.9 |
| Bud 2 | T−>G | 10 | 3103 | 2904 | 3004 | 141 | 27 | 2.7 |
| Bud 3 | T−>G | 10 | 3633 | 3405 | 3519 | 161 | 29 | 3.0 |
| Bud 1 | T−>C | 11 | 4066 | 3434 | 3750 | 447 | 37 | 3.9 |
| Bud 2 | T−>C | 10 | 4024 | 3308 | 3666 | 506 | 37 | 3.7 |
| Bud 3 | T−>C | 10 | 4297 | 3321 | 3809 | 690 | 38 | 3.8 |
| Bud 1 | G−>C | 9 | 2786 | 2545 | 2666 | 170 | 34 | 2.9 |
| Bud 2 | G−>C | 8 | 2393 | 2190 | 2292 | 144 | 34 | 2.7 |
| Bud 3 | G−>C | 8 | 2687 | 2502 | 2595 | 131 | 33 | 2.8 |
| Bud 1 | T−>G−>C | 9 | 4117 | 2002 | 3060 | 1496 | 44 | 4.2 |
| Bud 2 | T−>G−>C | 7 | 3065 | 824 | 1945 | 1585 | 45 | 3.3 |
| Bud 3 | T−>G−>C | 8 | 3392 | 2524 | 2958 | 614 | 45 | 3.7 |
| | Mean | 1 | 991 | 787 | 836 | 439 | 10 | 1 |
| | SD | 1 | 967 | 769 | 816 | 428 | 10 | 1 |
| | Min | 7.391 | 1200 | 824 | 1173 | 8.49 | 14.7195 | 1.27398 |
| | Max | 12.155 | 4297 | 3434 | 3809 | 1585 | 45.1894 | 4.19837 |

^{a}these percentages were obtained by dividing the mean of the number of MS/MS events by the mean of the number of MS peaks;
^{b}these percentages were obtained by dividing the mean of the number of annotated MS/MS spectra by the mean of the number of MS/MS events;
^{c}these percentages were obtained by dividing the mean of the number of annotated MS/MS spectra by the mean of the number of MS peaks.

The MS data was searched against a C. sativa database using SEQUEST algorithm for protein identification purpose. Of all the MS/MS spectra generated from medicinal cannabis digests, between 824 (47% of the 1,770 MS/MS spectra for Bud 2 T→G→C rep 2) and 4,297 (38% of the 11,238 MS/MS spectra for Bud 3 T→C rep 1) are successfully annotated (Table 17). On average, 29% of the MS/MS spectra yield positive database hits, which amounts to an average of 2.7% of MS1 peaks.

The percentages of Table 17 are presented as a histogram in FIG. 26. As observed before for BSA samples, the proportion of MS peaks fragmented by MS/MS remains fairly constant across the medicinal cannabis digests, ranging from 7-12% as it is set by the duty cycle. The proportion of MS/MS spectra annotated in SEQUEST (i.e., successful hits), however, shows even more variation across proteases than BSA, fluctuating from 15 to 45%. Higher percentages are reached when chymotrypsin is employed on its own or in combination with trypsin/LysC and/or GluC (FIG. 26). In the case of medicinal cannabis protein extracts, the strategy involving sequential enzymatic digestions using two or three proteases proves very successful with high annotation rates: 28% for T→G, 34% for G→C, 37% for T→C and 45% for T→G→C (FIG. 26).

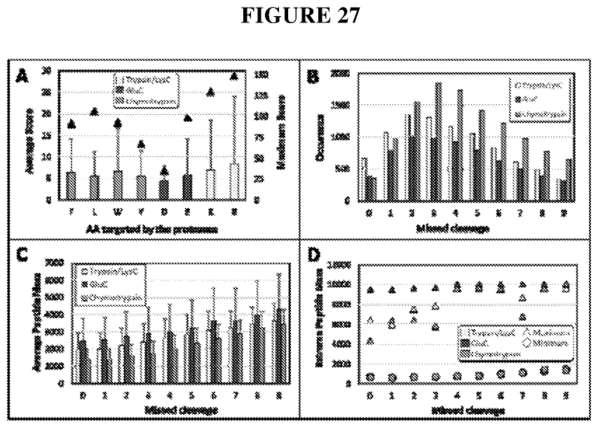

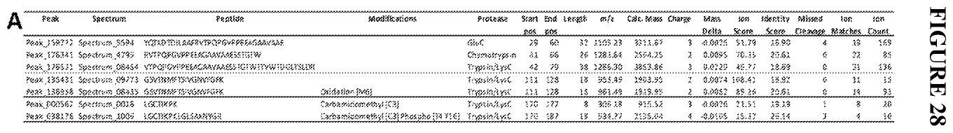

A total of 22,046 unique peptides from cannabis samples are identified. This improves upon the results achieved using bottom-up proteomics based on trypsin digestion. In view of these results, it is demonstrated that proteases behave differently. For instance, the highest peptide ion scores are found among the peptides generated by trypsin/LysC, in particular when arginine residues (R) are targeted, whereas the lowest scores belong to peptides resulting from the cleavage of aspartic acid residues (D) upon the action of GluC (FIG. 27A).

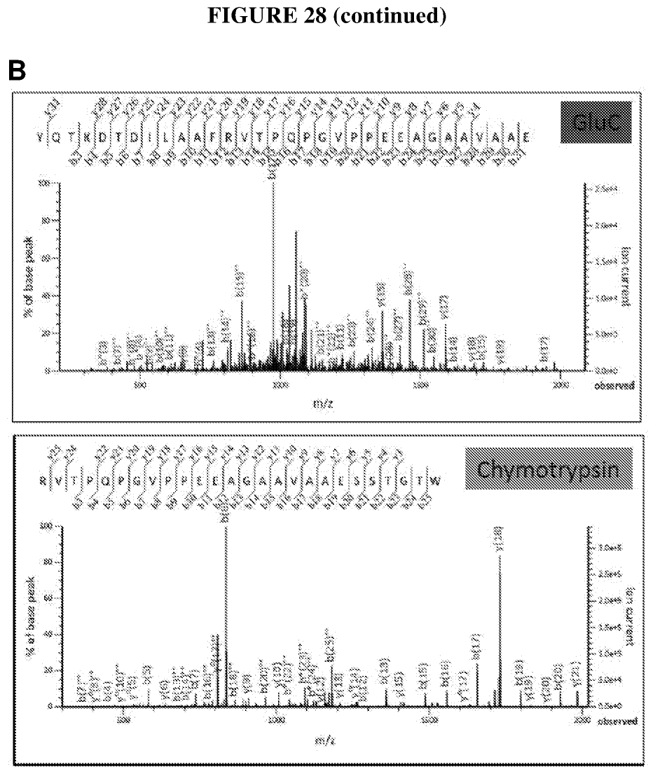

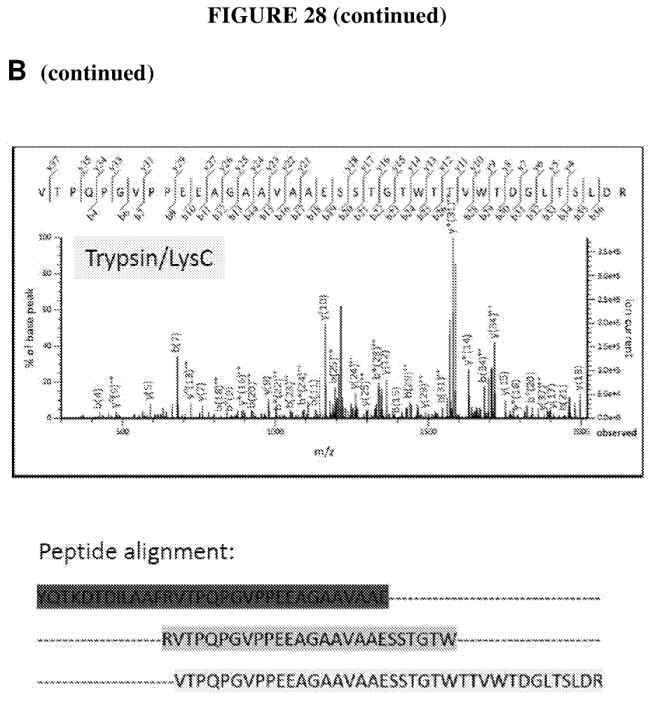

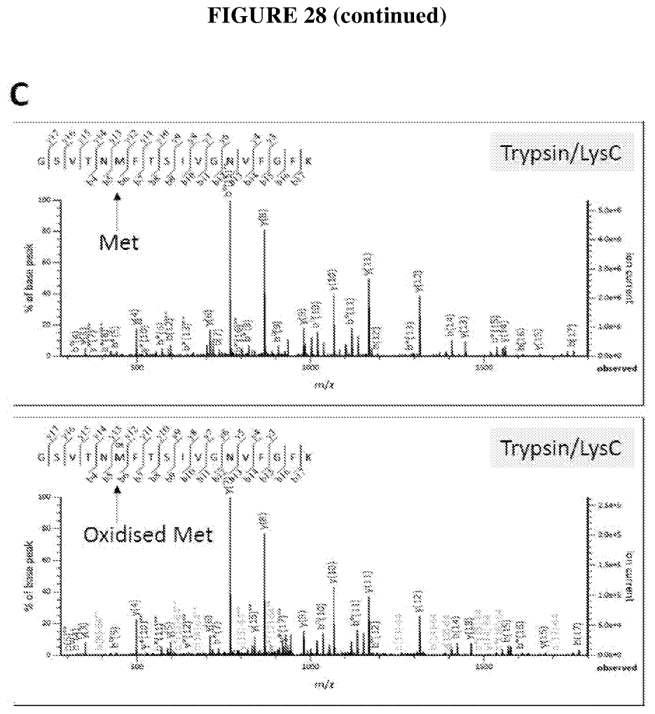

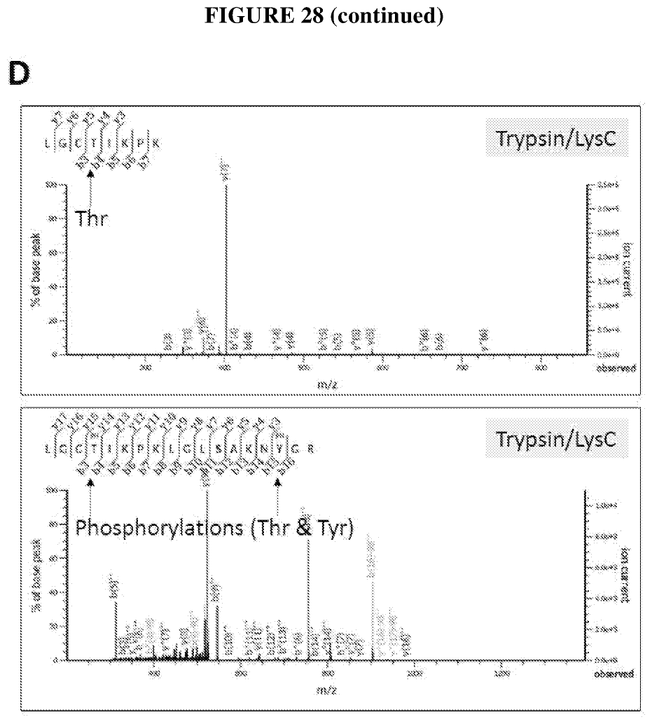

Ion scores average around 6.1±9.6 and reach up to 148. Apart from the expected (fixed) PTMs due to the carbamidomethylation of reduced/alkylated cysteine residues during sample preparation, dynamic PTMs such as oxidation, phosphorylations and N-terminus acetylations are also found. Annotated MS/MS spectra can be viewed in FIG. 28. In these examples, peptides from ribulose bisphosphate carboxylase large chain (RBCL) are identified with high scores from GluC, chymotrypsin and trypsin/LysC (FIG. 28A). MS/MS annotation from SEQUEST in FIG. 28B illustrates how each enzyme helps extend the coverage of RBCL spanning the region Tyr29 to Arg79 (YQTKDTDILAAFRVTPQPGVPPEEAGAAVAAESSTGTWTTVWTDGLTSLDR) with chymotrypsin covering residues 41-66, GluC extending the coverage to the left down to residue 29 and Trypsin/LysC extending it to the right up to residue 79. MS/MS spectra display almost complete b- and y-series ions (FIG. 28B). RBCL is adorned with several dynamic PTMs, for instance oxidation of Met116 (FIG. 28C) and phosphorylation of Thr173 and Tyr185 (FIG. 28D).

The distribution of identified cannabis peptides according to the number of missed cleavages also reveals differences among proteases. Our method specified a maximum of ten missed cleavage sites, which is highest number allowed in Proteome Discoverer program and SEQUEST algorithm. 5% of the peptides present no missed cleavage and up to nine missed cleavages are detected in the MS/MS data (FIG. 27B). The greatest numbers of peptides resulting from trypsin/LysC or GluC present two missed cleavages while the largest number of chymotrypsin-released peptides possess three missed cleavages. Average masses of cannabis peptides steadily increase with the number of enzymatic cleaving sites missed, in a similar manner for each of the proteases (FIG. 27C). When we observe the minimum masses, we can see that they increase with the number of missed cleavages, very similarly across all three proteases (FIG. 27D). The shortest cannabis peptide has a mass of 627.3956 Da (7 AAs, position 286-292, from Photosystem II protein D2), presents one miscleavage and arises from the action of chymotrypsin, which is the least specific of the proteases tested. When we observe the maximum masses, GluC systematically produce the largest peptides, fluctuating from 9,479.692 to 10,0027.014 Da, regardless of the number of missed cleavages (FIG. 27D). Trypsin/LysC and chymotrypsin display similar patterns, namely the maximum masses increase as the number of missed cleavages go from 0 to 4, and then plateau around 9.6 kDa for subsequent numbers of missed cleavages. The longest peptide has a mass of 10,0027.014 Da (88 AAs, position 57 to 144, from CBDA synthase), bears six missed cleavage sites and arise from the action of GluC which is the most specific of the proteases tested.

A total of 494 unique accessions corresponding to 229 unique proteins from C. sativa and close relatives were identified (Table 18).

TABLE 18
Proteins identified in medicinal cannabis mature apical buds

| | Protein | Number of | | MW | | Seen in |
|---|---|---|---|---|---|---|
| Protein annotation | score | peptides | Coverage | (Da) | Pathway | Table 4 |
| 3,5,7-trioxododecanoyl-CoA | 2824 | 149 | 100 | 42585 | Cannabinoid | yes |
| Cannabidiolic acid synthase | 3403 | 660 | 100 | 62268 | Cannabinoid | yes |
| Geranylpyrophosphate:olivetola | 17 | 3 | 11 | 44514 | Cannabinoid | yes |
| Olivetolic acid cyclase | 767 | 40 | 100 | 12002 | Cannabinoid | yes |
| Polyketide synthase 1 | 69 | 13 | 16 | 42507 | Cannabinoid | no |
| Polyketide synthase 2 | 81 | 20 | 72 | 42610 | Cannabinoid | no |
| Polyketide synthase 3 | 94 | 2 | 11 | 42571 | Cannabinoid | no |
| Polyketide synthase 4 | 53 | 7 | 12 | 42604 | Cannabinoid | no |
| Polyketide synthase 5 | 56 | 14 | 21 | 42571 | Cannabinoid | no |
| Tetrahydrocannabinolic acid | 10696 | 2204 | 100 | 62108 | Cannabinoid | yes |
| Tetrahydrocannabinolic acid | 9 | 3 | 10 | 10774 | Cannabinoid | no |
| Tetrahydrocannabinolic acid | 37 | 5 | 20 | 33101 | Cannabinoid | no |
| Tetrahydrocannabinolic acid | 77 | 16 | 89 | 49047 | Cannabinoid | no |
| Cellulose synthase | 878 | 187 | 99 | 12192 | Cell wall | no |
| Putative kinesin heavy chain | 160 | 41 | 100 | 15826 | Cytoskeleton | yes |
| Betv1-like protein | 2076 | 86 | 96 | 17608 | Defence | yes |
| ATP synthase CF0 A subunit | 292 | 60 | 100 | 27206 | Energy | no |
| ATP synthase CF0 B subunit | 10 | 3 | 14 | 21037 | Energy | no |
| ATP synthase CF0 C subunit | 58 | 18 | 54 | 7990 | Energy | no |
| ATP synthase CF1 epsilon | 876 | 44 | 100 | 14648 | Energy | yes |
| ATP synthase epsilon chain, | 4 | 2 | 39 | 14647 | Energy | no |
| ATP synthase subunit 4 | 323 | 71 | 99 | 22199 | Energy | yes |
| ATP synthase subunit 8 | 148 | 29 | 100 | 18231 | Energy | no |
| ATP synthase subunit 9, | 237 | 49 | 100 | 13828 | Energy | no |
| ATP synthase subunit a | 442 | 98 | 95 | 26500 | Energy | no |
| ATP synthase subunit a, | 39 | 10 | 47 | 27161 | Energy | no |
| ATP synthase subunit alpha | 7748 | 452 | 100 | 55324 | Energy | yes |
| ATP synthase subunit alpha, | 232 | 41 | 79 | 55336 | Energy | no |
| ATP synthase subunit b, | 486 | 71 | 95 | 21773 | Energy | no |
| ATP synthase subunit beta | 6851 | 276 | 100 | 53766 | Energy | yes |
| ATP synthase subunit beta, | 112 | 24 | 86 | 53665 | Energy | yes |
| ATP synthase subunit c, | 10 | 3 | 14 | 7990 | Energy | no |
| Cytochrome b | 265 | 53 | 98 | 44352 | Energy | no |
| Cytochrome c | 410 | 50 | 100 | 12044 | Energy | yes |
| Cytochrome c biogenesis B | 287 | 57 | 100 | 22916 | Energy | no |
| Cytochrome c biogenesis FC | 552 | 115 | 100 | 50562 | Energy | yes |
| Cytochrome c biogenesis FN | 597 | 146 | 98 | 64755 | Energy | yes |
| Cytochrome c biogenesis protein | 805 | 135 | 99 | 36850 | Energy | yes |
| Cytochrome c oxidase subunit 1 | 872 | 162 | 99 | 59034 | Energy | no |
| Cytochrome c oxidase subunit 2 | 253 | 60 | 100 | 29465 | Energy | no |
| Cytochrome c oxidase subunit 3 | 326 | 60 | 98 | 29864 | Energy | no |
| NADH dehydrogenase subunit | 902 | 180 | 100 | 53480 | Energy | no |
| NADH dehydrogenase subunit | 281 | 52 | 100 | 11159 | Energy | no |
| NADH dehydrogenase subunit | 521 | 135 | 100 | 44457 | Energy | yes |
| NADH dehydrogenase subunit | 142 | 38 | 94 | 22667 | Energy | yes |
| NADH-plastoquinone | 36 | 11 | 60 | 85480 | Energy | no |
| NADH-quinone oxidoreductase | 132 | 24 | 98 | 13798 | Energy | no |
| NADH-quinone oxidoreductase | 591 | 110 | 100 | 25529 | Energy | no |
| NADH-quinone oxidoreductase | 93 | 20 | 96 | 18752 | Energy | yes |
| NADH-quinone oxidoreductase | 445 | 99 | 100 | 45497 | Energy | no |
| NADH-quinone oxidoreductase | 655 | 129 | 100 | 40394 | Energy | yes |
| NADH-quinone oxidoreductase | 137 | 30 | 99 | 11276 | Energy | yes |
| NADH-quinone oxidoreductase | 1126 | 224 | 100 | 56578 | Energy | yes |
| NADH-ubiquinone | 772 | 156 | 99 | 35591 | Energy | yes |
| NADH-ubiquinone | 909 | 166 | 100 | 54897 | Energy | no |
| NADH-ubiquinone | 1586 | 301 | 100 | 74182 | Energy | yes |
| NADH-ubiquinone | 428 | 84 | 100 | 23568 | Energy | no |
| Putative cytochrome c | 481 | 107 | 98 | 27659 | Energy | no |
| Succinate dehydrogenase | 121 | 19 | 97 | 12122 | Energy | no |
| Succinate dehydrogenase | 196 | 42 | 100 | 20940 | Energy | no |
| 1-deoxy-D-xylulose-5-phosphate | 754 | 126 | 100 | 51629 | Isoprenoid | yes |
| 2-C-methyl-D-erythritol 4- | 513 | 92 | 100 | 35881 | Isoprenoid | no |
| 3-hydroxy-3-methylglutaryl | 1411 | 313 | 100 | 63352 | Isoprenoid | yes |
| 3-hydroxy-3-methylglutaryl | 731 | 145 | 100 | 50029 | Isoprenoid | no |
| 4-hydroxy-3-methylbut-2-en-1- | 1737 | 121 | 100 | 46398 | Isoprenoid | yes |
| Diphosphomevalonate | 689 | 140 | 100 | 50403 | Isoprenoid | yes |
| Isopentenyl-diphosphate delta- | 869 | 98 | 100 | 34848 | Isoprenoid | yes |
| Mevalonate kinase | 878 | 162 | 100 | 44769 | Isoprenoid | yes |
| Phosphomevalonate kinase | 800 | 161 | 100 | 52543 | Isoprenoid | yes |
| Transferase FPPS1 | 340 | 75 | 100 | 39266 | Isoprenoid | yes |
| Transferase FPPS2 | 424 | 96 | 99 | 39162 | Isoprenoid | yes |
| Transferase GPPS large subunit | 606 | 131 | 100 | 42738 | Isoprenoid | yes |
| Transferase GPPS small subunit | 361 | 69 | 100 | 36249 | Isoprenoid | yes |
| Transferase GPPS small | 194 | 51 | 100 | 31157 | Isoprenoid | yes |
| Acetyl-coenzyme A carboxylase | 649 | 119 | 99 | 56437 | Lipid | no |
| Acetyl-coenzyme A carboxylase | 140 | 50 | 47 | 56204 | Lipid | yes |
| Delta 12 desaturase | 328 | 72 | 95 | 44611 | Lipid | no |
| Delta 15 desaturase | 229 | 48 | 99 | 46061 | Lipid | no |
| Non-specific lipid-transfer | 376 | 22 | 87 | 9038 | Lipid | yes |
| 4-coumarate:CoA ligase | 929 | 189 | 98 | 60351 | Phenylpropanoi | yes |
| Naringenin-chalcone synthase | 679 | 101 | 100 | 42720 | Phenylpropanoi | no |
| Phenylalanine ammonia-lyase | 958 | 185 | 98 | 76959 | Phenylpropanoi | yes |
| Chloroplast envelope membrane | 298 | 62 | 100 | 27370 | Photosynthesis | no |
| Cytochrome b559 subunit alpha | 444 | 30 | 100 | 9387 | Photosynthesis | yes |
| Cytochrome b559 subunit beta | 52 | 12 | 100 | 4424 | Photosynthesis | no |
| Cytochrome b6 | 382 | 84 | 100 | 26282 | Photosynthesis | no |
| Cytochrome b6-f complex | 443 | 69 | 100 | 18975 | Photosynthesis | no |
| Cytochrome b6-f complex | 60 | 10 | 81 | 4170 | Photosynthesis | no |
| Cytochrome b6-f complex | 122 | 17 | 100 | 3301 | Photosynthesis | no |
| Cytochrome b6-f complex | 147 | 27 | 100 | 3388 | Photosynthesis | no |
| Cytochrome f | 727 | 87 | 99 | 35269 | Photosynthesis | yes |
| envelope membrane protein, | 24 | 8 | 34 | 27332 | Photosynthesis | no |
| NAD(P)H-quinone | 1049 | 227 | 100 | 56235 | Photosynthesis | no |
| NAD(P)H-quinone | 172 | 28 | 75 | 56522 | Photosynthesis | no |
| NAD(P)H-quinone | 13 | 4 | 29 | 13756 | Photosynthesis | no |
| NAD(P)H-quinone | 14 | 5 | 27 | 11145 | Photosynthesis | no |
| NAD(P)H-quinone | 1950 | 414 | 99 | 86098 | Photosynthesis | yes |
| NAD(P)H-quinone | 23 | 8 | 88 | 19363 | Photosynthesis | no |
| NAD(P)H-quinone | 29 | 8 | 31 | 19977 | Photosynthesis | yes |
| NAD(P)H-quinone | 2 | 1 | 6 | 18723 | Photosynthesis | no |
| NAD(P)H-quinone | 32 | 7 | 26 | 25579 | Photosynthesis | yes |
| NADH dehydrogenase subunit | 214 | 48 | 95 | 19407 | Photosynthesis | no |
| NADH-quinone oxidoreductase | 150 | 26 | 100 | 19995 | Photosynthesis | no |
| Photosystem I assembly protein | 170 | 41 | 100 | 19730 | Photosynthesis | no |
| Photosystem I assembly protein | 223 | 50 | 95 | 21438 | Photosynthesis | yes |
| Photosystem I iron-sulfur center | 757 | 23 | 100 | 9038 | Photosynthesis | yes |
| Photosystem I P700 chlorophyll | 820 | 140 | 100 | 83138 | Photosynthesis | yes |
| Photosystem I P700 chlorophyll | 860 | 125 | 100 | 82402 | Photosynthesis | yes |
| Photosystem I reaction center | 115 | 19 | 100 | 4973 | Photosynthesis | no |
| Photosystem I reaction center | 98 | 21 | 100 | 4011 | Photosynthesis | no |
| Photosystem II CP43 reaction | 1356 | 136 | 100 | 51848 | Photosynthesis | yes |
| Photosystem II CP47 reaction | 1437 | 119 | 96 | 56013 | Photosynthesis | yes |
| Photosystem II phosphoprotein | 11 | 4 | 100 | 2762 | Photosynthesis | no |
| Photosystem II protein D1 | 446 | 68 | 97 | 38979 | Photosynthesis | yes |
| Photosystem II protein D2 | 623 | 72 | 99 | 39580 | Photosynthesis | yes |
| Photosystem II reaction center | 258 | 43 | 100 | 7650 | Photosynthesis | no |
| Photosystem II reaction center | 51 | 12 | 75 | 4168 | Photosynthesis | no |
| Photosystem II reaction center | 49 | 11 | 90 | 4131 | Photosynthesis | no |
| Photosystem II reaction center | 39 | 8 | 77 | 6862 | Photosynthesis | no |
| Photosystem II reaction center | 84 | 10 | 100 | 4497 | Photosynthesis | no |
| Photosystem II reaction center | 60 | 11 | 100 | 3756 | Photosynthesis | no |
| Photosystem II reaction center | 103 | 28 | 100 | 4165 | Photosynthesis | no |
| Photosystem II reaction center | 62 | 13 | 97 | 6497 | Photosynthesis | no |
| Protein PsbN | 131 | 25 | 100 | 4722 | Photosynthesis | no |
| Ribulose bisphosphate | 15356 | 749 | 99 | 52797 | Photosynthesis | yes |
| Small auxin up regulated | 7731 | 1811 | 100 | 20806 | Phytohormone | yes |
| 30S ribosomal protein S11 | 180 | 38 | 99 | 14940 | Protein | no |
| 30S ribosomal protein S12 | 17 | 5 | 17 | 13893 | Protein | no |
| 30S ribosomal protein S12, | 268 | 65 | 94 | 14656 | Protein | yes |
| 30S ribosomal protein S14 | 103 | 21 | 85 | 11717 | Protein | no |
| 30S ribosomal protein S14, | 80 | 11 | 49 | 11727 | Protein | yes |
| 30S ribosomal protein S15 | 25 | 8 | 48 | 10839 | Protein | no |
| 30S ribosomal protein S15, | 338 | 44 | 100 | 10867 | Protein | yes |
| 30S ribosomal protein S16, | 459 | 52 | 79 | 10413 | Protein | no |
| 30S ribosomal protein S18 | 149 | 32 | 100 | 12010 | Protein | no |
| 30S ribosomal protein S19 | 21 | 8 | 32 | 10543 | Protein | no |
| 30S ribosomal protein S19, | 94 | 18 | 95 | 10511 | Protein | no |
| 30S ribosomal protein S2 | 220 | 54 | 100 | 26726 | Protein | no |
| 30S ribosomal protein S2, | 17 | 3 | 11 | 26769 | Protein | no |
| 30S ribosomal protein S3, | 371 | 86 | 96 | 24961 | Protein | yes |
| 30S ribosomal protein S4 | 305 | 54 | 96 | 23628 | Protein | no |
| 30S ribosomal protein S4, | 86 | 18 | 89 | 23651 | Protein | yes |
| 30S ribosomal protein S7, | 20 | 5 | 31 | 17403 | Protein | no |
| 30S ribosomal protein S8 | 524 | 71 | 100 | 15469 | Protein | no |
| 30S ribosomal protein S8, | 113 | 22 | 49 | 15582 | Protein | yes |
| 50S ribosomal protein L16 | 42 | 13 | 19 | 15357 | Protein | no |
| 50S ribosomal protein L16, | 182 | 31 | 100 | 13312 | Protein | yes |
| 50S ribosomal protein L2 | 65 | 15 | 23 | 29880 | Protein | no |
| 50S ribosomal protein L2, | 507 | 72 | 94 | 29981 | Protein | no |
| 50S ribosomal protein L20 | 81 | 24 | 98 | 14602 | Protein | yes |
| 50S ribosomal protein L20, | 7 | 3 | 13 | 14554 | Protein | yes |
| 50S ribosomal protein L22 | 192 | 47 | 100 | 14768 | Protein | no |
| 50S ribosomal protein L22, | 69 | 17 | 99 | 15178 | Protein | no |
| 50S ribosomal protein L23 | 156 | 47 | 100 | 10719 | Protein | no |
| 50S ribosomal protein L32 | 58 | 18 | 100 | 6078 | Protein | no |
| 50S ribosomal protein L33 | 26 | 5 | 74 | 7687 | Protein | no |
| 50S ribosomal protein L36 | 33 | 8 | 84 | 4460 | Protein | no |
| ATP-dependent Clp protease | 326 | 68 | 99 | 21936 | Protein | no |
| Protein TIC 214 | 2063 | 481 | 100 | 22545 | Protein | yes |
| Ribosomal protein L10 | 232 | 47 | 90 | 17514 | Protein | no |
| Ribosomal protein L14 | 157 | 26 | 100 | 13565 | Protein | yes |
| Ribosomal protein L16 | 214 | 43 | 100 | 16078 | Protein | no |
| Ribosomal protein L2 | 291 | 79 | 98 | 37499 | Protein | yes |
| Ribosomal protein L32 | 1 | 1 | 100 | 6078 | Protein | no |
| Ribosomal protein L5 | 232 | 48 | 99 | 21072 | Protein | no |
| Ribosomal protein S10 | 125 | 30 | 100 | 14102 | Protein | no |
| Ribosomal protein S12 | 112 | 22 | 99 | 14193 | Protein | yes |
| Ribosomal protein S13 | 121 | 21 | 99 | 13563 | Protein | yes |
| Ribosomal protein S16 | 22 | 6 | 38 | 8530 | Protein | no |
| Ribosomal protein S19 | 33 | 15 | 97 | 11106 | Protein | yes |
| Ribosomal protein S3 | 665 | 165 | 99 | 63062 | Protein | yes |
| Ribosomal protein S4 | 296 | 79 | 100 | 41622 | Protein | yes |
| Ribosomal protein S7 | 386 | 72 | 97 | 17440 | Protein | yes |
| Small ubiquitin-related modifier | 78 | 11 | 100 | 8734 | Protein | yes |
| 7S vicilin-like protein | 783 | 183 | 100 | 55890 | Seed | yes |
| Edestin 1 | 276 | 65 | 100 | 58523 | Seed | yes |
| Edestin 2 | 426 | 92 | 100 | 55986 | Seed | no |
| Edestin 3 | 522 | 114 | 99 | 56080 | Seed | no |
| (−)-limonene synthase, | 1013 | 180 | 100 | 72385 | Terpenoid | yes |
| (+)-alpha-pinene synthase, | 706 | 172 | 100 | 71842 | Terpenoid | no |
| 1-deoxy-D-xylulose-5-phosphate | 1918 | 334 | 100 | 78767 | Terpenoid | yes |
| 2-acylphloroglucinol 4- | 526 | 129 | 97 | 45481 | Terpenoid | no |
| 4-(cytidine 5′-diphospho)-2-C- | 412 | 90 | 100 | 45086 | Terpenoid | yes |
| 4-hydroxy-3-methylbut-2-en-1- | 2259 | 277 | 100 | 82920 | Terpenoid | yes |
| Terpene synthase | 6717 | 1432 | 98 | 75307 | Terpenoid | yes |
| DNA-directed RNA polymerase | 404 | 82 | 98 | 39004 | Transcription | no |
| DNA-directed RNA polymerase | 5129 | 1080 | 100 | 12089 | Transcription | yes |
| Maturase K | 1198 | 253 | 100 | 60623 | Transcription | yes |
| Maturase R | 737 | 164 | 100 | 72891 | Transcription | yes |
| RNA polymerase beta subunit | 27 | 8 | 92 | 14495 | Transcription | no |
| RNA polymerase C | 11 | 3 | 25 | 17867 | Transcription | no |
| Acyl-activating enzyme 1 | 773 | 156 | 100 | 79715 | Unknown | yes |
| Acyl-activating enzyme 10 | 783 | 157 | 99 | 61538 | Unknown | yes |
| Acyl-activating enzyme 11 | 330 | 62 | 98 | 36708 | Unknown | no |
| Acyl-activating enzyme 12 | 1070 | 198 | 100 | 83743 | Unknown | yes |
| Acyl-activating enzyme 13 | 877 | 170 | 100 | 78902 | Unknown | yes |
| Acyl-activating enzyme 14 | 154 | 32 | 87 | 80353 | Unknown | no |
| Acyl-activating enzyme 15 | 924 | 200 | 100 | 86725 | Unknown | no |
| Acyl-activating enzyme 2 | 920 | 177 | 100 | 74107 | Unknown | yes |
| Acyl-activating enzyme 3 | 896 | 182 | 99 | 59500 | Unknown | yes |
| Acyl-activating enzyme 4 | 970 | 186 | 100 | 80008 | Unknown | yes |
| Acyl-activating enzyme 5 | 916 | 192 | 100 | 63333 | Unknown | yes |
| Acyl-activating enzyme 6 | 722 | 159 | 100 | 62313 | Unknown | yes |
| Acyl-activating enzyme 7 | 781 | 156 | 100 | 66590 | Unknown | no |
| Acyl-activating enzyme 8 | 647 | 135 | 100 | 56197 | Unknown | yes |
| Acyl-activating enzyme 9 | 723 | 150 | 100 | 61501 | Unknown | no |
| Albumin | 126 | 25 | 86 | 16742 | Unknown | no |
| Cannabidiolic acid synthase-like | 575 | 109 | 98 | 62390 | Unknown | no |
| Cannabidiolic acid synthase-like | 77 | 19 | 76 | 62296 | Unknown | yes |
| Chalcone isomerase-like protein | 729 | 155 | 100 | 23715 | Unknown | no |
| Chalcone synthase-like protein 1 | 579 | 129 | 100 | 43175 | Unknown | no |
| Inactive tetrahydrocannabinolic | 307 | 55 | 83 | 61990 | Unknown | no |
| Prenyltransferase 1 | 513 | 107 | 97 | 44500 | Unknown | no |
| Prenyltransferase 2 | 241 | 58 | 87 | 45105 | Unknown | no |
| Prenyltransferase 3 | 406 | 79 | 99 | 45147 | Unknown | no |
| Prenyltransferase 4 | 332 | 88 | 99 | 44928 | Unknown | no |
| Prenyltransferase 5 | 540 | 108 | 98 | 42610 | Unknown | no |
| Prenyltransferase 6 | 569 | 107 | 95 | 44392 | Unknown | no |
| Prenyltransferase 7 | 498 | 99 | 98 | 44753 | Unknown | no |
| Protein Ycf2 | 3168 | 643 | 99 | 27118 | Unknown | yes |
| Putative calcium dependent | 37 | 12 | 100 | 8116 | Unknown | no |
| Putative LOV domain- | 4899 | 1081 | 99 | 11838 | Unknown | yes |
| Putative LysM domain | 635 | 143 | 100 | 66028 | Unknown | yes |
| Putative permease | 64 | 14 | 100 | 10243 | unknown | no |
| Putative rac-GTP binding | 135 | 24 | 100 | 7145 | unknown | no |
| Transport membrane protein | 326 | 63 | 100 | 32085 | Unknown | no |
| Uncharacterized protein | 46 | 11 | 100 | 4657 | Unknown | no |
| Uncharacterized protein | 1 | 1 | 9 | 20410 | Unknown | no |
| Uncharacterized protein | 727 | 161 | 53 | 18318 | Unknown | yes |

The MW of these cannabis proteins average 38±34 kDa, ranging from 2.8 kDa (Photosystem II phosphoprotein) to 271.2 kDa (Protein Ycf2). The AA sequence coverage varies from 6% (NAD(P)H-quinone oxidoreductase subunit J, chloroplastic) to 100% (108 out of 229 identities, 47%). The vast majority of the proteins (187/229, 82%) display a sequence coverage greater than 80%. These data demonstrate that using proteases asdie from trypsin, either on their own or in combination, further improves the identification of more proteins with greater confidence.

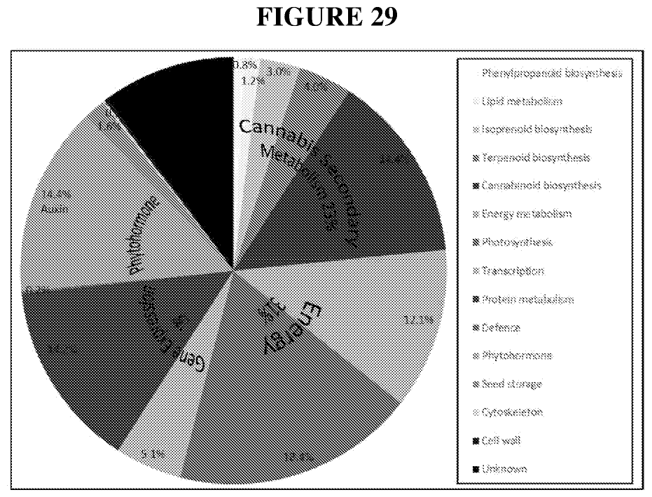

The 494 cannabis protein accessions are predominantly involved in cannabis secondary metabolism (23%), energy production (31%) including 18% of photosynthetic proteins, and gene expression (19%), in particular protein metabolism (14%) (FIG. 28). Ten percent of the proteins are of unknown function, including Cannabidiolic acid synthase-like 1 and 2 which display 84% similarity with CBDA synthase. Most of the additional functions belong to the energy/photosynthesis pathway, translation mechanisms with many ribosomal proteins identified here (Table 18), as well as a plethora (14.4%, 71 out of 494 accessions) of small auxin up regulated (SAUR) proteins. More significantly, all the enzymes involved in the cannabinoid biosynthetic pathway are identified and account for 14.4% of all the accessions (FIG. 29). Additional proteins from this pathway are three truncated products from THCA synthase of 11, 33 and 49 kDa, as well as polyketide synthases 1 to 5 whose AA sequences show 95% similarity to that of OLS. Newly identified proteins include enzymes from the isoprenoid biosynthetic pathway: 2-C-methyl-D-erythritol 4-phosphate cytidylyltransferase, 3-hydroxy-3-methylglutaryl coenzyme A synthase and a naringenin-chalcone synthase involved in the biosynthesis of phenylpropanoids. Finally, novel elements of the terpenoid pathway include (+)-alpha-pinene synthase and 2-acylphloroglucinol 4-prenyltransferase found in the chloroplast (Table 18). Together, these data demonstrate that combining different proteases improves recovery and allows for the thorough analysis of the proteins involved in the secondary metabolism of C. sativa and the diverse biological mechanisms occurring in the mature buds.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

1-31. (canceled)

32. A method of extracting cannabis-derived proteins from cannabis plant material, the method comprising:
(a) suspending cannabis plant material in a solution comprising a charged chaotropic agent for a period of time to allow for extraction of cannabis-derived proteins into the solution; and
(b) separating the solution comprising the cannabis-derived proteins from residual plant material.

33. The method of claim 32, wherein the charged chaotropic agent is selected from the group consisting of guanidine isothiocyanate and guanidine hydrochloride.

34. The method of claim 33, wherein the charged chaotropic agent is guanidine hydrochloride, optionally wherein the solution comprises from about 5.5M to about 6.5M guanidine hydrochloride.

35. The method of claim 32, wherein the solution further comprises a reducing agent; optionally wherein the reducing agent is dithiothreitol.

36. The method of claim 35, wherein the solution comprises:
(i) from about 5 mM to about 20 mM dithiothreitol (DTT); and/or
(ii) from about 5.5M to about 6.5M guanidine hydrochloride.

37. The method of claim 32, wherein the cannabis plant material is pre-treated with an organic solvent before step (a) for a period of time to precipitate the cannabis-derived proteins.

38. The method of claim 37, wherein the organic solvent is selected from the group consisting of trichloroacetic acid (TCA)/acetone and TCA/ethanol, optionally wherein the organic solvent comprises from about 5% to about 20% TCA/acetone or from about 5% to about 20% TCA/ethanol.

39. The method of claim 32, wherein the cannabis-derived proteins separated in step (b) are digested by a protease in preparation for proteomic analysis.

40. The method of claim 39, wherein the cannabis-derived proteins separated by step (b) are digested by two or more proteases; optionally wherein:
(i) the cannabis-derived proteins separated by step (b) are digested by the two or more proteases sequentially; or
(ii) the cannabis-derived proteins separated by step (b) are digested by the two or more proteases simultaneously.

41. The method of claim 40, wherein the protease is selected from the group consisting of trypsin, trypsin/LysC, chymotrypsin, GluC and pepsin; optionally wherein the protease is selected from the group consisting of trypsin/LysC, GluC and chymotrypsin.

42. The method of claim 32, wherein the cannabis-derived proteins separated by step (b) are alkylated in preparation for proteomic analysis; optionally wherein the cannabis-derived proteins are alkylated with iodoacetamide (IAA).

43. The method of claim 39, wherein the proteomic analysis is selected from the group consisting of liquid chromatography-mass spectroscopy (LC-MS), ultra-performance LC-MS (UPLC-MS), and nano liquid chromatography-tandem mass spectrometry (nLC-MS/MS).

44. The method of claim 32, wherein the cannabis plant material is selected from the group consisting of leaves, stems, roots, apical buds, and trichomes, or parts thereof; optionally wherein the plant material comprises apical buds and/or trichomes.

45. A method of extracting cannabis-derived proteins from cannabis plant material, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent for a period of time to allow for extraction of cannabis-derived proteins into the solution; and
(c) separating the solution comprising the cannabis-derived proteins from residual plant material.

46. The method of claim 45, further comprising:
(d) digesting the solution of (c) with a protease.

47. The method of claim 46, further comprising:
(e) subjecting the digested solution of step (d) to proteomic analysis.

48. The method of claim 47, wherein the proteomic analysis comprises a parameter setting the maximum number of missed cleavages to between about 2 and about 10.

49. The method of claim 48, wherein the proteomic analysis comprises a parameter setting the maximum number of missed cleavages of between about 6 and about 10.

50. A method of preparing a sample of cannabis-derived proteins from cannabis plant material for proteomic analysis, the method comprising:
(a) pre-treating the cannabis plant material with an organic solvent to precipitate the cannabis-derived proteins;
(b) suspending the precipitated cannabis-derived proteins of (a) in a solution comprising a charged chaotropic agent for a period of time to allow for extraction of cannabis-derived proteins into the solution;
(c) separating the solution comprising the cannabis-derived proteins from residual plant material; and
(d) optionally subjecting the sample to proteomic analysis.

51. The method of claim 50, further comprising alkylating the cannabis-derived proteins separated in (c).
